SEQUENCE LISTING (189 sequences)

SEQ ID NO: 1 - protein, 116 aa, Artificial Sequence
  Synthetic construct
QVQLVESGGGLVQAGGSLRLSCAASGRTFSIYNMGWFRQAPGKEREFVIHMNWSNGNTATAASVKDRFTISRDNAKNTVYLQMSSLKPEDTAVYYCHATTNDYDYWGQGTQVTVSS

SEQ ID NO: 2 - protein, 122 aa, Artificial Sequence
  Synthetic construct
HVQLVESGGGLVQAGGSLRLSCVASGSGFEPNAMGWYRQAPGKQRELVAGITSGGNTNYADSVKGRFTISRDNVKNTVYLQMNSLKPEDTAVYYCNAVAPRINPRGSPTNYWGQGTQVTVSS

SEQ ID NO: 3 - protein, 122 aa, Artificial Sequence
  Synthetic construct
QVQLVESGGGLVQAGGSLRLSCVASGSGFEPNAMGWYRQAPGKQRELVAGITSGGNANYADSVKGRFTISRDNVKNTVYLQMNSLKPEDTAVYYCNAVGPRFNPRGSPTNYWGQGTQVTVSS

SEQ ID NO: 4 - protein, 125 aa, Artificial Sequence
  Synthetic construct
QVQLVESGGGLAQSGDSLRLSCAASGRTFDINYMAWFRRAPGKEREFVAASSWTGVGTYYADSVKGRFTISRDNNKNTLYLQMDSLKPEDTAVYYCAADSNIDRLHSAGSDYRYWGQGTQVTVSS

SEQ ID NO: 5 - protein, 129 aa, Artificial Sequence
  Synthetic construct
HVQLVESGGGLVQAGGSLRLSCAASGRTFSTHATGWFRQAPGKEREFLATSVWLDDISRDSTYYAESVKGRFSISRDNAKDTVYLQMNSLKPEDTAVYYCAVKQRVGPGIMSAPTYDYWGQGTQVTVSS

SEQ ID NO: 6 - protein, 127 aa, Artificial Sequence
  Synthetic construct
HVQLVESGGGLVQTGGSLRLSCAFSGYTFSTFPTAWFRQAPGKEREFVAGIRWNGGSRDYTEYADFVKGRFTISRDNAKNMIYLQMISLKPEDTALYYCAASHGVVDGTSVNGYRYWGQGTQVTVSS

SEQ ID NO: 7 - protein, 120 aa, Artificial Sequence
  Synthetic construct
DVQLVESGGGLVQAGGSLKLSCTASESTFNFNHIDWYRQAPGKQRESVAVITNTDVTNYADSVKGRFTISRDNAKNTVYLQMNSLKPEDTAVYLCNAWRFGPTGVKVDYWGQGTQVTVSS

SEQ ID NO: 8 - protein, 117 aa, Artificial Sequence
  Synthetic construct
HVQLVESGGGLVQAGGSLRLSCAVSGASAERVGWFRRAPGKEREFVAAIGANGGSTNYLDSVKGRFTISRDNAKNTVYLQMNSLKPQDTAVYYCAARNLETFDYTYWGRGTQVTVSS

SEQ ID NO: 9 - protein, 120 aa, Artificial Sequence
  Synthetic construct
HVQLVESGGGLVQAGGSLRLTCAASGGTFSTSTMDMGWFRQAPGKEREFVAVDSWNDGRAYYADSVKGRFTISRDSAKTTLYLQMNSLKPEDTAVYYCAARTERGVYDYWGRGTQVTVSS

SEQ ID NO: 10 - protein, 120 aa, Artificial Sequence
  Synthetic construct
EVQLVESGGGLVAAGGSLRLSCVPSTNIFSANAMGWYRQAPGKQRELVASITSGGNTNYADSVKGRFTISRDNADTVILQMNSLKPEDTAVYYCKGTAYSTTYNSVRDYWGQGTQVTVSS

SEQ ID NO: 11 - protein, 121 aa, Artificial Sequence
  Synthetic construct
DVQLVESGGGLVQAGESLRLSCAASGDMFSSNAMAWYRQAPGKQRELVASITSGGNTNYADSVKGRFTISRDNAKNTVYLQMDSLKPEDTAVYYCNGVAYDYSWGRPRNFWGQGTQVTVSS

SEQ ID NO: 12 - protein, 119 aa, Artificial Sequence
  Synthetic construct
HVQLVESGGGLVQAGGSLRLSCAASGSIFSSNAMSWYRQAPGKQRELVASITSGGNADYADSVKGRFTISRDKNTVYPEMSSLKPADTAVYYCHAVGQEASAYAPRAYWGQGTQVTVSS

SEQ ID NO: 13 - protein, 127 aa, Artificial Sequence
  Synthetic construct
DVQLVESGGGLVQAGGSLRLSCAASGRTFSSYNVGWFRQAPGKEREFVAFIRWSGTSTYYADSVKGRFTISRDNAKNTVYLQMNSLKPEDTAVYYCAATSRTMIHTSQAMPINWDYWGQGTQVTVSS

SEQ ID NO: 14 - protein, 117 aa, Artificial Sequence [sequence omitted]

SEQ ID NO: 15 - protein, 117 aa, Artificial Sequence
  Synthetic construct
QVQLVESGGGLVQAGGSLRLSCAVSGLGAHRVGWFRRAPGKEREFVAAIGANGGNTNYLDSVKGRFTISRDNAKNTIYLQMNSLKPQDTAVYYCAARDIETAEYTYWGQGTQVTVSS

SEQ ID NO: 16 - protein, 125 aa, Artificial Sequence
  Synthetic construct
QVQLVESGGGLVQAGGSLRLTCAASGGTFSTSTMDMGWFRQAPGKEREFVALDSWNGGRTYYADSVKGRFTISRDNAKNTLYLQMNSLKPEDTAVYYCAADTYSNYEKDDSWGYWGQGTQVTVSS

SEQ ID NO: 17 - protein, 125 aa, Artificial Sequence
  Synthetic construct
HVQLVESGGGLVQAGGSLRLTCAASGRTFSISTMDMGWFRQAPGKEREFVAADAWNDGRTYYADSVKGRFTISRDNAKNTLYLQMNSLKPEDTAVYYCTADTYSNYKKDDSWGYWGQGTQVTVSS

SEQ ID NO: 18 - protein, 125 aa, Artificial Sequence
  Synthetic construct
HVQLVESGGGLVQAGGSLRLSCAASGRTFSTSTMDMGWFRQAPGKEREFVGADSWNDGRTYYADSVKGRFTISRDNAKNTLYLQMNNLKPEDTAVYYCAAETYSLYEKSDSWGYWGQGTQVTVSS

SEQ ID NO: 19 - protein, 125 aa, Artificial Sequence
  Synthetic construct
HVQLVESGGGLVQAGGSLRLTCAASGGTFSTSTMDMGWFRQAPGKEREFVAVDSWNDGRAYYADSVKGRFTISRDNAKNTLYLQMNSLKPEDTAVYYCAAETYSNYEKAYSWGYWGQGTQVTVSS

SEQ ID NO: 20 - protein, 125 aa, Artificial Sequence
  Synthetic construct
DVQLVESGGGLVQAGGSLRLSCAASGRTFSSDTMDMGWFRQAPGKEREFVALHSWNGGKTYYADSVKGRFTISRDNAKNTLYLQMNNLKPEDTAVYYCAAETYTLYEKDSSWGYWGQGTQVTVSS

SEQ ID NO: 21 - protein, 119 aa, Artificial Sequence
  Synthetic construct
HVQLVESGGGLVQAGGSLRLSCAASGRTFSSYAMGWFRQAPGKQRELVAGITSGGNANYEDSVKGRFTISRDKNTVYLEMSSLKPADTAVYYCNAVGQEGSARAPRAYWGQGTQVTVSS

SEQ ID NO: 22 - protein, 126 aa, Artificial Sequence
  Synthetic construct
DVQLVESGGGLVQAGGSLRLSCAASGFTFSNYVMYWGRQAPGKGREWVSGIDSDGSDTAYASSVKGRFTISRDNAKNTLYLQMNNLKPEDTALYYCVKSKDPYGSPWTRSEFDDYWGQGTQVTVSS

SEQ ID NO: 23 - protein, 125 aa, Artificial Sequence
  Synthetic construct
HVQLVESGGGLVQAGGSLTLTCAASGRTFSSETMDMGWFRQAPGKEREFVAADSWNDGSTYYADSVKGRFTISRDSAKNTLYLQMNSLKPEDTAVYYCAAETYSIYEKDDSWGYWGQGTQVTVSS

SEQ ID NO: 24 - protein, 125 aa, Artificial Sequence
  Synthetic construct
QVQLVESGGGLVQTGGSLRLSCAASGRTFSNYVMGWFRQAPGKEREFVSSIEWNSENTFYANSVKGRFTISRDNAKNTVYLQMNSLKAEDTAVYYCAADRGSSYYYTRASEYTYWGQGTQVTVSS

SEQ ID NO: 25 - protein, 119 aa, Artificial Sequence
  Synthetic construct
QVQLVESGGGLVQAGGSLRLSCAASGRTFRFSAIDWYRQAPGQQREFVASISTSGRISYGHFVKGRFTISRDNAKNTMYLQMNSLKPEDTAVYYCHLAQPDVFGGGDYWGQGTQVTVSS

SEQ ID NO: 26 - protein, 126 aa, Artificial Sequence
  Synthetic construct
DVQLVESGGGLVQAGGSLRLSCAASGFTFSNYVMYWGRQAPGKGREWVSGIDSDGSDTAYASSVKGRFTISRDNAKKTLYLQMNDLKPEDTALYYCVKSKDRYGSPWTRSEFEDYWGQGTQVTVSS

SEQ ID NO: 27 - protein, 120 aa, Artificial Sequence
  Synthetic construct
HVQLVESGGGLVQAGESLRLSCVASGSIFSINAMGWFRQAPGKQRELVASISNIGTTIYAESVKGRFTISRDNAKNTADLQMNSLKPEDTAIYYCNLRQGCSYNGCDEYWGQGTQVTVSS

SEQ ID NO: 28 - protein, 123 aa, Artificial Sequence
  Synthetic construct
EVQLVESGGGLVQAGGSLRLTCAASGRTFSSSTMDMGWFRQAPGKEREFVAVDAWNDGRTYYADSVKGRFTISRDNAKNTLYLQMNSLKPEDTAIYYCNLRQGCSYNGCDEYWGQGTQVTVSS

SEQ ID NO: 29 - protein, 119 aa, Artificial Sequence
  Synthetic construct
DVQLVESGGGLVQAGGSLRLTCAASGRTFSSSTMDMGWFRQAPGKEREFVAASSWGFTNYADPVKGRFTISRDNAKNTVYLEMSSLKPEDTAVYYCAARTPETGQYDYWGQGTQVTVSS

SEQ ID NO: 30 - protein, 116 aa, Artificial Sequence
  Synthetic construct
QVQLVESGGGLVQAGGSLRLSCAVSGSTLGINAIGWYRQPPGKQRELVASISSLGTTLYAESVKGRFAISRDNAKNTGYLQMNSLKPDDTAVYYCNMRTPYMRDYWGQGTQVTVSS

SEQ ID NO: 31 - protein, 127 aa, Artificial Sequence
  Synthetic construct
HVQLVESGGGLVQAGGSLRLSCAASGSIFRHYNMGWFRQAPGKEREFVSAISWTNIGTYYADSVKGRFSISRDNNRNTAYLEMNSLKPEDTAVYYCAADRSEFGSRLYSDENEYDYWGQGTQVTVSS

SEQ ID NO: 32 - protein, 121 aa, Artificial Sequence
  Synthetic construct
QVQLVESGGGLVQAGGSLRLSCAASGSTFSINAIGWYRQAPGKQRELVAGITSGGNTNYADSVKGRFTISRDNAKNTVDLQMNSLKPEDTAVYYCNTVAPVEGTGRIRAYWGQGTQVTVSS

SEQ ID NO: 33 - protein, 123 aa, Artificial Sequence
  Synthetic construct
DVQLVESGGGLVQAGGSLRLSCAASGRTFGSTMAWFRQAPGKERDFVAAITWSADRISYADSVKGRFTISRDNDKNIVHLQMNSLKPEDTAVYVCAARASSNTYDDPRSFAFWGQGTQVTVSS

SEQ ID NO: 34 - protein, 121 aa, Artificial Sequence
  Synthetic construct
QVQLVESGGGLVQAGGSLRLSCAASGRDFSSTVMGWFRQAPGKERELVAWMTVTYDITYYADSVKGRFTISKDNAKNTVYLQANSLKPDDTAVYYCASGALRVGSFSPDYWGQGTQVTVSS

SEQ ID NO: 35 - protein, 125 aa, Artificial Sequence
  Synthetic construct
HVQLVESGGGLVQAGGSLRLSCAASGRTFSSYAMSWVRQAPGKGQEWVAGIQREGTETNYADSVKGRFTISRDNAKRTLYLQMNNLKPEDTGVYYCAKVLGRPYGSRWLDDVDSWGRGTQVTVSS

SEQ ID NO: 36 - protein, 126 aa, Artificial Sequence
  Synthetic construct
HVQLVESGGGLVQAGDSLRLSCAASERTNINYAMGWFRQAPGKERDFVAAIGWNGSIIKYAESVKGRFTISRDDAKNTVSLQMNSLKPEDTAVYYCAARIGSIANLQPREYEFAYWGQGTQVTVSS

SEQ ID NO: 37 - protein, 120 aa, Artificial Sequence
  Synthetic construct
HVQLVESGGGLVQAGGSLRLTCAASGRTFSSSTMDMGWFRQAPGKEREFVAVDAWNDGRTYYADSVKGRFTISRDNAKNTLYLQMNSLKPGDTAVYYCHAVDVSVGGDYWGQGTQVTVSS

SEQ ID NO: 38 - protein, 119 aa, Artificial Sequence
  Synthetic construct
QVQLVESGGGLVQAGGSLRLSCVASGSGFEPNAMGWYRQAPGKQRELVAGITSGGNANYADSVKGRFTISRDNVKNTVYLQMNSLKPEDTAVYYCQGPACSGSGCRNYWGQGTQVTVSS

SEQ ID NO: 39 - protein, 114 aa, Artificial Sequence
  Synthetic construct
QVQLVESGGGLVQAGGSLRLSCSASGFDFSHNTWAWYRQAPGKQRELVATISNSGSTNYADSVKGRFTIARDNAKNTVSLQMNTLKPEDTAVYFCKKESGVNYWGQGTQVTVSS

SEQ ID NO: 40 - protein, 125 aa, Artificial Sequence
  Synthetic construct
QVQLVESGGGLVQAGGSLRLSCAASGGTLRSYNMGWFRQGPGKEREFVAAISWTGLGTYYGDSVKGRFTISRDVIKNTLYLQMDSLKPEDTAIYYCAADSSTSRLHSAGSDYRYWGQGTQVTVSS

SEQ ID NO: 41 - protein, 122 aa, Artificial Sequence
  Synthetic construct
QVQLVESGGGLVQAGGSLRLSCAASGRTFSSHAMAWFRQAPGKEREFVAGISWSGPYYVDSVKGRFTISADNAKNIVYLQMNSLTPEDTAVYYCASNRGPNLYPHIGDIEYWGQGTQVTVSS

SEQ ID NO: 42 - protein, 122 aa, Artificial Sequence
  Synthetic construct
HVQLVESGGGLVQAGGSLRLSCAASGRTFSSHAMAWFRQAPGKEREFVAGISWSGPYYVDSVKGRFTISTDNAKNTVYLQMNSLTPEDTAVYYCASSRGPNLYPHIGDVEYWGQGTQVTVSS

SEQ ID NO: 43 - protein, 122 aa, Artificial Sequence
  Synthetic construct
DVQLVESGGGLVQAGGSLRLSCAVSGRTFSISTMGWFRQAPGKERLFVAGITWHDGSTNYADSVKGRFTISRDNAKNTVYLQMNSLKPEDTALYYCAARKSINYVQEYDYWGRGTQVTVSSE

SEQ ID NO: 44 - protein, 117 aa, Artificial Sequence
  Synthetic construct
QVQLVESGGGLVQAGGSLRLSCAISGASANRVGWFRRAPGKEREFVAAIGASGGMTNYLDSVKGRFTISRDNAKNTVYLQMNSLKPQDTAVYYCAARNIETAEYTYWGQGTQVTVSS

SEQ ID NO: 45 - protein, 119 aa, Artificial Sequence
  Synthetic construct
HVQLVESGGGLVQPGGSLRLSCAASGNFFLINVMSWWRQAPGNEHELHELVATITSDGRTNYGDAVQGRFTISRDNAKNTLYLQMNSLKPEDTAVYYCYAQGQGNREYWGQGTQVTVSS

SEQ ID NO: 46 - protein, 127 aa, Artificial Sequence
  Synthetic construct
DVQLVESGGGLVQAGGSLRLSCAASGRTFSSYSMGWFRQAPGKEREFVAFIRWSGTSTYYADSVKGRFTISRDNAKNTVYLQMNSLKPDDTAVYYCAASSRTMNTISQTMRINWDYWGQGTQVTVSS

SEQ ID NO: 47 - protein, 121 aa, Artificial Sequence
  Synthetic construct
HVQLVESGGGLVQAGGSLRLSCAASGSDFSILAIGWYRQAPGKQRELVAAISRVGSTNYADSVKGRFTISRDNAKNTVSLQMNSLKPEDTAVYYCNAGIPMSTVLLGQGFWGQGTQVTVSS

SEQ ID NO: 48 - protein, 117 aa, Artificial Sequence [sequence omitted]

SEQ ID NO: 49 - protein, 122 aa, Artificial Sequence
  Synthetic construct
HVQLVESGGGLVQAGGSLRLSCAVSGRTVSSYVMGWFRQAPGKEREFVAGIGRTGMTITADSVKGRFTISRDNAKNLVYLQMNSLKSEDTAVYWCAAGVRDFAPRIDSYDYWGQGTQVTVSS

SEQ ID NO: 50 - protein, 127 aa, Artificial Sequence
  Synthetic construct
HVQLVESGGGLVQPGGSLRLSCAASGLTLNDYAIGWFRQAPGKEREGVSCISSSDGTTYYADSVKGRFTISRDNAKNTVYLQMNSLKPEDTAVYYCATDIAPFYCSWPLRTRLVEYWGQGTQVTVSS

SEQ ID NO: 51 - protein, 126 aa, Artificial Sequence
  Synthetic construct
HVQLVESGGGLVQAGGSLRLSCAASGRTFSAYGMGWFRQAPGKEREFVAGIARSGSEYYGDSVKGRFTISRDNAKNTVYLQMASLKAEDTGVYYCAADPTILYHALSQRTQNYRYWGPGTQVTVSS

SEQ ID NO: 52 - protein, 127 aa, Artificial Sequence
  Synthetic construct
EVQLVESGGGLVQPGGSLRLSCAASGFTFSSYAMSWVRQAPGKGREWVAGITPGSGTFYADSVKGRFTISRDNAKNTLSLEINSLKPEDTALYYCAKCRQEFSWDFSSRDPDDFDYWGQGTQVTVSS

SEQ ID NO: 53 - protein, 125 aa, Artificial Sequence
  Synthetic construct
QVQLVESGGGLVQAGGSLRLSCTASGGTFRSFTMAWFRQAPGKEREFVATLSWTGVGTYYADSVKGRFTISRDIAKNTVYLEMNSLIPEDTAVYYCAASPYTYSLLSRTGDYGYWGQGTQVTVSS

SEQ ID NO: 54 - protein, 122 aa, Artificial Sequence
  Synthetic construct
HVQLVESGGGLVQAGDSLRLSCAASGRDFSSYAMGWFRQAPGKEREFVGVISSGDVGTFYEDSVKGRFTISRDDAKNTVDLQMNSLKPEDTAVYYCAAALYVTSTRGSYAYWGQGTQVTVSS

SEQ ID NO: 55 - protein, 127 aa, Artificial Sequence
  Synthetic construct
EVQLVESGGGLVQAGGSLRLSCAASGLTFSSYNMGWFRQAPGKEREFVAFIRWSGTSTYYADSVKGRFTISRDTAKNTVYLQMNSLKLEDTAVYYCAATSRTMIHTSQTMRINWDYWGQGTQVTVSS

SEQ ID NO: 56 - protein, 124 aa, Artificial Sequence
  Synthetic construct
QVQLVESGGGLVQAGGSLRLSCAASGRTFSNSHMGWFRQAPGKEREFVAVISRSGGSTLYADSVKGRFTISRDNAKNTVYLQMNSLKPEDTAVYYCAASRTAGLTSNRSLYDYWGRGTQVTVSS

SEQ ID NO: 57 - protein, 123 aa, Artificial Sequence
  Synthetic construct
HVQLVESGGGLVQAGGSLRLSCAASGRTFSSYAMGWFRQAPGKEREFVARVSAAGSITDYADSVKGRFTIAKDNAKSTVYLRMNSLRLEDTAVYYCAADVATIGSRLANYDYWGQGTQVTVSS

SEQ ID NO: 58 - protein, 116 aa, Artificial Sequence
  Synthetic construct
QVQLVESGGGLVQAGGSLRLSCAVSGSTLGINAIGWYRQPPGKQRELVASISSLGTTLYAESVKGRFAISRDNAKNTAYLQMNSLKPDDTAVYYCNMRTPYRRDYWGQGTQVTVSS

SEQ ID NO: 59 - protein, 126 aa, Artificial Sequence
  Synthetic construct
HVQLVESGGGLVQAGGSLRLSCAVSGRTFSTYGMAWFRQAPGKERDFVATITRSGETTLYADSVKGRFTISRDNAKNTVYLQMNSLKIEDTAVYYCAVRRDSSWGYSRDLFEYDYWGQGTQVTVSS

SEQ ID NO: 60 - protein, 122 aa, Artificial Sequence
  Synthetic construct
QVQLVESGGGLVQAGGSLRLSCAASGRTFSNYAMGWFRQAPGKGREFVAGISWSTRNTYYADSVKGRFTVSRDNEQTTMSLQMNRLKPEDTAVYYCAARFGIVTTRDEYEFYGQGTQVTVSS

SEQ ID NO: 61 - protein, 119 aa, Artificial Sequence
  Synthetic construct
HVQLVESGGGLVQAGGSLRLSCAASGRAIGIHRMAWFRQAPGKERAFVAAIQWTGDMPTYADSLKGRFTISADNAKNTVSLQMTSLKPDDTAVYYCAARSATTALYDYWGQGTQVTVSS

SEQ ID NO: 62 - protein, 118 aa, Artificial Sequence
  Synthetic construct
EVQLVESGGGLVQAGGSLRLSCAASGSIFSILNMGWYRQAPGKQRDLVARITPGGSTDYADSVKGRFTISRDNAKNTLYLQMNSLKPEDTAVYLCYAKIFGPGSGSYWGQGTQVTVSS

SEQ ID NO: 63 - protein, 120 aa, Artificial Sequence
  Synthetic construct
DVQLVESGGGLVQAGGSLGLSCAASGSIFRLTAMGWYRQAPGKQRDLVTTITSAGIPHYADSVKGRFTISRDNAKNTVYLQMNGLKPEDTAVYYCYANTLNSQLLPRAYWGQGTQVTVSS

SEQ ID NO: 64 - protein, 122 aa, Artificial Sequence
  Synthetic construct
HVQLVESGGGLVQPGGSLRLSCAASGRSFNSYLMGWFRQAPGKEREFVAWISGSPHDIIRYRDSVKDRFTISRDNAKNTVYLQMNSLKPVDTAVYYCAVGSLRVGSFSPDYWGQGTQVTVSS

SEQ ID NO: 65 - protein, 122 aa, Artificial Sequence
  Synthetic construct
QVQLVESGGGLVQAGGSLRLSCAVSGRTFSIAGMGWFRQAPGKDREFLGGITWNDGTTWYADSVKGRFTISRDNAKNMLSLRMNSLKPEDTAVYYCAAGPRLGSTPRAYDYWGQGTQVTVSS

SEQ ID NO: 66 - protein, 126 aa, Artificial Sequence
  Synthetic construct
QVQLVESGGGLVQAGGSLRLSCAASGRTFSSYSMGWFRQAQGKEREFVATINGNGRDTYYTNSVKGRFTISRDDATNTVYLQMNSLKPEDTAIYYCAADKDVYYGYTSFPNEYEYWGQGTQVTVSS

SEQ ID NO: 67 - protein, 123 aa, Artificial Sequence
  Synthetic construct
HVQLVESGGGLVQAGGSLRLSCAASGRTFSVNSIAWFRQAPGKEREFVATINWSGGDIYYADSVKGRFTISRDNGKNTVYLQMNSLKPTDTAVYYCAAARFYGRSIAQDYDSWGQGTQVTVSS

SEQ ID NO: 68 - protein, 122 aa, Artificial Sequence
  Synthetic construct
HVQLVESGGGLVQAGGSLRLSCAAPGRTFSTSAMGWFRQAPGKEREFVAAIDWSNTNIHYADTVKGRFTISTDTAKNTVYLQMNNLKPEDTAVYYCAQGGWGLTQPISVDYWGKGTQVTVSS

SEQ ID NO: 69 - protein, 122 aa, Artificial Sequence
  Synthetic construct
QVQLVESGGGLVQAGGSLRLSCAASGGTFSSYAMGWFRQTPGKEREFVGAISRSGGGTSYLDSVKGRFTISRDNAKNTMYLQMNSLKPEDSAIYYCAGGTYGSTSVRDFGPWGQGTQVTVSS

SEQ ID NO: 70 - protein, 115 aa, Artificial Sequence
  Synthetic construct
QVQLVESGGGLVQTGGSLRLSCAASGSTRSIWYSGWYRQAPGKQRELVASLTSGGTTYYPDSMKGRFTISGSSGLNMVYLQMNSLRPEDTAVYSCNVRTGSGVYWGQGTQVTVSS

SEQ ID NO: 71 - protein, 123 aa, Artificial Sequence
  Synthetic construct
HVQLVESGGGLVQAGGSLRLSCAASERTFTSYVVGWYRQAPGKGREFVARINEIGDITYYADSVKGRFTISRDNAKNTVYLQMSALKPEDTAVYYCGARKDSVVLSSVGYDYWGQGTQVTVSS

SEQ ID NO: 72 - protein, 403 aa, Artificial Sequence
  Synthetic construct
QVQLVESGGGLVQAGGSLRLSCAVSGAGAHRVGWFRRAPGKEREFVAAIGASGGMTNYLDSVKGRFTISRDNAKNTIYLQMNSLKPQDTAVYYCAARDIETAEYIYWGQGTQVTVSSKLGGGGSGGGGSGGGGSGGGGSGGGGSQVQLVESGGGLVQAGGSLRLSCAVSGAGAHRVGWFRRAPGKEREFVAAIGASGGMTNYLDSVKGRFTISRDNAKNTIYLQMNSLKPQDTAVYYCAARDIETAEYIYWGQGTQVTVSSGGGGSGGGGSGGGGSGGGGSGGGGSQVQLVESGGGLVQAGGSLRLSCAVSGAGAHRVGWFRRAPGKEREFVAAIGASGGMTNYLDSVKGRFTISRDNAKNTIYLQMNSLKPQDTAVYYCAARDIETAEYIYWGQGTQVTVSS

SEQ ID NO: 73 - protein, 424 aa, Artificial Sequence
  Synthetic construct
QVQLVESGGGLVQAGGSLRLSCAVSGAGAHRVGWFRRAPGKEREFVAAIGASGGMTNYLDSVKGRFTISRDNAKNTIYLQMNSLKPQDTAVYYCAARDIETAEYIYWGQGTQVTVSSGSSEGKSSGSGESKSTGGGGSEGKSSGSGESKSTSNSQVQLVESGGGLVQAGGSLRLSCAVSGAGAHRVGWFRRAPGKEREFVAAIGASGGMTNYLDSVKGRFTISRDNAKNTIYLQMNSLKPQDTAVYYCAARDIETAEYIYWGQGTQVTVSSKLEGKSSGSGESKSTGGGGSEGKSSGSGESKSTAAAQVQLVESGGGLVQAGGSLRLSCAVSGAGAHRVGWFRRAPGKEREFVAAIGASGGMTNYLDSVKGRFTISRDNAKNTIYLQMNSLKPQDTAVYYCAARDIETAEYIYWGQGTQVTVSS

SEQ ID NO: 74 - protein, 404 aa, Artificial Sequence
  Synthetic construct
QVQLVESGGGLVQAGGSLRLSCAVSGLGAHRVGWFRRAPGKEREFVAAIGANGGNTNYLDSVKGRFTISRDNAKNTIYLQMNSLKPQDTAVYYCAARDIETAEYTYWGQGTQVTVSSKLGGGGSGGGGSGGGGSGGGGSGGGGSSQVQLVESGGGLVQAGGSLRLSCAVSGLGAHRVGWFRRAPGKEREFVAAIGANGGNTNYLDSVKGRFTISRDNAKNTIYLQMNSLKPQDTAVYYCAARDIETAEYTYWGQGTQVTVSSGGGGSGGGGSGGGGSGGGGSGGGGSQVQLVESGGGLVQAGGSLRLSCAVSGLGAHRVGWFRRAPGKEREFVAAIGANGGNTNYLDSVKGRFTISRDNAKNTIYLQMNSLKPQDTAVYYCAARDIETAEYTYWGQGTQVTVSS

SEQ ID NO: 75 - protein, 424 aa, Artificial Sequence
  Synthetic construct
QVQLVESGGGLVQAGGSLRLSCAVSGLGAHRVGWFRRAPGKEREFVAAIGANGGNTNYLDSVKGRFTISRDNAKNTIYLQMNSLKPQDTAVYYCAARDIETAEYTYWGQGTQVTVSSGSSEGKSSGSGESKSTGGGGSEGKSSGSGESKSTSNSQVQLVESGGGLVQAGGSLRLSCAVSGLGAHRVGWFRRAPGKEREFVAAIGANGGNTNYLDSVKGRFTISRDNAKNTIYLQMNSLKPQDTAVYYCAARDIETAEYTYWGQGTQVTVSSKLEGKSSGSGESKSTGGGGSEGKSSGSGESKSTAAAQVQLVESGGGLVQAGGSLRLSCAVSGLGAHRVGWFRRAPGKEREFVAAIGANGGNTNYLDSVKGRFTISRDNAKNTIYLQMNSLKPQDTAVYYCAARDIETAEYTYWGQGTQVTVSS

SEQ ID NO: 76 - protein, 270 aa, Artificial Sequence
  Synthetic construct
QVQLVESGGGLVQAGGSLRLSCAVSGAGAHRVGWFRRAPGKEREFVAAIGASGGMTNYLDSVKGRFTISRDNAKNTIYLQMNSLKPQDTAVYYCAARDIETAEYIYWGQGTQVTVSSKLEGKSSGSGESKSTGGGGSEGKSSGSGESKSTAAAQVQLVESGGGLVQAGGSLRLSCAVSGAGAHRVGWFRRAPGKEREFVAAIGASGGMTNYLDSVKGRFTISRDNAKNTIYLQMNSLKPQDTAVYYCAARDIETAEYIYWGQGTQVTVSS

SEQ ID NO: 77 - protein, 270 aa, Artificial Sequence
  Synthetic construct
QVQLVESGGGLVQAGGSLRLSCAVSGLGAHRVGWFRRAPGKEREFVAAIGANGGNTNYLDSVKGRFTISRDNAKNTIYLQMNSLKPQDTAVYYCAARDIETAEYTYWGQGTQVTVSSKLEGKSSGSGESKSTGGGGSEGKSSGSGESKSTAAAQVQLVESGGGLVQAGGSLRLSCAVSGLGAHRVGWFRRAPGKEREFVAAIGANGGNTNYLDSVKGRFTISRDNAKNTIYLQMNSLKPQDTAVYYCAARDIETAEYTYWGQGTQVTVSS

SEQ ID NO: 78 - protein, 255 aa, Artificial Sequence
  Synthetic construct
QVQLVESGGGLVQAGGSLRLSCAVSGLGAHRVGWFRRAPGKEREFVAAIGANGGNTNYLDSVKGRFTISRDNAKNTIYLQMNSLKPQDTAVYYCAARDIETAEYTYWGQGTQVTVSSKLGGGGSGGGGSDVQLVESGGGLVQAGGSLRLSCAASGFTFSNYVMYWGRQAPGKGREWVSGIDSDGSDTAYASSVKGRFTISRDNAKNTLYLQMNNLKPEDTALYYCVKSKDPYGSPWTRSEFDDYWGQGTQVTVSS

SEQ ID NO: 79 - protein, 255 aa, Artificial Sequence
  Synthetic construct
QVQLVESGGGLVQAGGSLRLSCAVSGLGAHRVGWFRRAPGKEREFVAAIGANGGNTNYLDSVKGRFTISRDNAKNTIYLQMNSLKPQDTAVYYCAARDIETAEYTYWGQGTQVTVSSKLGGGGSGGGGSHVQLVESGGGLVQAGGSLRLSCAVSGRTFSTYGMAWFRQAPGKERDFVATITRSGETTLYADSVKGRFTISRDNAKNTVYLQMNSLKIEDTAVYYCAVRRDSSWGYSRDLFEYDYWGQGTQVTVSS

SEQ ID NO: 80 - protein, 255 aa, Artificial Sequence
  Synthetic construct
QVQLVESGGGLVQAGGSLRLSCAVSGLGAHRVGWFRRAPGKEREFVAAIGANGGNTNYLDSVKGRFTISRDNAKNTIYLQMNSLKPQDTAVYYCAARDIETAEYTYWGQGTQVTVSSKLGGGGSGGGGSQVQLVESGGGLVQAGGSLRLSCAASGRTFSSYSMGWFRQAQGKEREFVATINGNGRDTYYTNSVKGRFTISRDDATNTVYLQMNSLKPEDTAIYYCAADKDVYYGYTSFPNEYEYWGQGTQVTVSS

SEQ ID NO: 81 - protein, 254 aa, Artificial Sequence
  Synthetic construct
QVQLVESGGGLVQAGGSLRLSCAVSGLGAHRVGWFRRAPGKEREFVAAIGANGGNTNYLDSVKGRFTISRDNAKNTIYLQMNSLKPQDTAVYYCAARDIETAEYTYWGQGTQVTVSSKLGGGGSGGGGSHVQLVESGGGLVQAGGSLTLTCAASGRTFSSETMDMGWFRQAPGKEREFVAADSWNDGSTYYADSVKGRFTISRDSAKNTLYLQMNSLKPEDTAVYYCAAETYSIYEKDDSWGYWGQGTQVTVSS

SEQ ID NO: 82 - protein, 7 aa, Artificial Sequence
  Synthetic construct
TTNDYDY

SEQ ID NO: 83 - protein, 14 aa, Artificial Sequence
  Synthetic construct
VAPRINPRGSPTNY

SEQ ID NO: 84 - protein, 14 aa, Artificial Sequence
  Synthetic construct
VGPRFNPRGSPTNY

SEQ ID NO: 85 - protein, 16 aa, Artificial Sequence
  Synthetic construct
DSNIDRLHSAGSDYRY

SEQ ID NO: 86 - protein, 16 aa, Artificial Sequence
  Synthetic construct
KQRVGPGIMSAPTYDY

SEQ ID NO: 87 - protein, 15 aa, Artificial Sequence
  Synthetic construct
SHGVVDGTSVNGYRY

SEQ ID NO: 88 - protein, 12 aa, Artificial Sequence
  Synthetic construct
WRFGPTGVKVDY

SEQ ID NO: 89 - protein, 10 aa, Artificial Sequence
  Synthetic construct
RNLETFDYTY

SEQ ID NO: 90 - protein, 9 aa, Artificial Sequence
  Synthetic construct
RTERGVYDY

SEQ ID NO: 91 - protein, 13 aa, Artificial Sequence
  Synthetic construct
TAYSTTYNSVRDY

SEQ ID NO: 92 - protein, 13 aa, Artificial Sequence
  Synthetic construct
VAYDYSWGRPRNF

SEQ ID NO: 93 - protein, 13 aa, Artificial Sequence
  Synthetic construct
VGQEASAYAPRAY

SEQ ID NO: 94 - protein, 18 aa, Artificial Sequence
  Synthetic construct
TSRTMIHTSQAMPINWDY

SEQ ID NO: 95 - protein, 10 aa, Artificial Sequence
  Synthetic construct
RDIETAEYIY

SEQ ID NO: 96 - protein, 10 aa, Artificial Sequence
  Synthetic construct
RDIETAEYTY

SEQ ID NO: 97 - protein, 11 aa, Artificial Sequence
  Synthetic construct
DTYSNYEKDDS

SEQ ID NO: 98 - protein, 11 aa, Artificial Sequence
  Synthetic construct
DTYSNYKKDDS

SEQ ID NO: 99 - protein, 11 aa, Artificial Sequence
  Synthetic construct
ETYSLYEKSDS

SEQ ID NO: 100 - protein, 11 aa, Artificial Sequence
  Synthetic construct
ETYSNYEKAYS

SEQ ID NO: 101 - protein, 11 aa, Artificial Sequence
  Synthetic construct
ETYTLYEKDSS

SEQ ID NO: 102 - protein, 13 aa, Artificial Sequence
  Synthetic construct
VGQEGSARAPRAY

SEQ ID NO: 103 - protein, 17 aa, Artificial Sequence
  Synthetic construct
SKDPYGSPWTRSEFDDY

SEQ ID NO: 104 - protein, 11 aa, Artificial Sequence
  Synthetic construct
ETYSIYEKDDS

SEQ ID NO: 105 - protein, 16 aa, Artificial Sequence
  Synthetic construct
DRGSSYYYTRASEYTY

SEQ ID NO: 106 - protein, 11 aa, Artificial Sequence
  Synthetic construct
AQPDVFGGGDY

SEQ ID NO: 107 - protein, 17 aa, Artificial Sequence
  Synthetic construct
SKDRYGSPWTRSEFEDY

SEQ ID NO: 108 - protein, 12 aa, Artificial Sequence
  Synthetic construct
RQGCSYNGCDEY

SEQ ID NO: 109 - protein, 12 aa, Artificial Sequence
  Synthetic construct
RQGCSYNGCDEY

SEQ ID NO: 110 - protein, 10 aa, Artificial Sequence
  Synthetic construct
RTPETGQYDY

SEQ ID NO: 111 - protein, 8 aa, Artificial Sequence
  Synthetic construct
RTPYMRDY

SEQ ID NO: 112 - protein, 18 aa, Artificial Sequence
  Synthetic construct
DRSEFGSRLYSDENEYDY

SEQ ID NO: 113 - protein, 13 aa, Artificial Sequence
  Synthetic construct
VAPVEGTGRIRAY

SEQ ID NO: 114 - protein, 15 aa, Artificial Sequence
  Synthetic construct
RASSNTYDDPRSFAF

SEQ ID NO: 115 - protein, 12 aa, Artificial Sequence
  Synthetic construct
GALRVGSFSPDY

SEQ ID NO: 116 - protein, 16 aa, Artificial Sequence
  Synthetic construct
VLGRPYGSRWLDDVDS

SEQ ID NO: 117 - protein, 17 aa, Artificial Sequence
  Synthetic construct
RIGSIANLQPREYEFAY

SEQ ID NO: 118 - protein, 9 aa, Artificial Sequence
  Synthetic construct
VDVSVGGDY

SEQ ID NO: 119 - protein, 11 aa, Artificial Sequence
  Synthetic construct
PACSGSGCRNY

SEQ ID NO: 120 - protein, 6 aa, Artificial Sequence
  Synthetic construct
ESGVNY

SEQ ID NO: 121 - protein, 16 aa, Artificial Sequence
  Synthetic construct
DSSTSRLHSAGSDYRY

SEQ ID NO: 122 - protein, 15 aa, Artificial Sequence
  Synthetic construct
NRGPNLYPHIGDIEY

SEQ ID NO: 123 - protein, 15 aa, Artificial Sequence
  Synthetic construct
SRGPNLYPHIGDVEY

SEQ ID NO: 124 - protein, 12 aa, Artificial Sequence
  Synthetic construct
RKSINYVQEYDY

SEQ ID NO: 125 - protein, 10 aa, Artificial Sequence
  Synthetic construct
RNIETAEYTY

SEQ ID NO: 126 - protein, 8 aa, Artificial Sequence
  Synthetic construct
QGQGNREY

SEQ ID NO: 127 - protein, 18 aa, Artificial Sequence
  Synthetic construct
SSRTMNTISQTMRINWDY

SEQ ID NO: 128 - protein, 8 aa, Artificial Sequence
  Synthetic construct
GIPMSTVL

SEQ ID NO: 129 - protein, 10 aa, Artificial Sequence
  Synthetic construct
RNPGTGQYDY

SEQ ID NO: 130 - protein, 14 aa, Artificial Sequence
  Synthetic construct
GVRDFAPRIDSYDY

SEQ ID NO: 131 - protein, 18 aa, Artificial Sequence
  Synthetic construct
DIAPFYCSWPLRTRLVEY

SEQ ID NO: 132 - protein, 18 aa, Artificial Sequence
  Synthetic construct
DPTILYHALSQRTQNYRY

SEQ ID NO: 133 - protein, 19 aa, Artificial Sequence
  Synthetic construct
CRQEFSWDFSSRDPDDFDY

SEQ ID NO: 134 - protein, 16 aa, Artificial Sequence
  Synthetic construct
SPYTYSLLSRTGDYGY

SEQ ID NO: 135 - protein, 13 aa, Artificial Sequence
  Synthetic construct
ALYVTSTRGSYAY

SEQ ID NO: 136 - protein, 18 aa, Artificial Sequence
  Synthetic construct
TSRTMIHTSQTMRINWDY

SEQ ID NO: 137 - protein, 15 aa, Artificial Sequence
  Synthetic construct
SRTAGLTSNRSLYDY

SEQ ID NO: 138 - protein, 14 aa, Artificial Sequence
  Synthetic construct
DVATIGSRLANYDY

SEQ ID NO: 139 - protein, 8 aa, Artificial Sequence
  Synthetic construct
RTPYRRDY

SEQ ID NO: 140 - protein, 17 aa, Artificial Sequence
  Synthetic construct
RRDSSWGYSRDLFEYDY

SEQ ID NO: 141 - protein, 13 aa, Artificial Sequence
  Synthetic construct
RFGIVTTRDEYEF

SEQ ID NO: 142 - protein, 10 aa, Artificial Sequence
  Synthetic construct
RSATTALYDY

SEQ ID NO: 143 - protein, 10 aa, Artificial Sequence
  Synthetic construct
KIFGPGSGSY

SEQ ID NO: 144 - protein, 12 aa, Artificial Sequence
  Synthetic construct
NTLNSQLLPRAY

SEQ ID NO: 145 - protein, 12 aa, Artificial Sequence
  Synthetic construct
GSLRVGSFSPDY

SEQ ID NO: 146 - protein, 13 aa, Artificial Sequence
  Synthetic construct
GPRLGSTPRAYDY

SEQ ID NO: 147 - protein, 17 aa, Artificial Sequence
  Synthetic construct
DKDVYYGYTSFPNEYEY

SEQ ID NO: 148 - protein, 14 aa, Artificial Sequence
  Synthetic construct
ARFYGRSIAQDYDS

SEQ ID NO: 149 - protein, 13 aa, Artificial Sequence
  Synthetic construct
GGWGLTQPISVDY

SEQ ID NO: 150 - protein, 13 aa, Artificial Sequence
  Synthetic construct
GTYGSTSVRDFGP

SEQ ID NO: 151 - protein, 7 aa, Artificial Sequence
  Synthetic construct
RTGSGVY

SEQ ID NO: 152 - protein, 14 aa, Artificial Sequence
  Synthetic construct
RKDSVVLSSVGYDY

SEQ ID NO: 153 - protein, 27 aa, Artificial Sequence
  Synthetic construct
DNAKNTIYLQMNSLKPQDTAVYYCAAR

SEQ ID NO: 154 - protein, 30 aa, Artificial Sequence
  Synthetic construct
LPDDFTGCVIAWNSNNLDSKVGGNYNYLYR

SEQ ID NO: 155 - protein, 22 aa, Artificial Sequence
  Synthetic construct
EFVAAIGASGGMTNYLDSVKGR

SEQ ID NO: 156 - protein, 16 aa, Artificial Sequence
  Synthetic construct
QIAPGQTGKIADYNYK

SEQ ID NO: 157 - protein, 11 aa, Artificial Sequence
  Synthetic construct
DTYYTNSVKGR

SEQ ID NO: 158 - protein, 9 aa, Artificial Sequence
  Synthetic construct
KSNLKPFER

SEQ ID NO: 159 - protein, 8 aa, Artificial Sequence
  Synthetic construct
STNLVKNK

SEQ ID NO: 160 - protein, 11 aa, Artificial Sequence
  Synthetic construct
SKDPYGSPWTR

SEQ ID NO: 161 - protein, 7 aa, Artificial Sequence
  Synthetic construct
KSTNLVK

SEQ ID NO: 162 - protein, 22 aa, Artificial Sequence
  Synthetic construct
EWVSGIDSDGSDTAYASSVKGR

SEQ ID NO: 163 - protein, 29 aa, Artificial Sequence
  Synthetic construct
ISNCVADYSVLYNSASFSTFKCYGVSPTK

SEQ ID NO: 164 - protein, 6 aa, Artificial Sequence
  Synthetic construct
NKCVNF

SEQ ID NO: 165 - protein, 14 aa, Artificial Sequence
  Synthetic construct
SGETTLYADSVKGR

SEQ ID NO: 166 - protein, 15 aa, Artificial Sequence
  Synthetic construct
DNAKNTVYLQMNSLK

SEQ ID NO: 167 - protein, 7 aa, Artificial Sequence
  Synthetic construct
QAPGKER

SEQ ID NO: 168 - protein, 8 aa, Artificial Sequence
  Synthetic construct
SNLKPFER

SEQ ID NO: 169 - protein, 21 aa, Artificial Sequence
  Synthetic construct
EWVAGITPGSGTFYADSVKGR

SEQ ID NO: 170 - protein, 26 aa, Artificial Sequence
  Synthetic construct
DNAKNTLSLEINSLKPEDTALYYCAK

SEQ ID NO: 171 - protein, 11 aa, Artificial Sequence
  Synthetic construct
VRQAPGKGREW

SEQ ID NO: 172 - protein, 21 aa, Artificial Sequence
  Synthetic construct
VIRGDEVRQIAPGQTGKIADY

SEQ ID NO: 173 - protein, 15 aa, Artificial Sequence
  Synthetic construct
SLEINSLKPEDTALY

SEQ ID NO: 174 - protein, 13 aa, Artificial Sequence
  Synthetic construct
NSNNLDSKVGGNY

SEQ ID NO: 175 - protein, 5 aa, Artificial Sequence
  Synthetic construct
GGGGS

SEQ ID NO: 176 - protein, 33 aa, Artificial Sequence
  Synthetic construct
EGKSSGSGSESKSTGGGGSEGKSSGSGSESKST

SEQ ID NO: 177 - DNA, 69 bp, Artificial Sequence
  Synthetic construct
ccgctcgagtgctgcggccgcggtgcttttgctttcgccgctaccgctgcttttaccttcgctgccacc

SEQ ID NO: 178 - DNA, 69 bp, Artificial Sequence
  Synthetic construct
cccaagcttgaaggtaaaagcagcggtagcggcgaaagcaaaagcaccggtggcggtggcagcgaaggt

SEQ ID NO: 179 - protein, 5 aa, Artificial Sequence
  Synthetic construct
YYCAA

SEQ ID NO: 180 - protein, 4 aa, Artificial Sequence
  Synthetic construct
WGQG

SEQ ID NO: 181 - DNA, 20 bp, Artificial Sequence
  Synthetic construct
ttacaaacattggccgcaaa

SEQ ID NO: 182 - DNA, 18 bp, Artificial Sequence
  Synthetic construct
gcgcgacattccgaagaa

SEQ ID NO: 183 - DNA, 23 bp, Artificial Sequence
  Synthetic construct
acaatttgcccccagcgcttcag

SEQ ID NO: 184 - protein, 33 aa, Artificial Sequence; identical to SEQ ID NO: 176
  Synthetic construct

SEQ ID NO: 185 - protein, 14 aa, Artificial Sequence
  Synthetic construct
ETYSIYEKDDSWGY

SEQ ID NO: 186 - protein, 424 aa, Artificial Sequence; identical to SEQ ID NO: 75
  Synthetic construct

SEQ ID NO: 187 - protein, 256 aa, Artificial Sequence
  Synthetic construct
QVQLVESGGGLVQAGGSLRLSCAVSGLGAHRVGWFRRAPGKEREFVAAIGANGGNTNYLDSVKGRFTISRDNAKNTIYLQMNSLKPQDTAVYYCAARDIETAEYTYWGQGTQVTVSSKLGGGGSGGGGSEVQLVESGGGLVQPGGSLRLSCAASGFTFSSYAMSWVRQAPGKGREWVAGITPGSGTFYADSVKGRFTISRDNAKNTLSLEINSLKPEDTALYYCAKCRQEFSWDFSSRDPDDFDYWGQGTQVTVSS

SEQ ID NO: 188 - protein, 255 aa, Artificial Sequence; identical to SEQ ID NO: 79
  Synthetic construct

SEQ ID NO: 189 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

(19) United States
(12) Patent Application Publication
(10) Pub. No.: US 2024/0043505 A1
(43) Pub. Date: Feb. 8, 2024

(54) CORONAVIRUS NANOBODIES AND METHODS FOR THEIR USE AND IDENTIFICATION

(71) Applicant: UNIVERSITY OF PITTSBURGH-OF THE COMMONWEALTH SYSTEM OF HIGHER EDUCATION, Pittsburgh, PA (US)

(72) Inventors: Yi SHI, I, Wexford, PA (US); Yufei XIANG, Pittsburgh, PA (US)

(21) Appl. No.: 18/022,033

(22) PCT Filed: Aug. 19, 2021
(86) PCT No.: PCT/US2021/046685

§ 371 Date: Feb. 17, 2023

Related U.S. Application Data
(60) Provisional application No. 63/067,567, filed on Aug. 19, 2020.

(51) Int. Cl. C07K 16/10; A61P 31/14

(52) U.S. Cl. CPC C07K 16/1003; A61P 31/14; C07K 2317/569; C07K 2319/31

(57) ABSTRACT

Disclosed herein are coronavirus neutralizing antibodies and uses thereof for treating and preventing a coronavirus infection in a subject.

### CROSS-REFERENCE TO RELATED APPLICATIONS

This application claims the benefit of U.S. Provisional Application No. 63/067,567, filed Aug. 19, 2020, which is expressly incorporated herein by reference in its entirety.

### BACKGROUND

Nanobodies (Nbs) are natural antigen-binding fragments derived from the V_{H}H domain of camelid heavy-chain only antibodies (HcAbs). They are characterized by their small size and outstanding structural robustness, excellent solubility and stability, ease of bioengineering and manufacturing, low immunogenicity in humans and fast tissue penetration. For these reasons, Nbs have emerged as promising agents for cutting-edge biomedical, diagnostic and therapeutic applications.

A novel, highly transmissible coronavirus SARS-COV-2 (severe acute respiratory syndrome coronavirus 2) {Zhu, 2020; Zhou, 2020} has infected more than 20 million people and has claimed over 700,000 lives, with the numbers still on the rise. Despite preventive measures, such as quarantines and lock-downs that help curb viral transmission, the virus often rebounds following the lifts on social restrictions. Safe and effective therapeutics and vaccines remain in dire need.

Like other zoonotic coronaviruses, SARS-COV-2 produces the surface spike glycoprotein (S), which is then cleaved into S1 and S2 subunits forming the homotrimeric viral spike to interact with host cells. The interaction is mediated by the S1 receptor-binding domain (RBD), which binds the peptidase domain (PD) of angiotensin-converting enzyme-2 (hACE2) as a host receptor {Wrapp, 2020}. Structural studies have revealed different stages of the spike trimer {Walls, 2020; Cai, 2020}. In the prefusion stage, the RBD switches between an inactive, closed conformation, and an active open structure necessary for interacting with hACE2. In the post-fusion stage, S1 dissociates from the trimer, and S2 undergoes a dramatic conformational change to trigger host membrane fusion. Most recently, investigations into COVID-19 convalescence individuals' sera have led to the identifications of highly potent neutralizing IgG antibodies (NAbs) primarily targeting the RBD but also the N-terminal domain (NTD) of the spike trimer {Cao, 2020; Robbiani, 2020; Hansen, 2020; Liu, 2020; Brouwer, 2020; Chi, 2020}. High-quality NAbs may overcome the risks of the Fc-associated antibody-dependent enhancement (ADE) and are promising therapeutic and prophylactic candidates {Zohar, 2020; Eroshenko, 2020}.

The VHH antibodies or nanobodies (Nbs) are minimal, monomeric antigen-binding fragments derived from camelid single-chain antibodies {Muyldermans, 2013}. Unlike IgG antibodies, Nbs are characterized by small sizes (˜15 kDa), high solubility and stability, ease of bioengineering into bi/multivalent forms, and low-cost microbial productions. Because of the robust physicochemical properties, Nbs are flexible for drug administration such as aerosolization, making their use against the respiratory, viral targets appealing {Vanlandschoot, 2011; Detalle, 2016}. Previous efforts have yielded broadly neutralizing Nbs for different challenging viruses, including Dengue, RSV, and HIV {Vanlandschoot, 2011}. However, highly potent camelid Nbs comparable to the human NAbs remain unavailable {Hilo, 2020; Wrapp, 2020; Konwarh, 2020}. The development of highly effective anti-SARS-COV-2 Nbs can provide a novel means for efficient and economic strategies for therapeutics and point-of-care diagnosis.

### SUMMARY

Provided herein are coronavirus (e.g., SARS-CoV-2 or SARS-CoV) neutralizing nanobodies and uses thereof for preventing or treating coronavirus infection. The nanobodies disclosed herein are surprisingly effective on reducing coronavirus viral load and preventing and treating a coronavirus infection. In some embodiments, the coronavirus neutralizing nanobody comprises an amino acid sequence selected from the group consisting of SEQ ID NO:1 through SEQ ID NO:152, SEQ ID NO: 185, and SEQ ID NO: 186. In some embodiments, the nanobody comprises a multimer of one or more amino acid sequences comprising a sequence selected from the group consisting of SEQ ID NO:82 through SEQ ID NO:152, SEQ ID NO: 185, and SEQ ID NO: 186. In some embodiments, the nanobody comprises a multimer (including, for example, a homodimer, a heterodimer, a homotrimer, or a heterotrimer) of one or more amino acid sequences comprising a sequence selected from the group consisting of SEQ ID NO:1 through SEQ ID NO:71. In some embodiments, the nanobody comprises a sequence of SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, or SEQ ID NO:186. In some embodiments, the coronavirus neutralizing nanobody is conjugated or linked to a human serum albumin binding nanobody or nanobody fragment. In some examples, the nanobody described herein exhibits high potency, with an IC50 of less than about 1 ng/1 ml.

The method provided herein comprises uses of the nanobodies described herein for treating or preventing a coronavirus infection (e.g., SARS-CoV-2 or SARS-CoV). In some examples, the method comprises administering the nanobody at a dose of about 0.2 mg/kg of body weight. The nanobody can be administered to a subject intratracheally, intranasally, or through an inhalation route. The nanobody has an increased serum half-life or in vivo stability as compared to a control.

### DESCRIPTION OF DRAWINGS

FIG. 1(A-F) shows production and characterizations of high-affinity RBD Nbs for SARS-CoV-2 neutralization. FIG. 1A shows the binding affinities of 71 Nbs towards RBD by ELISA. The pie chart shows the number of Nbs according to affinity and solubility. FIG. 1B shows screening of 49 soluble, high-affinity Nbs by SARS-CoV-2-GFP pseudovirus neutralization assay. n=1 for Nbs with neutralization potency IC50<=50 nM, n=2 for Nbs with neutralization potency IC50>50 nM. FIG. 1C shows that the neutralization potency of 18 highly potent Nbs was calculated based on the pseudotyped SARS-CoV-2 neutralization assay (luciferase). Purple, red, and yellow lines denote Nbs 20, 21, and 89 with IC50<0.2 nM. Two different purifications of the pseudovirus were used. The average neutralization percentage was shown for each data point (n=5 for Nbs 20, 21; n=2 for all other Nbs). FIG. 1D shows the neutralization potency of the top 14 neutralizing Nbs by SARS-CoV-2 plaque reduction neutralization test (PRNT). The average neutralization percentage was shown for each data point (n=4 for Nbs 20, 21, and 89; n=2 for other Nbs). FIG. 1E shows a table summary of pseudotyped and SARS-CoV-2 neutralization potency for 18 Nbs. N/A: not tested. FIG. 1F shows the SPR binding kinetics measurement of Nb21.

FIG. 2(A-G) shows Nb epitope mapping by integrative structural proteomics. FIG. 2A shows a summary of Nb epitopes based on size exclusion chromatography (SEC) analysis. Light salmon color: Nbs that bind the same RBD epitope. Sea green: Nbs of different epitopes. FIG. 2B shows a representation of SEC profiling of RBD, RBD-Nb21 complex, and RBD-Nb21-Nb105 complex. The y-axis represents UV 280 nm absorbance units (mAu). FIG. 2C shows a cartoon model showing the localization of five Nbs that bind different epitopes: Nb20 (medium purple), Nb34 (light sea green), Nb93 (salmon), Nb105 (pale goldenrod) and Nb95 (light pink) in complex with the RBD (gray). Blue and red lines represent DSS cross-links shorter or longer than 28 Å, respectively. FIG. 2D shows top 10 scoring cross-linking based models for each Nb (cartoons) on top of the RBD surface. FIG. 2E shows the surface display of different Nb neutralization epitopes on the RBD in complex with hACE2 (cartoon model in blue). FIG. 2e: Schematics of five unique RBD epitopes for Nb binding. The residue numbers of the RBD were shown (from aa 333 to aa 533). FIG. 2F shows schematics of five unique RBD epitopes for Nb binding. The residue numbers of the RBD were shown (from aa 333 to aa 533). FIG. 2G shows an overview of the Nb neutralization epitopes revealed by cross-linking.

FIG. 3(A-F) shows crystal structure analysis of an ultrahigh affinity Nb in complex with the RBD. FIG. 3A shows cartoon presentation of Nb20 in complex with the RBD. CDR1, 2, and 3 are in red, green, and orange, respectively. FIG. 3B shows zoomed-in view of an extensive polar interaction network that centers on R35 of Nb20. FIG. 3C shows zoomed-in view of hydrophobic interactions. FIG. 3D shows surface presentation of the Nb20-RBD and hACE2-RBD complex (PDB: 6M0J). FIG. 3E shows surface presentation of RBD with hACE2 binding epitope colored in steel blue and Nb20 epitope colored in medium purple. FIG. 3F shows the CDR1 and CDR3 residues (medium violet pink and light goldenrod in spheres, respectively) of Nb20 overlap with hACE2 binding site (light blue) on the RBD (gray).

FIG. 4(A-F) show mechanisms of SARS-CoV-2 neutralization by Nbs. FIG. 4A shows hACE2 (grey) binding to spike trimer conformation (wheat, plum, and light blue colors) with one RBD up (PDBs 6VSB, 6LZG). FIG. 4B shows that Nb20 (Epitope I, medium purple) partially overlaps with the hACE2 binding site and can bind the closed spike conformation with all RBDs down (PDB 6VXX). FIG. 4C shows a summary of spike conformations accessible (+) to the Nbs of different epitopes. FIG. 4D shows that Nb93 (Epitope II, salmon) partially overlaps with the hACE2 binding site and can bind to spike conformations with at least one RBD up (PDB 6VSB). FIG. 4(E-F) shows that Nb34 (Epitope III, light sea blue) and Nb95 (Epitope IV, light pink) do not overlap with the hACE2 binding site and bind to spike conformations with at least two open RBDs (PDB 6XCN).

FIG. 5(A-E) shows development of multivalent Nb cocktails for highly efficient SARS-CoV-2 neutralization. FIG. 5A shows schematics of the cocktail design. FIG. 5B show pseudotyped SARS-CoV-2 neutralization assay of multivalent Nbs. The average neutralization percentage of each data point was shown (n=2). FIG. 5C shows SARS-CoV-2 PRNT of monomeric and trimeric forms of Nbs 20 and 21. The average neutralization percentage of each data point was shown (n=2 for the trimers, n=4 for the monomers). FIG. 5D shows a summary table of the neutralization potency measurements of the multivalent Nbs. N/A: not tested. FIG. 5E shows mapping mutations to localization of Nb epitopes on the RBD. The x-axis corresponds to the RBD residue numbers (333 to 533). Rows in different colors represent different epitope residues. Epitope I: 351, 449-450, 452-453, 455-456, 470, 472, 483-486, 488-496; Epitope II: 403, 405-406, 408,409, 413-417, 419-421, 424, 427, 455-461, 473-478, 487, 489, 505; Epitope III: 53, 355, 379-383, 392-393, 396, 412-413, 424-431, 460-466, 514-520; Epitope IV: 333-349, 351-359, 361, 394, 396-399, 464-466, 468, 510-511, 516; Epitope V: 353, 355-383, 387, 392-394, 396, 420, 426-431, 457, 459-468, 514, 520.

FIG. 6(A-C) shows development of RBD-specific Nbs for potent SARS-COV-2 neutralization. FIG. 6A shows detection of strong and specific serologic activities after immunization of SARS-CoV-2 RBD. FIG. 6B shows neutralization potency of the immunized camelid's serum against pseudotyped SARS-CoV-2-Luciferase. FIG. 6C shows neutralization potency of Nbs against pseudotyped SARS-CoV-2-Luciferase.

FIG. 7(A-C) shows identification of a large repertoire of high-affinity Nbs by proteomics. FIG. 7A shows the schematic of high-affinity RBD-Nb identification by camelid immunization and quantitative Nb proteomics. Briefly, a camelid was immunized by the RBD. High-affinity, RBD-specific single-chain VHH antibodies were affinity isolated from the immunized serum, and analyzed by quantitative proteomics to identify the high-affinity, RBD-specific Nbs (see Methods). A VHH (Nb) cDNA library from the plasma B cells of the immunized camelid was created to facilitate proteomic analysis. FIG. 7B shows sequence logo and sequence logo of 120 high-affinity RBD Nbs. The amino acid occurrence at each position is shown. CDR: complementarity determining region. FR: framework. FIG. 7C shows the phylogenetic tree of the Nbs constructed by the maximum likelihood model.

FIG. 8 shows correlation analysis of 18 highly potent SARS-CoV-2 neutralizing Nbs. A plot showing a linear correlation of Nb neutralization IC50s between the pseudotyped virus neutralization assay and the SARS-CoV-2 PRNT.

FIG. 9(A-D) shows biophysical analysis of the outstanding neutralizing Nbs. FIG. 9(A-B) shows binding kinetics of Nbs 20 and 89 by surface plasmon resonance (SPR). FIG. 9C shows thermostability analysis of Nbs 20, 21, and 89. The values represent the average thermostability (Tm, ° C.) based on three replicates. The standard deviations (SD) of the measurements are 0.17, 0.93, and 0.8° C. for Nbs 20, 21, and 89. FIG. 9D shows stability analysis of Nb21 by SEC. Purified recombinant Nb21 was stored at room temperature for ˜6 weeks before subject to SEC analysis. The dominant peak represents Nb 21 monomer.

FIG. 10 shows SEC analysis of RBD-Nb complexes.

FIG. 11(A-I) shows SEC analysis of RBD-Nb complexes. The SEC profiles of RBD-Nb complexes showing 9 Nbs that have overlapping epitopes with Nb21. FIG. 11(A-H) shows the SEC profiles of RBD-Nb complexes showing five Nbs that have unique and non-overlapping epitopes with Nb21. FIG. 11I shows sequence alignment of the CDR3s of 18 highly potent neutralizing Nbs and CDR3 lengths comparing Nbs from epitope I and others.

FIG. 12(A-C) shows competitive ELISA analysis of hACE2 and Nbs for RBD binding and conversation analysis of RBD across different coronaviruses. FIG. 12A shows competitive ELISA of hACE2 and Nbs (20, 21, 93, and 95) for RBD binding. Y-axis: percentage of the normalized ACE2 signal. X-axis: Nb concentration (nM). FIG. 12B shows the surface display of different Nb neutralization epitopes on RBD in complex with hACE2 (cartoon model in blue). FIG. 12C shows the conservation analysis of the spike protein RBD using the ConSurf web server, oriented as in FIG. 12B. The conservation is based on 150 sequences automatically extracted by the ConSurf server.

FIG. 13 shows structural comparisons of Nb20 with published RBD Nb structures. Overlays of Nb20 (purple ribbon) and three other RBD-Nbs (PDBs 6YZ5, 7C8V, and 7C8 W) in complex with RBD (yellow/grey ribbon).

FIG. 14(A-D) shows structural modeling of Nb 21-RBD interaction based on the Nb20-RBD crystal structure. In FIG. 14A, alignment of Nb21 with Nb20. The four residue differences between the two Nbs were shown. FIG. 14B shows zoom-in views showing the addition of new polar interaction between N52 (Nb21) and N450 (RBD). The model of Nb21 is superimposed based on the crystal structure of Nb20. FIG. 14C shows surface presentation of RBD. The hACE2 binding epitope is in steel blue and the Nb20 epitope is in medium purple. FIG. 14D shows structural alignment of Nb20-RBD complex with hACE2-RBD complex. The CDR1 and CDR3 residues (medium violet pink and goldenrod in spheres, respectively) of Nb20 overlap with the hACE2 binding site (steel blue) on RBD (grey ribbon).

FIG. 15(A-D) shows the biophysical properties of multivalent Nbs. FIG. 15A shows the expression levels of multivalent Nbs from E. coli whole cell lysates. FIG. 15B shows SDS-PAGE analysis of the purified multivalent Nbs. FIG. 15C shows thermostability analysis of ANTE-CoV2-Nab21T_{EK}, ANTE-CoV2-Nab20T_{EK}, ANTE-CoV2-Nab21T_{GS}, and ANTE-CoV2-Nab20T_{GS}. The values represent the average thermostability (Tm, ° C.) based on three replicates. The standard deviations of the measurements are 0.6, 0.27, 0.169, and 0.72° C., respectively. FIG. 15D shows high stability of the multivalent Nbs under the pseudovirus neutralization condition. Different Nb constructs were incubated under the pseudovirus neutralization assay condition without the virus for 72 hours. An anti-His6 mouse monoclonal antibody (Genscript) was used to detect the Nb constructs (His6 tag at the C terminus) by western blot.

FIG. 16(A-G) shows stability test of the multivalent Nbs. FIG. 16A shows SARS-CoV-2 PRNT of the homo-trimeric forms of Nbs 20 and 21 with an EK linker. The average neutralization percentage and the standard deviation of each data point were shown (n=2). FIG. 16(B-C) shows the SEC analysis of ANTE-CoV2-Nab20TGS and ANTE-CoV2-Nab21TEK before and after lyophilization or aerosolization. FIG. 16(D-E) shows pseudotyped SARS-CoV-2 neutralization assay using ANTE-CoV2-Nab20T G s and ANTE-CoV2-Nab21T EK before and after lyophilization or aerosolization (n=2). FIG. 16F shows a summary table of the neutralization potency measurements of the homo-trimeric Nbs. FIG. 16G shows a portable mesh nebulizer (producing ≤5 μm aerosol particles) used in the study.

FIG. 17 shows the neutralization epitopes and virus mutations mapped on the RBD crystal structure. The dashed line (upper panel) indicates epitope V that partially overlaps with epitopes III and IV. The mutations (lower panel) are colored in gradient blue (0-100 mutation count from the GISAID), where darker blue indicates more frequent mutations.

FIG. 18(A-D) shows composite 2Fc-Fo electron density maps of the representative areas of RBD-Nb20 complex contoured at 1.0σ. FIG. 18A shows map of the whole complex shown as purple mesh. FIG. 18B shows map of the three CDRs of Nb20 as purple mesh. FIG. 18C shows map of the extended external loop region of RBD shown as purple mesh. FIG. 18D shows map of residues involved in the interactions between RBD and Nb20 shown as red mesh. RBD is colored in gray and Nb20 is colored in blue.

FIG. 19(A-C) shows correlation analysis of 18 highly potent SARS-CoV-2 neutralizing Nbs. FIG. 19A depicts a plot showing a linear correlation of Nb neutralization potency (IC50s) between two different SARS-COV-2 viral assays (pseudotype virus vs. authentic virus). FIG. 19B shows a heat map showing the correlation between SHM and Nb pseudovirus neutralization potency, Pearson r=−0.408. FIG. 19C shows a heatmap showing the correlation between ELISA affinity and Nb pseudovirus neutralization potency, Pearson r=−0.639.

FIG. 20(A-B) shows structural modeling of Nb 21 based on the Nb 20-RBD crystal structure. FIG. 20A shows a structural model of Nb 21 in complex with RbD. FIG. 20B shows zoom-in views showing the addition of new polar interaction between N52 (Nb21) and N450 (RBD). The model of Nb21 is superimposed based on the crystal structure of Nb20.

FIG. 21(A-B) shows structural comparisons of Nb 20 with published RBD Nb structures. FIG. 21A shows an zoom-in view showing the cacodylate ion that is embedded in the interaction of Nb20-RBD. FIG. 21B shows structural overlays of Nb 20 and other RBD-Nbs.

FIG. 22 depicts an example of a computing system that executes methods and procedures described in certain embodiments of the present disclosure.

FIG. 23(A-E) shows PiN-21 protects Syrian hamsters from SARS-CoV-2 infection. FIG. 23A shows overview of the experimental design. 9×10⁴ p.f.u. of SARS-CoV-2 was intratracheally inoculated followed by intranasal delivery of 100 μg PiN-21 (shown in blue dots) or a control Nb (shown in grey circles). Animal weight changes were monitored daily. Nasal washes and throat swabs were collected on 2 and 4 d.p.i. Animals were euthanized for necropsy on 5 (n=3) and 10 d.p.i (n=3) with viral titers and gRNAs of lung tissues measured. FIG. 23B shows the protection of weight loss of infected hamsters treated with PiN-21. *** indicates a p-value of <0.001. FIG. 23(C-E) shows measurement of viral titers by the plaque assay. ** indicates a p-value of <0.01. The dashed line indicates the detection limit of the assay. The color scheme is consistent across all the panels.

FIG. 24(A-D) shows assessment of Nb delivery in the hamster respiratory system. FIG. 24A shows schematic design of PiN-21 (shown in red triangles) and PiN-21_{Alb} (shown in blue squares) aerosolization in hamster models. FIG. 24B shows Nb neutralization potency before and after aerosolization measured by PRNT₅₀ assay. FIG. 24(C-D) shows normalized overall neutralization activity by plaque assay of PiN-21 and PiN-21_{Alb} of different time points post-aerosolization.

FIG. 25(A-F) shows treatment efficacy of aerosolized PiN-21 in the hamster model of SARS-CoV-2. FIG. 25A shows overview of the experiment design. 3×10⁴ p.f.u. of SARS-CoV-2 was intranasally inoculated. PiN-21 (shown in red triangles) or a control Nb (shown in grey circles) was aerosolized to hamsters in the cage 6 h.p.i. Animal weight changes were monitored, nasal washes and throat swabs were taken daily. Animals were euthanized for necropsy on 3 d.p.i with viral titers and gRNA of lung tissues measured. FIG. 25B shows the percentage of body weight change of PiN-21 aerosol-treated animals compared to the control (n=6). FIG. 25C shows reduction of viral titers in hamster lungs (3 d.p.i.). Significant differences were observed between treated and control groups. **, P<0.01; *, P<0.05. The dashed line indicates the detection limit of the assay. FIG. 25D shows lung pathology scores of treated and control groups. Significant difference was denoted by ****, P<0.0001. FIG. 25E shows H&E staining of necrotizing bronchointerstitial pneumonia affiliate with abundant SARS-CoV-2 S antigen in bronchiole epithelium and alveolar type 1 and 2 pneumocytes in the control group. All images acquired at 20×, scale bar=100 μm. Areas marked by boxes are shown at higher magnification in the right-most panel (scale bar=25 μm). FIG. 25F shows immunostainings of bronchointerstitial compartments (3 d.p.i.). Orange, SARS-CoV-2 S; magenta, CD68/macrophages; red, CD3e+ T cells; teal, ACE2; grey, DAPI. The bronchiole is outlined by white hash. Total magnification 200×, scale bar=100 μm.

FIG. 26(A-D) shows the efficacy of PiN-21 for protecting SARS-CoV-2 infection in hamsters. FIG. 26(A-B) shows measurement of gRNA by RT-qPCR on 5 and 10 d.p.i. (n=3 in each group). FIG. 26(C-D) shows measurement of gRNA by RT-qPCR on 2 and 4 d.p.i. * indicates a p-value of <0.05. **** indicates a p value of <0.0001. The dashed line indicates the detection limit of the assay.

FIG. 27(A-E) shows the efficacy of PiN-21 for the treatment of SARS-CoV-2 infection in hamsters. FIG. 27A shows schematic design of intranasal delivery of PiN-21 in hamsters for treatment. 3×10⁴ p.f.u. of SARS-CoV-2 was intranasally inoculated. 100 μg of PiN-21 (shown in black dots) or a control Nb (shown in grey circle) was intranasally delivered 6 h.p.i Animal weight changes were monitored daily and were euthanized for necropsy on 6 d.p.i with viral titers of lung tissues measured. FIG. 27B shows protection of weight loss in the PiN-21 treatment group. Significant differences were denoted as **, P<0.01; ***, P<0.001. FIG. 27(C-D) shows measurement of viral titers in nasal washes and throat swabs by the plaque assay on 2 and 4 d.p.i. (n=6). FIG. 27E shows measurement of viral titers in lung tissues by the plaque assay on 6 d.p.i. (n=6).

FIG. 28(A-B) shows characterization of Nb constructs after aerosolization by a portable mesh nebulizer. FIG. 28A shows protein recovery after aerosolization. FIG. 28B shows Nb neutralization potency before and after aerosolization measured by pseudovirus neutralization assay.

FIG. 29 (A-C) shows treatment efficacy of aerosolized PiN-21 in the Syrian hamster model of SARS-CoV-2 infection. FIG. 29(A-B) shows measurement of viral titers in nasal washes and throat swabs using the plaque assay. Significant differences were denoted using *, P<0.05, ****, P<0.0001. FIG. 29C shows measurement of gRNA in lung tissues by RT-qPCR on 3 d.p.i. (n=6).

FIG. 30(A-H) shows aerosolization treatment of PiN-21 in SARS-CoV-2 infected Syrian hamsters prevents severe histopathological manifestations in the LRT. Hematoxylin and Eosin stain. Total magnification 200×, scale bar=100 μm (A & E) or 400×, scale bar=50 μm in FIG. 30(B, C, D, F, G, H). FIG. 30A shows tracheal hyperplasia and hypertrophy. Figure shows bronchiole hyperplasia, degeneration, and necrosis with syncytial cells (arrows). FIG. 30C shows perivascular edema and inflammation infiltrate with reactive endothelium (arrowheads). FIG. 30D shows severe interstitial pneumonia with intra-alveolar fibrin and hemorrhage. FIG. 30E shows histologically normal trachea. FIG. 30F shows mild bronchiole degeneration with denuded intraluminal epithelium. FIG. 30G shows mild perivascular mononuclear infiltrate. FIG. 30H shows mild focal interstitial pneumonia.

FIG. 31(A-B) shows correlation analysis of weight loss with virus titer in the hamster model on 3 d.p.i. (FIG. 31A) and 5 d.p.i. (FIG. 31B).

FIG. 32(A-B) shows the impact of RBD circulating variants on Nb binding and neutralization. FIG. 32A shows ELISA binding of the spike variants (a summary heatmap). Data shown as binding affinity fold change relative to that of RBD WT. FIG. 32B shows the fold change in neutralizing potencies of the Nbs against two dominant circulating variants (UK and SA strains) relative to that of the wild-type SARS-CoV-2 pseudovirus particles. Negative values represent loss in affinity or neutralization potency, and positive values represent gain in affinity or neutralization potency. Based on the highest Nb concentration tested, reduction in affinity or neutralization potency greater than 1000 fold is represented as “<−1000”.

FIG. 33 (A-D) shows structure of an ultrapotent class I Nb (21). FIG. 33A shows Cryo-EM structure of the Nb21:S complex reveals “1-up and 2-down” RBD conformations. FIG. 33B shows the involvement of three CDRs of Nb21 for RBD binding. FIG. 33C shows additional Nb21:RBD interactions: side chains of R97, N52, and N55 (Nb21) form hydrogen bonds with the main chain carbonyl groups of L492 and Y449 and the side chain of T470 (RBD), respectively. The main-chain carbonyl group of A29 (Nb21) also forms a hydrogen bond with Q493 (RBD). Besides these polar interactions, F45 and L59 of Nb21 and V483 of RBD form a cluster of hydrophobic interactions, together, providing ultrahigh-affinity and selectivity for RBD binding. FIG. 33D shows structural overlap of hACE2 with Nb21:RBD complex.

FIG. 34(A-F) shows structures of class II Nbs (95, 34 and 105). FIG. 34A shows cryo-EM structures of Nbs 95 and 34 in complex with S. FIG. 34B shows cryo-EM structure of the Nb105:Nb21:RBD complex. FIG. 34C shows Nb95: RBD interactions. Residues in pink denote Nb95 for RBD binding. FIG. 34D shows Nb105: RBD interactions. Residues in yellow denote Nb105 for RBD binding. FIG. 34E shows that class II Nb:RBD interactions are predominantly mediated by CDR3. Nbs are represented as ribbons. The CDR3 loops are shown as surface representations. FIG. 34F shows steric effects of class II Nbs on hACE2:RBD interactions. N322 glycosylation (ACE2) is presented in red density.

FIG. 35(A-H) shows structures of class III Nbs (17 and 36). FIG. 35A shows cryo-EM structures of Nb17 in complex with S. FIG. 35B shows that Nb17:RBD interactions are mediated by all three CDRs. FIG. 35C shows cryo-EM structure of the Nb17:Nb105:RBD complex. FIG. 35D shows that Nb17 structurally does not overlap with ACE2. FIG. 35E shows cryo-EM structure of the Nb36:Nb21:RBD complex. FIG. 35F shows epitope of Nb36 on the RBD surface. FIG. 35G shows Nb17 stacks on NTD via its framework, while isolated Nb36:RBD complex indicates Nb36 would clash with neighboring NTD on S. FIG. 35H shows ACE2 competition assay with the S.

FIG. 36(A-E) shows that class III Nbs bind novel and semi-conserved neutralizing epitopes unique to Nbs. FIG. 36A shows epitope clustering analysis of RBD Nbs and correlation with RBD sequence conservation and ACE2 binding sites. The conservation scores of SARS-CoV-2 Spike RBD amino acids were computed by ConSurf server using the empirical bayesian method from the multiple sequence alignment and normalized by z-score method. FIG. 36B shows overview of three Nb classes binding to the RBD, RBD surface was colored based on conservation (ConSurf score). FIG. 36(C-E) shows structural comparison of different classes of Nbs with the closest mAbs for RBD binding.

FIG. 37(A-F) shows that mAbs and Nbs binding to RBD are differently affected by mutations in the circulating variants. FIG. 37A shows localization of six RBD residues where major circulating variants mutate. FIG. 37B shows buried surface area of Nbs by different RBD residues. FIG. 37C shows buried surface area of Fabs by different RBD residues. FIG. 37(D-E) shows representative structures of different classes of Nbs with major variant residues shown as spheres. Two Fab structures that bind similarly to Class I Nbs were shown on the side. FIG. 37F shows the boxplot showing the probability of epitope residues coinciding with the variant mutations.

FIG. 38(A-G) shows comparisons of RBD neutralizing Nbs and mAbs. FIG. 38A shows buried surface areas of RBD: Nb and RBD: Fab complexes. VH: heavy chain. VL: light chain. FIG. 38B shows buried surface areas per-interface residue for Nbs and Fabs. FIG. 38C shows the contact contribution of CDRs and FRs of Nbs and Fabs in RBD binding (using a 6 Å cutoff). Contact contribution % was calculated as #of contacting residues on CDR or FR region/total #of contacting residues. FIG. 38D shows quantification of interface cavity. Y-axis is the curvature value. FIG. 38E shows comparison of contributions from CDRs and FRs for RBD binding between in vivo matured Nbs and in vitro selected Nbs. FIG. 38F shows representative structures of 7d showing different binding modes (epitope curvature) of an Nb and a Fab. Nbs target concave RBD surfaces to achieve high-affinity binding. FIG. 38G shows representative structures of 7e showing the direct involvement of FR2 from an in vitro selected Nb (PDB #7A29) for RBD interaction.

FIG. 39(A-G) shows ELISA curves of Nbs for RBD mutant binding.

FIG. 40(A-B) shows structure representations of SARS-CoV-2 spike trimer glycoprotein and mutations for two prevalent circulating strains. FIG. 40A shows all mutations for VOC B.1.1.7 and 501Y.V2 highlighted in red. Mutations for B.1.1.7 UK include del69-70, delY144, N501Y, A570D, D614G, P681H, T716I, S982A, and D1118H. Mutations for SA 501Y.V2 include L18F, D80A, del241-243, D215G, R246I, K417N, E484K, N501Y, D614G, AND A701V. FIG. 40B shows all mutations for ACE2 affinity matured RBD B62 highlighted in red. Mutations include I358F, V445K, N460K, I468T, T470M, S477N, E484K, Q498R, and N501Y.

FIG. 41(A-G) shows pseudovirus assay results for individual Nbs.

FIG. 42(A-F) shows cryo-EM Structure determination of S with Nb21, local refinement of RBD with Nb21 and comparison of Nb21 with Nb20. FIG. 42A shows cryo-EM data processing workflow showing the strategies and particle cohort sizes used to generate the maps discussed in this work. ˜900K particles were picked based on the 2D class averages of S with Nb21 for 3D classification. Two major classes with the largest proportions were further refined. One class refined to 3.6 Å corresponds to S with 1-up-2-down RBDs and the other class refined to 3.9 Å corresponds to S with 2-up-1-down RBDs. S is colored in dark gray. Nb21 is colored blue. FIG. 42B shows fourier shell correlation and local resolution estimations for S and NB21 complexes. The red line represents FSC=0.143. FIG. 42C shows focused refinement of one down RBD with Nb21. FIG. 42(D-F) shows structural comparison of RBD with Nb21 and with Nb20. Nb21 is colored blue while Nb20 is colored yellow. RBD is colored dark gray and cyan in the structures with Nb21 and Nb20, respectively. Nb21 differs from Nb20 by four residues (all on CDRs). Its RBD binding is very similar to that of Nb20. The two structures can be well aligned with a root mean square deviation (RMSD) of 1.8 Å (all atoms). Here, S52 and M55 on CDR2 in Nb20, are replaced by N52 and N55 in Nb21, which form additional polar interactions with the RBD (e). A27 (CDR1) and 1105 (CDR3) of Nb20 are replaced by L27 and T105 in Nb21 (f). While the two residues do not bind RBD directly, the side chain of L27 is buried inside Nb21 to form additional hydrophobic interactions with V24, V32, and I77. The small short side chain of T105 allows the neighboring residue Y106 to point towards the first N-terminal residue Q1 to form a hydrogen bond. This interaction, which is missing in the structure of Nb20:RBD as it is impeded by the presence of the large side chain of I105 in the analogous position of Nb20 I105. These additional interactions may help stabilize CDR1 and CDR3 loops to strengthen Nb21:RBD interactions.

FIG. 43(A-B) shows assessment of the RBD:Nb21 interactions using both computational binding energy calculation and experimental mutagenesis. FIG. 43A shows decomposition of relative binding free energy contribution from individual residues of RBD (top, gray) and Nb21 (bottom, blue) for these more than −1 kcal/mol. FIG. 43B depicts ELISA assay showing Nb21 point mutant R31D fails to bind RBD.

FIG. 44(A-C) shows cryo-EM Structure determination of S with Nb95 and focused refinement. FIG. 44A shows that ˜880K particles were picked based on the 2D class averages of S with Nb95 for 3D classification. Two major classes with the largest proportions were further refined. One class refined to 3.8 Å corresponds to S with 2-up-1-down RBDs and the other class refined to 3.7 Å corresponds to S with 3-up RBDs. S is colored in dark gray. Nb95 is colored in teal. FSC estimations for these two complexes are shown at the lower-left corner. The red line represents FSC=0.143. FIG. 44B shows local resolution distribution for the two S and Nb95 complexes. FIG. 44C shows focused refinement of one down RBD with NB95. The down RBD showed better density compared to up RBDs.

FIG. 45(A-C) shows cryo-EM Structure determination of S with Nb34 and focused refinement. FIG. 45A shows that ˜756K particles were picked based on the 2D class averages of S with Nb34 for 3D classification. Two major classes were observed with clear features of 3-up RBDs and 2-up-1-down RBDs. We focused on the 2-up-1-down class for 3D refinement and obtained a structure with a global resolution of 3.5 Å corresponding to 0.143FSC shown at the lower-left panel. FIG. 45B shows local resolution distribution for the S and Nb34 complex. FIG. 45C shows focused refinement of one down RBD with NB34. The down RBD showed better density compared to up RBDs.

FIG. 46(A-F) shows cryo-EM analysis of Nb105:S and Nb105:RBD:Nb21 complexes. FIG. 46A shows representative micrograph and 2D class averages of Nb105:S complex. FIG. 46B shows gold-standard fourier shell correlation (FSC) and Euler angular distribution. FIG. 46C shows representative micrograph and 2D class averages of Nb105:RBD:Nb21 complex. FIG. 46D shows gold-standard fourier shell correlation (FSC) and Euler angular distribution. FIG. 46E shows local resolution estimation for Nb105:RBD:Nb21 complex. FIG. 46F shows rigid docking of Nb105:RBD complex to the interface of the dimeric S. The interface highlighted with the green line is between the Nb framework and RBS.

FIG. 47(A-H) shows cryo-EM analysis of Nb17:S and Nb17:RBD:Nb105 complexes. FIG. 47A shows representative micrograph and 2D class averages of Nb17:S complex. FIG. 47B shows gold-standard fourier shell correlation (FSC) and Euler angular distribution. FIG. 47C shows local resolution estimation for Nb17:S complex. FIG. 47D shows focused classification of the flexible region in Nb17:S complex. The density of Nb17 in class 1 (cyan) is smeared due to motion along the y-direction, class 2 (magenta) has well resolved RBD, Nb17, and NTD density, and both densities of RBD and Nb17 is lost due to motion along the x-direction. FIG. 47E shows representative micrograph and 2D class averages of Nb17:RBD:Nb105 sample. FIG. 47F shows local resolution estimation for Nb105:RBD:Nb21 sample. FIG. 47G shows interface residues of Nb17:RBD complex. FIG. 47H shows alignment of Nb17:RBD to Nb21:RBD showing the large overlap between Nb17 CDR3 with Nb21 CDR2 and partially Nb21 CDR1.

FIG. 48(A-C) shows structure models with cryo-EM density for the interface region between RBD and Nbs after local refinement. FIG. 48A shows the density map of Nb21:RBD interactions. FIG. 48B shows the density map of Nb95:RBD interactions. FIG. 48C shows the density map of Nb105:RBD interactions.

FIG. 49(A-E) shows EM Analysis of Nb36 with S and RBD. FIG. 49A shows representative negative stain EM micrographs of spike protein in the presence of an increased concentration of Nb36. An example of an intact trimeric spike particle is highlighted by a blue arrow, and an example of a disrupted spike particle is highlighted by a red arrow. FIG. 49B shows thermal melting profile of S protein in the presence of an increased concentration of Nb36. FIG. 49C shows representative micrograph and 2D class averages of Nb36:RBD:Nb21 complex. FIG. 49D shows gold-standard fourier shell correlation (FSC) and Euler angular distribution. FIG. 49E shows local resolution estimation for Nb36:RBD:Nb21 complex.

FIG. 50(A-D). FIG. 50A shows that Nb17 promotes the SARS-CoV-2 S transition to post-fusion state by Western Blot. The stable SARS-CoV-2 S trimer (hexapro) was digested with proteinase K either directly, or after incubation with hACE2 or Nbs for 15 min or 60 min at room temperature. anti-S2 SARS-CoV-2 polyclonal antibodies were used for western blot analysis. FIG. 50B shows hydrophobic interactions formed between L452(RBD) and S30, V96, Q98(Nb17). FIG. 50C shows hydrodynamic radius distribution by intensity for S, S immediately upon addition of Nb36, S with Nb36 incubated at room temperature for 2 hours and S with Nb21 at 0.6 mg/mL concentration of S and 6:1 molar ratio of Nbs in PBS buffer. Figure shows size exclusion chromatography profiles of S (black), S with Nb21 (green) and S with Nb36 (red) with superdex 200 GL 10/300 column on Shimadzu HPLC at 0.25 mL/min flow rate in PBS buffer. The protein standard profile is shown in gray overlapping spike only profile.

FIG. 51(A-D) shows analysis of binding of 7 Nbs against _{RBDSARS-CoV} FIG. 51A shows RBD Sequence alignment from the Sarbecovirus family Major epitopes of three classes of Nbs were highlighted and epitope identities on different RBDs were shown. FIG. 51B shows analysis of RBD sequence identity of 12 representative sarbecoviruses for different classes of Nbs. FIG. 51C shows sequence alignment of SARS-CoV-2 and SARS-CoV, with non-conserved SARS-CoV amino acid residues highlighted in red letters. Individual Nb epitope footprints on SARS-CoV-2 RBD are illustrated in color coded dots along the primary sequence. FIG. 51D shows binding affinity (IC50) of different Nbs towards SARS-CoV and SARS-CoV-2 measured by ELISA. IC50 values reported in nM units. ND: signal not detected.

FIG. 52(A-C) shows comparison of neutralizing Nbs and mAbs for RBD binding. FIG. 52A shows the heatmap showing the binding difference between Nbs and Fabs in terms of paratope residue utility despite overall similar epitope regions. FIG. 52B shows heatmaps showing the difference in preference of epitope-paratope residues between Nbs and Fabs. The comparisons were made separately for RBS binders and non-RBS binders. Nbs with at least 30% overlapping residues with ACE2 binding sites were considered RBS binders. FIG. 52C shows illustrations of dominated electrostatic interactions formed between arginine from Nb CDRs and RBD residues. RBD was colored in dark gray, Nbs were colored in khaki, E484 (RBD) was colored in red, F490 (RBD) was colored in teal and R (Nb CDRs) was colored in blue.

FIG. 53 shows analysis of interactions of E484 (RBD) with neutralizing Nbs and mAbs. Superposition of Fab-RBD structures showing E484 (RBD) forms hydrogen and/or hydrophobic interactions with the respective residues of Fabs. The side chains of residues tyrosine, serine, threonine and arginine in close contact with E484 are shown in stick representation. RBD: dark gray, Fab VH: light blue, Fab LH: light green, and residue E484 (RBD): red.

### DETAILED DESCRIPTION

Described herein is the discovery of novel nanobodies (Nbs) specific for a SARS-CoV-2 spike protein. These nanobodies were elucidated through an application of our integrative proteomic platform for in-depth discovery, classification, and high-throughput structural characterization of antigen-engaged Nb repertoires. A collection of diverse, soluble, stable, and high-affinity camelid Nbs that target the SARS-CoV-2 spike protein receptor-binding domain (RBD) were developed and characterized. The majority of the high-affinity RBD Nbs efficiently neutralize SARS-CoV-2, and in some embodiments, efficiently neutralize another coronavirus. A subset has exceptional neutralization potency, comparable with, or better than the most potent human NAbs. An integrative structural approach was used and multiple epitopes of neutralizing Nbs were mapped. An atomic structure of an elite Nb of sub-ng/ml neutralization potency was determined in complex with the RBD. The results revealed that while highly potent neutralizing Nbs predominantly recognize the concave, hACE2 binding site, efficient neutralization can also be accomplished through other RBD epitopes. Finally, structural characterizations facilitated the bioengineering of multivalent Nbs into multi-epitope cocktails that achieved remarkable neutralization potencies of as low as 0.058 ng/ml (1.3 pM), which can be sufficient to prevent the generation of escape mutants.

### Terminology

As used in the specification and claims, the singular form “a,” “an,” and “the” include plural references unless the context clearly dictates otherwise. For example, the term “a cell” includes a plurality of cells, including mixtures thereof.

The term “about” as used herein when referring to a measurable value such as an amount, a percentage, and the like, is meant to encompass variations of ±20%, ±10%, ±5%, or ±1% from the measurable value.

“Administration” to a subject or “administering” includes any route of introducing or delivering to a subject an agent. Administration can be carried out by any suitable route, including oral, intravenous, intraperitoneal, intranasal, inhalation and the like. Administration includes self-administration and the administration by another.

The terms “antibody” and “antibodies” are used herein in a broad sense and include polyclonal antibodies, monoclonal antibodies, and bi-specific antibodies. In addition to intact immunoglobulin molecules, also included in the term “antibodies” are fragments or polymers of those immunoglobulin molecules, and human or humanized versions of immunoglobulin molecules or fragments thereof. Antibodies are usually heterotetrameric glycoproteins of about 150,000 daltons, composed of two identical light (L) chains and two identical heavy (H) chains. Each heavy chain has at one end a variable domain (V_{H}) followed by a number of constant domains. Each light chain has a variable domain at one end (V_{L}) and a constant domain at its other end.

The antibodies can be tested for their desired activity using the in vitro assays described herein, or by analogous methods, after which, their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The terms “antigenic determinant” and “epitope” may also be used interchangeably herein, referring to the location on the antigen or target recognized by the antigen-binding molecule (such as the nanobodies of the invention). Epitopes can be formed both from contiguous amino acids (a “linear epitope”) or noncontiguous amino acids juxtaposed by tertiary folding of a protein. The latter epitope, one created by at least some noncontiguous amino acids, is described herein as a “conformational epitope.” An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

The terms “antigen binding site”, “binding site” and “binding domain” refer to the specific elements, parts or amino acid residues of a polypeptide, such as a nanobody, that bind the antigenic determinant or epitope.

The terms “CDR” and “complementarity determining region” are used interchangeably and refer to a part of the variable chain of an antibody that participates in binding to an antigen. Accordingly, a CDR is a part of, or is, an “antigen binding site.” In some embodiments, the nanobody comprises three CDR that collectively form an antigen binding site.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a bacterium, a vector, polynucleotide, cells, salts, esters, amides, proagents, active metabolites, isomers, fragments, analogs, and the like. When the terms “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc. In some aspects, the composition disclosed herein comprises a recombinant polypeptide comprising a human serum albumin (HSA) binding polypeptide and an IL-2 polypeptide.

“Effective amount” encompasses, without limitation, an amount that can ameliorate, reverse, mitigate, prevent, or diagnose a symptom or sign of a medical condition or disorder (e.g., cancer). Unless dictated otherwise, explicitly or by context, an “effective amount” is not limited to a minimal amount sufficient to ameliorate a condition. The severity of a disease or disorder, as well as the ability of a treatment to prevent, treat, or mitigate, the disease or disorder can be measured, without implying any limitation, by a biomarker or by a clinical parameter. In some embodiments, the term “effective amount of a recombinant nanobody” refers to an amount of a recombinant nanobody sufficient to prevent, treat, or mitigate a cancer.

The “fragments” or “functional fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the functional fragment must possess a bioactive property, such as binding to HSA and/or ameliorating cancer.

As used herein, a “functional selection step” is a method by which nanobodies are divided into different fractions or groups based upon a functional characteristic. In some embodiments, the functional characteristic is nanobody or CD3 region antigen affinity. In other embodiments, the functional characteristic is nanobody thermostability. In other embodiments, the functional characteristic is nanobody intracellular penetration. Accordingly, the present invention includes a method of identifying a group of complementarity determining region (CDR)3 region nanobody amino acid sequences (CDR3 sequences) wherein a reduced number of the CDR3 sequences are false positives as compared to a control, the method comprising: obtaining a blood sample from a camelid immunized with the antigen; using the blood sample to obtain a nanobody cDNA library; identifying the sequence of each cDNA in the library; isolating nanobodies from the same or a second blood sample from the camelid immunized with the antigen; performing a functional selection step; digesting the nanobodies with trypsin or chymotrypsin to create a group of digestion products; performing a mass spectrometry analysis of the digestion products to obtain mass spectrometry data; selecting sequences identified in step c. that correlate with the mass spectrometry data; identifying sequences of CDR3 regions in the sequences from step g.; and excluding from the CDR3 region sequences from step h. those sequences having less than a calculated fragmentation coverage percentage; wherein the non-excluded sequences comprise a group having the reduced number of false positive CDR3 sequences. It should be understood that the method steps following the functional selection step can be performed separately on each different fraction or group created by the functional selection.

The “half-life” of an amino acid sequence, compound or polypeptide of the invention can generally be defined as the time taken for the serum concentration of the amino acid sequence, compound or polypeptide to be reduced by 50%, in vivo, for example due to degradation of the sequence or compound and/or clearance or sequestration of the sequence or compound by natural mechanisms. The in vivo half-life of a nanobody, amino acid sequence, compound or polypeptide of the invention can be determined in any manner known, such as by pharmacokinetic analysis. these, for example, Kenneth, A et al., Chemical Stability of Pharmaceuticals: A Handbook for Pharmacists; Peters et al., Pharmacokinete analysis: A Practical Approach (1996); “Pharmacokinetics”, M Gibaldi & D Perron, published by Marcel Dekker, 2nd Rev. edition (1982).

The term “identity” or “homology” shall be construed to mean the percentage of nucleotide bases or amino acid residues in the candidate sequence that are identical with the bases or residues of a corresponding sequence to which it is compared, after aligning the sequences and introducing gaps, if necessary to achieve the maximum percent identity for the entire sequence, and not considering any conservative substitutions as part of the sequence identity. A polynucleotide or polynucleotide region (or a polypeptide or polypeptide region) that has a certain percentage (for example, 80%, 85%, 90%, or 95%) of “sequence identity” to another sequence means that, when aligned, that percentage of bases (or amino acids) are the same in comparing the two sequences. In some embodiments, the identity or homology is determined over the entirety of the compared sequences, or in other words, the full length of the sequences are compared. This alignment and the percent homology or sequence identity can be determined using software programs known in the art. Such alignment can be provided using, for instance, the method of Needleman et al. (1970) J. Mol. Biol. 48: 443-453, implemented conveniently by computer programs such as the Align program (DNAstar, Inc.).

As used herein, the terms “nanobody”, “V_{H}H”, “V_{H}H antibody fragment” and “single domain antibody” are used indifferently and designate a variable domain of a single heavy chain of an antibody of the type found in Camelidae, which are without any light chains, such as those derived from Camelids as described in PCT Publication No. WO 94/04678, which is incorporated by reference in its entirety.

As used herein, “operatively linked” refers to the arrangement of polypeptide segments within a single polypeptide chain, where the individual polypeptide segments can be, without limitation, a protein, fragments thereof, linking peptides, and/or signal peptides. The term operatively linked can refer to direct fusion of different individual polypeptides within the single polypeptides or fragments thereof where there are no intervening amino acids between the different segments as well as when the individual polypeptides are connected to one another via a “linker” that comprises one or more intervening amino acids. In some embodiments, the linker is between about 10 and about 40 amino acids. In some embodiments, the linker is between about 15 and about 35 amino acids. In some embodiments, the linker is about 25 amino acids. In some embodiments, the linker is about 31 amino acids. In some embodiments, the linker comprises SEQ ID NO:184 (EGKSSGSGSESKSTGGGGSEGKSSGSGSESKST).

The term “neutralize” refers to a nanbody's ability to reduce infectivity of SARS CoV-2 or another coronavirus. It should be understood that “neutralizing” does not require a 100% neutralization and only requires a partial neutralization. In some embodiments, an about 30%, about 40%, about 50%, about 60%, about 70%, about 80%, or about 90% neutralization is obtained. In some embodiments, infectivity is reduced about 100%, about 90%, about 80%, about 70% or about 60%. “Infectivity” refers to the ability of a virus to bind to and enter a cell. As an example, a nanobody that reduces infectivity by a virus by 100% reduces the virus' entry into a cell by 100% as compared to a control. Accordingly, included herein are embodiments where the nanobody reduces infectivity of SARS CoV-2 or a coronavirus by about 100%, about 99%, about 98%, about 97%, about 96%, about 95%, about 94%, about 93%, about 92%, about 91%, about 90%, about 85%, about 80%, about 75%, about 70%, about 65%, about 60%, about 55%, or about 50% as compared to a control. In some embodiments, the concentration of nanobody required to achieve neutralization of SARS CoV-2 or a coronavirus by about 50% (e.g., an IC50) is less than 1 ng/1 ml.

The term “nucleic acid” as used herein means a polymer composed of nucleotides, e.g. deoxyribonucleotides (DNA) or ribonucleotides (RNA). The terms “ribonucleic acid” and “RNA” as used herein mean a polymer composed of ribonucleotides. The terms “deoxyribonucleic acid” and “DNA” as used herein mean a polymer composed of deoxyribonucleotides.

The terms “pharmaceutically effective amount”, “therapeutically effective amount” or “therapeutically effective dose” refer to the amount of a compound such as a SARS-CoV-2 or coronavirus neutralizing nanobody that will elicit the biological or medical response of a tissue, system, animal, or human that is being sought by the researcher, veterinarian, medical doctor or other clinician. In some embodiments, a desired response is a clinical improvement of, or reduction of an undesired symptom associated with, a SARS-CoV-2 or coronavirus infection. In some embodiments, a desired response is a prevention of a SARS-CoV-2 or coronavirus infection. In some instances, a desired biological or medical response is achieved following administration of multiple dosages of the composition to the subject over a period of days, weeks, or years. The terms “pharmaceutically effective amount”, “therapeutically effective amount” or “therapeutically effective dose” include that amount of a compound such as a SARS-CoV-2 or coronavirus neutralizing nanobody that, when administered, is sufficient to prevent development of, or alleviate to some extent, one or more of the symptoms of the condition or disorder being treated. The therapeutically effective amount will vary depending on the compound such as a selective bacterial β-glucuronidase inhibitor, the disorder or conditions and its severity, the route of administration, time of administration, rate of excretion, drug combination, judgment of the treating physician, dosage form, and the age, weight, general health, sex and/or diet of the subject to be treated. In the context of the present method, a pharmaceutically or therapeutically effective amount or dose of a SARS-CoV-2 or coronavirus neutralizing nanobody includes an amount that is sufficient to reduce one or more of shortness of breath, pneumonia, cough, fatigue, muscle or body aches, headache, loss of taste or smell, sore throat, nausea, vomiting, diarrhea, persistent pain or pressure in the chest, trouble breathing, and death caused by a SARS-CoV-2 or coronavirus infection.

The terms “polynucleotide” and “oligonucleotide” are used interchangeably, and refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

The term “polypeptide” is used in its broadest sense to refer to a compound of two or more subunit amino acids, amino acid analogs, or peptidomimetics. The subunits may be linked by peptide bonds. In another embodiment, the subunit may be linked by other bonds, e.g. ester, ether, etc. As used herein the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein.

“Recombinant” used in reference to a polypeptide refers herein to a combination of two or more polypeptides, which combination is not naturally occurring.

The term “required fragmentation coverage percentage” refers to a percentage obtained using the following formula:
f(x,Enzyme) is the function to calculate fragmentation coverage (%) of peptides digested by Enzyme
x is the length of CDR3 that the peptide mapped

f(x,chymotrypsin)=0.0023x²−0.0497x+0.7723,x[5,30]

f(x,trypsin)=0.00006x²−0.00444x+0.9194,x[5,30]

The terms “specific binding,” “specifically binds,” “selective binding,” and “selectively binds” mean that a nanobody exhibits appreciable affinity for a particular antigen or epitope and, generally, does not exhibit significant cross-reactivity with other non-spike protein receptor binding domain antigens and epitopes. Appreciable binding affinity includes binding with an affinity of at least 10⁶ M⁻¹, specifically at least 10⁷ M⁻¹, more specifically at least 10⁸ M⁻¹, yet more specifically at least 10⁹ M⁻¹, or even yet more specifically at least 10¹⁰ M⁻¹. A binding affinity can also be indicated as a range of affinities, for example, 10⁶ M⁻¹ to 10¹⁰ M⁻¹, specifically 10⁷ M⁻¹ to 10¹⁰ M⁻¹, more specifically 10⁸ M⁻¹ to 10¹⁰ M⁻¹. A nanobody that “does not exhibit significant cross reactivity” is one that will not appreciably bind to an undesirable entity (e.g., an undesirable proteinaceous entity such as a non-spike protein receptor binding domain) A nanobody specific for a particular epitope will, for example, not significantly cross react with other epitopes on the same protein or peptide. Specific binding can be determined according to any art-recognized means for determining Such binding. In some embodiments, specific binding is determined according to Scatchard analysis and/or competitive binding assays.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

The terms “treat,” “treating,” “treatment,” and grammatical variations thereof as used herein, include partially or completely delaying, alleviating, mitigating or reducing the intensity of one or more attendant symptoms of a SARS-CoV-2 or coronavirus infection and/or alleviating, mitigating or impeding one or more causes of a SARS-CoV-2 or coronavirus infection. In some instances, the terms “treat”, “treating”, “treatment” and grammatical variations thereof, refer to reducing detectable SARS-CoV-2 or coronavirus in the subject. In some instances, the terms “treat”, “treating”, “treatment” and grammatical variations thereof, refer to achieving a negative test result for SARS-CoV-2 or coronavirus in the subject. In some instances, the terms “treat”, “treating”, “treatment” and grammatical variations thereof, refer to reducing SARS-CoV-2 or coronavirus viral load in the subject. In some instances, the terms “treat”, “treating”, “treatment” and grammatical variations thereof, refer to reducing one or more of shortness of breath, pneumonia, cough, fatigue, muscle or body aches, headache, loss of taste or smell, sore throat, nausea, vomiting, diarrhea, persistent pain or pressure in the chest, trouble breathing and death caused by a SARS-CoV-2 or coronavirus infection.

### Compositions and Methods

Provided herein is the development and characterization of a large collection of diverse, high-affinity camelid Nbs that target the S1-receptor binding domain (RBD) of a SARS-CoV-2 spike protein or a coronavirus spike protein. In particular, the coronavirus Nbs (e.g., SARS-CoV-2 Nbs) described herein are capable of neutralizing coronavirus (e.g., SARS-CoV-2) infectivity in a cell culture system. Therefore, the coronavirus Nbs (e.g., SARS-CoV-2 Nbs) of the present invention are useful for treating or preventing a coronavirus infection (e.g., SARS-COV-2 infection), and included herein are methods of treating a coronavirus infection (e.g., SARS-COV-2 infection) in a subject comprising administering to the subject a therapeutically effective amount of a neutralizing nanobody described herein. Also included herein are methods of preventing a coronavirus infection (e.g., SARS-COV-2 infection) in a subject comprising administering to the subject a therapeutically effective amount of a neutralizing nanobody described herein.

The present invention includes SARS-CoV-2 neutralizing nanobodies that comprise an amino acid sequence selected from the group consisting of SEQ ID NO:1 through SEQ ID NO:152. In some embodiments, the SARS-CoV-2 nanobody comprises a sequence that has about 80%, about 85%, about 90%, about 95%, about 96%, about 97%, about 98%, or about 99% homology with an amino acid sequence selected from the group consisting of SEQ ID NO:1 through SEQ ID NO:152, SEQ ID NO: 185, and SEQ ID NO: 186. In some embodiments, the nanobody comprises a sequence that has about 80%, about 85%, about 90%, about 95%, about 96%, about 97%, about 98%, or about 99% homology with an amino acid sequence selected from the group consisting of SEQ ID NO: 95, SEQ ID NO: 96, SEQ ID NO: 103, SEQ ID NO: 140, SEQ ID NO: 147, SEQ ID NO: 93, SEQ ID NO: 104, or SEQ ID NO: 185. In some embodiments, the nanobody comprises the sequence selected from the group consisting of SEQ ID NO: 95, SEQ ID NO: 96, SEQ ID NO: 103, SEQ ID NO: 140, SEQ ID NO: 147, SEQ ID NO: 93, SEQ ID NO: 104, or SEQ ID NO: 185. In some embodiments, the nanobody comprises a sequence that has about 80%, about 85%, about 90%, about 95%, about 96%, about 97%, about 98%, or about 99% homology with an amino acid sequence selected from the group consisting of SEQ ID NO: 14, SEQ ID NO: 15, SEQ ID NO: 22, SEQ ID NO: 59, SEQ ID NO: 66, SEQ ID NO: 12, and SEQ ID NO: 23. In some embodiments, the nanobody comprises the sequence selected from the group consisting of SEQ ID NO: 14, SEQ ID NO: 15, SEQ ID NO: 22, SEQ ID NO: 59, SEQ ID NO: 66, SEQ ID NO: 12, and SEQ ID NO: 23.

In some embodiments, the nanobody comprises a multimer (e.g., homodimer or homotrimer) of an amino acid sequence that has about 80%, about 85%, about 90%, about 95%, about 96%, about 97%, about 98%, or about 99% homology with an amino acid sequence selected from the group consisting of SEQ ID NO: 95, SEQ ID NO: 96, SEQ ID NO: 103, SEQ ID NO: 140, SEQ ID NO: 147, SEQ ID NO: 93, SEQ ID NO: 104, and SEQ ID NO: 185. In some embodiments, the nanobody comprises a multimer (e.g., homodimer or homotrimer) of an amino acid sequence that has about 80%, about 85%, about 90%, about 95%, about 96%, about 97%, about 98%, or about 99% homology with an amino acid sequence selected from the group consisting of SEQ ID NO: 14, SEQ ID NO: 15, SEQ ID NO: 22, SEQ ID NO: 59, SEQ ID NO: 66, SEQ ID NO: 12, and SEQ ID NO: 23.

In some embodiments, the nanobody comprises a multimer (e.g., homodimer or homotrimer) of the amino acid sequence set forth in SEQ ID NO: 95, SEQ ID NO: 96, SEQ ID NO: 103, SEQ ID NO: 140, SEQ ID NO: 147, SEQ ID NO: 93, SEQ ID NO: 104, or SEQ ID NO: 185. In some embodiments, the nanobody comprises a multimer (e.g., homodimer or homotrimer) of the amino acid sequence set forth in SEQ ID NO: 95. In some embodiments, the nanobody comprises a multimer (e.g., homodimer or homotrimer) of the amino acid sequence set forth in SEQ ID NO: 96. In some embodiments, the nanobody comprises a multimer (e.g., homodimer or homotrimer) of the amino acid sequence set forth in SEQ ID NO: 103. In some embodiments, the nanobody comprises a multimer (e.g., homodimer or homotrimer) of the amino acid sequence set forth in SEQ ID NO: 140. In some embodiments, the nanobody comprises a multimer (e.g., homodimer or homotrimer) of the amino acid sequence set forth in SEQ ID NO: 147. In some embodiments, the nanobody comprises a multimer (e.g., homodimer or homotrimer) of the amino acid sequence set forth in SEQ ID NO: 93. In some embodiments, the nanobody comprises a multimer (e.g., homodimer or homotrimer) of the amino acid sequence set forth in SEQ ID NO: 104. In some embodiments, the nanobody comprises a multimer (e.g., homodimer or homotrimer) of the amino acid sequence set forth in SEQ ID NO: 185.

In some embodiments, the nanobody comprises a multimer of one or more amino acid sequences comprising a sequence selected from the group consisting of SEQ ID NO:82 through SEQ ID NO:152 and SEQ ID NO: 185. In some embodiments, the nanobody comprises a multimer (e.g., homodimer or homotrimer) of the amino acid sequence set forth in SEQ ID NO: 95, SEQ ID NO: 96, SEQ ID NO: 103, SEQ ID NO: 140, SEQ ID NO: 147, SEQ ID NO: 93, SEQ ID NO: 104, or SEQ ID NO: 185. In some embodiments, the nanobody comprises a multimer (e.g., homodimer or homotrimer) of the amino acid sequence set forth in SEQ ID NO: 95. In some embodiments, the nanobody comprises a multimer (e.g., homodimer or homotrimer) of the amino acid sequence set forth in SEQ ID NO: 96. In some embodiments, the nanobody comprises a multimer (e.g., homodimer or homotrimer) of the amino acid sequence set forth in SEQ ID NO: 103. In some embodiments, the nanobody comprises a multimer (e.g., homodimer or homotrimer) of the amino acid sequence set forth in SEQ ID NO: 140. In some embodiments, the nanobody comprises a multimer (e.g., homodimer or homotrimer) of the amino acid sequence set forth in SEQ ID NO: 147. In some embodiments, the nanobody comprises a multimer (e.g., homodimer or homotrimer) of the amino acid sequence set forth in SEQ ID NO: 93. In some embodiments, the nanobody comprises a multimer (e.g., homodimer or homotrimer) of the amino acid sequence set forth in SEQ ID NO: 104. In some embodiments, the nanobody comprises a multimer (e.g., homodimer or homotrimer) of the amino acid sequence set forth in SEQ ID NO: 185. In some embodiments, the SARS-CoV-2 nanobody comprises a sequence that has about 80%, about 85%, about 90%, about 95%, about 96%, about 97%, about 98%, or about 99% homology with an amino acid sequence selected from the group consisting of SEQ ID NO:1 through SEQ ID NO:71. In some embodiments, the nanobody comprises a multimer of one or more amino acid sequences comprising a sequence selected from the group consisting of SEQ ID NO:1 through SEQ ID NO:71, wherein each of the sequences is separated by a linker sequence. In some embodiments, the nanobody comprises a multimer of one or more amino acid sequences comprising a sequence selected from the group consisting of SEQ ID NO: 14, SEQ ID NO: 15, SEQ ID NO: 22, SEQ ID NO: 59, SEQ ID NO: 66, SEQ ID NO: 12, and SEQ ID NO: 23. In some embodiments, the nanobody comprises a multimer of one or more amino acid sequences comprising a sequence set forth in SEQ ID NO: 14. In some embodiments, the nanobody comprises a multimer of one or more amino acid sequences comprising a sequence set forth in SEQ ID NO: 15. In some embodiments, the nanobody comprises a multimer of one or more amino acid sequences comprising a sequence set forth in SEQ ID NO: 22. In some embodiments, the nanobody comprises a multimer of one or more amino acid sequences comprising a sequence set forth in SEQ ID NO: 59. In some embodiments, the nanobody comprises a multimer of one or more amino acid sequences comprising a sequence set forth in SEQ ID NO: 66. In some embodiments, the nanobody comprises a multimer of one or more amino acid sequences comprising a sequence set forth in SEQ ID NO: 12. In some embodiments, the nanobody comprises a multimer of one or more amino acid sequences comprising a sequence set forth in SEQ ID NO: 23. In some embodiments, the multimer is a dimer or a trimer. The multimer can be, for example, a homodimer, homotrimer, heterodimer or heterotrimer.

In some embodiments, the linker is between about 10 and about 40 amino acids. In some embodiments, the linker is between about 15 and about 35 amino acids. In some embodiments, the linker is about 25 amino acids. In some embodiments, the linker is about 31 amino acids. In some embodiments, the linker comprises SEQ ID NO:184 (EGKSSGSGSESKSTGGGGSEGKSSGSGSESKST) or a fragment thereof.

Accordingly, included herein is a homotrimer nanobody comprising three copies of one amino acid sequence, wherein the one amino acid sequence comprises a sequence selected from the group consisting of SEQ ID NO:1 through SEQ ID NO:71 or a fragment thereof, and wherein the three copies are separated by linker sequences. Also included is a heterotrimer nanobody comprising three different amino acid sequences, wherein each different amino acid sequence comprises a sequence selected from the group consisting of SEQ ID NO:1 through SEQ ID NO:71 or a fragment thereof, and wherein the different amino acid sequences are separated by linker sequences. Also included is a homodimer nanobody comprising two copies of one amino acid sequence, wherein the one amino acid sequence comprises a sequence selected from the group consisting of SEQ ID NO:1 through SEQ ID NO:71 or a fragment thereof, and wherein the two copies are separated by linker sequences. Further included is a heterodimer nanobody comprising two different amino acid sequences, wherein each different amino acid sequence comprises a sequence selected from the group consisting of SEQ ID NO:1 through SEQ ID NO:71 or a fragment thereof, and wherein the different amino acid sequences are separated by linker sequences.

In some embodiments, the SARS-CoV-2 neutralizing nanobody comprises a sequence of SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, or SEQ ID NO: 186 or fragment thereof. In some embodiments, the SARS-CoV-2 nanobody comprises a sequence that has about 80%, about 85%, about 90%, about 95%, about 96%, about 97%, about 98%, or about 99% homology with an amino acid sequence selected from the group consisting of SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, and SEQ ID NO: 186.

In some embodiments, the SARS-CoV-2 neutralizing nanobody is conjugated or linked to a nanobody, or nanobody fragment, that specifically binds to human serum albumin for the purpose of increasing the half-life of the SARS-CoV-2 neutralizing nanobody.

In some embodiments, the SARS-CoV-2 neutralizing nanobody reduces infectivity of SARS-CoV-2 by about 100%, about 90%, about 80%, about 70%, about 60%, or about 50%. In some embodiments the SARS-CoV-2 Nb reduces infectivity of SARS CoV-2 by about 100%, about 99%, about 98%, about 97%, about 96%, about 95%, about 94%, about 93%, about 92%, about 91%, about 90%, about 85%, about 80%, about 75%, about 70%, about 65%, about 60%, about 55%, or about 50%. Reduced infectivity can be determined using any method, including a cell culture system used to determine viral neutralization. In some embodiments, the concentration of nanobody required to achieve a reduced infectivity of SARS CoV-2 by about 50% (e.g., an IC50) is less than 1 ng/1 ml.

In some embodiments, the SARS-CoV-2 neutralizing nanobody binds specifically to the concave, hACE2 binding sites. In some embodiments, the binding affinity is a femtomolar binding.

As noted above, also included herein are methods of treating and/or preventing a SARS-CoV-2 infection in a subject comprising administering to the subject a therapeutically effective amount of a SARS-CoV-2 neutralizing nanobody disclosed herein. In some embodiments of the methods, the SARS-CoV-2 neutralizing nanobody comprises an amino acid sequence selected from the group consisting of SEQ ID NO:1 through SEQ ID NO:152. In some embodiments of the methods, the SARS-CoV-2 neutralizing nanobody comprises two or more amino acid sequences selected from the group consisting of SEQ ID NO:1 through SEQ ID NO:152, SEQ ID NO: 185, and SEQ ID NO: 186.

In some embodiments, the nanobodies disclosed herein comprise paratopes that specifically bind to epitopes on a viral protein (e.g., a SARS-CoV-2 spike protein). In some embodiments, the nanobodies specifically bind to a SARS-CoV-2 spike protein (SEQ ID NO: 189). In some embodiments, the nanobodies specifically bind to the receptor binding domain (RBD) of a SARS-CoV-2 spike protein, wherein the RBD comprises an amino acid sequence of residue numbers 334-527 of SEQ ID NO: 189. The term “epitope”, also known as antigenic determinant, refers to the part of an antigen that is recognized by the immune system (e.g., antibodies). The part of an antibody that binds to the epitope is referred herein as a “paratope”. Antibody-antigen interactions occur between the sequence regions on the antibody (paratope) and the antigen (epitope) at the binding interface. Consequently, paratopes and epitopes can manifest in two ways: (1) as a continuous stretch of interacting residues or (2) discontinuously, separated by one or more non-interacting residues (gaps) due to protein folding.

In some embodiments, the nanobody comprises an amino acid sequence having the same amino acids residues at positions 28, 30, 31, 32, 33, 34, 35, 37, 47, 49, 50, 51, 52, 53, 54, 55, 56, 57, 58, 59, 71, 73, 74, 94, 95, 96, 97, 98, 99, 100, 101, 102, 104, 105, 106, 107, and 109 relative to SEQ ID NO: 12, and wherein the nanobody specifically binds to amino acids at positions 345, 346, 347, 348, 349, 351, 352, 353, 354, 355, 356, 399, 448, 449, 450, 451, 452, 453, 454, 455, 466, 467, 468, 469, 470, 471, 472, 482, 483, 484, 489, 490, 491, 492, 493, 493, and 494 of SEQ ID NO: 189.

In some embodiments, the nanobody comprises an amino acid sequence having the same amino acids residues at positions 44, 45, 46, 47, 57, 58, 59, 60, 62, 65, 99, 100, 101, 102, 103, 104, 105, 106, 107, 108, 109, 110, 111, 112, and 113 relative to SEQ ID NO: 66, and wherein the nanobody specifically binds to amino acids at positions 369, 371, 372, 373, 374, 375, 376, 377, 378, 379, 380, 381, 382, 383, 384, 403, 404, 405, 407, 408, 411, 412, 414, 432, 435, 501, 502, 503, 504, 505, 508, and 510 of SEQ ID NO: 189.

In some embodiments, the nanobody comprises an amino acid sequence having the same amino acids residues at positions 53, 60, 100, 101, 102, 103, 104, 105, 106, 107, 108, 109, 110, 111, 112, 113, and 114 relative to SEQ ID NO: 59, and wherein the nanobody specifically binds to amino acids at positions 368, 369, 370, 371, 372, 374, 375, 376, 377, 378, 379, 380, 381, 382, 383, 384, 404, 405, 406, 407, 408, 409, 414, 435, 436, and 508 of SEQ ID NO: 189.

In some embodiments, the nanobody comprises an amino acid sequence having the same amino acids residues at positions 38, 43, 44, 45, 46, 47, 48, 59, 60, 61, 62, 63, 65, 102, 103, 109, 110, 111, 112, 113, and 116 relative to SEQ ID NO: 22, and wherein the nanobody specifically binds to amino acids at positions 366, 369, 370, 371, 372, 373, 374, 375, 376, 377, 378, 379, 380, 381, 382, 383, 384, 385, 412, 436, and 437 of SEQ ID NO: 189.

In some embodiments, the nanobody comprises an amino acid sequence having the same amino acids residues at positions 28, 33, 39, 40, 104, 105, 106, 107, 108, 109, 110, 111, 113, 114, 115, 116, and 118 relative to SEQ ID NO: 23, and wherein the nanobody specifically binds to amino acids at positions 344, 345, 346, 347, 348, 349, 351, 352, 353, 354, 355, 356, 357, 396, 451, 457, 464, 465, 466, 467, 468, and 470 of SEQ ID NO: 189.

In some embodiments, the nanobody comprises an amino acid sequence having the same amino acids residues at positions 27, 28, 29, 30, 31, 33, 35, 44, 45, 46, 47, 48, 50, 51, 52, 55, 56, 57, 58, 59, 70, 72, 97, 98, 99, 100, 101, 102, 103, and 104 relative to SEQ ID NO: 15, and wherein the nanobody specifically binds to amino acids at positions 351, 446, 447, 448, 449, 450, 451, 452, 453, 455, 456, 470, 472, 482, 483, 484, 485, 486, 487, 488, 489, 490, 491, 492, 493, 494, 495, 496, 497, 498, 505, and 531 of SEQ ID NO: 189.

In some embodiments, the nanobody comprises an amino acid sequence having the same amino acids residues at positions 27, 28, 29, 30, 31, 32, 33, 35, 45, 47, 48, 49, 51, 52, 55, 56, 57, 58, 59, 60, 72, 97, 98, 99, 100, 102, 103, 104 relative to SEQ ID NO: 14, and wherein the nanobody specifically binds to amino acids at positions 351, 417, 449, 450, 451, 452, 453, 455, 456, 470, 472, 481, 482, 483, 484, 485, 486, 487, 488, 489, 490, 491, 492, 493, 494, 495, and 496 of SEQ ID NO: 189.

It should be understood that in some embodiments, the SARS-CoV-2 nanobodies disclosed herein can cross-react with other coronavirus spike proteins. Accordingly, the invention includes treatment of coronaviruses other than SARS-CoV-2 with the nanobodies disclosed herein. Coronaviruses constitute the subfamily Orthocoronavirinae, in the family Coronaviridae, order Nidovirales, and realm Riboviria. They are enveloped viruses with a positive-sense single-stranded RNA genome and a nucleocapsid of helical symmetry. The genome size of coronaviruses ranges from approximately 27 to 34 kilobases. The structure of coronavirus generally consists of the following: spike protein, hemagglutinin-esterease dimer (HE), a membrane glycoprotein (M), an envelope protein (E) a nucleoclapid protein (N) and RNA. The coronavirus family comprises genera including, for example, alphacoronavius (e.g., Human coronavirus 229E, Human coronavirus NL63, Miniopterus bat coronavirus 1, Miniopterus bat coronavirus HKU8, Porcine epidemic diarrhea virus, Rhinolophus bat coronavirus HKU2, Scotophilus bat coronavirus 512), betacoronavirus (e.g., SARS-CoV-2, Betacoronavirus 1, Human coronavirus HKU1, Murine coronavirus, Pipistrellus bat coronavirus HKU5, Rousettus bat coronavirus HKU9, Severe acute respiratory syndrome-related coronavirus, Tylonycteris bat coronavirus HKU4, Middle East respiratory syndrome-related coronavirus (MERS), Human coronavirus OC43, Hedgehog coronavirus 1 (EriCoV)), gammacoronavirus (e.g., Beluga whale coronavirus SW1, Infectious bronchitis virus), and deltacoronavirus (e.g., Bulbul coronavirus HKU11, Porcine coronavirus HKU15). In some embodiments, the nanobodies disclosed herein can cross-react with other coronaviruses or other coronavirus spike proteins. In some embodiments, the nanobody cross reacts with Ratq13, panq17, SARS-CoV, WIVI, SHC014, Rs4081, RmYNo2, RF1, Yun11, BtKy72, BM4831. Accordingly, in some aspects, disclosed herein are nanobodies and uses thereof for treating and/or preventing an infection with a coronavirus. In some embodiments, the coronavirus is SARS-CoV. In some embodiments, the coronavirus is MERS-CoV.

In some embodiments, the nanobody is administered at a dose of about 0.01 mg/kg of body weight, about 0.05 mg/kg of body weight, about 0.1 mg/kg of body weight, about 0.15 mg/kg of body weight, about 0.2 mg/kg of body weight, about 0.25 mg/kg of body weight, about 0.3 mg/kg of body weight, about 0.35 mg/kg of body weight, about 0.4 mg/kg of body weight, about 0.45 mg/kg of body weight, about 0.5 mg/kg of body weight, about 0.55 mg/kg of body weight, about 0.6 mg/kg of body weight, about 0.65 mg/kg of body weight, about 0.7 mg/kg of body weight, about 0.75 mg/kg of body weight, about 0.8 mg/kg of body weight, about 0.85 mg/kg of body weight, about 0.9 mg/kg of body weight, about 0.95 mg/kg of body weight, about 1 mg/kg of body weight, about 2 mg/kg of body weight, about 3 mg/kg of body weight, about 4 mg/kg of body weight, about 5 mg/kg of body weight, about 6 mg/kg of body weight, about 7 mg/kg of body weight, about 8 mg/kg of body weight, about 9 mg/kg of body weight, about 10 mg/kg of body weight, or about 20 mg/kg of body weight. In some embodiments, the nanobody is administered at a dose of at least about 0.01 mg/kg of body weight (e.g., at least about 0.1 mg/kg of body weight, at least about 0.2 mg/kg of body weight, at least about 0.3 mg/kg of body weight, at least about 0.5 mg/kg of body weight, at least about 1.0 mg/kg of body weight, at least about 2 mg/kg of body weight, at least about 5 mg/kg of body weight, at least about 10 mg/kg of body weight, at least about 50 mg/kg of body weight, or at least about 100 mg/kg of body weight).

The disclosed methods can be employed 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 years; 12, 11, 10, 9, 8, 7, 6, 5, 4, 3, 2, or 1 months; 30, 29, 28, 27, 26, 25, 24, 23, 22, 21, 20, 19, 18, 17, 16, 15, 14, 13, 12, 11, 10, 9, 8, 7, 6, 5, 4, or 3 days; 60, 48, 36, 30, 24, 18, 15, 12, 10, 9, 8, 7, 6, 5, 4, 3, or 2 hours prior to the onset of a COVID-19 symptom; or 1, 2, 3, 4, 5, 6, 7, 8, 9, 10, 15, 20, 25, 30, 35, 40, 45, 50, 55, 60, 75, 90, 105, 120 minutes; 3, 4, 5, 6, 7, 8, 9, 10, 11, 12, 15, 18, 24, 30, 36, 48, 60 hours; 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 or more days; 4, 5, 6, 7, 8, 9, 10, 11, 12 or more months; 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1 years after the onset of COVID-19 symptom. In some embodiments, the disclosed methods can be employed prior to or following the administering of another anti-SARS-CoV-2 agent.

A coronavirus or SARS-CoV-2 neutralizing nanobody described herein can be administered to the subject via any route including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. In some embodiments, the nanobody is administered intratracheally, intranasally, or through an inhalation route. In some embodiments, the coronavirus or SARS-COV2 neutralizing nanobody is in an aerosol form. The term “parenteral” includes subcutaneous, intravenous, intramuscular, intra-articular, intra-synovial, intrasternal, intrathecal, intrahepatic, intralesional, and intracranial injections or infusion techniques.

Dosing frequency for a coronavirus or SARS-CoV-2 neutralizing nanobody of any preceding aspects, includes, but is not limited to, at least once every year, once every two years, once every three years, once every four years, once every five years, once every six years, once every seven years, once every eight years, once every nine years, once every ten year, at least once every two months, once every three months, once every four months, once every five months, once every six months, once every seven months, once every eight months, once every nine months, once every ten months, once every eleven months, at least once every month, once every three weeks, once every two weeks, once a week, twice a week, three times a week, four times a week, five times a week, six times a week, daily, twice a day, three times a day, four times a day, or five times a day. Administration can also be continuous and adjusted to maintaining a level of the compound within any desired and specified range.

### EXAMPLES

### Example 1. Versatile, Multivalent Nanobody Cocktails Efficiently Neutralize SARS-CoV-2

A collection of diverse, soluble, stable, and high-affinity camelid Nbs that target the RBD were developed and characterized. The majority of the high-affinity RBD Nbs efficiently neutralize SARS-CoV-2. A subset has exceptional neutralization potency, comparable with, or better than the most potent human NAbs. Integrative structural approach was used and multiple epitopes of neutralizing Nbs were mapped. An atomic structure of an elite Nb of sub-ng/ml neutralization potency was determined in complex with the RBD. The results revealed that while highly potent neutralizing Nbs predominantly recognize the concave, hACE2 binding site, efficient neutralization can also be accomplished through other RBD epitopes. Finally, structural characterizations facilitated the bioengineering of multivalent Nbs into multi-epitope cocktails that achieved remarkable neutralization potencies of as low as 0.058 ng/ml (1.3 pM), which can be sufficient to prevent the generation of escape mutants

### Development of Highly Potent SARS-COV-2 Neutralizing Nbs

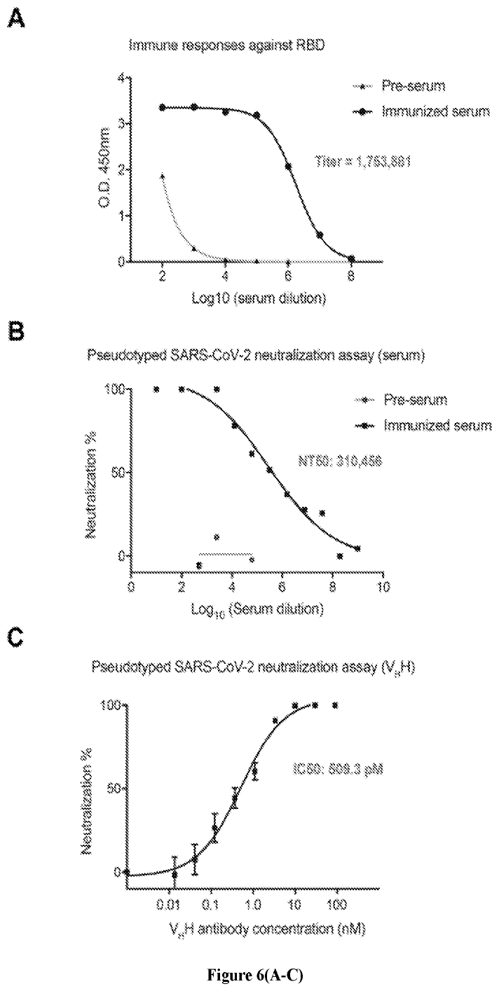

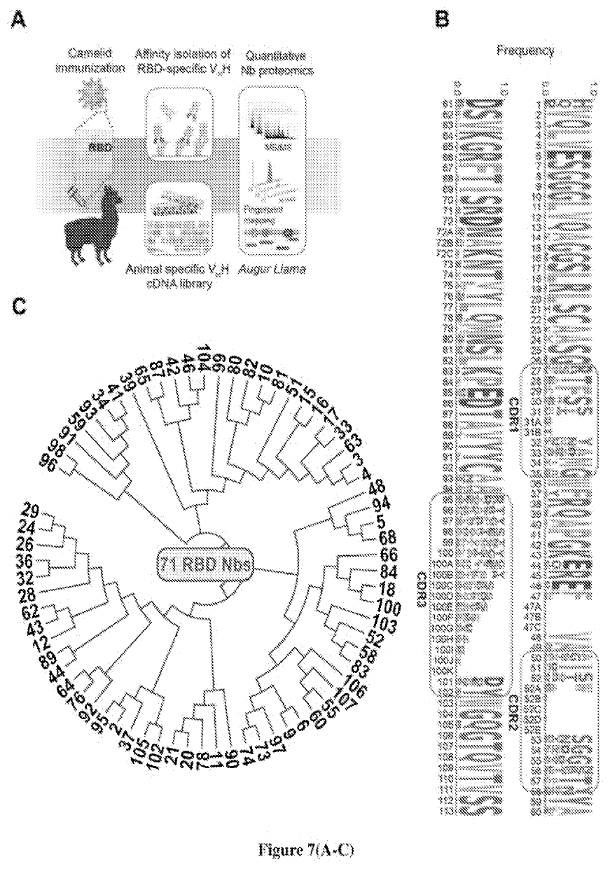

To produce high-quality SARS-CoV-2 neutralizing Nbs, a llama was immunized with the recombinant RBD protein expressed in human 293T cells. Compared to the pre-bleed, after affinity maturation, the post-immunized serum showed potent and specific serologic activities towards RBD binding with a titer of 1.75×10⁶ (FIG. 6A). The serum efficiently neutralized the pseudotyped SARS-CoV-2 at the half-maximal neutralization titer (NT50) of 310,000 (FIG. 6B), orders of magnitude higher than the convalescent sera obtained from recovered COVID-19 patients (Y. Cao et al., 2020; D. F. Robbiani et al., 2020). To further characterize these activities, the single-chain V_{H}H antibodies were separated from the IgG antibodies from the serum. It was confirmed that the single-chain antibodies achieve specific, high-affinity binding to the RBD and possess sub-nM half-maximal inhibitory concentration (IC50=509 pM) against the pseudotyped virus (FIG. 6C).

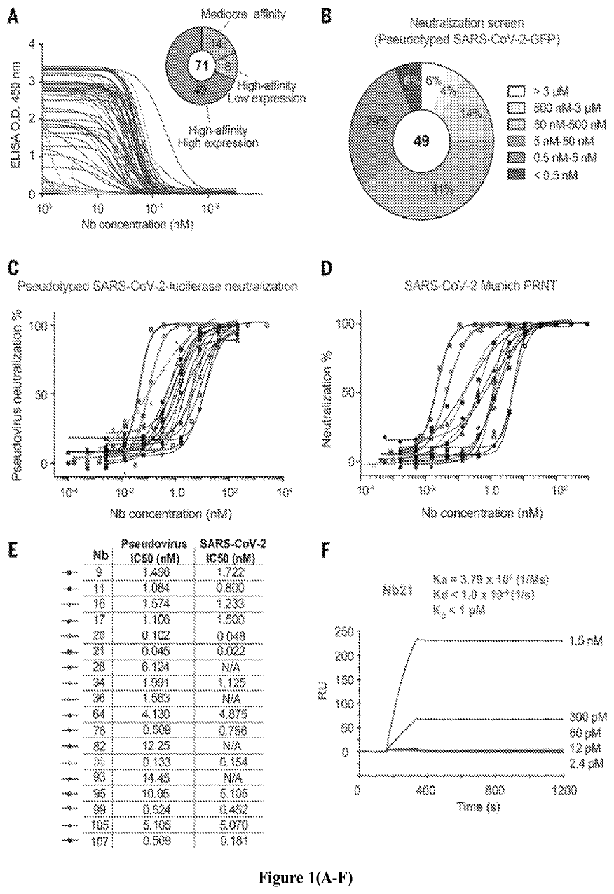

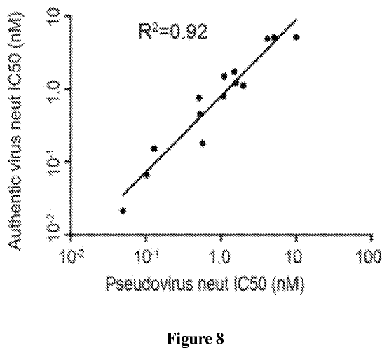

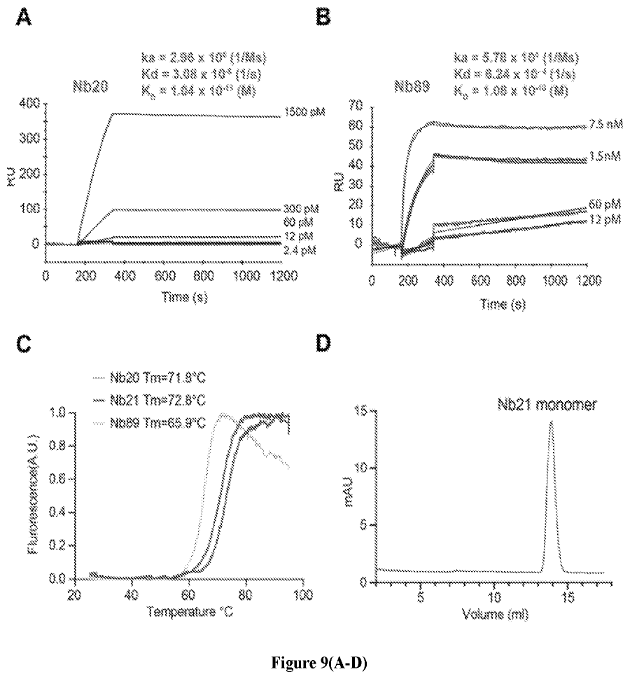

Thousands of high-affinity V_{H}H Nbs from the RBD-immunized llama serum were identified using a robust proteomic strategy (Y. Xiang et al., 2020) (FIG. 7a). This repertoire includes ˜350 unique CDR3s (complementarity-determining regions). For E. coli expression, 109 highly diverse Nb sequences were selected from the repertoire with unique CDR3s to cover various biophysical, structural, and different antiviral properties of the Nb repertoire. Ninety four Nbs were purified and tested for RBD binding by ELISA, from which 71 RBD-specific binders were confirmed (FIG. 7b-7c, Table 1). Of these RBD-specific binders, 49 Nbs presented high solubility and high-affinity (ELISA IC50 below 30 nM, FIG. 1a), and were candidates for functional characterizations. A SARS-CoV-2-GFP pseudovirus neutralization assay was used to screen and characterize the antiviral activities of these high-affinity Nbs. The vast majority (94%) of the tested Nbs can neutralize the pseudotype virus below 3 μM (FIG. 1b). 90% of them blocked the pseudovirus below 500 nM. Only 20-40% of high-affinity RBD-specific mAbs identified from the patients' sera have been reported to possess comparable potency (Y. Cao et al., 2020; D. F. Robbiani et al., 2020). Over three quarters (76%) of the Nbs efficiently neutralized the pseudovirus below 50 nM, and 6% had neutralization activities below 0.5 nM. Finally, the potential of 14 to neutralize SARS-CoV-2 Munich strain was tested using the PRNT50 assay (W. B. Klimstra et al., 2020). All the Nbs reached 100% neutralization and neutralized the virus in a dose-dependent manner. The IC50s span from single-digit ng/ml to sub-ng/ml, with three unusual neutralizers of Nbs 89, 20, and 21 to be 2.0 ng/ml (0.129 nM), 1.6 ng/ml (0.102 nM), and 0.7 ng/ml (0.045 nM), respectively, based on the pseudovirus assay (FIG. 1c, FIG. 1e). Similar values (0.154 nM, 0.048 nM, and 0.021 nM, for Nbs 89, 20, and 21) were reproducibly obtained using SARS-CoV-2 (FIG. 1d, FIG. 1e). There was an excellent correlation between the two neutralization assays (R 2=0.92, FIG. 8).

The binding kinetics of Nbs 89, 20, and 21 were measured by surface plasmon resonance (SPR) (FIG. 9a-9b). While Nbs 89 and 20 have an affinity of 108 pM and 10.4 pM, the best-neutralizing Nb21 did not show detectable dissociation from the RBD during 20 min SPR analysis. The femtomolar affinity of Nb21 potentially explains its unusual neutralization potency (FIG. 1f). The experiment determined thermostability of the top three neutralizing Nbs (89, 20, and 21) from the E. coli periplasmic preparations to be 65.9, 71.8, and 72.8° C., respectively (FIG. 9c). Finally, a the on-shelf stability of Nb21, which remained soluble after ˜6 weeks of storage at room temperature after purification and can well tolerate lyophilization. No multimeric forms or aggregations were detected by size-exclusion chromatography (SEC) (FIG. 9d). Together these results show that these neutralizing Nbs have the necessary physicochemical properties required for advanced therapeutic applications.

### Integrative Structural Characterization of the Nb Neutralization Epitopes

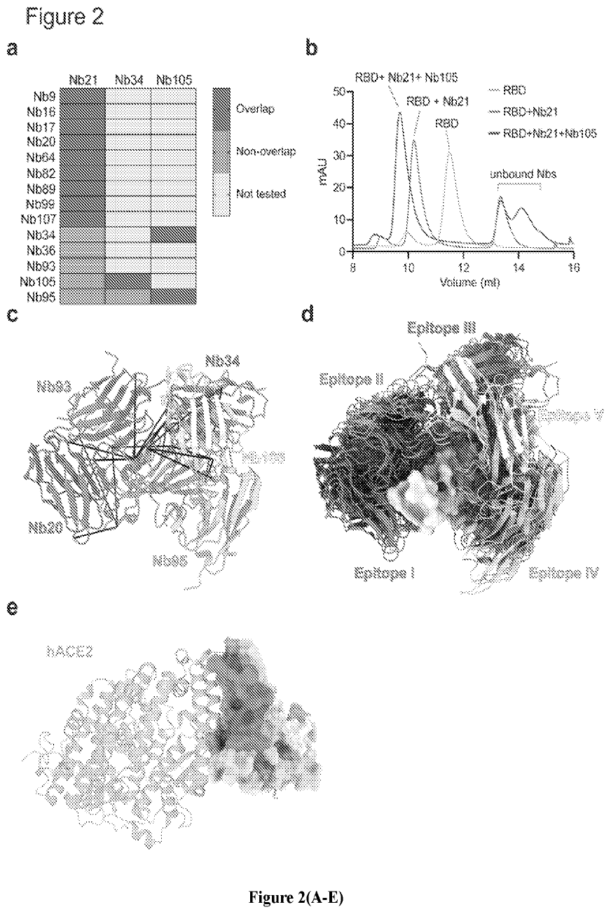

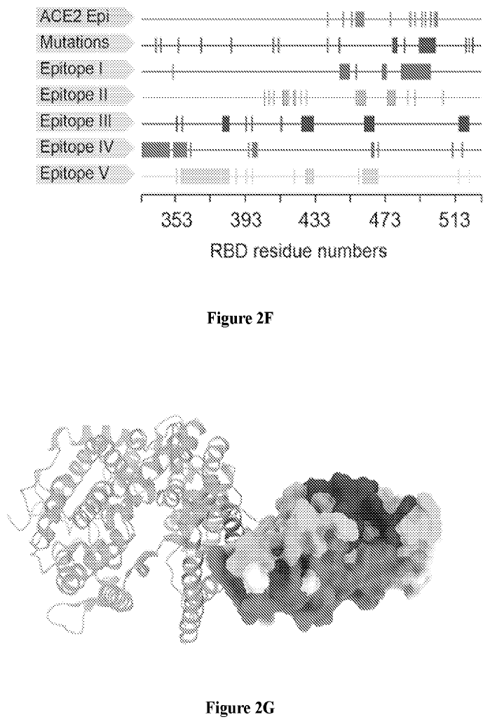

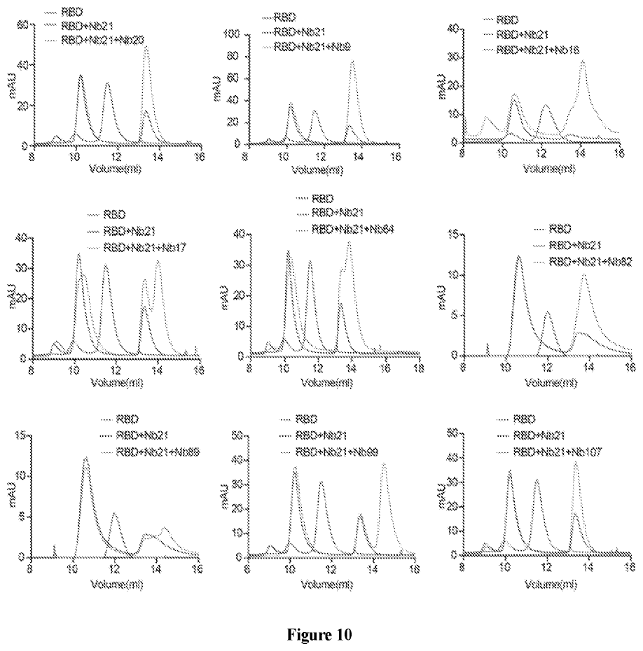

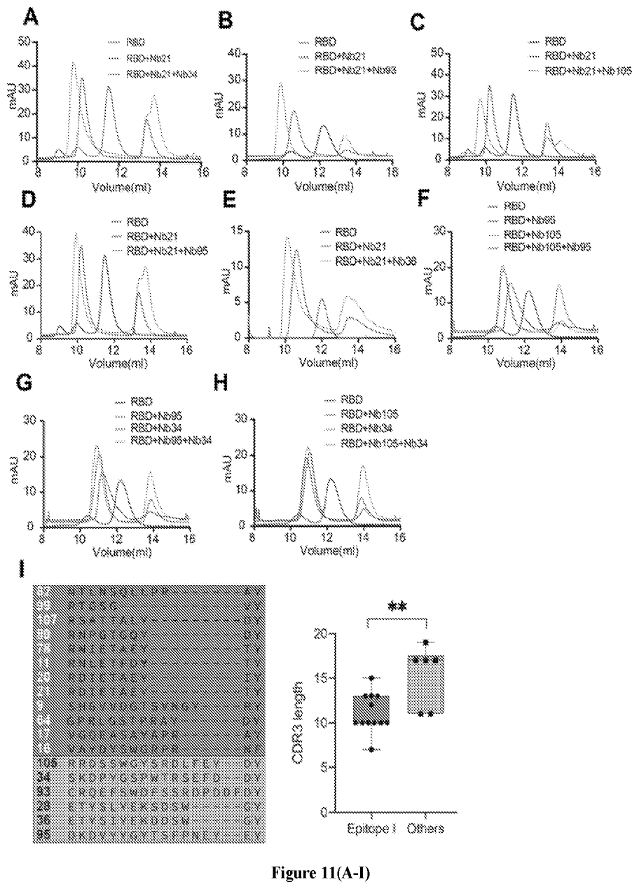

Epitope mapping based on atomic resolution structure determination by X-ray crystallography and Cryo-Electron Microscopy (CryoEM) is highly accurate but low-throughput. Here, information from SEC, cross-linking mass spectrometry (CXMS), shape and physicochemical complementarity, and statistics was integrated to determine structural models of RBD-Nb complexes (M. P. Rout, A. Sali, 2019; C. Yu et al., 2017; A. Leitner, M. Faini, F. 2016; B. T. Chait et. al., 2016). First, SEC experiments were performed to distinguish between Nbs that share the same epitope as Nb21 (thus complete with Nb 21 on an SEC) and those that bind to non-overlapping epitopes. Nbs 9, 16, 17, 20, 64, 82, 89, 99 and 107 competed with Nb21 for RBD binding based on SEC profiles (FIG. 2a, FIG. 10), indicating that their epitopes significantly overlap. In contrast, higher mass species (from early elution volumes) corresponding to the trimeric complexes composed of Nb21, RBD, and one of the Nbs (34, 36, 93, 105, and 95) were evident (FIGS. 2b, 11a-11h). Moreover, Nb105 competed with Nb34 and Nb95, which did not compete for RBD interaction, indicating the presence of two distinct and non-overlapping epitopes. Second, Nb-RBD complexes were cross-linked by DSS (disuccinimidyl suberate) and were identified on average, four intermolecular cross-links by MS for Nbs 20, 93, 34, 95, and 105. The cross-links were used to map the RBD epitopes derived from the SEC data (Methods). The cross-linking models identified five epitopes (I, II, III, IV, and V corresponding to Nbs 20, 93, 34, 95, and 105) (FIG. 2c). The models satisfied 90% of the cross-links with an average precision of 7.8 Å (FIG. 2d, Table 2). The analysis confirmed the presence of a dominant Epitope I (e.g., epitopes of Nbs 20 and 21) overlapping with the hACE2 binding site. Epitope II also co-localized with the hACE2 binding site, while epitopes III-V did not (FIG. 2e). Epitope I Nbs had significantly shorter CDR3 (four amino acids shorter, p=0.005) than other epitope binders (FIG. 11i). Despite this, the vast majority of the selected Nbs potently inhibited the virus with an IC50 below 30 ng/ml (2 nM) (Table 1).

### Crystal Structure of RBD-Nb20 and MD Analysis of Nbs 20 and 21 for RBD Binding

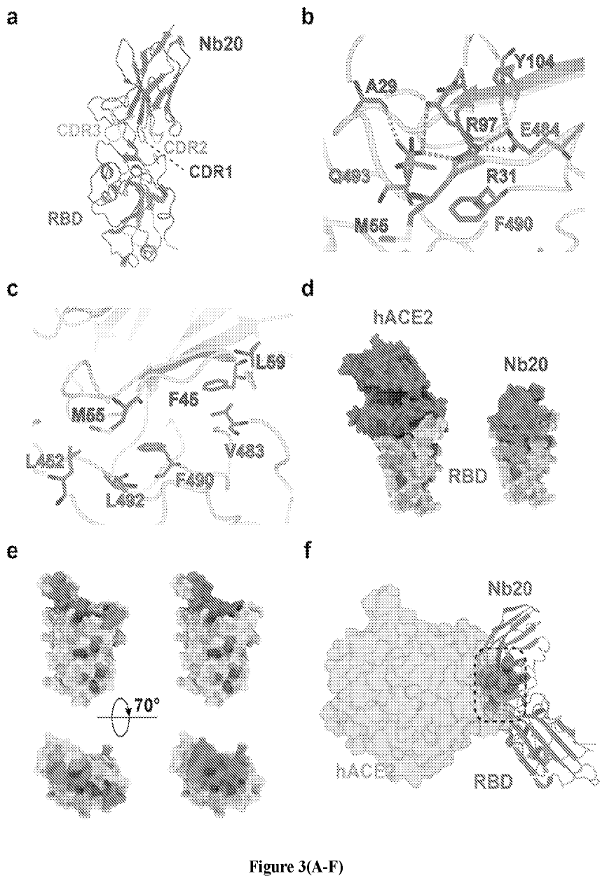

To explore the molecular mechanisms that underlie the unusually potent neutralization activities of Epitope I Nbs, a crystal structure of the RBD-Nb20 complex at a resolution of 3.3 Å was determined by molecular replacement (Methods, Table 3). Most of the residues in RBD (N334-G526) and the entire Nb20, particularly those at the protein interaction interface, are well resolved. There are two copies of RBD-Nb20 complexes in one asymmetric unit, which are almost identical with an RMSD of 0.277 Å over 287 Cα atoms. In the structure, all three CDRs of Nb20 interact with the RBD by binding to its large extended external loop with two short β-strands (FIG. 3a) (Q. H. Wang et al., 2020). E484 of RBD forms hydrogen bonding and ionic interactions with the side chains of R31 (CDR1) and Y104 (CDR3) of Nb20, while Q493 of RBD forms hydrogen bonds with the main chain carbonyl of A29 (CDR1) and the side chain of R97 (CDR3) of Nb20. These interactions constitute a major polar interaction network at the RBD and Nb20 interface. R31 of Nb20 also engages in a cation-π interaction with the side chain of F490 of the RBD (FIG. 3b). In addition, M55 from the CDR2 of Nb20 packs against residues L452, F490, and L492 of RBD to form hydrophobic interactions at the interface). Another small patch of hydrophobic interactions is formed among residues V483 of RBD and F45 and L59 from the framework β-sheet of Nb20 (FIG. 3c).

A small cavity with strong electron density at the RBD and Nb20 interface was observed surrounded by charged residues R31 and R97 of Nb20 and E484 of the RBD. A cacodylate group was modeled a from the protein crystallization conditions to fit the density, forming hydrogen bonds with two main chain amine groups of Nb20 and the RBD. Under physiological conditions, this cavity is occupied by ordered water molecules to mediate extensive hydrogen-bonding interactions with surrounding residues, contributing to the interactions between the RBD and Nb20. Similarly, in two crystal structures of the RBD bound to other Nbs (PDB IDs 6YZ5 and 7C8V0), there are also small molecules such as glycerol modeled at the RBD and Nb interfaces.

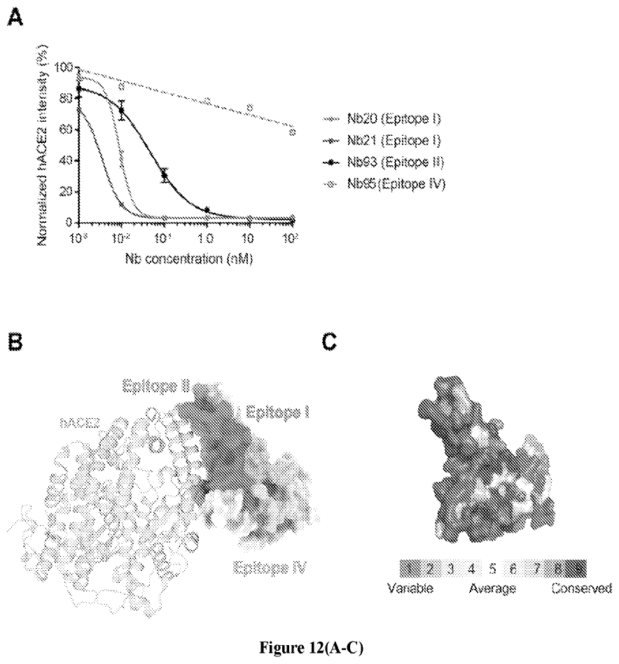

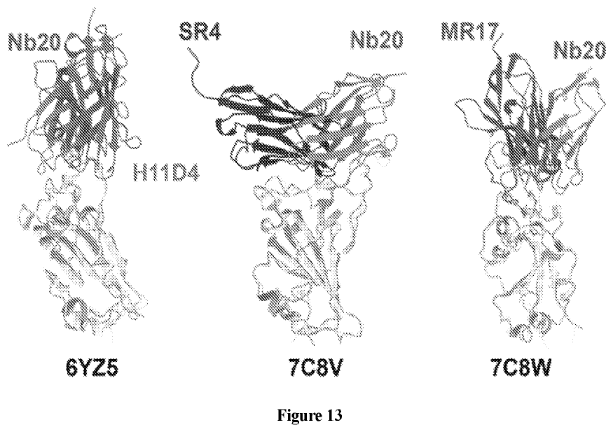

The binding mode of Nb20 to the RBD is distinct from all other reported SARS-CoV-2 neutralizing Nbs, which generally recognize similar epitopes in the RBD external loop region (T. Li et al., 2020; J. D. Walter et al., 2020; J. Huo et al., 2020) (FIG. 12). The extensive hydrophobic and polar interactions (FIGS. 3b-3c) between the RBD and Nb20 stem from the remarkable shape complementarity (FIG. 3d) between all the CDRs and the external RBD loop, leading to ultrahigh-affinity (˜10 pM). The structure of the best neutralizer Nb21 with RBD was further modeled based on the crystal structure (Methods). Only four residues vary between Nb20 and Nb21 (FIG. 13a), all of which are on CDRs. Two substitutions are at the RBD binding interface. S52 and M55 in the CDR2 of Nb20 are replaced by two asparagine residues N52 and N55 in Nb21. In this modeled structure, N52 forms a new H-bond with N450 of RBD (FIG. 13b). While N55 does not engage in additional interactions with RBD, it creates a salt bridge with the side chain of R31, which stabilizes the polar interaction network among R31 and Y104 of Nb21 and Q484 of RBD (FIG. 13b). All of those can contribute to a slower off-rate of Nb21 vs. Nb20 (FIGS. 2f, 9a) and stronger neutralization potency. Structural comparison of RBD-Nb20/21 and RBD-hACE2 (PDB 6LZG) (Q. H. Wang et al., 2020) clearly showed that the interfaces for Nb20/21 and hACE2 partially overlap (FIGS. 3d-3e). Notably, the CDR1 and CDR3 of Nb20/21 can severely clash with the first helix of hACE2, the primary binding site for RBD (FIG. 3f). This high-resolution structural study shows the exceptional binding affinities of epitope I Nbs that contribute to the sub-ng/ml neutralization capability.

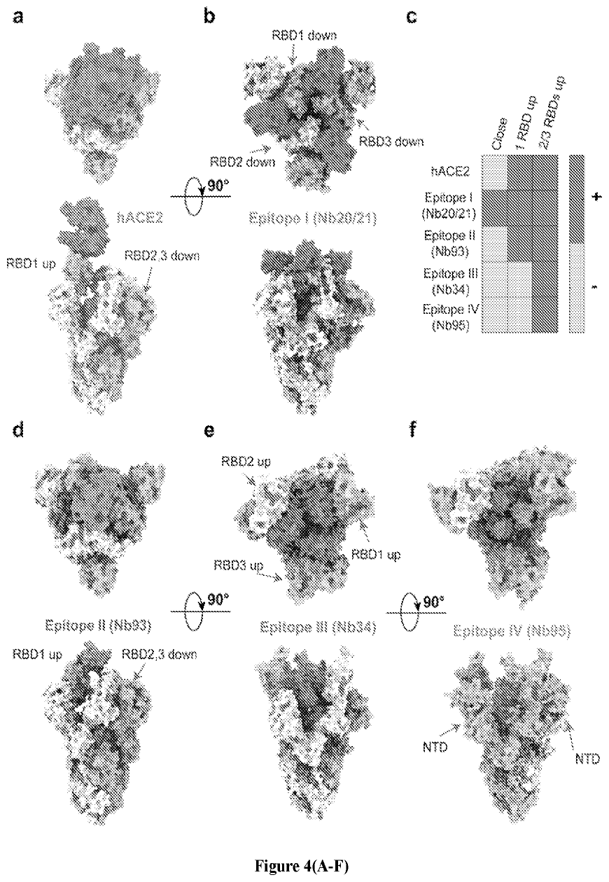

To further explore the molecular mechanisms that underlie the exceptional neutralization activities of our Nbs, we determined an X-ray crystallographic structure of the RBD-Nb20 complex at a resolution of 3.3 Å in which all the residue side chains were resolved. Our structure reveals that Nb20 employs an extensive network of hydrophobic and polar interactions to enable sub-nM, high-affinity RBD binding. Consistent to cross-linking, Nb20 interacts with the large cavity on the RBD formed by an extended loop (region 1: residues 432-438 and region 2: residues 464-484, FIG. 4). All three CDR loops are involved in binding. For example, A29 of CDR1 forms hydrophobic interactions with both Y436 and 5481, and R31 (the last CDR1 residue) inserts itself into the deep pocket of the RBD cavity, creating a salt bridge with E471 and a cation-π interaction with F477. 5 out of 9 residues on the CDR2 contact the RBD loop by hydrophobic interactions (i.e., pairs of A 48-E 471, A 51-S481, S 52-N436, M55-L479, and N57-F477) facilitating the penetration of its loop into the RBD groove. Despite relatively short CDR3, at least three residues (R94, 196, and Y101) bind directly to the RBD cavity by H-bonds and hydrophobic interactions. Moreover, the interaction between Nb20 and the RBD is further stabilized by a conserved F47 on the FR2 and a residue X on the FR3 that pairs with V483 and XYZ. Together, Nb 20 employs a remarkable array of hydrophobic and polar interactions that enable perfection of shape complementarity between the convex Nb and the RBD groove.

### Potential Mechanisms of SARS-CoV-2 Neutralization by Nbs

To understand the outstanding antiviral efficacy of these Nbs better, RBD-Nb complexes were superimposed to different spike conformations based on cryoEM structures. It was found that three copies of Nb20/21 can simultaneously bind all three RBDs in their “down” conformations (PDB 6VXX) (A. C. Walls et al., 2020) that correspond to the inactive spike (FIG. 4b). This analysis indicates a mechanism by which Nbs 20 and 21 (Epitope I) lock RBDs in their down conformation with ultrahigh affinity. Combined with the steric interference with hACE2 binding in the RBD open conformation (FIG. 4a), these mechanisms can explain the exceptional neutralization potencies of Epitope I Nbs.

Other epitope-binders do not fit into this inactive conformation without steric clashes and appear to utilize different neutralization strategies (FIG. 4c). For example, Epitope II: Nb 93 co-localizes with hACE2 binding site and can bind the spike in the one RBD “up” conformation (FIG. 4d, PDB 6VSB) (D. Wrapp et al., 2020). It can neutralize the virus by blocking the hACE2 binding site. Epitope III and IV Nbs can only bind when two or three RBDs are at their “up” conformations (PDB 6XCN) (C. O. Barnes et al., 2020) where the epitopes are exposed. In the all RBDs “up” conformation, three copies of Nbs can directly interact with the trimeric spike. Through RBD binding, Epitope III: Nb34 can be accommodated on top of the trimer to lock the helices of S2 in the prefusion stage, preventing their large conformational changes for membrane fusion (FIG. 4e). When superimposed onto the all “up” conformation, Epitope IV: Nb95 is proximal to the rigid NTD of the trimer, presumably restricting the flexibility of the spike domains (FIG. 4f).

### Development of Flexible, Multivalent Nb Formats for Highly Efficient Viral Neutralization

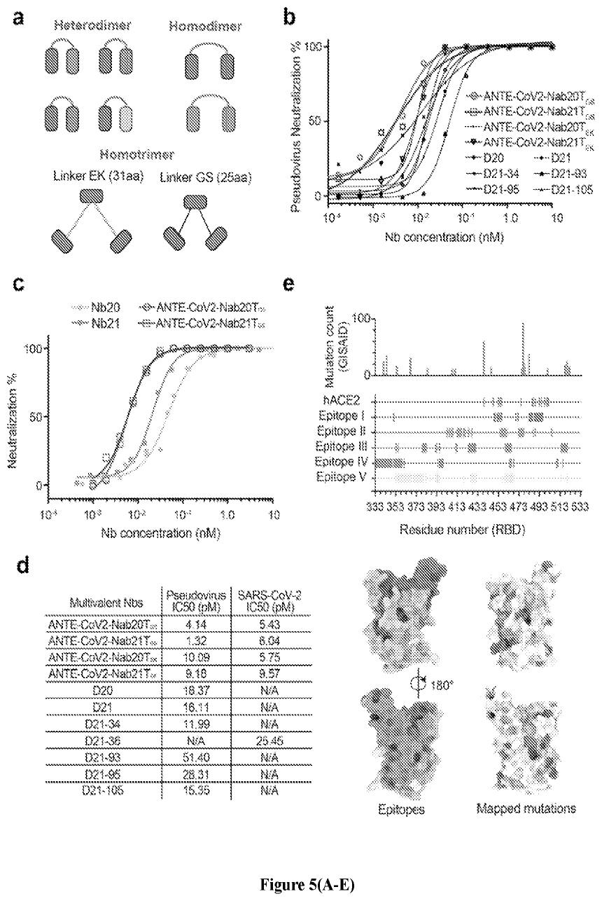

Epitope mapping enabled us to bioengineer a series of multivalent Nbs (FIG. 5a). Specifically, two sets of constructs that build upon the most potent Nbs were designed. The homotrimeric Nbs, in which a flexible linker sequence (either 31 or 25 amino acids, Methods) separates each monomer Nb (such as Nb21 or Nb20), were designed to increase the antiviral activities through avidity binding to the trimeric spike. The heterodimeric forms that conjugate two Nbs of unique, non-overlapping epitopes, through a flexible linker of 12 residues.

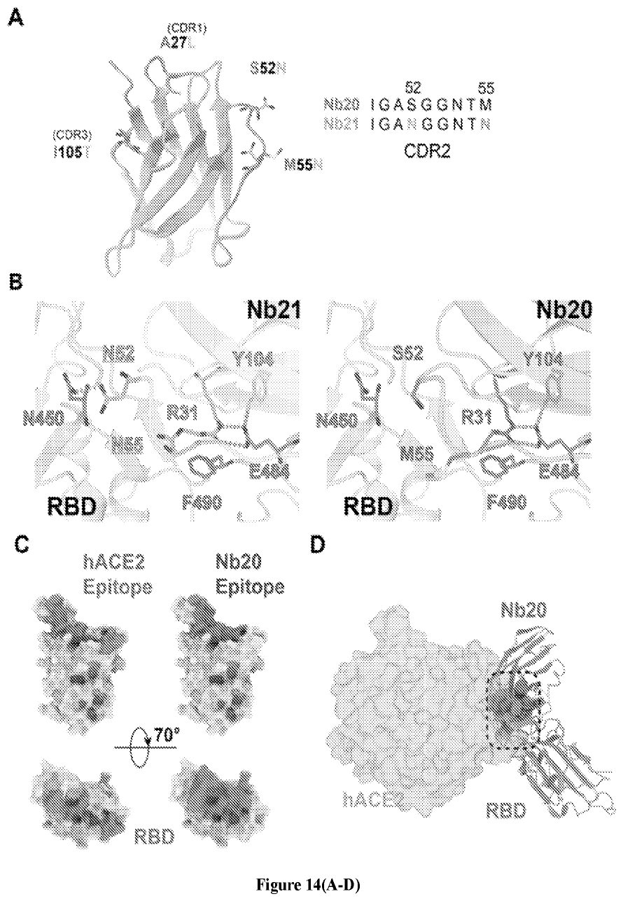

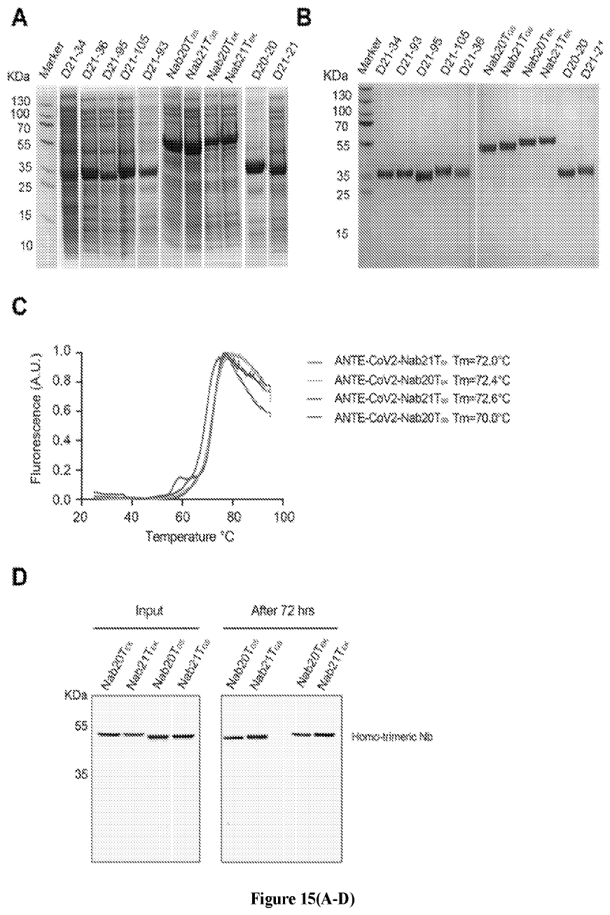

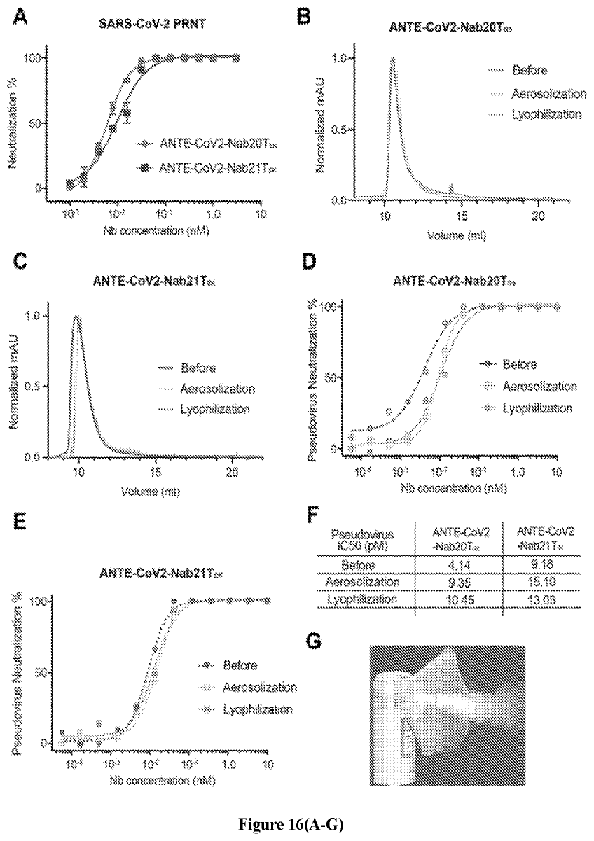

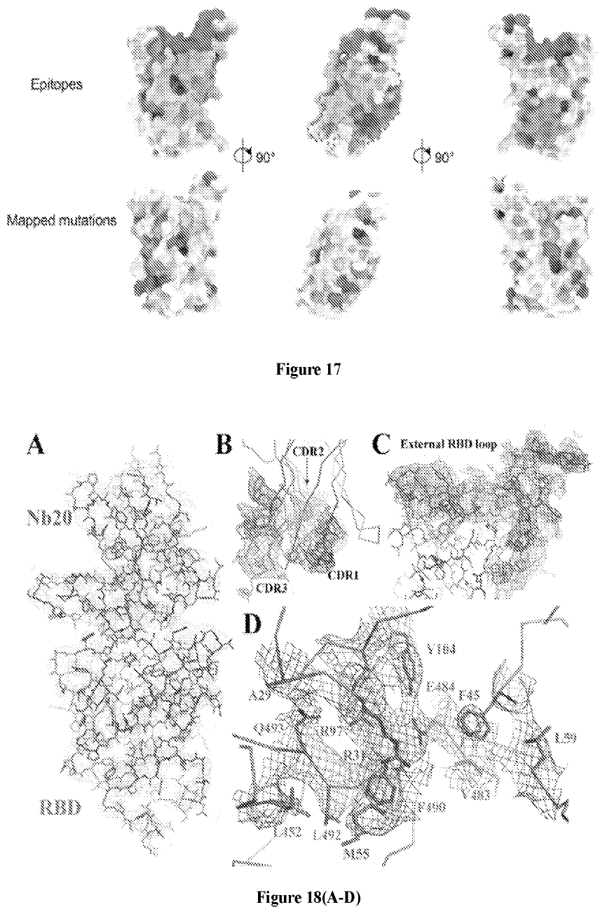

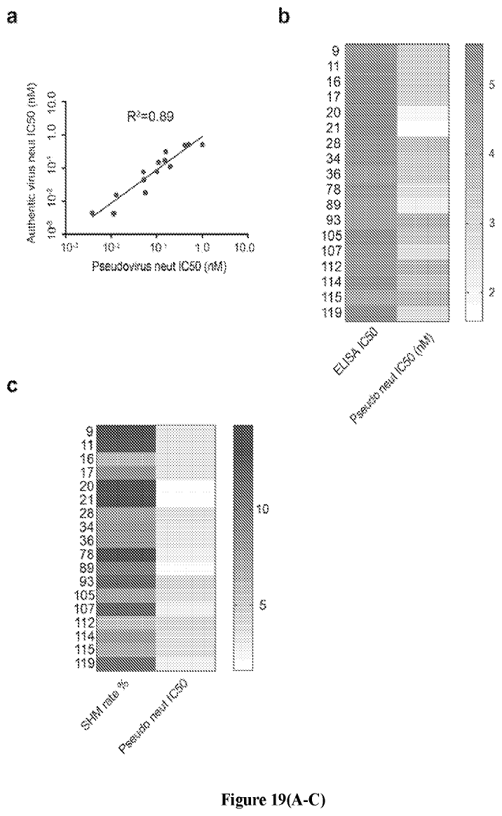

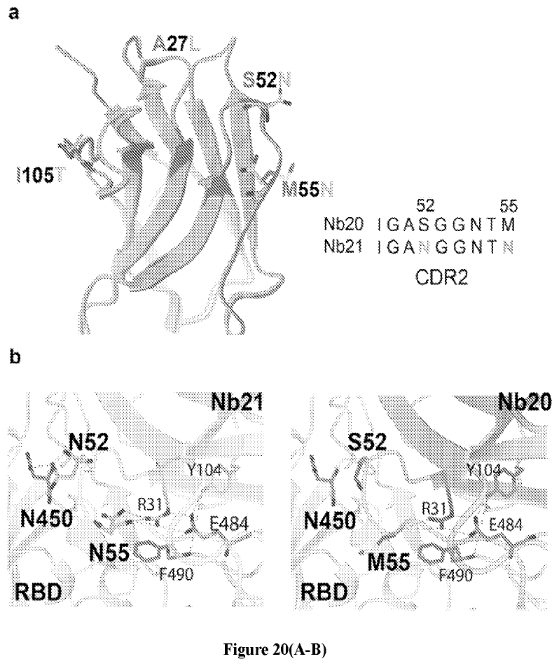

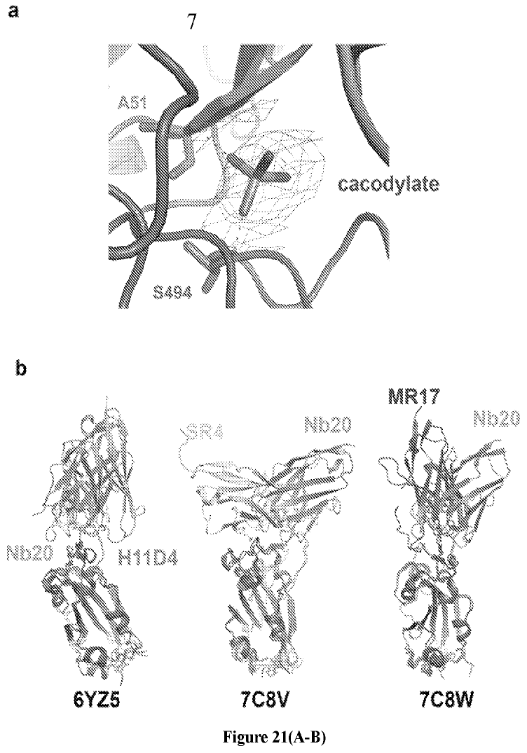

A variety of constructs were synthesized and their neutralization potency was tested. Up to ˜30 fold improvement was found for the homotrimeric constructs of Nb21₃ (IC50=1.3 pM) and Nb20₃ (IC50=3 pM) compared to the respective monomeric form by the pseudovirus luciferase assay (FIG. 5b, FIG. 5d). Similar results were obtained from the SARS-CoV-2 PRNT (FIGS. 5c, 5d, 15a). The improvements are greater than these values indicate, as the measured values can reflect the assay's lower detection limits. For the heterodimeric constructs, up to a 4-fold increase of potency (i.e., Nb21-Nb34) was observed. Importantly, the multivalent constructs retained outstanding physicochemical properties of the monomeric Nbs, including high solubility, yield, and thermostability (FIG. 14). They remained fully active after standard lyophilization and nebulization (Methods, FIGS. 15b-15e), indicating the outstanding stability and flexibility of administration. The majority of the RBD mutations observed in GISAID (Y. L. Shu et al., 2017) are very low in frequency (<0.0025). Therefore, the probability of mutational escape with a cocktail consisting of 2-3 Nbs covering different epitopes is extremely low (FIG. 5e) (J. Hansen et al., 2020).

The development of effective, safe, and inexpensive vaccines and therapeutics are critical to end the COVID-19 pandemic. Here, in vivo (camelid) antibody affinity maturation followed by advanced proteomics (Y. Xiang et al., 2020) enabled rapid identification of a large repertoire of diverse, high-affinity RBD Nbs for the neutralization of SARS-CoV-2. The majority of the high-affinity Nbs efficiently neutralize SARS-CoV-2 and some elite Nbs in their monomeric forms can inhibit the viral infection at single-digit to sub-ng/ml concentrations.

Multiple neutralization epitopes were identified through the integration of biophysics, structural proteomics, modeling, and X-ray crystallography. This study shows the mechanisms by which Nbs target the RBD with femtomolar affinity to achieve remarkable neutralization potency of a low-passage, clinical isolate of SARS-CoV-2. Structural analysis revealed that the hACE2 binding site correlates with immunogenicity and neutralization. While the most potent Nbs inhibit the virus by high-affinity binding to the hACE2 binding site, other neutralization mechanisms through non-hACE2 epitopes were also observed.

A preprint reported an extensively bioengineered, homotrimeric Nb construct reaching antiviral activity comparable to our single monomeric Nb20 (M. Schoof et al., 2020). Here, the present study has developed a collection of novel multivalent Nb constructs with outstanding stability and neutralization potency at double-digit pg/ml. This represents the most potent biotherapeutics for SARS-CoV-2 available to date. The use of multivalent, multi-epitope Nb cocktails can prevent virus escape (A. Baum et al., 2020; Y. Bar-On et al., 2018; M. Marovich et al., 2020). Flexible and efficient administration, such as direct inhalation can be used to improve antiviral drug efficacy and minimize the dose, cost, and potential toxicity for clinical applications. The high sequence similarity between Nbs and IgGs can restrain the immunogenicity (I. Jovcevska et. al., 2020). For intravenous drug delivery, it is possible to fuse our antiviral Nbs with the albumin-Nb constructs (Z. Shen et al., 2020) t already developed to improve the in vivo half-lives. These Nbs can also be applied as rapid point-of-care diagnostics due to the high stability, specificity, and low cost of manufacturing. These high-quality Nb agents contributes to curbing the current pandemic.

### Methods

Camelid immunization and proteomic identification of high-affinity RBD-Nbs. A male Llama “Wally” was immunized with an RBD-Fc fusion protein (Acro Biosystems, Cat #SPD-c5255) at a primary dose of 0.2 mg (with complete Freund's adjuvant), followed by three consecutive boosts of 0.1 mg every 2 weeks. ˜480 ml blood from the animal was collected 10 days after the final boost. All the above procedures were performed by the Capralogics, Inc. following the IACUC protocol. ˜1×10⁹ peripheral mononuclear cells were isolated using Ficoll gradient (Sigma). The mRNA was purified from the mononuclear cells using an RNeasy kit (Qiagen) and was reverse-transcribed into cDNA by the Maxima™ H Minus cDNA Synthesis kit (Thermo). The V_{H}H genes were PCR amplified, and the P5 and P7 adapters were added with the index before sequencing (Y. Xiang et al., 2020). Next-generation sequencing (NGS) of the V_{H}H repertoire was performed by Illumina MiSeq with the 300 bp paired-end model in the UPMC Genome Center. For proteomic analysis of RBD-specific Nbs, plasma was first purified from the immunized blood by the Ficoll gradient (Sigma). V_{H}H antibodies were then isolated from the plasma by a two-step purification protocol using protein G and protein A sepharose beads (Marvelgent) (P. C. Fridy et al., 2014). RBD-specific V_{H}H antibodies were affinity isolated and subsequentially eluted by either increasing stringency of high pH buffer or salt. All the eluted V_{H}Hs were neutralized and dialyzed into 1×DPBS before the quantitative proteomics analysis. RBD-specific V_{H}H antibodies were reduced, alkylated and in-solution digested using either trypsin or chymotrypsin (Y. Xiang et al., 2020). After proteolysis, the peptide mixtures were desalted by self-packed stage-tips or Sep-Pak C18 columns (Waters) and analyzed with a nano-LC 1200 that is coupled online with a Q Exactive™ HF-X Hybrid Quadrupole Orbitrap™ mass spectrometer (Thermo Fisher). Proteomic analysis was performed as previously described and by using the Augur Llama—a dedicated software that we developed to facilitate reliable identification, label-free quantification, and classification of high-affinity Nbs (25). This analysis led to thousands of RBD-specific, high-affinity Nb candidates that belong to ˜350 unique CDR3 families From these, we selected 109 Nb sequences with unique CDR3s for DNA synthesis and characterizations.

Nb DNA synthesis and cloning. The monomeric Nb genes and the homotrimeric Nbs 20 and 21 with the (GGGGS, (SEQ ID NO: 175)) 5 linkers were codon-optimized and synthesized (Synbio). All the Nb DNA sequences were cloned into a pET-21b(+) vector using EcoRI and HindIII restriction sites. The monomeric Nbs 20, 21, and 89, as well as the homotrimeric Nbs 20 and 21, were also cloned into a pET-22b(+) vector at the BamHI and XhoI sites for periplasmic purification.

The Nb genes were codon-optimized for expression in E. coli and in vitro synthesized (Synbio). Different synthesized Nb genes were cloned into a pET-21b (+) vector at BamHI and XhoI restriction sites or pET-22b (+) vector. To produce the heterodimeric Nb formats, DNA fragments of Nbs (such as Nb34), were amplified from the pET21(a+) Nb constructs while new XhoI/HindIII restriction sites plus (GGGGS, (SEQ ID NO: 175))₂ linker sequence were introduced. The fragments were then inserted into pET21(a+)_Nb21 at the XhoI and HindIII restriction sites to produce a heterodimer form [Nb21-(GGGGS, (SEQ ID NO: 175))2-Nb]. The homotrimeric constructs were either directly synthesized or produced in house by recombinant DNA methods. The DNA fragment of the linker sequence EGKSSGSGSESKSTGGGGSEGKSSGSGSESKST (SEQ ID NO: 176) was annealed and extended using the following two oligos: CCGCTCGAGTGCTGCGGCCGCGGTGCTTTTGCTTTCGCCGCTACCGCTGCTTTTACCT TCGCTGCCACC (SEQ ID NO: 177), and CCCAAGCTTGAAGGTAAAAGCAGCGGTAGCGGCGAAAGCAAAAGCACCGGTGGCG GTGGCAGCGAAGGT (SEQ ID NO: 178) (Integrated DNA Technologies).

The digested XhoI/HindIII linker fragment was then inserted into the corresponding sites on pET21(a+)_Nb21 or pET21(a+)_Nb20. To shuffle the second Nb21 or 20 to this Nb_linker vector, we amplified Nb21 or 20 from pET21(a+) and introduced the XhoI/NotI restriction sites. After digestion, the XhoI/NotI Nb fragment was inserted into the Nb_linker vector to produce a homodimer construct. The new Nb constructs were subsequently sequence verified.

Expression and purification of proteins. Nb DNA constructs were transformed into BL21(DE3) cells and plated on Agar with 50 μg/ml ampicillin at 37° C. overnight. Cells were cultured in an LB broth to reach an O.D. of ˜0.5-0.6 before IPTG (0.5 mM) induction at 16° C. overnight. Cells were then harvested, sonicated, and lysed on ice with a lysis buffer (1×PBS, 150 mM NaCl, 0.2% TX-100 with protease inhibitor). After cell lysis, protein extracts were collected by centrifugation at 15,000×g for 10 mins and the his-tagged Nbs were purified by Cobalt resin and natively eluted by imidazole buffer (Thermo). Eluted Nbs were subsequently dialyzed in a dialysis buffer (e.g., lx DPBS, pH 7.4). For the periplasmic preparation of Nbs (Nbs 20, 21, and 89 and the homotrimeric constructs), cell pellets were resuspended in the TES buffer (0.1 M Tris-HCl, pH 8.0; 0.25 mM EDTA, pH 8.0; 0.25 M Sucrose) and incubated on ice for 30 mM. The supernatants were collected by centrifugation and subsequently dialyzed to DPBS. The resulting Nbs were then purified by Cobalt resin as described above.

The RBD (residues 319-541) of the SARS-Cov-2 S protein was expressed as a secreted protein in Spodoptera frugiperda Sf9 cells (Expression Systems) using the Bac-to-bac baculovirus method (Invitrogen). To facilitate protein purification, a FLAG-tag and an 8×His-tag were fused to its N terminus, and a tobacco etch virus (TEV) protease cleavage site was introduced between the His-tag and RBD. Cells were infected with baculovirus and incubated at 27° C. for 60 h before harvesting. The conditioned media was added with 20 mM Tris pH 7.5 and incubated at RT for 1 h in the presence of 1 mM NiSO4 and 5 mM CaCl₂. The supernatant was collected by centrifugation at 25,000 g for 30 min and then incubated with Nickel-NTA agarose resin (Clontech) overnight at 4° C. After washing with buffer containing 20 mM Hepes pH 7.5, 200 mM NaCl, and 50 mM imidazole, the RBD protein was eluted with the same buffer containing 400 mM Imidazole. Eluted protein was treated by TEV protease overnight to remove extra tags and further purified by size exclusion chromatography using the Superdex 75 column (Fisher) with a buffer containing 20 mM HEPES pH 7.5 and 150 mM NaCl. To obtain RBD and Nb20 complex, purified RBD was mixed with purified Nb20 in a molar ratio of 1:1.5 and then incubated on ice for 2 hours. The complex was further purified using the Superdex 75 column with a buffer containing 20 mM Hepes pH 7.5 and 150 mM NaCl. Purified RBD-Nb20 complex was concentrated to 10-15 mg/ml for crystallization.

ELISA (Enzyme-linked immunosorbent assay). Indirect ELISA was carried out to measure the relative affinities of Nbs. RBD was coated onto a 96-well ELISA plate (R&D system) at two ng/well in coating buffer (15 mM sodium carbonate, 35 mM sodium bicarbonate, pH 9.6) overnight at 4° C. and was blocked with a blocking buffer (DPBS, 0.05% Tween 20, 5% milk) at room temperature for 2 hrs. Nbs were serially 10× diluted in the blocking buffer, starting from 1 μM to 0.1 pM, and 100 μl of each concentration was incubated with RBD-coated plates for 2 hrs. HRP-conjugated secondary antibodies against T7-tag (Thermo) were diluted 1:7500 and incubated with the well for 1 hr at room temperature. After PBST (DPBS, 0.05% Tween 20) washes, the samples were further incubated under dark with freshly prepared w3,3′,5,5′-Tetramethylbenzidine (TMB) substrate for 10 mins to develop the signals. After the STOP solution (R&D system), the plates were read at multiple wavelengths (the optical density at 550 nm wavelength subtracted from the density at 450 nm) on a plate reader (Multiskan GO, Thermo Fisher). A non-binder was defined if any of the following two criteria were met: i) The ELISA signal was under detected at one 1 μM concentration. ii) The ELISA signal could only be detected at a concentration of 1 μM and was under detected at 0.1 μM concentration. The raw data was processed by Prism 7 (GraphPad) to fit into a 4PL curve and to calculate log IC50.

Competitive ELISA with hACE2. The COVID-19 spike-ACE2 binding assay kit was purchased from RayBiotech (Cat #CoV-SACE2-1). A 96-well plate was pre-coated with recombinant RBD. Nbs were 10-fold diluted (from 1 μM to 1 pM) in the assay buffer containing a saturating amount of hACE2 and then incubated with the plate at room temperature for 2.5 hrs. The plate was washed by the washing buffer to remove the unbound hACE2. Goat anti-hACE2 antibodies were incubated with the plate for 1 hr at room temperature. HRP-conjugated anti-goat IgG was added to the plate and incubated for an hour. TMB solution was added to react with the HRP conjugates for 0.5 hr. The reaction was then stopped by the Stop Solution. The signal corresponding to the amount of the bound hACE2 was measured by a plate reader at 450 nm. The resulting data were analyzed by Prism 7 (GraphPad) and plotted.

Pseudotyped SARS-CoV-2 neutralization assay. The 293T-hsACE2 stable cell line (Cat #C-HA101, Lot #TA060720C) and the pseudotyped SARS-CoV-2 (Wuhan-Hu-1 strain) particles with GFP (Cat #RVP-701G, Lot #CG-113A) or luciferase (Cat #RVP-701L, Lot #CL109A, and CL-114A) reporters were purchased from the Integral Molecular. The neutralization assay was carried out according to the manufacturers' protocols. In brief, 10-fold serially diluted Nbs were incubated with the pseudotyped SARS-CoV-2-GFP for 1 hr at 37° C. for screening, while 3- or 5-fold serially diluted Nbs/immunized serum/immunized VHH mixture was incubated with the pseudotyped SARS-CoV-2-luciferase for accurate measurements. At least eight concentrations were tested for each Nb. Pseudovirus in culture media without Nbs was used as a negative control. 100 μl of the mixtures were then incubated with 100 μl 293T-hsACE2 cells at 2.5×10e5 cells/ml in the 96-well plates. The infection took ˜72 hrs at 37° C. with 5% CO2. The GFP signals (ex488/em530) were read using the Tecan Spark 20M with auto-optimal settings, while the luciferase signal was measured using the Renilla-Glo luciferase assay system (Promega, Cat #E2720) with the luminometer at 1 ms integration time. The obtained relative fluorescent/luminescence signals (RFU/RLU) from the negative control wells were normalized and used to calculate the neutralization percentage at each concentration. For SARSCoV-2-GFP screening, the 49 tested Nbs were divided into 6 groups based on their lowest tested concentration of 100% neutralization. For SARS-CoV-2-luciferase, data was processed by Prism7 (GraphPad) to fit into a 4PL curve and to calculate the log IC50 (half-maximal inhibitory concentration).

SARS-CoV-2 Munich plaque reduction neutralization test (PRNT). Nbs were diluted in a 2- or 3-fold series in Opti-MEM (Thermo). Each Nb dilution (110 μl) was mixed with 110 μl of SARS-CoV-2 (Munich strain) containing 100 plaque-forming units (p.f.u.) of the virus in Opti-MEM. The serum-virus mixes (220 μl total) were incubated at 37° C. for 1 h, after which they were added dropwise onto confluent Vero E6 cell (ATCC® CRL-1586™) monolayers in the six-well plates. After incubation at 37° C., 5% (v/v) CO2 for 1 h, 2 ml of 0.1% (w/v) immunodiffusion agarose (MP Biomedicals) in Dulbecco's modified eagle medium (DMEM) (Thermo) with 10% (v/v) FBS and 1× pen-strep was added to each well. The cells were incubated at 37° C., 5% CO2 for 72 hrs. The agarose overlay was removed and the cell monolayer was fixed with 1 ml/well formaldehyde (Fisher) for 20 min at room temperature. Fixative was discarded and 1 ml/well of 1% (w/v) crystal violet in 10% (v/v) methanol was added. Plates were incubated at room temperature for 20 min and rinsed thoroughly with water. Plaques were then enumerated and the 50% plaque reduction neutralization titer (PRNT50) was calculated. A validated SARS-CoV-2 antibody-negative human serum control, a validated NIBSC SARS-CoV-2 plasma control, was obtained from the National Institute for Biological Standards and Control, UK) and an uninfected cells control were also performed to ensure that virus neutralization by antibodies was specific.

Thermostability analysis of Nbs. Nb thermostabilities were measured by differential scanning fluorimetry (DSF). To prepare DSF samples, Nbs were mixed with SYPRO orange dye (Invitrogen) in PBS to reach a final concentration of 2.5-15 μM. The samples were analyzed in triplicate using a 7900HT Fast Real-Time PCR System (Applied Biosystems) as previously described {cite Allen's paper}. The melting point was then calculated by the first derivatives method {Niesen, 2007}.

Surface plasmon resonance (SPR). Surface plasmon resonance (SPR, Biacore 3000 system, GE Healthcare) was used to measure Nb affinities. Briefly, recombinant RBD was immobilized to the flow channels of an activated CMS sensor-chip. RBD was diluted to 10 μg/ml in 10 mM sodium acetate, pH 4.5, and injected into the SPR system at 5 μl/min for 420 s. The surface was then blocked by 1 M ethanolamine-HCl (pH 8.5). For each Nb analyte, a series of dilution (spanning ˜1,000-fold concentration range) was injected in duplicate, with HBS-EP+ running buffer (GE-Healthcare) at a flow rate of 20-30 μl/min for 120-180 s, followed by a dissociation time of 10-20 mins. Between each injection, the sensor chip surface was regenerated twice with a low pH buffer containing 10 mM glycine-HCl (pH 1.5-2.5) at a flow rate of 40-50 μl/min for 30 s-1 mM. Binding sensorgrams for each Nb were processed and analyzed using BIAevaluation by fitting with 1:1 Langmuir model.

Phylogenetic tree analysis and sequence logo. Sequences were first aligned and numbered according to Martin's numbering scheme by ANARCI (J. Dunbar et al., 2016). The phylogenetic tree was constructed from aligned sequences by Molecular Evolutionary Genetics Analysis (MEGA) (S. Kumar et al., 2018) using the Maximum Likelihood method. The sequence logo was plotted from aligned sequences by logomaker (A. Tareen et al., 2020).

Epitope screening by size exclusion chromatography (SEC). Recombinant RBD and Nb proteins were mixed at a ratio of 1:1 (w:w) and incubated at 4° C. for 1 hr. The complexes were analyzed by the SEC (Superdex75, GE Healthcare) at a low rate of 0.4 ml/min for 1 hr using a running buffer of 20 mM HEPES, 150 mM NaCl, pH 7.5. Protein signals were detected by ultraviolet light absorbance at 280 nm.

Clustering and phylogenetic tree analysis. A phylogenetic tree was generated by Clustal Omega {Sievers, 2014} with the input of unique NbHSA CDR3 sequences and the adjacent framework sequences (i.e., YYCAA (SEQ ID NO: 179) to the N-terminus and WGQG (SEQ ID NO: 180) to the C-terminus of CDR3s) to help alignments. The data was plotted by ITol (Interactive Tree of Life) {Letunic, 2007}. Isoelectric points and hydrophobicities of the CDR3s were calculated using the BioPython library. The sequence logo was plotted using WebLogo {Crooks, 2004}.

Chemical cross-linking and mass spectrometry (CXMS). Recombinant Nbs were first pre-incubated with the trypsin resin for approximately 2-5 mins to remove the N terminal T7 tag, which is highly reactive to the crosslinker. Nb was incubated with RBD in PBS at 4° C. for 1 hr to allow the formation of the complex. The reconstituted complexes were then cross-linked with 2 mM disuccinimidyl suberate (DSS, ThermoFisher Scientific) for 25 min at 25° C. with gentle agitation. The reaction was then quenched with 50 mM ammonium bicarbonate (ABC) for 10 min at room temperature. After protein reduction and alkylation, the cross-linked samples were separated by a 4-12% SDS-PAGE gel (NuPAGE, Thermo Fisher). The regions corresponding to the monomeric, cross-linked species (˜45-50 kDa) were sliced and digested in-gel with trypsin and Lys-C, or chymotrypsin {Shi, 2014; Shi, 2015; Xiang, 2020}. After efficient proteolysis, the cross-link peptide mixtures were desalted and analyzed with a nano-LC 1200 (Thermo Fisher) coupled to a Q Exactive™ HF-X Hybrid Quadrupole-Orbitrap™ mass spectrometer (Thermo Fisher). The cross-linked peptides were loaded onto a Picochip column (C18, 3 μm particle size, 300 Å pore size, 50 μm×10.5 cm; New Objective) and eluted using a min LC gradient: 5% B-8% B, 0-5 min; 8% B-32% B, 5-45 min; 32% B-100% B, 45-49 min; 100% B, 49-54 min; 100% B-5% B, 54 min-54 min 10 sec; 5% B, 54 min 10 sec-60 min 10 sec; mobile phase A consisted of 0.1% formic acid (FA), and mobile phase B consisted of 0.1% FA in 80% acetonitrile. The QE HF-X instrument was operated in the data-dependent mode. The top 8 most abundant ions (with the mass range of 380 to 2,000 and the charge state of +3 to +7) were fragmented by high-energy collisional dissociation (normalized HCD energy 27). The target resolution was 120,000 for MS and 15,000 for MS/MS analyses. The quadrupole isolation window was 1.8 Th and the maximum injection time for MS/MS was set at 120 ms. After the MS analysis, the data was searched by pLink for the identification of cross-linked peptides. The mass accuracy was specified as 10 and 20 p.p.m. for MS and MS/MS, respectively. Other search parameters included cysteine carbamidomethylation as a fixed modification and methionine oxidation as a variable modification. A maximum of three trypsin missed-cleavage sites was allowed. Initial search results were obtained using the default 5% false discovery rate, estimated using a target-decoy search strategy. The crosslink spectra were manually checked as previously described {Shi, 2014; Shi, 2015; Xiang, 2020}.

Integrative structural modeling. Structural models for Nbs were obtained using a multi-template comparative modeling protocol of MODELLER {Sali, 1993}. Next, the CDR3 loop {Fiser, 2003} was refined and the top 5 scoring loop conformations were selected for the downstream docking in addition to 5 models from comparative modeling. Each Nb model was then docked to the RBD structure (PDB 6lzg) by an antibody-antigen docking protocol of PatchDock software that focuses the search to the CDRs and optimizes CXMS-based distance restraints satisfaction {Schneidman-Duhovny, 2012; Schneidman-Duhovny, 2020}. A restraint was considered satisfied if the Ca-Ca distance between the cross-linked residues was within 28A for DSS cross-linkers. The models were then re-scored by a statistical potential SOAP {Dong, 2013}. The antigen interface residues (distance <6A from Nb atoms) among the top 10 scoring models, according to the SOAP score, were used to determine the epitopes. The convergence was measured as the average RMSD among the ten top-scoring models.

Crystallization, data collection, and structure determination of RBD-Nb20 complex. Crystallization trials were performed with the Crystal Gryphon robot (Art Robbins). The RBD-Nb20 complex was crystallized using the sitting-drop vapor diffusion method at 17° C. The crystals were obtained in conditions containing 100 mM sodium cacodylate pH 6.5 and 1 M sodium citrate. For data collection, the crystals were transferred to the reservoir solution supplemented with 20% glycerol before freezing in liquid nitrogen. X-ray diffraction data were collected at the Advanced Photon Source (APS) beamline 23IDB of GM/CA with a 10 μm diameter microbeam. The data were processed using HKL2000 (A. J. McCoy et al., 2007). Diffraction data from six crystals were merged to obtain a complete dataset with a resolution of 3.3 Å.

The structure was determined by the molecular replacement method in Phaser (P. D. Adams et al., 2010) using the crystal structures of RBD (PDB 6LZG) and an Nb (VHH-72, PDB 6WAQ) as search models. The initial model was refined in Phenix (P. Emsley et al., 2004) and adjusted in COOT (C. J. Williams et al., 2018). The model quality was checked by MolProbity (T. D. Goddard et al., 2018). The final refinement statistics were listed in Table 3.

Nb21 comparative modeling was done using the Nb20 structure as a template in MODELLER. All structure visualization figures were prepared using UCSF ChimeraX (F. H. Niesen et al., 2007).

Nb stability test. For the stability test, Nb was eluted and collected in the SEC running buffer (20 mM HEPES, 150 mM NaCl, pH 7.5) and then concentrated to 1 ml (1 mg/ml). 0.5 ml of the concentrated Nb was lyophilized by snap freezing in liquid nitrogen before dried in a speed-vac. ddH2O was then used to reconstitute the Nb. The other 0.5 ml was aerosolized by using a portable mesh atomizer nebulizer (MayLuck). No obvious dead volume was observed. The aerosols were collected in a microcentrifuge tube. SEC analysis and pseudovirus neutralization assays were performed as described above.

Size exclusion chromatography (SEC). The recombinant RBD and selected Nbs were mixed at a molar ratio of 1:2 and incubated at 4° C. for 1 hr in the SEC buffer. The complexes were analyzed by SEC (Superdex75, GE LifeSciences). Protein signals were detected by ultraviolet light absorbance at 280 nm.

Data collection and structure determination. Crystals were collected and were frozen in liquid nitrogen. Data collection was performed at beamline 23-ID of GM/CA@APS at the Advanced Photon Source. Microbeams of 10 or 20 μM diameter were used to acquire all diffraction data.

TABLE 1
Summary of the biophysical, biochemical, and functional properties of the monomeric
RBDNbs and the multivalent constructs

| | | | | | | Pseudo-neut | Authentic | | | | | |
|---|---|---|---|---|---|---|---|---|---|---|---|---|
| | | SEQ | | ELISA | Pseudo- | IC50 | virus | | Thermo- | SPR | SPR | SPR |
| | | ID | Ex- | affinity | neutralization | Luciferase | PRNT IC50 | | stability | ka | kd | KD |
| # | CDR | NO | pression | (nM) | (GFP) | (nM) | (nM) | Epitope | (avg.Tm ° C.) | (1/Ms) | (1/s) | (M) |
| 2 | TTNDYDY | 82 | *, 0.5 | 4.82 | 5 nM- | / | / | / | / | | | |
| | | | mg/L | | 50 nM | | | | | | | |
| 3 | VAPRINPRGSPT | 83 | ***, 2.4 | 0.48 | 5 nM- | / | / | / | / | | | |
| | NY | | mg/L | | 50 nM | | | | | | | |
| 4 | VGPRFNPRGSPT | 84 | ****, | 0.67 | 5 nM- | / | / | / | / | | | |
| | NY | | 8.8 | | 50 nM | | | | | | | |
| | | | mg/L | | | | | | | | | |
| 5 | DSNIDRLHSAGS | 85 | **, 3.8 | >1000 | / | / | / | / | / | | | |
| | DYRY | | mg/L | | | | | | | | | |
| 6 | KQRVGPGIMSA | 86 | **, 1.8 | 0.57 | >3 μM | / | / | / | / | | | |
| | PTYDY | | mg/L | | | | | | | | | |
| 9 | SHGVVDGTSVN | 87 | ***, 7 | 0.89 | 0.5 nM- | 1.496 | 1.722 | Same as | / | | | |
| | GYRY | | mg/L | | 5 nM | | | 21 | | | | |
| | | | (0.8L) | | | | | | | | | |
| 10 | WRFGPTGVKVD | 88 | ****, 9 | 0.24 | 5 nM- | / | / | / | / | | | |
| | Y | | mg/L | | 50 nM | | | | | | | |
| 11 | RNLETFDYTY | 89 | ***, 5 | 0.39 | 0.5 nM- | 1.084 | 0.8 | Same as | / | | | |
| | | | mg/L | | 5 nM | | | 21 | | | | |
| | | | (0.8L) | | | | | | | | | |
| 12 | RTERGVYDY | 90 | **, 1.2 | 539.26 | | / | / | / | / | | | |
| | | | mg/L | | | | | | | | | |
| 15 | TAYSTTYNSVRD | 91 | ****, | 0.47 | 5 nM- | / | / | / | / | | | |
| | Y | | 8.9 | | 50 nM | | | | | | | |
| | | | mg/L | | | | | | | | | |
| 16 | VAYDYSWGRPR | 92 | ***, 7 | 0.37 | 0.5 nM- | 1.574 | 1.233 | Same as | / | | | |
| | NF | | mg/L | | 5 nM | | | 21 | | | | |
| 17 | VGQEASAYAPR | 93 | ***, 6.4 | 0.45 | 0.5 nM- | 1.107 | 1.5 | Residues | / | | | |
| | AY | | mg/L | | 5 nM | | | 345, 346, | | | | |
| | | | | | | | | 347, 348, | | | | |
| | | | | | | | | 349, 351, | | | | |
| | | | | | | | | 352, 353, | | | | |
| | | | | | | | | 354, 355, | | | | |
| | | | | | | | | 356, 399, | | | | |
| | | | | | | | | 448, 449, | | | | |
| | | | | | | | | 450, 451, | | | | |
| | | | | | | | | 452, 453, | | | | |
| | | | | | | | | 454, 455, | | | | |
| | | | | | | | | 466, 467, | | | | |
| | | | | | | | | 468, 469, | | | | |
| | | | | | | | | 470, 471, | | | | |
| | | | | | | | | 472, 482, | | | | |
| | | | | | | | | 483, 484, | | | | |
| | | | | | | | | 489, 490, | | | | |
| | | | | | | | | 491, 492, | | | | |
| | | | | | | | | 493, 493, | | | | |
| | | | | | | | | 494 of | | | | |
| | | | | | | | | SEQ ID | | | | |
| | | | | | | | | NO: 189 | | | | |
| 18 | TSRTMIHTSQA | 94 | **, 2.2 | 0.57 | 5 nM- | / | / | / | / | | | |
| | MPINWDY | | mg/L | | 50 nM | | | | | | | |
| 20 | RDIETAEYIY | 95 | ****, 8.5 | 0.4 | <0.5 nM | 0.102 | 0.048 | Residues | 71.8 | | | |
| | | | mg/L | | | | | 351, 417, | | | | |
| | | | | | | | | 449, 450, | | | | |
| | | | | | | | | 451, 452, | | | | |
| | | | | | | | | 453, 455, | | | | |
| | | | | | | | | 456, 470, | | | | |
| | | | | | | | | 472, 481, | | | | |
| | | | | | | | | 482, 483, | | | | |
| | | | | | | | | 484, 485, | | | | |
| | | | | | | | | 486, 487, | | | | |
| | | | | | | | | 488, 489, | | | | |
| | | | | | | | | 490, 491, | | | | |
| | | | | | | | | 492, 493, | | | | |
| | | | | | | | | 494, 495, | | | | |
| | | | | | | | | 496 of | | | | |
| | | | | | | | | SEQ ID | | | | |
| | | | | | | | | NO: 189 | | | | |
| 21 | RDIETAEYTY | 96 | ***, 5.6 | 0.3 | <0.5 nM | 0.0495 | 0.022 | Residues | 72.8 | 3.79E+ | <1.00E− | <1.00E− |
| | | | mg/L | | | | | 351, 446, | | 06 | 6 | 12 |
| | | | | | | | | 447, 448, | | | | |
| | | | | | | | | 449, 450, | | | | |
| | | | | | | | | 451, 452, | | | | |
| | | | | | | | | 453, 455, | | | | |
| | | | | | | | | 456, 470, | | | | |
| | | | | | | | | 472, 482, | | | | |
| | | | | | | | | 483, 484, | | | | |
| | | | | | | | | 485, 486, | | | | |
| | | | | | | | | 487, 488, | | | | |
| | | | | | | | | 489, 490, | | | | |
| | | | | | | | | 491, 492, | | | | |
| | | | | | | | | 493, 494, | | | | |
| | | | | | | | | 495, 496, | | | | |
| | | | | | | | | 497, 498, | | | | |
| | | | | | | | | 505, 531 | | | | |
| | | | | | | | | of SEQ ID | | | | |
| | | | | | | | | NO: 189 | | | | |
| 24 | DTYSNYEKDDS | 97 | ****, 8 | 0.77 | 5 nM- | / | / | / | / | 2.96E+ | 3.08E− | 1.04E− |
| | | | mg/L | | 50 nM | | | | | 06 | 05 | 11 |
| 26 | DTYSNYKKDDS | 98 | ***, 2.5 | 0.92 | 5 nM- | / | / | / | / | | | |
| | | | mg/L | | 50 nM | | | | | | | |
| 28 | ETYSLYEKSDS | 99 | *, 1.4 | 0.92 | 0.5 nM- | 6.124 | / | / | / | | | |
| | | | mg/L | | 5 nM | | | | | | | |
| 29 | ETYSNYEKAYS | 100 | **, 1.6 | 1.25 | 5 nM- | / | / | / | / | | | |
| | | | mg/L | | 50 nM | | | | | | | |
| 32 | ETYTLYEKDSS | 101 | ****, | 2.17 | 5 nM- | / | / | / | / | | | |
| | | | 18 mg/L | | 50 nM | | | | | | | |
| 33 | VGQEGSARAPR | 102 | **, 2 | >1000 | / | / | / | / | / | | | |
| | AY | | mg/L | | | | | | | | | |
| 34 | SKDPYGSPWTRS | 103 | *, 0.8 | 0.47 | 0.5 nM- | 1.991 | 1.125 | Residues | / | | | |
| | EFDDY | | mg/L | | 5 nM | | | 366, 369, | | | | |
| | | | | | | | | 370, 371, | | | | |
| | | | | | | | | 372, 373, | | | | |
| | | | | | | | | 374, 375, | | | | |
| | | | | | | | | 376, 377, | | | | |
| | | | | | | | | 378, 379, | | | | |
| | | | | | | | | 380, 381, | | | | |
| | | | | | | | | 382, 383, | | | | |
| | | | | | | | | 384, 385, | | | | |
| | | | | | | | | 412, 436, | | | | |
| | | | | | | | | 437 of | | | | |
| | | | | | | | | SEQ ID | | | | |
| | | | | | | | | NO: 189 | | | | |
| 36 | ETYSIYEKDDS | 104 | *, 1.4 | 1.04 | 0.5 nM- | 1.563 | / | Residues | / | | | |
| | | | mg/L | | 5 nM | | | 344, 345, | | | | |
| | | | | | | | | 346, 347, | | | | |
| | | | | | | | | 348, 349 | | | | |
| | | | | | | | | 351, 352, | | | | |
| | | | | | | | | 353, 354, | | | | |
| | | | | | | | | 355, 356, | | | | |
| | | | | | | | | 357, 396, | | | | |
| | | | | | | | | 451, 457, | | | | |
| | | | | | | | | 464, 465, | | | | |
| | | | | | | | | 466, 467, | | | | |
| | | | | | | | | 468, 470 | | | | |
| | | | | | | | | of SEQ ID | | | | |
| | | | | | | | | NO: 189 | | | | |
| 37 | DRGSSYYYTRAS | 105 | *, 0.2 | 0.38 | / | / | / | / | / | | | |
| | EYTY | | mg/L | | | | | | | | | |
| 39 | AQPDVFGGGDY | 106 | **, 1 | 1.49 | 50 nM- | / | / | / | / | | | |
| | | | mg/L | | 500 nM | | | | | | | |
| 41 | SKDRYGSPWTRS | 107 | *, 1 | 0.2 | 5 nM- | / | / | / | / | | | |
| | EFEDY | | mg/L | | 50 nM | | | | | | | |
| 42 | RQGCSYNGCDE | 108 | **, 0.77 | 0.66 | 5 nM- | / | / | / | / | | | |
| | Y | | mg/L | | 50 nM | | | | | | | |
| 43 | RQGCSYNGCDE | 109 | *, 0.4 | 664.2 | / | / | / | / | / | | | |
| | Y | | mg/L | | | | | | | | | |
| 44 | RTPETGQYDY | 110 | ***, 5.3 | >1000 | / | / | / | / | / | | | |
| | | | mg/L | | | | | | | | | |
| 46 | RTPYMRDY | 111 | **, 2.2 | 25.88 | 50 nM- | / | / | / | / | | | |
| | | | mg/L | | 500 nM | | | | | | | |
| 48 | DRSEFGSRLYSDE | 112 | **, 2.2 | 1.08 | 500 nM | / | / | / | / | | | |
| | NEYDY | | mg/L | | −3 μM | | | | | | | |
| 51 | VAPVEGTGRIRA | 113 | **, 2.2 | >1000 | / | / | / | / | / | | | |
| | Y | | mg/L | | | | | | | | | |
| 52 | RKDSVVLSSVGY | 152 | *, 1.8 | >1000 | / | / | / | / | / | | | |
| | DY | | mg/L | | | | | | | | | |
| 55 | RASSNTYDDPRS | 114 | ***, 3 | 2.24 | 5 nM- | / | / | / | / | | | |
| | FAF | | mg/L | | 50 nM | | | | | | | |
| 58 | GALRVGSFSPDY | 115 | **, 0.4 | 2.68 | / | / | / | / | / | | | |
| | | | mg/L | | | | | | | | | |
| 59 | VLGRPYGSRWL | 116 | **, 1.3 | >1000 | / | / | / | / | / | | | |
| | DDVDS | | mg/L | | | | | | | | | |
| 60 | RIGSIANLQPREY | 117 | ***, 5.4 | 137.72 | 5 nM- | / | / | / | / | | | |
| | EFAY | | mg/L | | 50 nM | | | | | | | |
| 62 | VDVSVGGDY | 118 | *, 0.2 | 85.7 | / | / | / | / | / | | | |
| | | | mg/L | | | | | | | | | |
| 63 | PACSGSGCRNY | 119 | ****, 9 | 0.84 | 5 nM- | / | / | / | / | | | |
| | | | mg/L | | 50 nM | | | | | | | |
| 64 | GPRLGSTPRAYD | 146 | ****, 14.5 | 0.45 | 0.5 nM- | 4.13 | 4.875 | Same as | / | | | |
| | Y | | mg/L | | 5 nM | | | 21 | | | | |
| 65 | ESGVNY | 120 | ****, 7.5 | >1000 | / | / | / | / | / | | | |
| | | | mg/L | | | | | | | | | |
| 66 | ARFYGRSIAQDY | 148 | *, 0.4 | 0.33 | / | / | / | / | / | | | |
| | DS | | mg/L | | | | | | | | | |
| 68 | DSSTSRLHSAGS | 121 | ***, 2.5 | 6.11 | 50 nM- | / | / | / | / | | | |
| | DYRY | | mg/L | | 500 nM | | | | | | | |
| 73 | NRGPNLYPHIGD | 122 | ****, | >1000 | / | / | / | / | / | | | |
| | IEY | | 10 mg/L | | | | | | | | | |
| 74 | SRGPNLYPHIGD | 123 | **, 2.5 | 67.14 | 50 nM- | / | / | / | / | | | |
| | VEY | | mg/L | | 500 nM | | | | | | | |
| 76 | RKSINYVQEYDY | 124 | *, 0.2 | 0.17 | / | / | / | / | / | | | |
| | | | mg/L | | | | | | | | | |
| 78 | RNIETAEYTY | 125 | ****, | 0.61 | 0.5 nM- | 0.509 | 0.766 | Same as | / | | | |
| | | | 14 mg/L | | 5 nM | | | 21 | | | | |
| 80 | QGQGNREY | 126 | ****, 9 | 5.09 | 50 nM- | / | / | / | / | | | |
| | | | mg/L | | 500 nM | | | | | | | |
| 81 | KIFGPGSGSY | 143 | ***, 4.2 | >1000 | / | / | / | / | / | | | |
| | | | mg/L | | | | | | | | | |
| 82 | NTLNSQLLPRAY | 144 | *, 0.5 | 0.86 | 0.5 nM- | 12.246 | / | Same as | / | | | |
| | | | mg/L | | 5 nM | | | 21 | | | | |
| 83 | GSLRVGSFSPDY | 145 | ***, 2.9 | 1.51 | 5 nM- | / | / | / | / | | | |
| | | | mg/L | | 50 nM | | | | | | | |
| 84 | SSRTMNTISQTM | 127 | **, 2 | 0.53 | 5 nM- | / | / | / | / | | | |
| | RINWDY | | mg/L | | 50 nM | | | | | | | |
| 87 | GIPMSTVL | 128 | **, 3 | >1000 | / | / | / | / | / | | | |
| | | | mg/L | | | | | | | | | |
| 89 | RNPGTGQYDY | 129 | **, 0.6 | 1.09 | <0.5 nM | 0.133 | 0.154 | Same as | 65.9 | 5.78E+ | 6.24E− | 1.08E− |
| | | | mg/L | | | | | 21 | | 06 | 04 | 10 |
| 90 | GVRDFAPRIDSY | 130 | ***, 5.6 | 4.03 | >3 μM | / | / | / | / | | | |
| | DY | | mg/L | | | | | | | | | |
| 91 | DIAPFYCSWPLR | 131 | *, 0.3 | 1.28 | / | / | / | / | / | | | |
| | TRLVEY | | mg/L | | | | | | | | | |
| 92 | DPTILYHALSQRT | 132 | ***, 9 | 4.92 | >3 μM | / | / | / | / | | | |
| | QNYRY | | mg/L | | | | | | | | | |
| 93 | CRQEFSWDFSSR | 133 | ***, 6.7 | 1.15 | 0.5 nM- | 14.454 | / | Diff from | / | | | |
| | DPDDFDY | | mg/L | | 5 nM | | | 21 | | | | |
| 94 | SPYTYSLLSRTGD | 134 | ***, 6 | 0.5 | 50 nM- | / | / | / | / | | | |
| | YGY | | mg/l | | 500 nM | | | | | | | |
| 95 | DKDVYYGYTSFP | 147 | ****, | 2.28 | 5 nM- | 10.046 | 5.105 | Residues | / | | | |
| | NEYEY | | 17.8 mg/L | | 50 nM | | | 369, 371, | | | | |
| | | | | | | | | 372, 373, | | | | |
| | | | | | | | | 374, 375, | | | | |
| | | | | | | | | 376, 377, | | | | |
| | | | | | | | | 378, 379, | | | | |
| | | | | | | | | 380, 381, | | | | |
| | | | | | | | | 382, 383, | | | | |
| | | | | | | | | 384, 403, | | | | |
| | | | | | | | | 404, 405, | | | | |
| | | | | | | | | 407, 408, | | | | |
| | | | | | | | | 411, 412, | | | | |
| | | | | | | | | 414, 432, | | | | |
| | | | | | | | | 435, 501, | | | | |
| | | | | | | | | 502, 503, | | | | |
| | | | | | | | | 504, 505, | | | | |
| | | | | | | | | 508, 510 | | | | |
| | | | | | | | | of SEQ ID | | | | |
| | | | | | | | | NO: 189 | | | | |
| 96 | ALYVTSTRGSYA | 135 | **, 0.7 | 0.04 | 5 nM- | / | / | / | / | | | |
| | Y | | mg/L | | 50 nM | | | | | | | |
| 97 | GGWGLTQPISV | 149 | *, 1.7 | 0.32 | 50 nM- | / | / | / | / | | | |
| | DY | | mg/L | | 500 nM | | | | | | | |
| 98 | GTYGSTSVRDFG | 150 | ****, 9.5 | >1000 | / | / | / | / | / | | | |
| | P | | mg/L | | | | | | | | | |
| 99 | RTGSGVY | 151 | ****, 8.2 | 0.35 | 0.5 nM- | 0.524 | 0.452 | Same as | / | | | |
| | | | mg/L | | 5 nM | | | 21 | | | | |
| 100 | TSRTMIHTSQTM | 136 | **, 2 | 1.04 | 5 nM- | / | / | / | / | | | |
| | RINWDY | | mg/L | | 50 nM | | | | | | | |
| 102 | SRTAGLTSNRSLY | 137 | ***, 7.3 | 28.38 | 500 nM | / | / | / | / | | | |
| | DY | | mg/L | | −3 μM | | | | | | | |
| 103 | DVATIGSRLANY | 138 | ***, 4 | >1000 | / | / | / | / | / | | | |
| | DY | | mg/L | | | | | | | | | |
| 104 | RTPYRRDY | 139 | ****, 7.2 | >1000 | / | / | / | / | / | | | |
| | | | mg/L | | | | | | | | | |
| 105 | RRDSSWGYSRDL | 140 | ***, 4.4 | 0.26 | 0.5 nM- | 5.105 | 5.07 | Residues | / | | | |
| | FEYDY | | mg/L | | 5 nM | | | 368, 369, | | | | |
| | | | | | | | | 370, 371, | | | | |
| | | | | | | | | 372, 374, | | | | |
| | | | | | | | | 375, 376 | | | | |
| | | | | | | | | 377, 378, | | | | |
| | | | | | | | | 379, 380, | | | | |
| | | | | | | | | 381, 382, | | | | |
| | | | | | | | | 383, 384, | | | | |
| | | | | | | | | 404, 405, | | | | |
| | | | | | | | | 406, 407, | | | | |
| | | | | | | | | 408, 409, | | | | |
| | | | | | | | | 414, 435, | | | | |
| | | | | | | | | 436, 508 | | | | |
| | | | | | | | | of SEQ ID | | | | |
| | | | | | | | | NO: 189 | | | | |
| 106 | RFGIVTTRDEYEF | 141 | *, 0.4 | >1000 | / | / | / | / | / | | | |
| | | | mg/L | | | | | | | | | |
| 107 | RSATTALYDY | 142 | 2.4 | 0.76 | 0.5 nM- | 0.569 | 0.181 | Same as | / | | | |
| | | | mg/L | | 5 nM | | | 21 | | | | |
| | | | (0.8L) | | | | | | | | | |
| ANTE- | / | 72 | ****, | / | / | 4.140 | 5.433 | / | 70 | | | |
| CoV2- | | | 9.4 | | | pM | pM | | | | | |
| Nab20 | | | mg/L | | | | | | | | | |
| TGS | | | | | | | | | | | | |
| ANTE- | / | 73 | ***, 6.2 | / | / | 10.093 | 5.754 | / | 72.4 | | | |
| CoV2- | | | mg/L | | | pM | pM | | | | | |
| Nab20 | | | | | | | | | | | | |
| TEK | | | | | | | | | | | | |
| ANTE- | / | 74 | ****, | / | / | 1.321 | 6.039 | / | 72.6 | | | |
| CoV2- | | | 9.5 | | | pM | pM | | | | | |
| Nab21 | | | mg/L | | | | | | | | | |
| TGS | | | | | | | | | | | | |
| ANTE- | / | 75 | ***, 6.3 | / | / | 9.183 | 9.572 | / | 72 | | | |
| CoV2- | | | mg/L | | | pM | pM | | | | | |
| Nab21 | | | | | | | | | | | | |
| TEK | | | | | | | | | | | | |
| D20- | / | 76 | ***, 7.5 | / | / | 12.740 | / | / | / | | | |
| 20 | | | mg/L | | | pM | | | | | | |
| D21- | / | 77 | ***, 6 | / | / | 16.107 | / | / | / | | | |
| 21 | | | mg/L | | | pM | | | | | | |
| 21-34 | | 78 | ***, | / | / | 11.376 | / | / | / | | | |
| | | | 5 | | | pM | | | | | | |
| | | | mg/L | | | | | | | | | |
| 21- | / | 81 | ***, 7.2 | / | / | 46.99 | 25.450 | / | / | | | |
| 36 | | | mg/L | | | pM | pM | | | | | |
| 21- | / | 187 | ***, 4.1 | / | / | 51.404 | / | / | / | | | |
| 93 | | | mg/L | | | pM | | | | | | |
| 21- | / | 80 | ****, | / | / | 28.314 | / | / | / | | | |
| 95 | | | 9.2 | | | pM | | | | | | |
| | | | mg/L | | | | | | | | | |
| 21- | / | 188 | **, 2.6 | / | / | 15.346 | / | / | / | | | |
| 105 | | | mg/L | | | pM | | | | | | |

TABLE 2
Summary of the cross-links of the Nbs from different epitopes

| | CX | Nb | RBD | | | | |
|---|---|---|---|---|---|---|---|
| No | Peptide | residue | residue | Modifications | Score | Folder | RMSD |
| 20 | DNAKNTIYLQMNSLKPQDTAVYYCAAR | V20(90) | RBD_Flag_ | Carbamidomethyl[C] | 5.14E−02 | seq_ | 5.56 |
| | (SEQ ID NO: 153) (4)- | | His(150) | (8); Carbamido- | | 627 | |
| | LPDDFTGCVIAWNSNNLDSKVGGNYNYL | | | [methyC](57); | | | |
| | YR (SEQ ID NO: 154)(20) | | | Oxidation[M](44) | | | |
| | EFVAAIGASGGMTNYLDSVKGR (SEQ ID | V20(79) | RBD_Flag_ | Oxidation[M](12) | 5.62E−01 | | |
| | NO: 155)(20)-QIAPGQTGKIADYNYK | | His(123) | | | | |
| | (SEQ ID NO: 156)(9) | | | | | | |
| 95 | DTYYTNSVKGR (SEQ ID NO: 157) | V95(81) | RBD_Flag_ | null | 6.75E−01 | seq_ | 5.05 |
| | (9)-KSNLKPFER (SEQ ID NO: 158) | | His(164) | | | 9010 | |
| | (1) | | | | | | |
| | DTYYTNSVKGR(SEQ ID NO: 157) | V95(81) | RBD_Flag | null | 7.87E−01 | | |
| | (9)-STNLVKNK(SEQ ID NO: 159) | | His(241) | | | | |
| | (6) | | | | | | |
| 34 | QIAPGQTGKIADYNYK (SEQ ID NO: | V34(116) | RBD_Flag_ | null | 1.10E−11 | seq | 5.64 |
| | 156)(9)-SKDPYGSPWTR (SEQ ID | | His(123) | | | 271 | |
| | NO: 160)(2) | | | | | | |
| | EWVSGIDSDGSDTAYASSVKGR (SEQ ID | V34(81) | RBD_Flag_ | null | 5.85E−08 | | |
| | NO: 162)(20)-KSTNLVK (SEQ ID | | His(235) | | | | |
| | NO: 161)(1) | | | | | | |
| | EWVSGIDSDGSDTAYASSVKGR (SEQ ID | V34(81) | RBD_Flag_ | null | 4.36E−06 | | |
| | NO: 162)(20)-STNLVKNK (SEQ ID | | His(241) | | | | |
| | NO: 159)(6) | | | | | | |
| | SKDPYGSPWTR (SEQ ID NO: 160) | V34(116) | RBD_Flag_ | Carbamidomethyl[C] | 5.25E−06 | | |
| | (2)-ISNCVADYSVLYNSASFSTFKCYGVS | | His(84) | (4); Carbamidomethy | | | |
| | PTK (SEQ ID NO: 163)(21) | | | [C](22) | | | |
| | SKDPYGSPWTR(SEQ ID NO: 160) | V34(116) | RBD_Flag_ | null | 2.96E−08 | | |
| | (2)-KSNLKPFER(SEQ ID NO: 158) | | His(164) | | | | |
| | (1) | | | | | | |
| 105 | SGETTLYADSVKGR(SEQ ID | V105(81) | RBD_Flag_ | null | 9.97E−14 | seq_ | 10.8 |
| | NO: 165)(12)-STNLVKNK(SEQ ID | | His(241) | | | 1098 | |
| | NO: 159)(6) | | | | | | |
| | DNAKNTVYLQMNSLK (SEQ ID NO: | V105(92) | RBD_Flag_ | null | 1.82E−08 | | |
| | 166)(4)-KSNLKPFER (SEQ ID NO: | | His(164) | | | | |
| | 158)(1) | | | | | | |
| | SGETTLYADSVKGR (SEQ ID NO: 165) | V105(81) | RBD_Flag_ | null | 1.15E−06 | | |
| | (12)-KSNLKPFER (SEQ ID NO: | | His(164) | | | | |
| | 158)(1) | | | | | | |
| | SGETTLYADSVKGR (SEQ ID | V105(81) | RBD_Flag_ | Carbamidomethyl[C] | 3.03E−05 | | |
| | NO: 165)(12)-NKCVNF (SEQ ID NO: | | His(243) | (20) | | | |
| | 164)(2) | | | | | | |
| | QAPGKER(SEQ ID NO: 167)(5)- | V105(59) | RBD_Flag_ | null | 4.18E−05 | | |
| | QIAPGQTGKIADYNYK (SEQ ID NO: | | His(123) | | | | |
| | 156)(9) | | | | | | |
| | DNAKNTVYLQMNSLK(SEQ ID NO: | V105(92) | RBD_Flag_ | null | 8.97E−05 | | |
| | 166)(4)-SNLKPFER (SEQ ID NO: | | His(168) | | | | |
| | 168)(4) | | | | | | |
| | DNAKNTVYLQMNSLK (SEQ ID NO: | V105(92) | RBD_Flag_ | null | 2.15E−04 | | |
| | 166)(4)-STNLVKNK (SEQ ID NO: | | His(241) | | | | |
| | 159)(6) | | | | | | |
| 93 | EWVAGITPGSGTFYADSVKGR (SEQ ID | V93(80) | RBD_Flag_ | null | 5.21E−05 | seq_ | 12 |
| | NO: 169)(19)-SNLKPFER(SEQ ID | | His(168) | | | 3693 | |
| | NO: 168)(4) | | | | | | |
| | EWVAGITPGSGTFYADSVKGR (SEQ ID | V93(80) | RBD_Flag_ | null | 1.58E−04 | | |
| | NO: 169)(19)- | | His(123) | | | | |
| | QIAPGQTGKIADYNYK (SEQ ID NO: | | | | | | |
| | 156)(9) | | | | | | |
| | EWVAGITPGSGTFYADSVKGR (SEQ ID | V93(80) | RBD_Flag_ | Carbamidomethyl[C] | 1.76E−04 | | |
| | NO: 169)(19)- | | His(150) | (8) | | | |
| | LPDDFTGCVIAWNSNNLDSKVGGNYNYL | | | | | | |
| | YR (SEQ ID NO: 154)(20) | | | | | | |
| | DNAKNTLSLEINSLKPEDTALYYCAK | V93(91) | RBD_Flag_ | Carbamidomethyl[C] | 5.45E−04 | | |
| | (SEQ ID NO: 170)(4)- | | His(123) | (24) | | | |
| | QIAPGQTGKIADYNYK (SEQ ID NO: | | | | | | |
| | 156)(9) | | | | | | |
| | VRQAPGKGREW (SEQ ID NO: 171) | V93(59) | RBD_Flag_ | null | 9.99E−22 | | |
| | (7)-VIRGDEVRQIAPGQTGKIADY | | His(123) | | | | |
| | (SEQ ID NO: 172)(17) | | | | | | |
| | SLEINSLKPEDTALY (SEQ ID NO: | V93(102) | RBD_Flag_ | null | 1.43E−12 | | |
| | 173)(8)-NSNNLDSKVGGNY(SEQ ID | | His(150) | | | | |
| | NO: 174)(8) | | | | | | |

TABLE 3
X-ray diffraction data collection and refinement statistics

SARS-CoV-2 RBD with Nb20
(PDB code: 7JVB)

Data collection

| | |
|---|---|
| Space group | P4₁2₁2 |

Cell dimensions

| a, b, c (Å) | 70.716, 70.716, 435.037 | |
|---|---|---|
| α, β, γ (°) | 90, 90, 90 | |
| Resolution (Å) | 40-3.3 | (3.36-3.30) |
| R_{merge} (%)^{a} | 15.6 | (59.1) |
| l/σ(l) | 7.8 | (1.1) |
| CC_{1/2}^{b} | 1 | (0.811) |
| Completeness (%) | 97.1 | (81.4) |
| Redundancy | 6.2 | (3.4) |

Refinement

| | | |
|---|---|---|
| Resolution (Å) | 38.96-3.287 | (3.405-3.287) |
| No. reflections | 17336 | (1377) |

| | |
|---|---|
| R_{work}/R_{free} (%)^{c} | 28.1 (34.9)/32.2 (37.0) |

No. atoms

| | |
|---|---|
| Protein | 4728 |
| Ligand/ion | 20 |
| Water | 0 |

B factors

| | |
|---|---|
| Protein | 115.51 |
| Ligand/ion | 96.92 |
| Water | N/A |

R.m.s. deviations

| | |
|---|---|
| Bond lengths (Å) | 0.005 |
| Bond angles (°) | 1.66 |

Ramachandran analysis

| | |
|---|---|
| Favored region (%) | 95.92 |
| Allowed region (%) | 4.08 |
| Outliers | 0 |

The numbers in parentheses represent values for the highest resolution shell.
^{a}Rmerge = Σ|lᵢ − lₘ|/Σlᵢ, where lᵢ is the intensity of the measured reflection and lₘ is the mean intensity of all symmetry related reflections.
^{b}CC_{1/2} is the correlation coefficient of the half datasets.
^{c}R_{work} = Σ||F_{obs}| − |F_{calc}||/Σ|F_{obs}|, where F_{obs} and F_{calc} are observed and calculated structure factors. R_{free} = ΣT||F_{obs}| − |F_{calc}||/ΣT|F_{obs}|, where T is a test data set of about 5% of the total reflections randomly chosen and set aside prior to refinement.

TABLE 4

| | | Original | | | | | | | | |
|---|---|---|---|---|---|---|---|---|---|---|
| | | Amino Acid | CDR3, | | | | | | ELISA | |
| | | Sequence, | SEQ | SHM | SHM(CDR) | SHM(FR) | | Yield | affinity | SPR ka |
| # | Nanobody | SEQ ID NO | ID NO | % | % | % | Germline | (E. coli) | (nM) | (1/Ms) |
| 2 | 31547-95 | 1 | 82 | 8.13 | 18.75 | 6.03 | IGHV3-3*01 | *, | 4.82 | |
| | | | | | | | | 0.5 mg/L | | |
| 3 | 1951231-8 | 2 | 83 | 5.86 | 20.28 | 3.35 | IGHV3S53*01 | ***, | 0.48 | |
| | | | | | | | | 2.4 mg/L | | |
| 4 | 41809-703 | 3 | 84 | 6.18 | 24.44 | 2.95 | IGHV3S53*01 | ****, | 0.67 | |
| | | | | | | | | 8.8 mg/L | | |
| 5 | 6956553-2 | 4 | 85 | 10.30 | 29.17 | 6.90 | IGHV3-3*01 | **, | >1000 | |
| | | | | | | | | 3.8 mg/L | | |
| 6 | 1794706-46 | 5 | 86 | 10.72 | 33.82 | 5.93 | IGHV3-3*01 | **, | 0.57 | |
| | | | | | | | | 1.8 mg/L | | |
| 9 | 2184610-31 | 6 | 87 | 13.25 | 31.58 | 9.21 | IGHV3-3*01 | ***, | 0.89 | |
| | | | | | | | | 7 mg/L | | |
| | | | | | | | | (0.8 L) | | |
| 10 | 5048920-4 | 7 | 88 | 9.19 | 31.11 | 5.22 | IGHV3S53*01 | ****, | 0.24 | |
| | | | | | | | | 9 mg/L | | |
| 11 | 2454220-8 | 8 | 89 | 12.71 | 39.58 | 7.64 | IGHV3-3*01 | ***, | 0.39 | |
| | | | | | | | | 5 mg/L | | |
| | | | | | | | | (0.8 L) | | |
| 12 | 1851065-6 | 9 | 90 | 9.33 | 25.00 | 6.53 | IGHV3-3*01 | **, | 539.26 | |
| | | | | | | | | 1.2 mg/L | | |
| 15 | 11208708-2 | 10 | 91 | 7.83 | 15.56 | 6.43 | IGHV3S53*01 | ****, | 0.47 | |
| | | | | | | | | 8.9 mg/L | | |
| 16 | 4642331-98 | 11 | 92 | 5.94 | 17.78 | 3.80 | IGHV3S53*01 | ***, | 0.37 | |
| | | | | | | | | 7 mg/L | | |
| 17 | 3131410-2 | 12 | 93 | 8.45 | 6.67 | 8.16 | IGHV3S53*01 | ***, | 0.45 | |
| | | | | | | | | 6.4 mg/L | | |
| 18 | 4850341-42 | 13 | 94 | 3.93 | 10.42 | 2.70 | IGHV3-3*01 | **, | 0.57 | |
| | | | | | | | | 2.2 mg/L | | |
| 20 | 25383-75 | 14 | 95 | 14.05 | 53.06 | 6.37 | IGHV3-3*01 | ****, | 0.40 | |
| | | | | | | | | 8.5 mg/L | | |
| 21 | 648132-22 | 15 | 96 | 13.71 | 51.02 | 6.38 | IGHV3-3*01 | ***, | 0.30 | 3.79E+06 |
| | | | | | | | | 5.6 mg/L | | |
| 24 | 104794-22 | 16 | 97 | 9.39 | 33.33 | 3.92 | IGHV3-3*01 | ****, | 0.77 | 2.96E+06 |
| | | | | | | | | 8 mg/L | | |
| 26 | 2235693-16 | 17 | 98 | 9.44 | 27.08 | 5.51 | IGHV3-3*01 | ***, | 0.92 | |
| | | | | | | | | 2.5 mg/L | | |
| 28 | 2279980-2 | 18 | 99 | 7.62 | 20.83 | 4.90 | IGHV3-3*01 | *, | 0.92 | |
| | | | | | | | | 1.4 mg/L | | |
| 29 | 10057391-2 | 19 | 100 | 9.27 | 25.00 | 6.12 | IGHV3-3*01 | **, | 1.25 | |
| | | | | | | | | 1.6 mg/L | | |
| 32 | 4911584-46 | 20 | 101 | 7.13 | 16.67 | 5.07 | IGHV3-3*01 | ****, | 2.17 | |
| | | | | | | | | 18 mg/L | | |
| 33 | 2455880-2 | 21 | 102 | 8.97 | 13.33 | 7.92 | IGHV3S53*01 | **, | >1000 | |
| | | | | | | | | 2 mg/L | | |
| 34 | 5748889-4 | 22 | 103 | 8.25 | 12.50 | 7.38 | IGHV3S6*01 | *, | 0.47 | |
| | | | | | | | | 0.8 mg/L | | |
| 36 | 1810230-200 | 23 | 104 | 8.34 | 18.75 | 6.20 | IGHV3-3*01 | *, | 1.04 | |
| | | | | | | | | 1.4 mg/L | | |
| 37 | 6250396-10 | 24 | 105 | 8.14 | 25.00 | 4.63 | IGHV3-3*01 | *, | 0.38 | |
| | | | | | | | | 0.2 mg/L | | |
| 39 | 249510-4 | 25 | 106 | 9.29 | 25.00 | 6.07 | IGHV3S53*01 | **, | 1.49 | |
| | | | | | | | | 1 mg/L | | |
| 41 | 13280689-2 | 26 | 107 | 9.12 | 12.50 | 8.44 | IGHV3S6*01 | *, | 0.20 | |
| | | | | | | | | 1 mg/L | | |
| 42 | 1773148-121 | 27 | 108 | 6.98 | 13.35 | 5.90 | IGHV3S53*01 | **, | 0.66 | |
| | | | | | | | | 0.77 mg/L | | |
| 43 | 11434433-2 | 28 | 109 | 8.90 | 22.92 | 6.15 | IGHV3S58*01 | *, | 664.20 | |
| | | | | | | | | 0.4 mg/L | | |
| 44 | 14079846-2 | 29 | 110 | 9.80 | 30.77 | 5.44 | IGHV3-3*01 | ***, | >1000 | |
| | | | | | | | | 5.3 mg/L | | |
| 46 | 11981-1171 | 30 | 111 | 9.44 | 24.16 | 6.76 | IGHV3S53*01 | **, | 25.88 | |
| | | | | | | | | 2.2 mg/L | | |
| 48 | 3362597-2 | 31 | 112 | 9.29 | 29.17 | 5.23 | IGHV3-3*01 | **, | 1.08 | |
| | | | | | | | | 2.2 mg/L | | |
| 51 | 1363631-2 | 32 | 113 | 4.11 | 4.44 | 3.77 | IGHV3S53*01 | **, | >1000 | |
| | | | | | | | | 2.2 mg/L | | |
| 55 | 12890826-3 | 33 | 114 | 10.65 | 27.08 | 7.59 | IGHV3-3*01 | ***, | 2.24 | |
| | | | | | | | | 3 mg/L | | |
| 58 | 40968-11 | 34 | 115 | 11.49 | 41.67 | 5.40 | IGHV3-3*01 | **, | 2.68 | |
| | | | | | | | | 0.4 mg/L | | |
| 59 | 9256558-2 | 35 | 116 | 11.46 | 22.92 | 9.21 | IGHV3S7*01 | **, | >1000 | |
| | | | | | | | | 1.3 mg/L | | |
| 60 | 9667098-4 | 36 | 117 | 8.25 | 25.00 | 5.13 | IGHV3-3*01 | ***, | 137.72 | |
| | | | | | | | | 5.4 mg/L | | |
| 62 | 2815174-2 | 37 | 118 | 9.97 | 22.92 | 6.50 | IGHV3-3*01 | *, | 85.70 | |
| | | | | | | | | 0.2 mg/L | | |
| 63 | 1286822-2 | 38 | 119 | 5.79 | 23.33 | 2.51 | IGHV3S53*01 | ****, | 0.84 | |
| | | | | | | | | 9 mg/L | | |
| 65 | 41479-3 | 39 | 120 | 10.10 | 28.89 | 6.69 | IGHV3S53*01 | ****, | >1000 | |
| | | | | | | | | 7.5 mg/L | | |
| 68 | 6962898-3 | 40 | 121 | 7.43 | 20.83 | 5.02 | IGHV3-3*01 | ***, | 6.11 | |
| | | | | | | | | 2.5 mg/L | | |
| 73 | 499886-2 | 41 | 122 | 8.59 | 20.83 | 6.28 | IGHV3-3*01 | ****, | >1000 | |
| | | | | | | | | 10 mg/L | | |
| 74 | 2105929-4 | 42 | 123 | 7.82 | 20.31 | 5.44 | IGHV3-3*01 | **, | 67.14 | |
| | | | | | | | | 2.5 mg/L | | |
| 76 | 4965692-7 | 43 | 124 | 6.25 | 18.75 | 3.89 | IGHV3-3*01 | *, | 0.17 | |
| | | | | | | | | 0.2 mg/L | | |
| 78 | 228450-23 | 44 | 125 | 14.38 | 50.00 | 7.59 | IGHV3-3*01 | ****, | 0.61 | |
| | | | | | | | | 14 mg/L | | |
| 80 | 1849998-62 | 45 | 126 | 11.37 | 17.78 | 10.08 | IGHV3S53*01 | ****, | 5.09 | |
| | | | | | | | | 9 mg/L | | |
| 84 | 5359400-9 | 46 | 127 | 4.32 | 13.26 | 2.60 | IGHV3-3*01 | **, | 0.53 | |
| | | | | | | | | 2 mg/L | | |
| 87 | 2080196-11 | 47 | 128 | 6.33 | 20.00 | 3.92 | IGHV3S53*01 | **, | >1000 | |
| | | | | | | | | 3 mg/L | | |
| 89 | 1863745-88 | 48 | 129 | 10.03 | 41.67 | 3.92 | IGHV3-3*01 | **, | 1.09 | 5.78E+06 |
| | | | | | | | | 0.6 mg/L | | |
| 90 | 9241710-3 | 49 | 130 | 11.04 | 29.17 | 7.67 | IGHV3-3*01 | ***, | 4.03 | |
| | | | | | | | | 5.6 mg/L | | |
| 91 | 8561148-17 | 50 | 131 | 3.13 | 10.42 | 1.67 | IGHV3S61*01 | *, | 1.28 | |
| | | | | | | | | 0.3 mg/L | | |
| 92 | 8778218-2 | 51 | 132 | 10.47 | 33.33 | 6.25 | IGHV3-3*01 | ***, | 4.92 | |
| | | | | | | | | 9 mg/L | | |
| 93 | 4603394-3 | 52 | 133 | 11.55 | 20.83 | 9.73 | IGHV3S31*01 | ***, | 1.15 | |
| | | | | | | | | 6.7 mg/L | | |
| 94 | 6539104-2 | 53 | 134 | 6.14 | 16.67 | 4.18 | IGHV3-3*01 | ***, | 0.50 | |
| | | | | | | | | 6 mg/L | | |
| 96 | 1980804-35 | 54 | 135 | 7.17 | 20.83 | 4.60 | IGHV3-3*01 | **, | 0.04 | |
| | | | | | | | | 0.7 mg/L | | |
| 100 | 4034805-10 | 55 | 136 | 4.96 | 14.58 | 3.12 | IGHV3-3*01 | **, | 1.04 | |
| | | | | | | | | 2 mg/L | | |
| 102 | 7619220-2 | 56 | 137 | 5.46 | 18.75 | 2.93 | IGHV3-3*01 | ***, | 28.38 | |
| | | | | | | | | 7.3 mg/L | | |
| 103 | 2285805-2 | 57 | 138 | 7.26 | 16.67 | 5.63 | IGHV3-3*01 | ***, | >1000 | |
| | | | | | | | | 4 mg/L | | |
| 104 | 698335-3 | 58 | 139 | 9.18 | 22.22 | 6.81 | IGHV3S53*01 | ****, | >1000 | |
| | | | | | | | | 7.2 mg/L | | |
| 105 | 8582551-2 | 59 | 140 | 7.53 | 20.83 | 5.00 | IGHV3-3*01 | ***, | 0.26 | |
| | | | | | | | | 4.4 mg/L | | |
| 106 | 1465219-2 | 60 | 141 | 8.84 | 15.63 | 7.74 | IGHV3-3*01 | *, | >1000 | |
| | | | | | | | | 0.4 mg/L | | |
| 107 | 1780411-98 | 61 | 142 | 11.73 | 35.42 | 7.23 | IGHV3-3*01 | **, | 0.76 | |
| | | | | | | | | 2.4 mg/L | | |
| 111 | 4104768-2 | 62 | 143 | 8.65 | 24.44 | 5.44 | IGHV3S53*01 | ***, | >1000 | |
| | | | | | | | | 4.2 mg/L | | |
| 112 | 4609841-633 | 63 | 144 | 5.95 | 13.34 | 4.25 | IGHV3S53*01 | *, | 0.86 | |
| | | | | | | | | 0.5 mg/L | | |
| 113 | 8712204-2 | 64 | 145 | 12.88 | 39.22 | 7.53 | IGHV3-3*01 | ***, | 1.51 | |
| | | | | | | | | 2.9 mg/L | | |
| 114 | 366864-14 | 65 | 146 | 8.00 | 20.83 | 5.60 | IGHV3-3*01 | ****, | 0.45 | |
| | | | | | | | | 14.5 mg/L | | |
| 115 | 6111231-5 | 66 | 147 | 7.10 | 22.92 | 4.18 | IGHV3-3*01 | ****, | 2.28 | |
| | | | | | | | | 17.8 mg/L | | |
| 116 | 2878304-24 | 67 | 148 | 7.99 | 18.92 | 5.65 | IGHV3-3*01 | *, | 0.33 | |
| | | | | | | | | 0.4 mg/L | | |
| 117 | 3048192-20 | 68 | 149 | 8.41 | 25.00 | 5.17 | IGHV3-3*01 | *, | 0.32 | |
| | | | | | | | | 1.7 mg/L | | |
| 118 | 6593113-2 | 69 | 150 | 7.29 | 14.58 | 5.63 | IGHV3-3*01 | ****, | >1000 | |
| | | | | | | | | 9.5 mg/L | | |
| 119 | 208839-123 | 70 | 151 | 10.75 | 24.46 | 8.39 | IGHV3S53*01 | ****, | 0.35 | |
| | | | | | | | | 8.2 mg/L | | |
| 122 | 8496010-3 | 71 | 152 | 11.04 | 29.17 | 7.25 | IGHV3-3*01 | *, | >1000 | |
| | | | | | | | | 1.8 mg/L | | |
| Multivalent | T20-GS | 72 | / | / | / | / | / | / | / | |
| Multivalent | T20-EK | 73 | / | / | / | / | / | / | / | |
| Multivalent | T21-GS | 74 | / | / | / | / | / | / | / | |
| Multivalent | T21-EK | 75 | / | / | / | / | / | / | / | |
| Multivalent | D20 | 76 | / | / | / | / | / | / | / | |
| Multivalent | D21 | 77 | / | / | / | / | / | / | / | |
| Multivalent | 21-34 | 78 | / | / | / | / | / | / | / | |
| Multivalent | 21-105 | 79 | / | / | / | / | / | / | / | |
| Multivalent | 21-115 | 80 | / | / | / | / | / | / | / | |
| Multivalent | 21-36 | 81 | / | / | / | / | / | / | / | |
| Multivalent | 21-93 | / | / | / | / | / | / | / | / | |

| | | | | | | | | | | Thermosta- | Thermosta- |
|---|---|---|---|---|---|---|---|---|---|---|---|
| | | | | | Pseudo- | | | | | bility | bility |
| | | | Pseudo- | | neutralization | | Authentic | | | pET21b | pET22b |
| | SPR kd | SPR | neutralization | | IC50 | | virus | | Epitope | (avg. Tm | (avg. Tm |
| # | (1/s) | KD (M) | (GFP) | | (Luciferase) | | PRNT IC50 | | by SEC | ° C.) | ° C.) |
| 2 | | | 5 nM-50 nM | | / | | / | | / | / | / |
| 3 | | | 5 nM-50 nM | | / | | / | | / | / | / |
| 4 | | | 5 nM-50 nM | | / | | / | | / | / | / |
| 5 | | | / | | / | | / | | / | / | / |
| 6 | | | >3 | μm | / | | / | | / | / | / |
| 9 | | | 0.5 nM-5 nM | | 1.5 | nM | 1.721 | nM | Same | 51.4 | / |
| | | | | | | | | | as 21 | | |
| 10 | | | 5 nM-50 nM | | / | | / | | / | / | / |
| 11 | | | 0.5 nM-5 nM | | 1 | nM | 780 | pM | / | 50.1 | / |
| 12 | | | / | | / | | / | | / | / | / |
| 15 | | | 5 nM-50 nM | | / | | / | | / | / | / |
| 16 | | | 0.5 nM-5 nM | | 1.57 | nM | 3.16 | nM | Same | 53.5 | / |
| | | | | | | | | | as 21 | | |
| 17 | | | 0.5 nM-5 nM | | 1.1 | nM | 1.5 | nM | Same | 53.7 | / |
| | | | | | | | | | as 21 | | |
| 18 | | | 5 nM-50 nM | | / | | / | | / | 61.3 | / |
| 20 | | | <0.5 | nM | 114 | pM | 42.17 | pM | Same | 55.1 | 71.8 |
| | | | | | | | | | as 21 | | |
| 21 | <1.00E−6 | <1.00E−12 | <0.5 | nM | 38 | pM | 44.8 | pM | / | 50.3 | 72.8 |
| 24 | 3.08E−05 | 1.04E−11 | 5 nM-50 nM | | / | | / | | / | / | / |
| 26 | | | 5 nM-50 nM | | / | | / | | / | / | / |
| 28 | | | 0.5 nM-5 nM | | 6 | nM | / | | / | 46.6 | / |
| 29 | | | 5 nM-50 nM | | / | | / | | / | / | / |
| 32 | | | 5 nM-50 nM | | / | | / | | / | / | / |
| 33 | | | / | | / | | / | | / | / | / |
| 34 | | | 0.5 nM-5 nM | | 2 | nM | 1.124 | nM | Different | 46.3 | / |
| | | | | | | | | | from 21 | | |
| 36 | | | 0.5 nM-5 nM | | 7 | nM | / | | Different | 42.6 | / |
| | | | | | | | | | from 21 | | |
| 37 | | | / | | / | | / | | / | / | / |
| 39 | | | 50 nM-500 nM | | / | | / | | / | 50 | / |
| 41 | | | 5 nM-50 nM | | / | | / | | / | / | / |
| 42 | | | 5 nM-50 nM | | / | | / | | / | / | / |
| 43 | | | / | | / | | / | | / | / | / |
| 44 | | | / | | / | | / | | / | / | / |
| 46 | | | 50 nM-500 nM | | / | | / | | / | / | / |
| 48 | | | 500 nM-3 μM | | / | | / | | / | / | / |
| 51 | | | / | | / | | / | | / | / | / |
| 55 | | | 5 nM-50 nM | | / | | / | | / | / | / |
| 58 | | | / | | / | | / | | / | / | / |
| 59 | | | / | | / | | / | | / | / | / |
| 60 | | | 5 nM-50 nM | | / | | / | | / | / | / |
| 62 | | | / | | / | | / | | / | / | / |
| 63 | | | 5 nM-50 nM | | / | | / | | / | / | / |
| 65 | | | / | | / | | / | | / | / | / |
| 68 | | | 50 nM-500 nM | | / | | / | | / | / | / |
| 73 | | | / | | / | | / | | / | / | / |
| 74 | | | 50 nM-500 nM | | / | | / | | / | / | / |
| 76 | | | / | | / | | / | | / | / | / |
| 78 | | | 0.5 nM-5 nM | | 509 | pM | 765.6 | pM | | 49 | |
| 80 | | | 50 nM-500 nM | | / | | / | | / | / | / |
| 84 | | | 5 nM-50 nM | | / | | / | | / | / | / |
| 87 | | | / | | / | | / | | / | / | / |
| 89 | 6.24E−04 | 1.08E−10 | <0.5 | nM | 129 | pM | 153.8 | pM | Same | 39.8 | 65.9 |
| | | | | | | | | | as 21 | | |

| 90 | >3 | μM | / | / | / | / | / |
|---|---|---|---|---|---|---|---|
| 91 | / | | / | / | / | / | / |
| 92 | >3 | μM | / | / | / | / | / |

| 93 | 0.5 nM-5 nM | 14.5 | nM | / | | Different | 50.5 | / |
|---|---|---|---|---|---|---|---|---|
| | | | | | | from 21 | | |
| 94 | 50 nM-500 nM | / | | / | | / | 53.4 | / |
| 96 | 5 nM-50 nM | / | | / | | / | / | / |
| 100 | 5 nM-50 nM | / | | / | | / | / | / |
| 102 | 500 nM-3 μM | / | | / | | / | / | / |
| 103 | / | / | | / | | / | / | / |
| 104 | / | / | | / | | / | / | / |
| 105 | 0.5 nM-5 nM | 5 | nM | 5.1 | nM | Different | 53.7 | / |
| | | | | | | from 21 | | |
| 106 | / | / | | / | | / | / | / |
| 107 | 0.5 nM-5 nM | 568.8 | pM | 181.23 | pM | Same | 42.5 | / |
| | | | | | | as 21 | | |
| 111 | / | / | | / | | / | / | / |
| 112 | 0.5 nM-5 nM | 12.2 | nM | / | | Same | / | / |
| | | | | | | as 21 | | |
| 113 | 5 nM-50 nM | / | | / | | / | / | / |
| 114 | 0.5 nM-5 nM | 4.13 | nM | 4.875 | nM | Same | 59 | / |
| | | | | | | as 21 | | |
| 115 | 5 nM-50 nM | 10 | nM | 5.105 | nM | Different | 45.6 | / |
| | | | | | | from 21 | | |
| 116 | / | / | | / | | / | / | / |
| 117 | 50 nM-500 nM | / | | / | | / | / | / |
| 118 | / | / | | / | | / | / | / |
| 119 | 0.5 nM-5 nM | 523 | pM | 451.8 | pM | Same | 53 | / |
| | | | | | | as 21 | | |

| 122 | / | / | | / | / | / | / |
|---|---|---|---|---|---|---|---|
| Multi-valent | / | / | | / | / | / | / |
| Multivalent | / | 10.1 | pM | / | / | / | / |
| Multivalent | / | / | | / | / | / | / |
| Multivalent | / | 9.4 | pM | / | / | / | / |
| Multivalent | / | / | | / | / | / | / |
| Multivalent | / | 16 | pM | / | / | / | / |
| Multivalent | / | 57.7 | pM | / | / | / | / |
| Multivalent | / | 35.5 | pM | / | / | / | / |
| Multivalent | / | 28.3 | pM | / | / | / | / |

| | | | | | | |
|---|---|---|---|---|---|---|
| Multivalent | / | / | / | / | / | / |
| Multivalent | / | / | / | / | / | / |

### Example 2. Inhalable Nanobody (PiN-21) Prevents and Treats SARS-CoV-2 Infections in Syrian Hamsters at Ultra-Low Doses

By January 2021, a year after the outbreak of severe acute respiratory syndrome coronavirus 2 (SARS-CoV-2) was first reported (P. Zhou et al., 2020), close to 100 million people have been infected by this highly transmissible virus resulting in significant morbidity and mortality worldwide. In addition to vaccines, there is an unparalleled quest to develop innovative and cost-effective therapeutics to combat the COVID-19 pandemic (L. DeFrancesco 2020; F. Krammer 2020). Early treatment using high-titer convalescent plasma (CP) can reduce the risk of severe disease in seniors (R. Libster et al., 2021), although CP is limited by supply. Potent neutralizing monoclonal antibodies (mAbs), predominantly isolated from COVID-19 patients for recombinant productions, have been developed for passive immunotherapy (J. Hansen et al., 2020; B. Ju et al., 2020; S. J. Zost et al., 2020; L. Liu et al., 2020; D. F. Robbiani et al., 2020; T. F. Rogers et al., 2020; Y. Cao et al., 2020; P. J. M. Brouwer et al., 2020; M. A. Tortorici et al., 2020; S. Jiang et al., 2020). In vivo evaluations of mAbs in animal models of COVID-19 disease such as murine, hamster, and nonhuman primates (NHPs), have provided critical insights into efficacy and the mechanisms by which they alter the course of infection (A. Baum et al., 2020; K. H. Dinnon, 3rd et al., 2020; J. F. Chan et al., 2020; V. J. Munster et al., 2020; J. Yu et al., 2020; W. Deng et al., 2020; C. Munoz-Fontela et al., 2020; A. L. Hartman et al., 2020; S. H. Sun et al., 2020; A. Chandrashekar et al., 2020). While mAb therapy lifts hopes to treat mild symptom onset in patients, they nevertheless require exceedingly high administration doses—typically several grams for intravenous (i.v.) injection (P. Chen et al., 2021; D. M. Weinreich et al., 2021). The requirement of high doses for efficient neutralization reflects SARS-CoV-2 virulence, pathogenesis, and the notoriously low efficiency of i.v. delivering these relatively large biomolecules across the plasma-lung barrier to treat pulmonary infections (J. S. Patton et al., 2007). Moreover, the associated high costs and challenges in bulk manufacturing can further limit the broad clinical use of mAbs worldwide (L. DeFrancesco, 2020).

Camelid single-domain antibody fragments or nanobodies (Nbs) were developed that primarily target the receptor-binding domain (RBD) of the SARS-CoV-2 spike (S) glycoprotein for virus neutralization (S. Jiang, C. Hillyer, L. Du, 2020; Y. Xiang et al., 2020; M. Schoof et al., 2020; R. Konwarh, 2020; D. Wrapp et al., 2020). Highly selected Nbs and the multivalent forms obtain high neutralization potency comparable to, or even better (per-mass) than, some of the most successful SARS-CoV-2 neutralizing mAbs. In particular, an ultrapotent homotrimeric construct, Pittsburgh inhalable Nanobody 21 (PiN-21), efficiently blocked SARS-CoV-2 infectivity at below 0.1 ng/ml in vitro (Y. Xiang et al., 2020). Compared to mAbs, Nbs are substantially cheaper to produce. Moreover, affinity-matured, ultrapotent Nbs are characterized by high solubility and stability (Y. Xiang et al., 2021) that facilitate drug scaling, storage, and transportation, all of which are critical in response to pandemics. The excellent physicochemical properties and small sizes of Nbs raise an exciting possibility of efficient pulmonary delivery by aerosolization with characteristics of rapid onset of action, high local drug concentration/bioavailability, and improved patient compliance (needle-free) that can benefit a large population of SARS-CoV-2 infected patients (J. S. Patton, P. R. Byron, 2007; Y. Xiang et al., 2020; M. Schoof et al., 2020; W. Liang et al., 2020). However, despite the promise, no successful in vivo studies have been reported to date. The inferior pharmacokinetics of monomeric Nbs due to their small size (˜15 kDa) and a lack of Fc-mediated immune effectors' function, which is often required to augment the in vivo neutralizing activities of mAbs (F. Nimmerjahn, 2005; A. Schafer et al., 2021; S. Bournazos, et al., 2017), drive concerns for Nb-based therapy. It remains unknown if the high in vitro neutralization potency of SARS-CoV-2 Nbs can be translated into in vivo therapeutic benefits.

This study systematically evaluated the efficacy of PiN-21 for prophylaxis and treatment of SARS-CoV-2 infected Syrian hamsters which model moderate to severe COVID-19 disease. This study provided direct evidence that ultra-low administration of PiN-21 efficiently treats the virus infection. Notably, PiN-21 aerosols can be inhaled to target respiratory infection which drastically reduces viral loads and prevents lung damage and viral pneumonia. This novel Nb-based therapy shows high potentials for the treatment of early infection and can provide a robust and affordable solution to address the current health crisis.

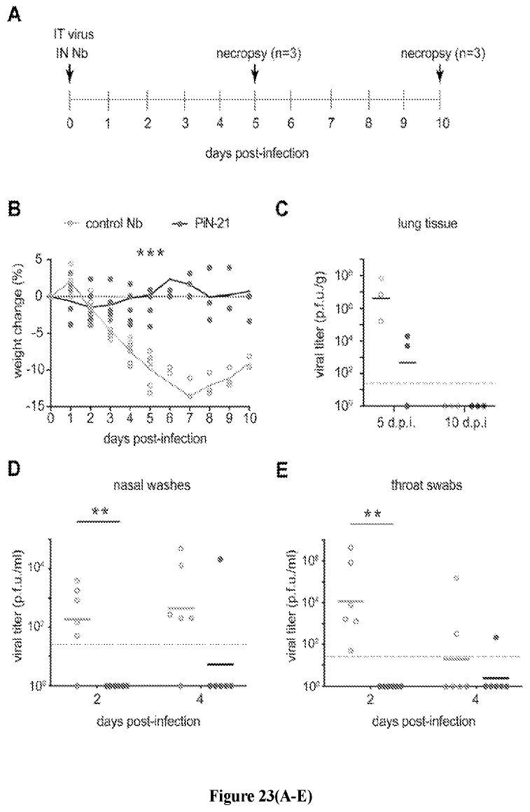

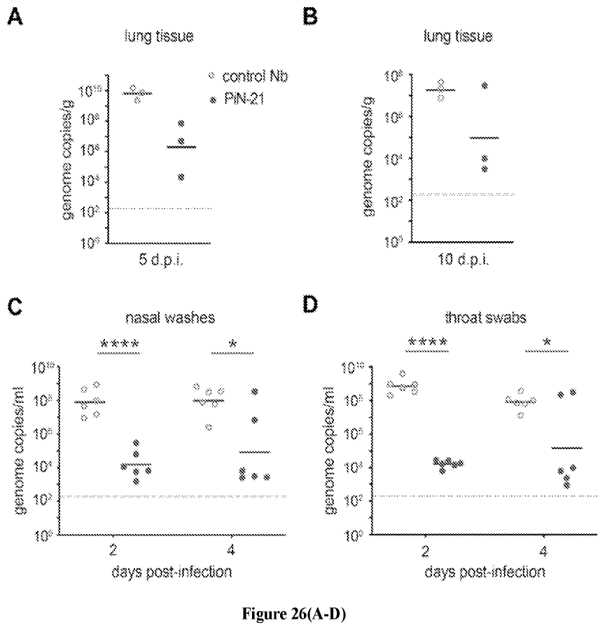

PiN21 efficiently protects and treats SARS-CoV-2 infection in Syrian hamsters. To assess the in vivo efficacy of PiN-21, 12 hamsters were divided into two groups and infected with 9×10⁴ plaque-forming units (p.f.u.) of SARS-CoV-2 via the intratracheal (IT) route. Shortly after infection, Nb was delivered intranasally (IN) at an average dose of 0.6 mg/kg (FIG. 23A). Animals were monitored daily for weight change and clinical signs of disease. Half of the animals were euthanized 5 days post-infection (d.p.i.) and the remaining were euthanized 10 d.p.i. Virus titers in lung samples from the euthanized animals were measured by plaque assay. Nasal washes and throat swabs were collected at 2 and 4 d.p.i. to determine viral loads in the upper respiratory tract. Consistent with published studies (T. F. Custodio et al., 2020; L. Hanke et al., 2020), IT inoculation of hamsters with SARS-CoV-2 resulted in a robust infection, rapid weight loss in all animals up to 16% at 7 d.p.i. and resulting recovery and reversal of weight loss by 10 d.p.i. before recovery. However, concurrent IN delivery of PiN-21 eliminated any significant weight loss in the infected animals (FIG. 23B). This dramatic protection was accompanied by a reduction of viral titer in the lungs, with an average decrease of 4 order of magnitude in the lung tissue, respectively, compared to control on 5 d.p.i. (FIG. 23C). Infectious virus was essentially cleared by 10 d.p.i. (FIG. 23C). Consistently, a 3-log reduction of the viral genomic RNA (gRNA) by reverse transcriptase (RT)-qPCR was evident on 5 and 10 d.p.i. (FIGS. 26A-26B).

Notably, the virus was undetectable in the upper respiratory tract (URT) including both nasal washes and throat swabs of all PiN-21-treated animals on 2 d.p.i. This is significantly different from the control group, where varying levels of infectious virus were present (FIGS. 23D-23E). Furthermore, five out of six PiN-21 treated animals remained protected from detectable infection 4 d.p.i. The results were further supported by a drastic decrease of gRNA in the URT (FIGS. 26C-26D). Together, this demonstrates that the high in vitro neutralization potency of PiN-21 can be translated into therapeutic benefits in vivo independent of Fc-mediated immune responses. PiN-21 can efficiently protect SARS-CoV-2 infection in hamsters by rapidly and drastically suppressing viral replication in both the URT and lower respiratory tract (LRT).

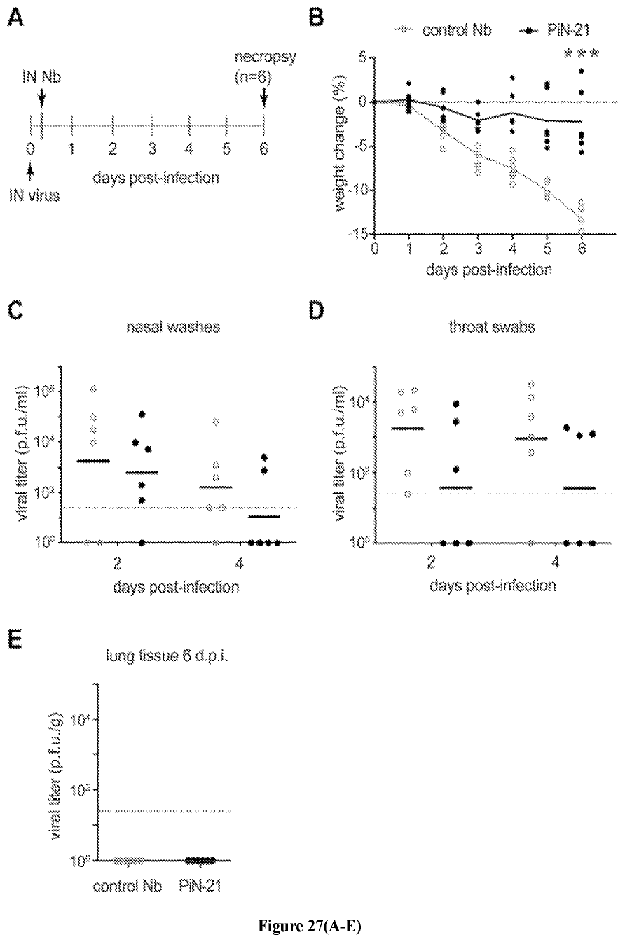

Previous studies reveal that clinical mAbs are less effective for COVID-19 treatment (post-infection) than for prophylaxis (pre-infection) in animal models, possibly reflecting the virulence of SARS-CoV-2, speed of virus replication, and rapid symptom onset (A. Baum et al., 2020; A. L. Hartman et al., 2020; W. Guan et al., 2020). Therefore, the therapeutic potential of PiN-21 was evaluated since it was highly effective when co-administered. To explore the second route of infection, hamsters were inoculated IN with 3×10⁴ p.f.u. of SARS-CoV-2. PiN-21 or a control Nb (0.6 mg/kg) was IN-delivered to animals 6 hours post-infection (h.p.i.). Animal weights were monitored daily, throat swabs and nasal washes were collected, before euthanized on 6 d.p.i. (FIG. 27A). Similar to the IT route, IN-infection of hamsters with SARS-CoV-2 resulted in precipitous weight losses in the control animals. Intranasal treatment using PiN-21 significantly reduced weight loss throughout the assessment period (FIG. 27B), paralleling the results of clinical mAbs in the same model albeit using substantially higher doses. Less than 100-fold reduction in virus titers were found in nasal washes and throat swabs on 2 and 4 d.p.i. (FIGS. 27C-27D). Moreover, infectivity was undetectable in lung tissues 6 d.p.i. (FIG. 27E), indicating that the virus has been predominantly cleared. Analysis of early time points can be needed to better understand virus suppression by Nb treatment.

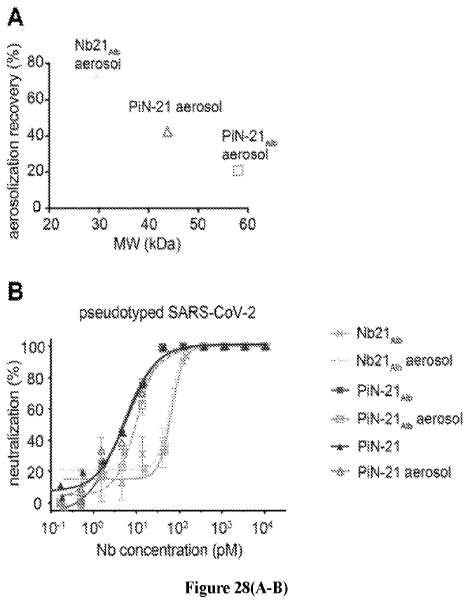

PiN21 aerosolization effectively treats SARS-CoV-2 infected hamsters at an ultra-low dose. Pulmonary delivery by inhalation was evaluated. To evaluate the impact of construct size and pharmacokinetics on lung uptake, monomeric Nb21 and PiN-21 were fused to an Nb that binds serum albumin (Alb) of both human and rodents with high affinity to generate two serum-stable constructs (Nb-21_{Alb} and PiN-21_{Alb}) (Z. Shen et al., 2020). Using a portable mesh nebulizer, Nb21_{Alb}, PiN-21, and PiN-21_{Alb} were aerosolized and evaluated for their post-aerosolization neutralization activities by pseudovirus neutralization assay. All constructs retained high neutralization potency in vitro (FIG. 28B). The amount of Nbs recovered post-aerosolization was inversely correlated with the size of constructs (FIG. 28A). Moreover, while Nb-21_{Alb} had the highest recovery, the post-aerosolization in vitro neutralization activity was substantially lower than for other constructs, Nb-21_{Alb} was therefore excluded from downstream therapeutic analyses.

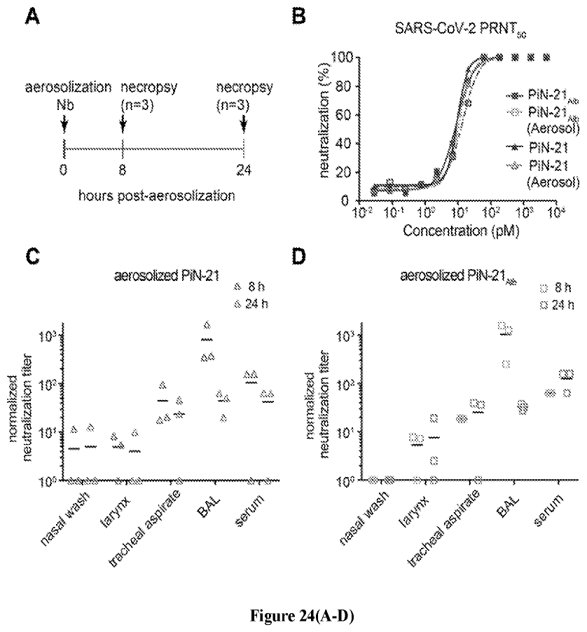

Next, two ultrapotent constructs PiN-21 and PiN-21_{Alb} were compared for targeted aerosolization delivery into hamsters. Nbs were aerosolized using a nebulizer (Aerogen, Solo) that produces small aerosol particles with a mass median aerodynamic diameter of ˜3 μm (Table 5). Animals were sacrificed 8 and 24 hours post-administration to assess Nb distribution and activities when recovered from various respiratory compartments and sera (FIG. 24A). Consistent with the result using the portable nebulizer, it was found that the inhalation dose of PiN-21 was approximately two times PiN-21_{Alb} (at 8 h, 41.0 μg or 0.24 mg/kg for PiN-21 v.s. 23.7 μg or 0.13 mg/kg for PiN-21_{Alb}) (Table 5) while neutralization activity as assessed by plaque reduction neutralization test (PRNT₅₀) of SARS-CoV-2 remained essentially unchanged after aerosolization of both Nb constructs (FIG. 24B).

The neutralization activities of both Nb constructs were detected throughout the respiratory tract and in sera. Within the airways the neutralizing activities were predominantly associated with bronchoalveolar lavage (BAL) fluid, followed by tracheal aspirate, larynx wash, and nasal wash samples (FIGS. 24C-24D). Compared to 8-hour post-inhalation, it was found that the amounts and activities of Nbs in BAL, but not in sera, were substantially lower 24-hour post-inhalation, indicating more rapid clearance. In addition, Nb conjugation to serum albumin did not impact the activities in the airways, whilst stability was enhanced in the serum. These data underscore the requirement of an ultra-low dose of the ultrapotent PiN-21 construct to neutralize SARS-CoV-2 infectivity in vivo efficiently. Finally, PiN-21 was preferentially selected for further evaluation owing to the high stability and resistance to aerosolization, which are critical for clinical applications.

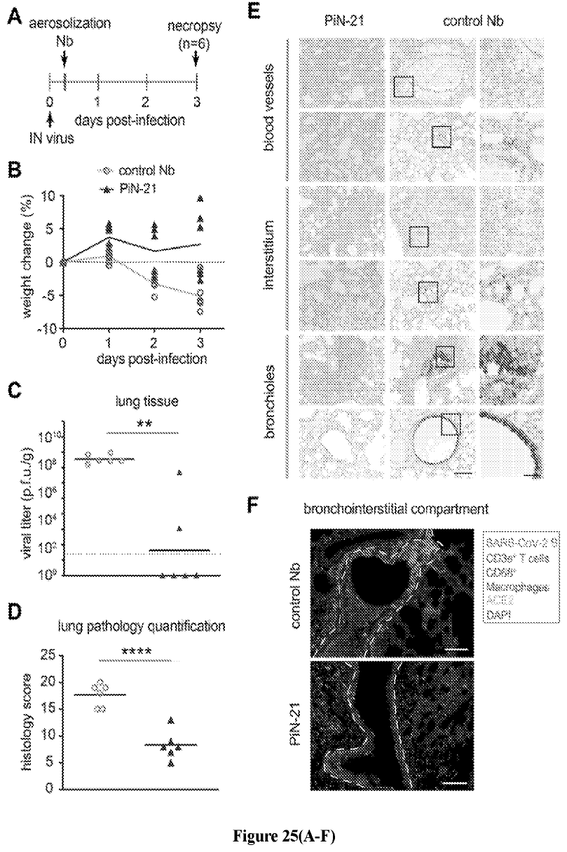

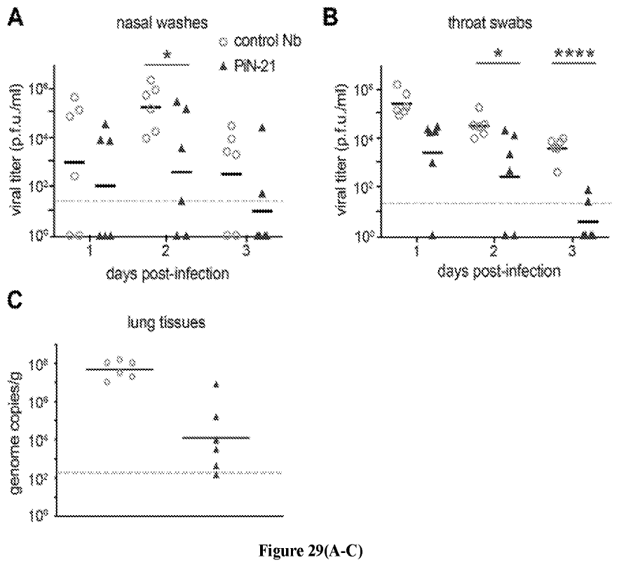

To assess the therapeutic efficacy of PiN-21 by inhalation, 12 hamsters were IN inoculated with SARS-CoV-2 (3×10⁴ p.f.u.) followed by single-dose aerosolization treatment (˜0.2 mg/kg) of either PiN-21 or the control Nb at 6 h.p.i. Animals were monitored for weight loss, throat swabs and nasal washes were collected daily. Animals were euthanized (3 d.p.i.) and lungs and trachea were collected for virological, histopathology, and immunohistochemical analysis (FIG. 25A). Notably, pulmonary delivery of PiN-21 aerosols, despite only a minute amount, led to a remarkable reverse of weight loss in the treated animals. The average weight gain was 2% in PiN-21 versus 5% loss in the control on 3 d.p.i. (FIG. 25B). The weight loss in the control group was highly reproducible when compared with the above experiments. Critically, aerosolization treatment diminished infectious viruses in lung tissue by 6 orders of magnitude (FIG. 25C). The treatment also substantially decreased virus gRNA in the lungs (FIG. 29C). Moreover, a substantial reduction of viral titers in nasal washes and throat swabs was observed (FIG. 29A-29B). This indicates that Nb administration by aerosolization can limit human-to-human transmission of SARS-CoV-2.

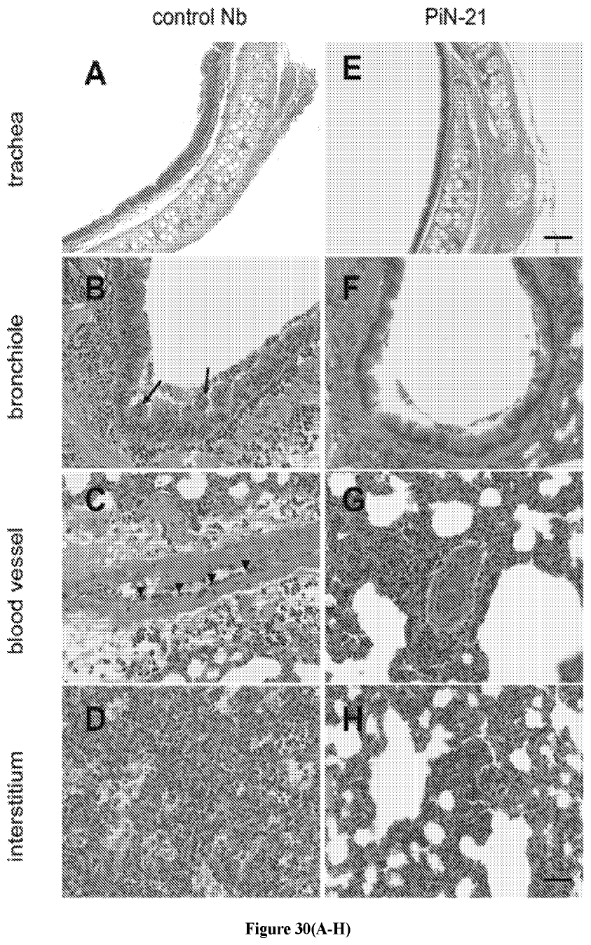

Effective control of SARS-CoV-2 infection in the LRT of PiN-21 aerosolized animals. To understand the mechanisms by which Nb aerosols prevent and/or ameliorate lower respiratory disease caused by SARS-CoV-2 infection better, whole lung semi-quantitative ordinal histologic analysis were performed on control (n=6) and PiN-21 (n=6) treated animals euthanized at 3 d.p.i (Tables 6-7). Cumulative scores encompassed pathologic features of airways, blood vessels, and alveoli/pulmonary interstitium. PiN-21 aerosolization protected most animals (5/6) from severe COVID-related histopathologic disease reflected by decreased ordinal scores (P<0.0001) when compared to Nb treated controls (FIG. 25D, Table 8). Histopathologic findings in the control group resembled previous reports of SARS-CoV-2 inoculation in Syrian hamsters (M Imai et al., 2020; S. F. Sia et al., 2020). Pulmonary disease observed in the PiN-21 treated animals was very mild (FIG. 3E, FIG. 30) being characterized by the absence of severe necrotizing bronchiolitis in the majority of animals (5/6), a pathological finding ubiquitously observed in all control animals. Furthermore, the single PiN-21 treated animal with necrotizing bronchiolitis had localized disease, compared to the multifocal and bilateral distribution observed in most Nb controls (4/6). Bronchiolitis was also affiliated with less severe bronchial hyperplasia and hypertrophy and absence of syncytial cells when compared to Nb controls. The predominant histologic finding in PiN-21 treated animals was minimal-to-mild perivascular and peribronchial mononuclear inflammation consisting of macrophages and lymphocytes. Furthermore, aside from the one animal already mentioned, the PiN-21 group had considerably less interstitial inflammation with decreased vascular permeability, as indicated by the absence of perivascular and intra-alveolar edema, hemorrhage, and fibrin exudation (FIG. 30).

In control animals, S antigen was abundant in the cytoplasm of the bronchiolar epithelium, with less common detection in alveolar type 1 and 2 pneumocytes. Interstitial and peribronchiolar infiltrates were composed of large numbers of CD3e+ T cells and CD68+macrophages, with a complete absence of angiotensin-converting enzyme 2 (ACE2) in the apical cytoplasm of bronchiole epithelium in areas with abundant viral S (FIG. 25F, upper panel). Consistent with a striking 6-log virus reduction after aerosolization, S antigen was extremely sparse (<1% of permissive cells) in all PiN-21 treated animals, with decreased T cell and macrophage immune cell infiltrate, and retention of native apical bronchiole ACE2 expression (FIG. 25F, lower panel).

To determine the impact of PiN-21 on the upper airways of the lower respiratory tract, the trachea was also examined histologically. In PiN-21 treated animals, tracheas for all animals were within normal limits, while mild to moderate neutrophilic and lymphohistiocytic tracheitis, with variable degrees of degeneration and necrosis, and segmental hyperplasia and hypertrophy were observed in all the control animals (FIG. 25F). In summary, these data clearly demonstrate that PiN-21 aerosolization given during early disease course is highly effective in decreasing SARS-CoV-2 entry, subsequent replication in permissive epithelial cells of the lower respiratory tract and this, in turn, has a major impact on viral shedding. The result is the prevention of disease, including decreased cytopathic effect on permissive epithelial cells, retention of ACE2 expression on permissive bronchioles, and decreased recruitment of inflammatory cells to sites of replication.

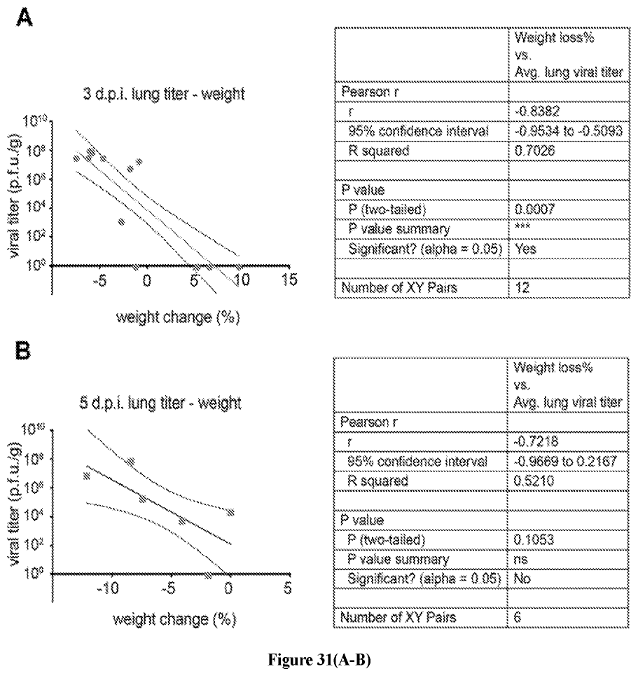

This work demonstrates the high therapeutic efficacy of a trimeric Nb against SARS-CoV-2 infection in Syrian hamsters. These investigations leverage both intranasal and aerosol delivery of PiN-21 and demonstrate Nb treatment effectively targets the deep and local pulmonary structures such as terminal alveoli, which are lined with alveolar cells rich in ACE2 receptor to block viral entry and replication efficiently. Moreover, infection-induced weight loss correlates with pulmonary virus titer (Pearson r=−0.7) (FIG. 31) and this clinical sign can be used to indicate the onset of infections (M. A. Tortorici et al., 2020). Notably, the ability of PiN-21 to eradicate viral replication and lung pathology almost completely in both the URT and LRT in hamsters contrasts the effects recently shown by clinical antibodies, which, despite administrating at high doses (e.g., from 10 to 50 mg/kg), remain particularly challenging to treat SARS-CoV-2 infection in the same model (A. Baum et al., 2020).

Significantly improved delivery upon aerosolization can be anticipated in NHPs and humans since airway anatomical structures differ considerably from small rodents in which a high degree of inertial impaction is seen using liquid droplets. Several inhalation therapeutics with excellent safety profiles are commercially available and many are under clinical trials (J. S. Patton et al., 2007; B. L. Laube, 2015). A combination of extremely low deposit doses can minimize potential adverse effects. Nevertheless, further preclinical analysis including an extensive toxicopathologic investigation, preferentially in a NHP model, can be needed before moving this technology into human trials. PiN-21 aerosolization treatment can provide both a convenient and cost-effective solution to alleviate disease onset and reduce virus transmission, especially for mild COVID-19 patients who constitute major populations of infections. It can also benefit high-risk groups, such as seniors, immunocompromised individuals, and infants, in both inpatient and outpatient settings. Finally, as prevalent circulating variants of SARS-CoV-2 have emerged to evade clinical antibodies and wane vaccine-elicited serologic responses (N. G. Davies et al., 2020; Z. Wang et al., 2021; H. Tegally et al., 2020; A. J. Greaney et al., 2021; E. C. Thomson et al., 2020), this proof-of-concept study shows the use of stable, multi-epitope and multivalent Nb constructs, in combination with PiN-21, as an aerosol cocktail which can be rapidly generated to block virus mutational escape (Y. Xiang et al., 2020).

Materials and Methods.

Ethics. The animal work performed adhered to the highest level of humane animal care standards. The University of Pittsburgh is fully accredited by the Association for Assessment and Accreditation of Laboratory Animal Care (AAALAC). All animal work was performed under the standards of the Guide for the Care and Use of Laboratory Animals published by the National Institutes of Health (NIH) and according to the Animal Welfare Act guidelines. All animal studies adhered to the principles stated in the Public Health Services Policy on Humane Care and Use of Laboratory Animals. The University of Pittsburgh Institutional Animal Care and Use Committee (IACUC) approved and oversaw the animal protocols for these studies (#20067405).

Biological safety. All work with SARS-CoV-2 was conducted under biosafety level-3 (BSL-3) conditions in the University of Pittsburgh Center for Vaccine Research (CVR) and the Regional Biocontainment Laboratory (RBL). Respiratory protection for all personnel when handling infectious samples or working with animals was provided by powered air-purifying respirators (PAPRs; Versaflo TR-300; 3M, St. Paul, MN). Liquid and surface disinfection was performed using Peroxigard disinfectant (1:16 dilution), while solid wastes, caging, and animal wastes were steam sterilized in an autoclave.

Nanobody production. The PiN-21 gene (ANTE-CoV2-Nab21T_{GS}) was synthesized from Synbio Biotechnologies and cloned into pET-21b vector as previously described (Y. Xiang et al., 2020; Y. Xiang et al., 2021). Nb21_{Alb} and PiN-21_{Alb} were generated by sub-cloning a human serum albumin binding Nb (Z. Shen et al., 2020) into the N-terminus of Nb21 and PiN-21 constructs. The plasmid was transformed into BL21(DE3) cells and plated on LB-agar with 50 μg/ml ampicillin at 37° C. overnight. Single bacterial colonies were picked and cultured in LB broth to reach an O.D. of ˜0.5-0.6 before IPTG induction (0.5 mM) at 16° C. overnight. Cells were then harvested, sonicated, and lysed on ice with a lysis buffer (1×PBS, 150 mM NaCl, TX-100 with protease inhibitor). After cell lysis, his-tagged Nbs were purified by Cobalt resin and natively eluted using the imidazole buffer. Eluted Nbs were subsequently dialyzed into 1×DPBS, pH 7.4. For animal studies, endotoxin was removed with the ToxinEraser™ Endotoxin Removal Kit (Genscript), and the endotoxin level was measured using the ToxinSensor™ Chromogenic LAL Endotoxin Assay Kit (Genscript) to make sure <1 EU/ml. The proteins were sterile-filtered using the 0.22 μm centrifuge filters (Costar) before use.

Virology. SARS-CoV-2/München-1.1/2020/929 (Munich) (MOI of 0.03) was added to confluent monolayers of Vero E6 cells in 25 T175. After 1 h of incubation at 37° C., 5% (v/v) CO2, 20 ml/flask of virus growth medium [DMEM (Dulbecco's Modified Eagle Medium; Gibco) supplemented with 10% (v/v) fetal bovine serum (FBS; Life technologies), 1% (v/v) 1-glutamine (Gibco) and 1% (v/v) penicillin/streptomycin (pen-strep; Life technologies) was added and incubation was continued for 66-72 h until cytopathic effect was observed. Virus-containing supernatant was collected and clarified by centrifugation at 3500 rpm for 30 mM at 4° C. The cleared virus supernatant was aliquoted and stored at −80° C.

Plaque assay: Samples were prepared in Opti-MEM (Gibco) and were added, in duplicate, to confluent Vero E6 monolayers in six-well plates (Fisher; 200 μl/well). After 1 h of incubation at 37° C., 5% CO₂, 2 ml/well of virus growth medium containing 0.1% (w/v) immunodiffusion agarose (MP Biomedicals) was added and incubation was continued for 72 h. Plates were fixed with 2 ml/well formaldehyde (37% (w/v) formaldehyde stabilized with 10-15% (v/v) methanol; Fisher Scientific) for 15 min at room temperature. Agarose and fixative were discarded and 1 ml/well 1% (w/v) crystal violet in 10% (v/v) methanol (both Fisher Scientific) was added. Plates were incubated at room temperature for 20 mM and then rinsed thoroughly with water. Plaques were then enumerated.

General animal procedures. Syrian hamsters (aged 3-6 months old both male and female) were obtained from the Charles River, MA. For procedures (virus infection, throat swab, and nasal wash collection), each animal was sedated with 3-5% Isoflurane. Baseline body weights were measured for all animals before infection. The animals were monitored twice daily for signs of COVID-19 disease (ruffled fur, hunched posture, labored breathing, anorexia, lethargy) post-challenge with SARS-CoV-2. Bodyweight was measured once daily during the study period. At necropsy, small pieces of the lung were collected for viral load determination. Throat swabs were collected using ultrathin swabs (Puritan™ PurFlock™ Ultra Sterile Flocked Swabs) which were placed in Opti-MEM (Invitrogen) containing double strength Antibiotic-Antimycotic (anti-anti; Life technologies). Nasal washes were collected using 500 μl of PBS with anti-anti. All samples were stored at −80° C. until viral load determination. The whole trachea and lungs were collected in Opti-MEM, Trizol, or 4% PFA respectively for virus titrations, RT-qPCR, and histopathological examinations.

Under Isoflurane anesthesia, hamsters were infected (300 μl) with 9×10⁴ p.f.u. (300 μl) of SARS-CoV-2 via IT administration, immediately followed by IN administration of 100 μg (50 μl per nare) PiN-21 or a control Nb.

Under Isoflurane anesthesia, hamsters were infected (50 μl per nare) with 3×10⁴ p.f.u. of SARS-CoV-2. At 6 h.p.i., animals were administered with 100 μg (50 μl per nare) PiN-21 (n=6) or a control Nb (n=6).

Hamsters were administered with PiN-21 and PiN-21_{Alb} via aerosol route and sacrificed at 8 (n=3) and 24 h (n=3) post-administration.

Under Isoflurane anesthesia, hamsters were infected intranasally (50 ul per nare) with 3×10⁴ p.f.u. of SARS-CoV-2. At 6 h.p.i., animals were administered with PiN-21 (n=6) or a control Nb (n=6) via the aerosol route.

Bronchoalveolar lavage (BAL) collection. Lungs with trachea were harvested from euthanized animals A Sovereign Feeding Tube (Covetrus) was cut to the optimal length and connected to a 5 ml syringe (BD) containing 3 ml PBS with anti-anti before placement into the trachea. The PBS was gently pushed into the lungs until they were fully inflated after which the liquid (BAL) was drawn back into the syringe.

Sample extraction and processing. For tissues, 100-200 mg of tissue was harvested, suspended in 1 ml Opti-MEM supplemented with 2× anti-anti, and homogenized using a D2400 homogenizer (Benchmark Scientific). The eluate from swabs and nasal washes were analyzed directly. Virus isolations were performed by inoculation of tissue homogenates (100 μl) onto Vero E6 cells (Hartman et al, 2020). For the preparation of RNA, tissue homogenate, swab eluate, or nasal wash (100 μl) was added to 400 μl of Trizol LS (Ambion) and thoroughly mixed by vortexing. To ensure virus inactivation, the samples were incubated for 10 minutes at room temperature and stored overnight at −80° C. prior to removal from the BSL-3 facility. Subsequent storage at −80° C. or RNA isolation and one-step RT-qPCR analyses were performed at BSL-2. RNA was extracted from these samples using Direct-zol RNA purification kits (Zymo Research) according to the manufacturer's instructions. Viral RNA was detected by RT-qPCR targeting the SARS-CoV-2 nucleocapsid (N) segment as previously described (W. B. Klimstra et al., 2020). The primers used are forward primer: 2019—nCoV_N2F (TTACAAACATTGGCCGCAAA, SEQ ID NO: 181), reverse primer: 2019—nCoV_N2R (GCGCGACATTCCGAAGAA, SEQ ID NO: 182) and probe: 2019—nCoV_N2probe (FAM—ACAATTTGCCCCCAGCGCTTCAG-BHQ1, SEQ ID NO: 183). The PCR conditions and the standard curve generation was carried out as described previously (W. B. Klimstra et al., 2020). Data were normalized by tissue weight and are reported as copies of RNA determined by comparing the cycle threshold (CT) values from the unknown samples to CT values from a positive-sense SARS-CoV-2 vRNA standard curve as previously described (W. B. Klimstra et al., 2020). Graphs were generated using GraphPad Prism, version 9.

Neutralization assay. Nbs or hamster serum dilution (100 μl) was mixed with 100 μl of SARS-CoV-2 (Munich: P3 virus) containing 75 p.f.u. of the virus in Opti-MEM. The serum-virus mixes (200 μl total) were incubated at 37° C. for 1 h, after which they were added dropwise onto confluent Vero E6 cell monolayers in six-well plates. After incubation at 37° C., 5% (v/v) CO₂ for 1 h, 2 ml of 0.1% (w/v) immunodiffusion agarose in DMEM supplemented with 10% (v/v) FBS and 2× anti-anti was added to each well. After incubation at 37° C., 5% (v/v) CO₂ for 72 h, the agarose overlay was removed and the cell monolayer was fixed with 1 ml/well formaldehyde [37% (w/v) formaldehyde stabilized with 10-15% (v/v) methanol] for 20 min at room temperature. Fixative was discarded and 1 ml/well 1% (w/v) crystal violet in 10% (v/v) methanol was added. Plates were incubated at room temperature for 20 min and rinsed thoroughly with water. Plaques were then enumerated and the 80% and/or 50% plaque reduction neutralization titer (PRNT80 or PRNT50) was calculated (Y. Xiang et al., 2020; W. B. Klimstra et al., 2020). A SARS-CoV-2 positive convalescent patient serum and naive human serum were used as positive and negative controls respectively and, an uninfected cell, were performed to ensure that virus neutralization was specific.

Nanobody aerosolization. Aerosol exposures of hamsters to nanobodies were performed under the control of the Aero3G aerosol management platform (Biaera Technologies, Hagerstown, MD) as previously described for rodents (S. A. Faith, 2019). Hamsters were loaded into metal exposure cages and transported via mobile transfer cart to the Aerobiology suite in the RBL. There they were transferred into a class III biological safety cabinet and placed inside a rodent whole-body exposure chamber. Hamsters were exposed for 12-15 minutes to small particle aerosols containing nanobodies generated by the Aerogen Solo vibrating mesh nebulizer (Aerogen, Chicago, IL)(J. Yu et al., 2020). The system was set in a push/pull configuration with an equal volume of input air (19.5 liters per minute (lpm) total: 7.5 lpm generator, 12 lpm dilution air) and exhaust (19.5 lpm total: 6 lpm sampler, 5 lpm particle sizer, 8.5 additional vacuum) equal to 0.5 air changes/minute in the exposure chamber. To determine inhaled dose, an all-glass impinger (AGI; Cat #7541-10, Ace Glass, Vineland, NJ) containing 10 ml of PBS+0.001% antifoam was attached to the chamber and operated at 6 lpm, −6 to −15 psi. Particle size was measured once during each exposure at 5 minutes using an Aerodynamic Particle Sizer (TSI, Shoreview, MN) operating at 5 lpm. A 5-minute air wash followed each aerosol, after which animals were returned to their cage. AGI samples were evaluated to determine the concentration of nanobodies recovered from the aerosol. The inhaled dose was determined as the product of the nanobody aerosol concentration, duration of exposure, and the minute volume of the individual hamster (J. D. Bowling et al., 2019). Minute volume was determined using Guyton's formula (A. C. Guyton, 1947).

Histologic processing and analysis. Tissue samples were fixed for a minimum of 24 h in 4% PFA before being removed from BSL-3 and subsequently processed in a Tissue-Tek VIP-6 automated vacuum infiltration processor (Sakura Finetek) and embedded in paraffin using a HistoCore Arcadia paraffin embedding machine (Leica). 5 μm tissue sections were generated using an RM2255 rotary microtome (Leica) and transferred to positively charged slides, deparaffinized in xylene, and dehydrated in graded ethanol. Tissue sections were stained with hematoxylin and eosin for histologic examination, with additional serial sections utilized for immunohistochemistry (IHC). A Ventana Discovery Ultra (Roche) tissue autostainer was used for IHC. Specific protocol details are outlined in Tables 9-11. The histomorphological analysis was performed by a single board-certified veterinary pathologist (N.A.C.), who developed an ordinal grading score encompassing the diversity and severity of histologic findings using isotype control administered animals as a baseline. Histologic criteria were broken down into three compartments: airways, blood vessels, and interstitium, with results utilized to generate a cumulative lung injury score. This score also incorporated the overall degree of immunoreactivity to the SARS-CoV-2 S antigen. A summary of individual animal scores and specific criteria utilized to score lungs is included in Tables 6-7.

Multispectral Whole Imaging. Brightfield and fluorescent images were acquired using a Mantra 2.0™ Quantitative Pathology Imaging System (Akoya Biosciences). To maximize signal-to-noise ratios, fluorescent images were spectrally unmixed using a synthetic library specific for the Opal fluorophores used for each assay and for DAPI. An unstained Syrian hamster lung section was used to create an autofluorescence signature that was subsequently removed from images using InForm software version 2.4.8 (Akoya Biosciences).

Nanobody aerosolization using the mesh nebulizer. Nb (Nb21_{Alb}, PiN-21 and PiN-21_{Alb}) was concentrated to 1 ml (1.5 mg/ml) in 1×DPBS. 0.5 ml was saved as a control for ELISA and pseudovirus neutralization assay. The other 0.5 ml was aerosolized by using a portable mesh atomizer nebulizer (MayLuck). No obvious dead volume was observed. The aerosolized droplets were collected in a microcentrifuge tube. The concentration was measured to calculate the recovery of the proteins.

Pseudotyped SARS-CoV-2 neutralization assay. Pseudotype neutralization assay was carried out and IC50 was calculated as previously described (Y. Xiang et al., 2020).

TABLE 5
Summary of aerosolization.

| | PiN21 | PiN21Alb |
|---|---|---|
| Exposure time | 10 | 10 |
| AGI vol (ml) | 10 | 10 |
| AGI conc (μg/ml) | 223.0 | 129.0 |
| Aerosol conc (ug/ml) | 37.2 | 21.5 |
| Spray Factor | 4.1E−06 | 2.4E−06 |
| Inhaled dose (μg/animal) | 41.0 | 23.7 |

TABLE 6
Semi-quantitative histology analysis.

| Feature | Grade | Histologic Description |
|---|---|---|
| Airways | 0 | Normal |
| (bronchi and | 1 | Mild rare multifocal infiltrates (intramural and/or peribronchiolar) |
| bronchioles) | | of neutrophils, macrophages, and lymphocytes. |
| | 2 | Moderate multifocal infiltrates (intramural, intraluminal, and |
| | | peribronchiolar) of neutrophils, macrophages, and lymphocytes |
| | | with epithelial degeneration. |
| | 3 | Severe multifocal infiltrates (intramural, intraluminal, and |
| | | peribronchiolar) of neutrophils, macrophages, and lymphocytes |
| | | with epithelial degeneration, necrosis, and squamous metaplasia. |
| Alveoli | 0 | Normal |
| and/or | 1 | Mild multifocal infiltrates of macrophages, neutrophils, and |
| interstitium | | lymphocytes within alveoli or septa. |
| | 2 | Moderate multifocal infiltrates of macrophages, neutrophils, and |
| | | lymphocytes within alveoli or septa with edema and/or |
| | | hemorrhage. |
| | 3 | Severe multifocal infiltrates of macrophages, neutrophils, and |
| | | lymphocytes within alveoli or septa with edema and fibrin. |
| Blood | 0 | Normal |
| vessels | 1 | Mild perivascular infliltrate of low numbers of inflammatory cells |
| | | (predominantly lymphocytes). |
| | 2 | Moderate multifocal intramural and/or perivascular infiltrate of |
| | | inflammatory cells with mild perivascular edema and moderate |
| | | segmental reactive endothelium. |
| | 3 | Severe multifocal intramural and/or perivascular infiltrate of |
| | | inflammatory cells with severe perivascular edema and prominent |
| | | reactive endothelium. |

TABLE 7
% of permissive cells with immunoreactivity
to SARS-CoV-2 Spike.

| | |
|---|---|
| 0 | none observed |

| | | |
|---|---|---|
| 1 | Mild | <5% |
| 2 | Moderate | 5 to 25 |
| 3 | Severe | >25 <50 |

TABLE 8
Animal histopathology scores.

| | | Airways | | Alveoli and/or interstitium | | Blood vessels | | INC | |
|---|---|---|---|---|---|---|---|---|---|
| | | Left lung | Right lung | Left lung | Right lung | Left lung | Right lung | SARS- | Cumulative |
| | Animal ID | lobe | lobes | Sobe | lobes | lobe | lobes | CoV 2 | score |
| Control isotype | 1 | 3 | 3 | 2 | 3 | 2 | 3 | 3 | 19 |
| | 2 | 3 | 3 | 2 | 2 | 2 | 3 | 3 | 18 |
| | 3 | 3 | 3 | 2 | 3 | 2 | 3 | 3 | 19 |
| | 4 | 1 | 3 | 1 | 3 | 1 | 3 | 3 | 15 |
| | 5 | 1 | 3 | 1 | 3 | 1 | 3 | 2 | 15 |
| | 6 | 3 | 3 | 3 | 3 | 2 | 3 | 3 | 20 |
| PIN-21 | 7 | 1 | 1 | 0 | 0 | 1 | 1 | 1 | 5 |
| | 8 | 1 | 2 | 1 | 2 | 1 | 1 | 1 | 9 |
| | 9 | 1 | 3 | 1 | 2 | 1 | 1 | 1 | 8 |
| | 10 | 3 | 1 | 3 | 2 | 2 | 1 | 1 | 13 |
| | 11 | 1 | 1 | 1 | 1 | 1 | 1 | 1 | 7 |
| | 12 | 1 | 1 | 2 | 1 | 1 | 1 | 1 | 8 |

TABLE 9
Monoplex SARS-CoV-2 Spike DAB IHC

| | | Manufacturer | Primary | |
|---|---|---|---|---|
| Species and | | and | Antibody | |
| clone | Antigen | Clone | Dilution | Chromogen |
| Ms Monoclonal | SARS-CoV | Cell Signaling | 1:1000 | DAB |
| | Spike | Technology | | |
| | | E7U60 | | |

Diaminobenzidine—DAB;
IHC—immunohistochemistry

TABLE 10
4-plex fluorescent IHC lung panel

| | | Manufacturer | Primary | |
|---|---|---|---|---|
| Species and | | and | Antibody | |
| clone | Antigen | Clone | Dilution | Fluorophore |
| Ms | SARS- | Cell | 1:1,000 | Opal 570 |
| Monoclonal | CoV- | Signaling | | (1:300) |
| (E7U60) | 2 Spike | Technology | | |
| Rb | ACE2 | Abcam | 1:200 | Opal 480 |
| Monoclonal | | | | (1:100) |
| (EPR34435) | | | | |
| Rb | CD3e+ | Dako | 1:100 | Opal 620 |
| Polyclonal | | | | (1:100) |
| Rb | Iba1 | Wako | 1:2,000 | Opal 690 |
| polycional | (CD68 + | | | (1:50) |
| | Macrophages) | | | |

ACE2—angiotensin converting enzyme-2,
IHC—immunohistochemistry

### Example 3. Potent Neutralizing Nanobodies Resist Convergent Circulating Variants of SARS-CoV-2 by Targeting Novel and Conserved Epitopes

The Coronavirus Disease 2019 (COVID-19) pandemic has caused devastating consequences to global health and the economy. In addition to vaccine development, potent neutralizing mAb isolated from convalescent plasma (Huang, A. T. et al. 2020) have been approved for emergency therapeutic use with more candidates in the pipeline (Cohen, M. S. 2021; Chen, P. et al. 2021; Weinreich, D. M. et al. 2021). Camelid VHH single-domain antibodies, or nanobodies (Nbs) have also been successfully developed for virus neutralization (Koenig, P. A. et al., 2021; Bracken, C. J. et al., 2021; Schoof, M. et al., 2021; Xiang, Y. et al., 2020; Walter, J. D. et al., 2020; Ahmad, J. et al., 2021; Custodio, T. F. et al., 2020; Huo, J. et al., 2020). Using camelid immunization with RBD_{SARS-CoV2} and an advanced proteomic pipeline identified >8,000 high-affinity RBD Nbs including a repertoire of ultrapotent Nbs (Xiang, Y. et al., 2020). Affinity-matured Nbs are highly soluble, stable, and can be rapidly produced in microbes at low costs (Xiang, Y. et al., 2021). Stable Nbs can be highly resistant to aerosolization for inhalation therapy of SARS-CoV-2 infection. Recently, the high preclinical efficacy of an ultrapotent Nb construct (PiN-21) has been successfully demonstrated in a sensitive COVID-19 animal model. At a very low dose (0.2 mg/kg), the PiN-21 inhalation treatment quickly protects animals' weight loss after SARS-CoV-2 infection, decreases lung viral titers by a million folds which leads to drastically mitigated lung pathology, while preventing viral pneumonia (Nambulli, S. et al., 2021). Potent neutralizing Nbs therefore represent a convenient and cost-effective therapeutic option to help mitigate the evolving pandemic.

The ACE2 receptor binding site (RBS) on the spike glycoprotein (S) is the major target of serologic response in COVID-19 patients. The RBS is the primary region of convergent mutations in circulating variants of SARS-CoV-2. The variants may enhance ACE2 binding leading to higher transmissibility, elude clinical mAbs, and reduce the neutralizing activities of both convalescent and vaccine-elicited polyclonal sera (Wang, P. et al. 2021; Wang, Z. et al., 2021; Zhou, D. et al., 2021). Particularly concerning variants include the B.1.1.7, B.1.351, and P.1 (Davies, N. G. et al., 2021; Wibmer, C. K. et al., 2021; Cele, S. et al., 2021). Long-term control of the pandemic requires the development of effective interventions with broadly neutralizing activities against the evolving strains (Davies, N. G. et al., 2021). Here the impact of the convergent variants of concern and the critical receptor-binding domain (RBD) point mutations on the ultrapotent neutralizing Nbs was assessed. Subsequent determination of 9 high-resolution structures, involving 6 Nbs bound to either S or RBD, by cryo-EM provided critical insights into the antiviral mechanisms of highly potent neutralizing Nbs. Structural comparisons between neutralizing mAbs and Nbs revealed marked differences between the two antibody species.

Potent Neutralizing Nbs are Highly Resistant to the Convergent Circulating Variants of SARS-CoV-2 and a Highly Evolved RBD Variant.

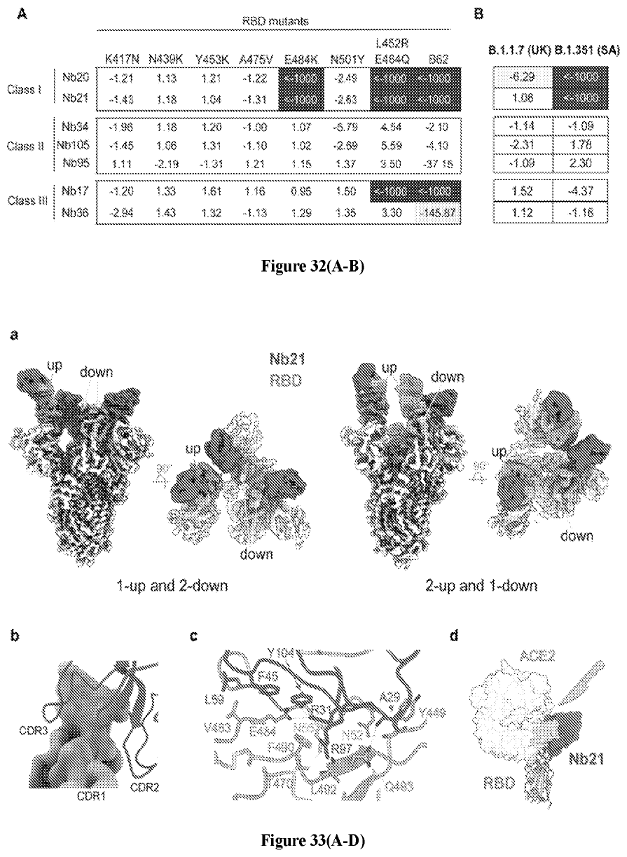

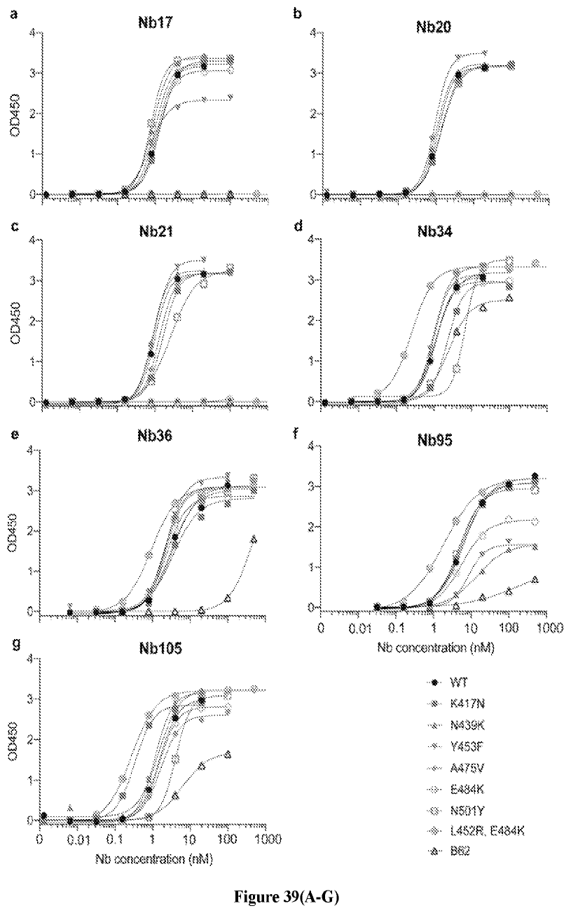

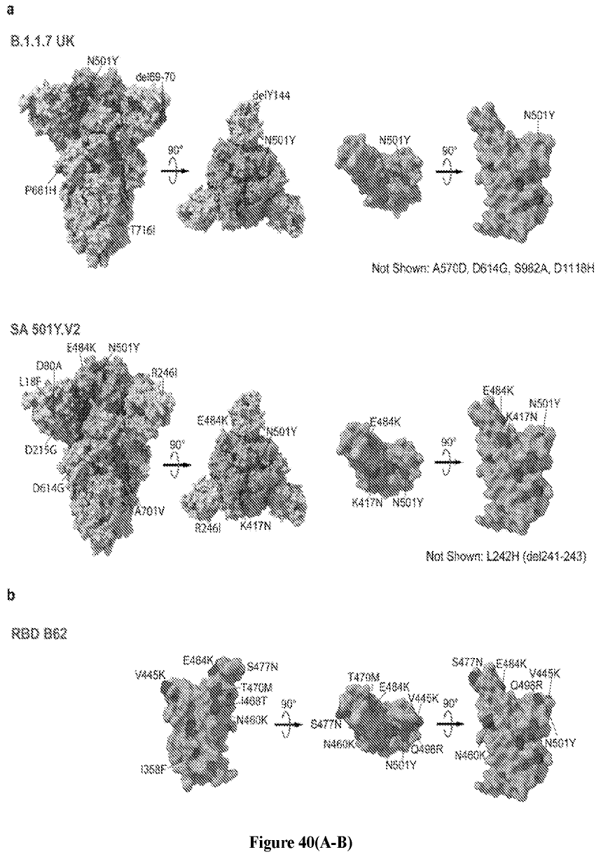

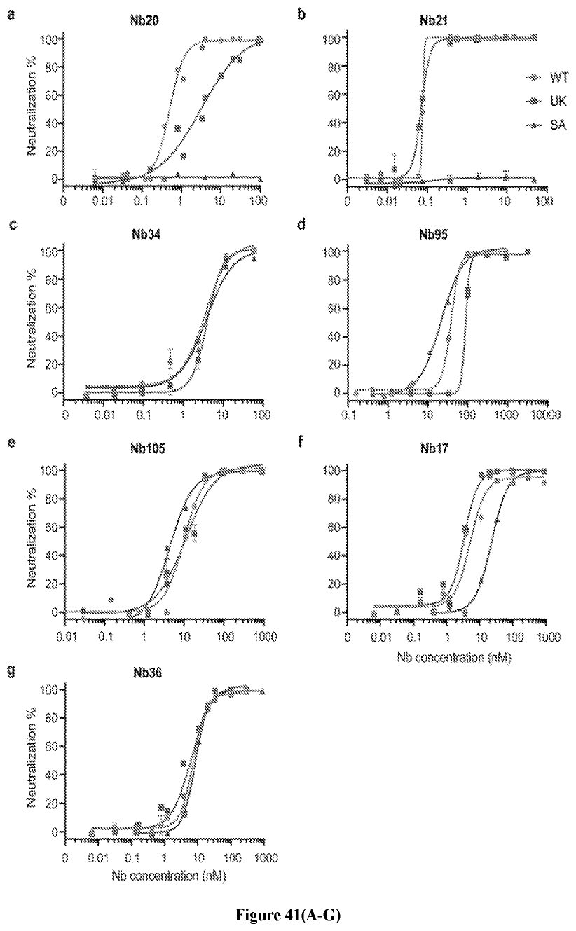

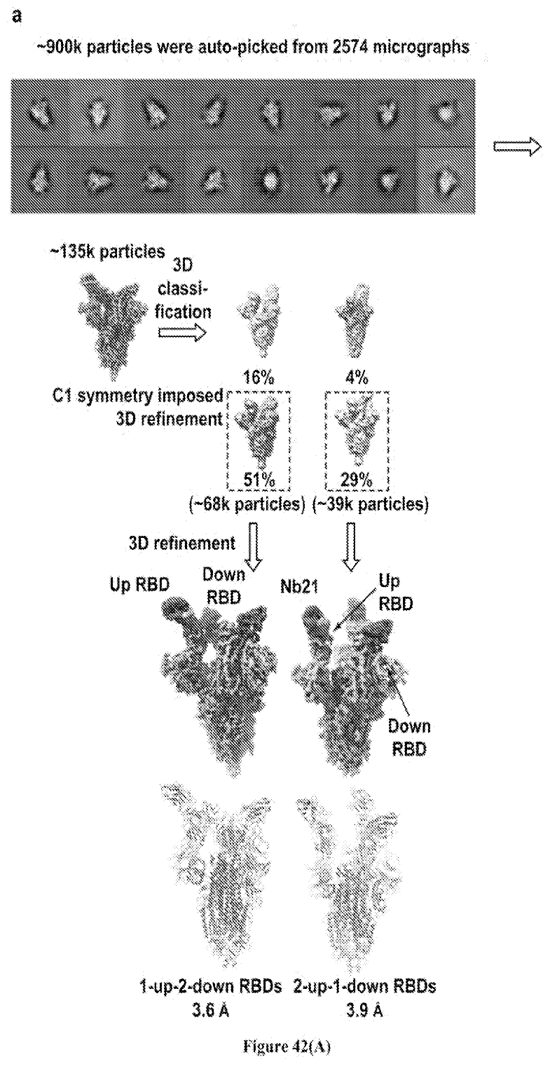

ELISA was performed to evaluate how 6 critical RBD mutations (Table 11) impact the binding of 7 diverse and potent neutralizing Nbs (Walter, J. D. et al., 2020). Interestingly, the neutralizing Nbs were generally unaffected by the mutations (FIG. 32A, FIG. 39). An exception was E484K, which abolished the ultra-high affinities of Nbs 20 and 21. In addition, it was found that three Nbs (17, 20 and 21) were affected by the double mutant L452R and E484Q, which are the critical RBD mutations recently found in Indian strain (Vaidyanathan, G., 2021). Moreover, two circulating variants of global concern (B.1.1.7 UK and B.1.351 SA) on Nb neutralization was evaluated using a pseudotyped virus neutralization assay. These pseudoviruses fully recapitulate the major mutations of the natural spike variants (FIG. 40). The initial SARS-CoV-2 strain (Wuhan-Hu-1) was used as a control. Consistent with the ELISA results, it was found that the UK strain (B.1.1.7), possessing a critical RBD mutation N501Y, has little effect on all 7 potent neutralizing Nbs (FIG. 32B, FIG. 41). The SA strain (B.1.351), containing three RBD mutations (K417N, E484K, and N501Y), drastically reduces the efficacy of Nbs 20 and 21 but has a very marginal impact on the efficacies of other Nbs. The results contrast with recent investigations of a repertoire of neutralizing mAbs derived from convalescent and vaccine-elicited polyclonal sera, where most of the isolated mAbs were significantly affected by at least one of mutations found in VOC (Wang, Z. et al., 2021).

To assess the potential to resist future mutations, highly neutralizing Nbs were evaluated for their binding to a potential future RBD variant (B62). B62 possesses unseen mutations which were evolved in vitro for high ACE2 binding affinity and potentially enhanced infectivity (FIG. 40). This highly evolved RBD variant contains 9 point mutations (I358F, V445K, N460K, I468T, T470M, S477N, E484K, Q498R, N501Y), including both established and potential mutations that together increase the affinity of ACE2 binding by 600 fold (Zahradnik, J. et al., 2021). While several Nbs were substantially affected by B62, Nbs 34 and 105 retained their high affinity against this evolved variant. These striking results prompted the further investigation on the structural basis for the broadly neutralizing activities of these Nbs. The high-resolution cryo-EM maps of Nbs in complex with either the prefusion-stabilized S (Hsieh, C. L. et al., 2020) or the RBD revealed three Nb classes that are affected differently by the variants and provided insights into their antiviral mechanisms.

Ultrapotent Class I Nbs and the “Achilles' Heel”.

Class I dominates high-affinity RBD Nbs and represents some of the most potent neutralizers for SARS-CoV-2. For example, Nb21 can neutralize a clinical isolate of SARS-CoV-2 at sub-ng/ml, which is unprecedented for monomeric antibody fragments. To better understand the neutralization mechanism of class I Nbs in the context of the S trimer, we solved the structure of the Nb21 bound to the S using cryo-EM.

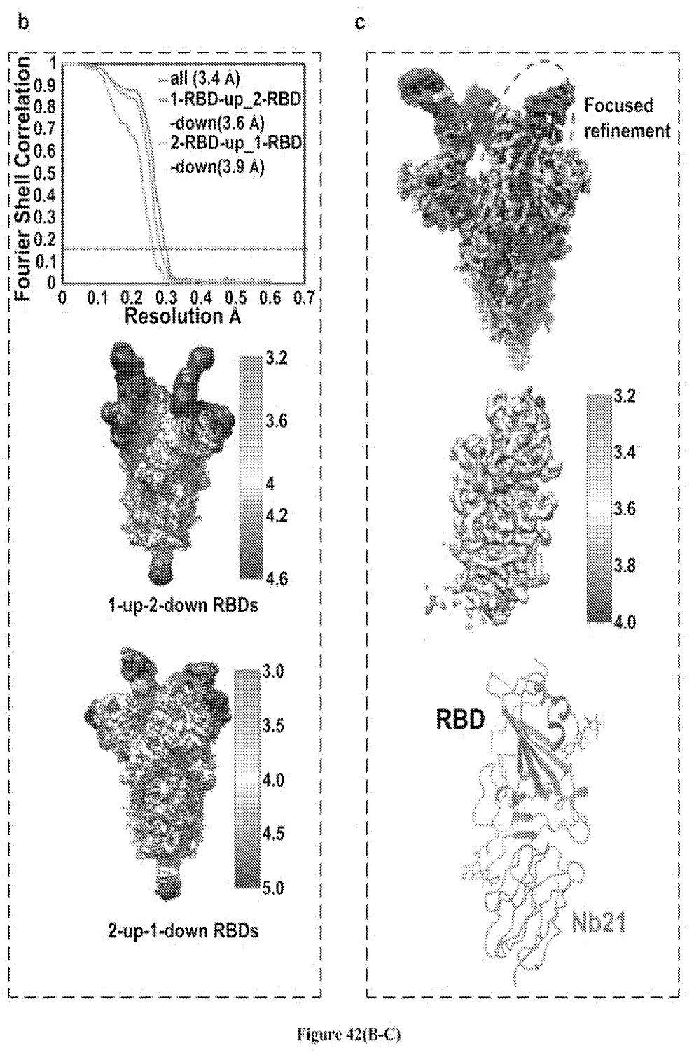

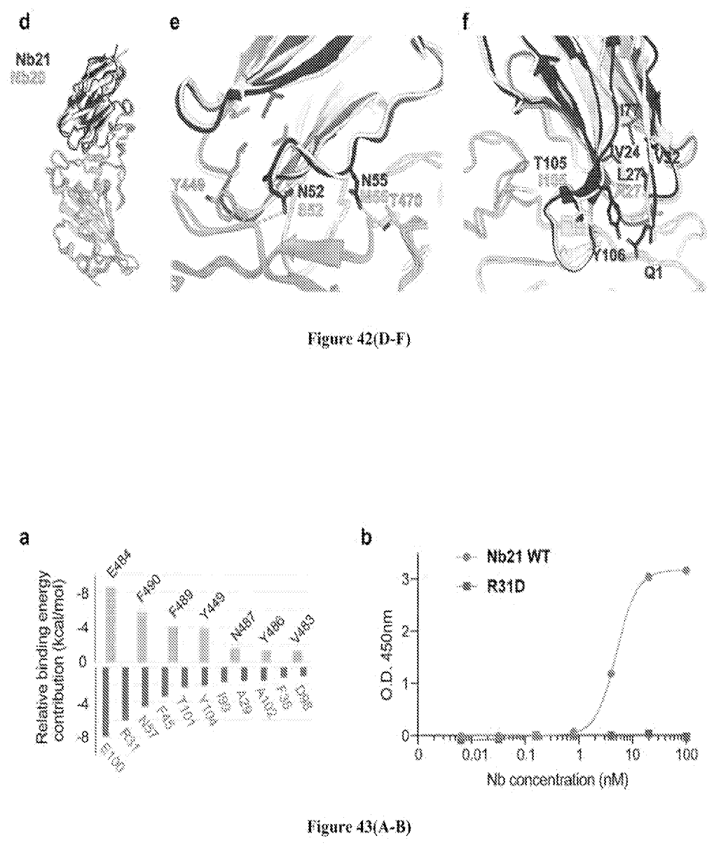

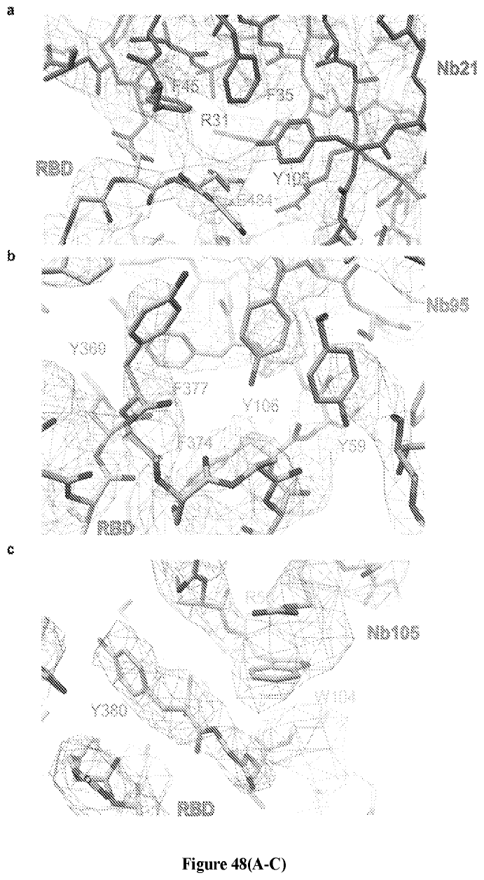

Nb21 binds RBDs in both up and down conformations. There are two major classes of the spike bound to Nb21: i) one up-RBD and two down-RBDs (resolution of 3.6 Å) and ii) two up-RBDs and one down-RBD (3.9 Å) (FIG. 33A, FIG. 42(A-C)). Due to the high flexibility of RBD, we performed local refinement of one down-RBD with Nb21 to resolve the binding interface (FIG. 42C). Nb21 binds the extended external loop region of the RBD with two β-strands. The interactions are mediated by all three CDR loops (FIG. 33B). The local density map shows potential cation-π interactions between R31 of Nb21 and F490 of RBD and a polar interaction network among R31 and Y104 of Nb21 and E484 of RBD. These four residues are located at the center of the Nb21:RBD interface, constituting a major site of interactions (FIG. 33C, FIG. 48A).

Consistent with the ELISA and pseudovirus assay results, relative binding energy calculation (Methods) revealed that E484 on the RBD is the “Achilles' heel” of the ultrapotent Nb21 (FIG. 43A). E484 provides the highest binding energy among all the interface residues on the RBD. In addition, it facilitates a network of adjacent residues such as F490, F489, N487, Y486, and V483 to participate in Nb21 binding. The E484K mutation can substantially destabilize the interface packing by electrostatic repulsion with R31 (CDR1), subsequently disrupting the cation-a stacking interaction between R31 and F490 (RBD). Simple charge reversal on R31(R31D) failed to recover the salt bridge and binding to the E484K mutant (FIG. 43B). Similarly, E484Q (from the Indian variants), can also disrupt the critical salt bridge of E484: R31 and the neighboring interaction network resulting in substantial loss of binding to RBD (FIG. 32).

In summary, Class I Nbs bind the RBS epitopes and can potently inhibit the virus by directly blocking ACE2 binding (FIG. 33D). Nevertheless, since the epitopes are among the least conserved regions on the spike, a critical point mutation (E484K/Q) can dramatically reduce the ultrahigh affinity of class I Nbs. The critical E484K/Q mutation is notably absent in the B.1.1.7 VOC, a variant that is dominating infection cases in the US and Europe, and can still be potently neutralized by Nbs 20 and 21 (Washington, N. L. et al., 2021).

Class II Nbs Bind Non-RBS Epitopes Yet Still Efficiently Block ACE2 Binding.

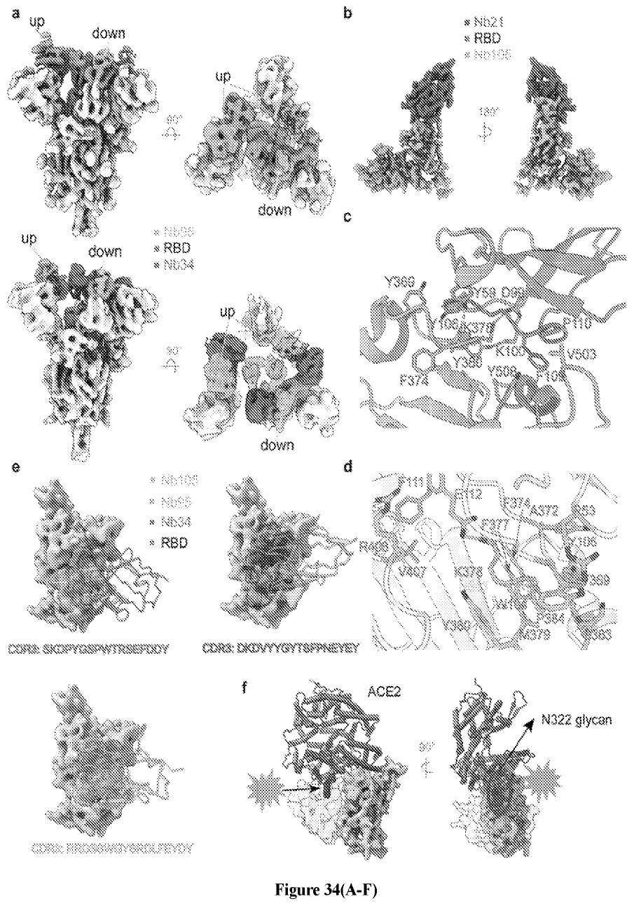

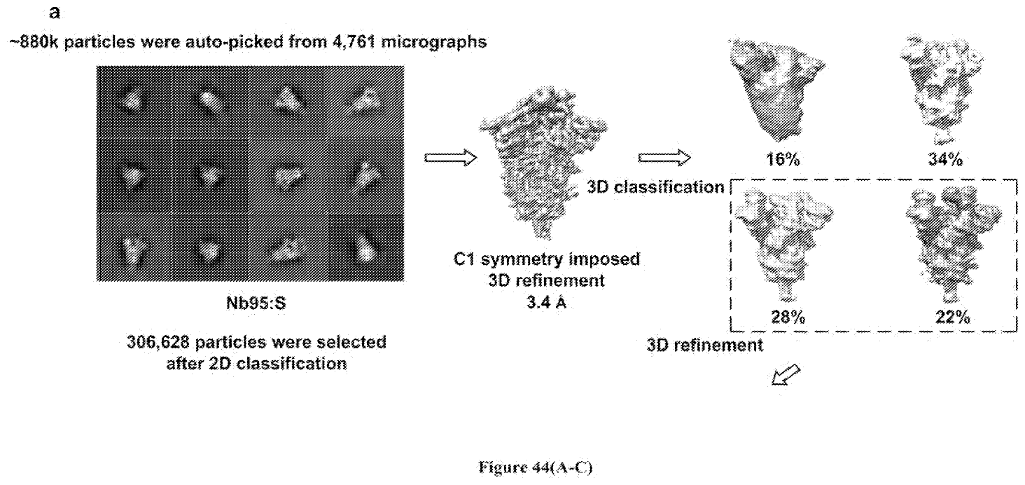

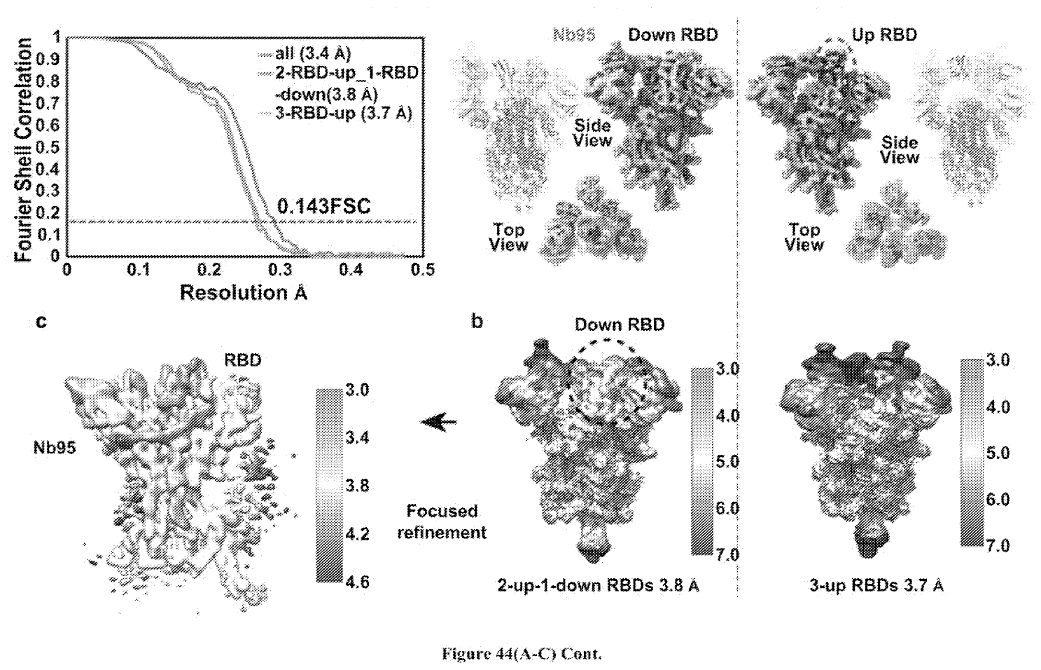

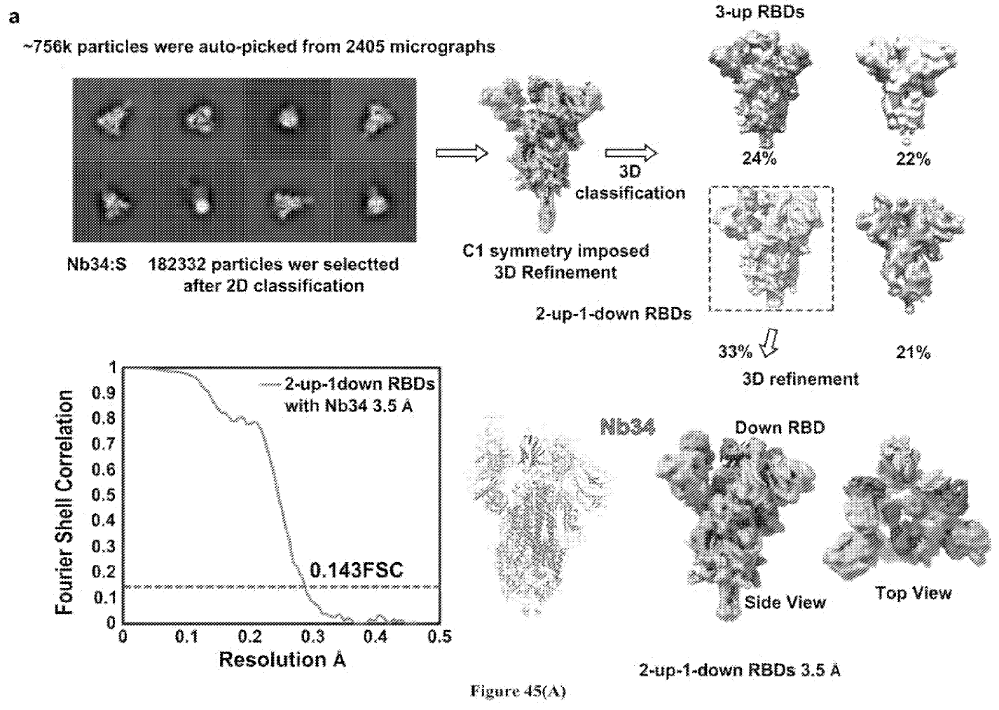

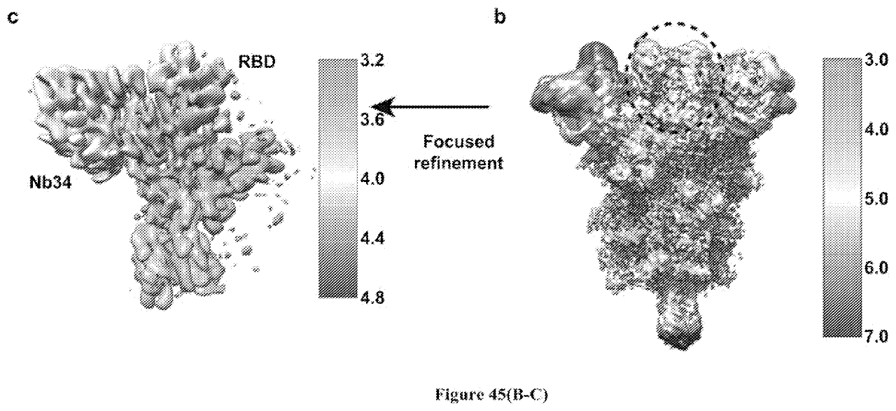

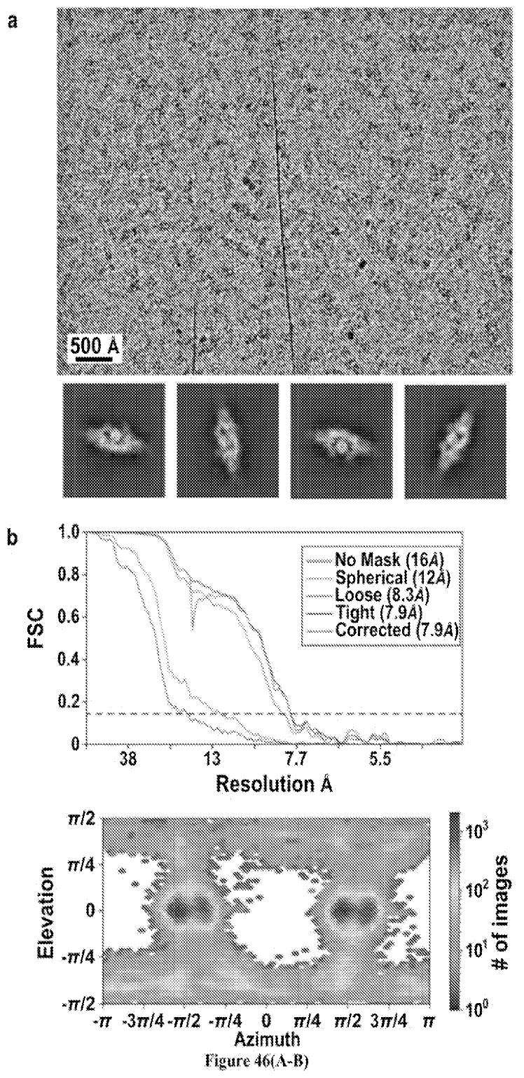

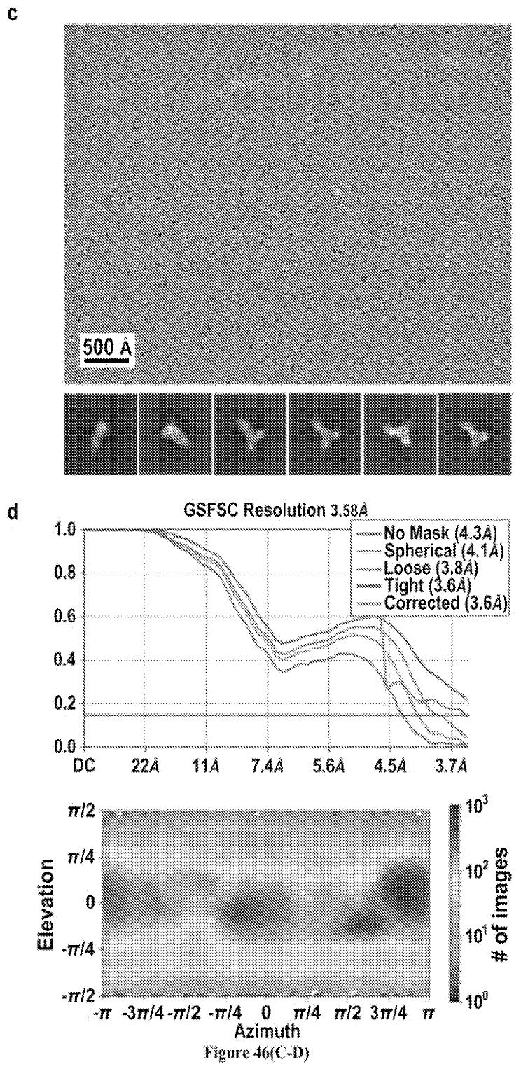

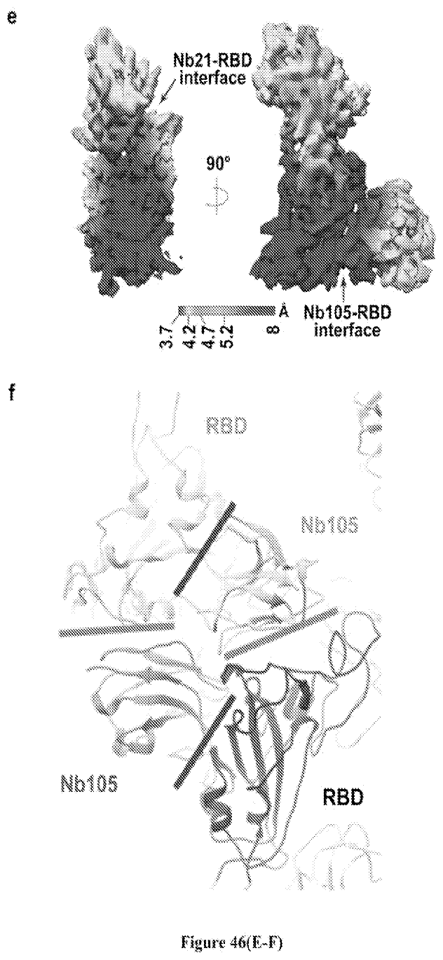

Class II Nbs (95, 34, and 105) can potently neutralize SARS-CoV-2 below 150 ng/ml. Cryo-EM analysis of Nbs 95 and 34 with S revealed two major classes of the complexes with an overall resolution of 3.4 Å and 3.5 Å, respectively: 1) two-up-one-down RBDs FIG. 34A, FIG. 44(A-B), FIG. 45A) and 2) three-up RBDs with high flexibility (FIG. 44C). Nb105, on the other hand, forms an elongated structure with two copies of S in all RBD-up conformations (FIG. 46(A-B)). The strong preferred orientation of this extended dimeric structure on the EM grids limits a high-resolution reconstruction to accurately define the Nb105:RBD interface. To map the epitope, therefore, a trimeric complex of Nb21:Nb105:RBD was assembled. The resulting ˜60 KDa complex was stable and was resolved by cryo-EM at 3.6 Å (FIG. 34B, FIG. 46(C-F)).

Class II Nbs bind RBD primarily through the hydrophobic residues on a relatively long CDR3 loop (17 or more residues) (FIG. 34(C-E)). Major interactions of Nb95:RBD and Nb105:RBD were resolved by local refinements (Methods). After the assignment of bulky side chains, such as Tyrosine and Phenylalanine (FIG. 48C), potential polar interactions can be inferred based on modeling. For Nb95, the side chains of CDR3 residues D99 and K100 form ionic or hydrogen-bonding interactions with the side chains of K378 and Y380 of RBD, respectively (FIG. 34C). The side chain of Y55 of Nb95 also forms a hydrogen bond with the main chain carbonyl of F374 of RBD. In addition to those polar interactions, the CDR3 residues P110 and F109 of Nb95 form hydrophobic interactions with the RBD residues V503 and Y508, and residues Y55 and Y106 of Nb95 cluster with the RBD residue Y369 to form aromatic interactions (FIG. 34C, FIG. 48B). Similar to Nb95, Nb105 recognizes RBD with CDR3 W104 and Y106 residing in two hydrophobic patches of M379-P384 and Y369-F377, respectively (FIG. 34D). Another hydrophobic residue F111 is clamped between V407 and R408 (RBD), forming a cation-π stacking interaction. The three patches of hydrophobic interactions surround an electrostatic interaction between E112 and K378 of RBD (FIG. 34D).

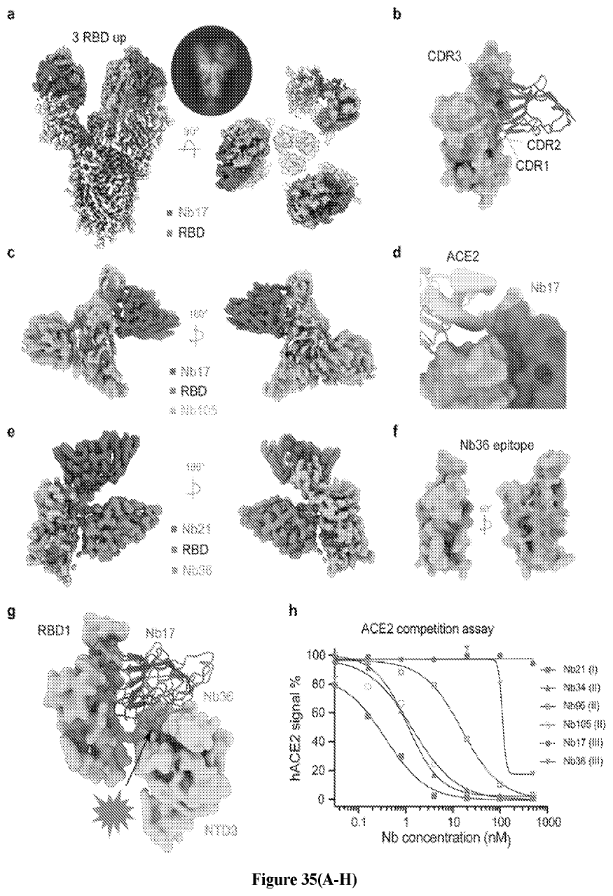

While class II Nbs do not compete with the ACE2 directly, they can still efficiently block ACE2 binding at low nM concentrations consistent with their high neutralization potency (FIG. 35H). Superposition of ACE2:RBD into the Nb: RBD complexes reveals that binding of Nbs 105 and 95 to the RBD overlaps with the subdomain II of ACE2 (residues 308-326) and the N322 glycan, wherever Nb34 can clash with the glycan (FIG. 3f). Recent crystal structures of camelid Nbs (Koenig, P. A. et al., 2021) and synthetic constructs (sybodies) (Walter, J. D. et al., 2021) have revealed a similar mode of binding with substantially lower neutralization potency (i.e., over 100-fold) compared to affinity matured class II Nbs (95, 34, and 105).

Class III Nbs Utilize Distinct Mechanisms to Neutralize the Virus Efficiently.

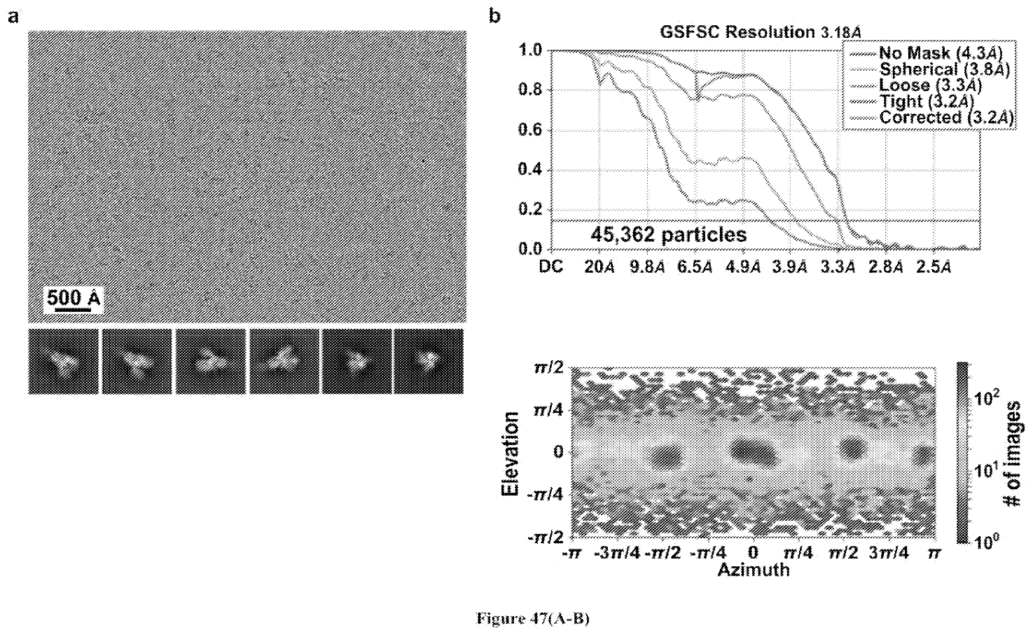

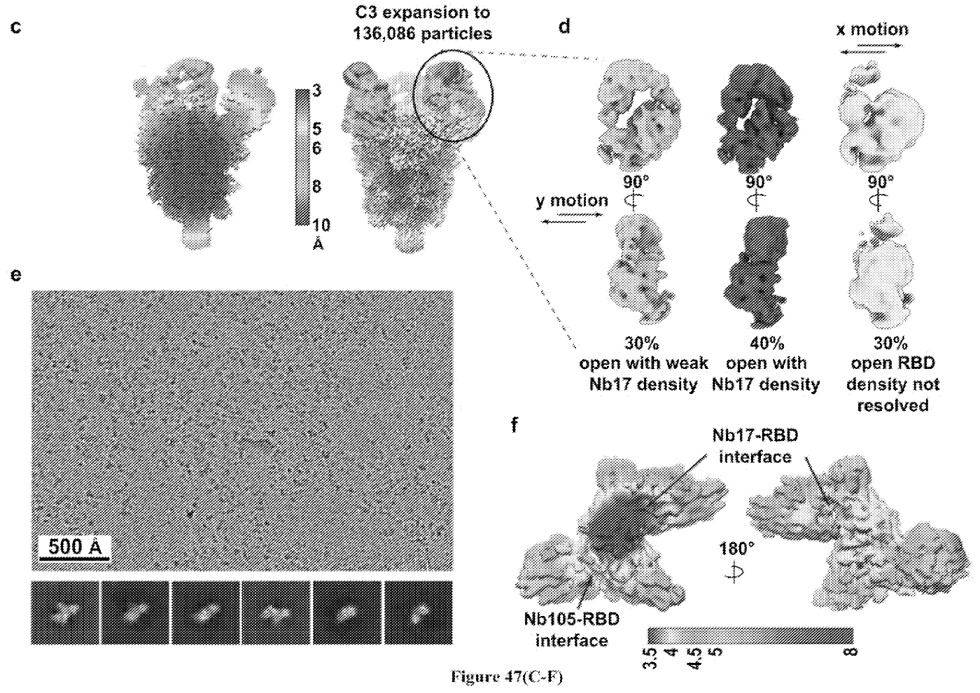

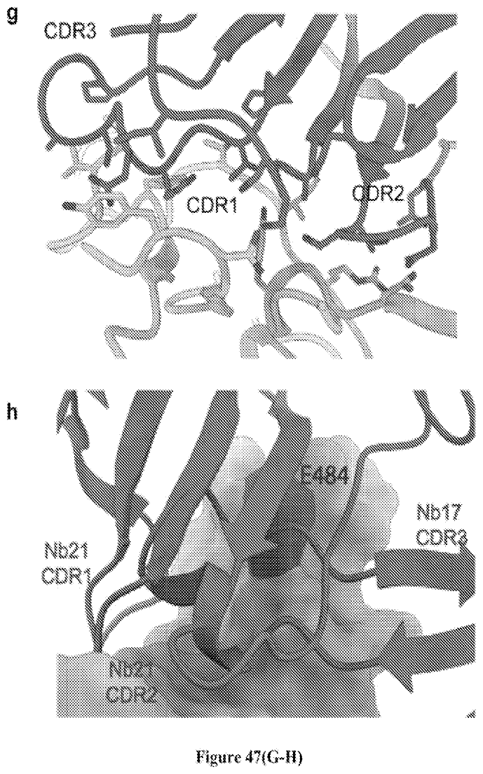

Nb17 locks the spike in all RBD-up conformations. Nb17 can neutralize the virus in vitro at an IC50 of ˜25 ng/ml. Notably, all three RBDs on the S trimer were in an open conformation with two having particularly strong densities (FIG. 35A, FIG. 47(A-D)). Nb17 binds a semi-conserved epitope including a segment spanning residues 345-356 and additional residues that generally do not overlap with the RBS. This epitope is localized on the opposite side of class II Nb epitopes (FIG. 35B). Similar to Nb21, Nb17 also utilizes all three CDRs for RBD recognition. No bulky side chains directly involve interface packing and contribute to the ultrahigh RBD binding affinity. Instead, small hydrophobic residues, such as Ala, Val, and Pro, and polar interactions are the primary contributors at the Nb17:RBD interface (FIG. 47G). 3D variability analysis shows that Nb17 density stacks on the adjacent NTD prefer the open conformation of all RBDs when Nb17 is bound.

The Nb17:Nb105:RBD complex was reconstituted to characterize the interface interactions (FIG. 35C, FIG. 47(E-F)). Superposition of the Nb17:RBD complex to the ACE2:RBD complex indicates that Nb17 can not interfere with ACE2 interactions (FIG. 35D). Structural alignment reveals that the CDR3 of Nb17 overlaps with Nb21 to compete for RBD binding (FIG. 47H).

To further explore the mechanism by which Nb17 efficiently neutralizes SARS-CoV-2, the Nb17:S (the super stable hexapro variant) complex was constituted and a limited proteolysis experiment was performed using proteinase K to assess the impact of all-RBD-up conformation (Methods). Compared to S itself or the S: Nb105 complex which is difficult to digest, Nb17 binding to S appears to increase the proteolysis rate of S in a manner similar to hACE2 binding. Here, with ultra-high affinity, Nb17 can lock S1 in a specific, open conformation that promotes the unprogrammed spike post-fusion transition and immature cleavage of S1 (FIG. 50A) (Zhou, H. et al., 2019; Walls, A. C. et al., 2019; Piccoli, L. et al., 2020).

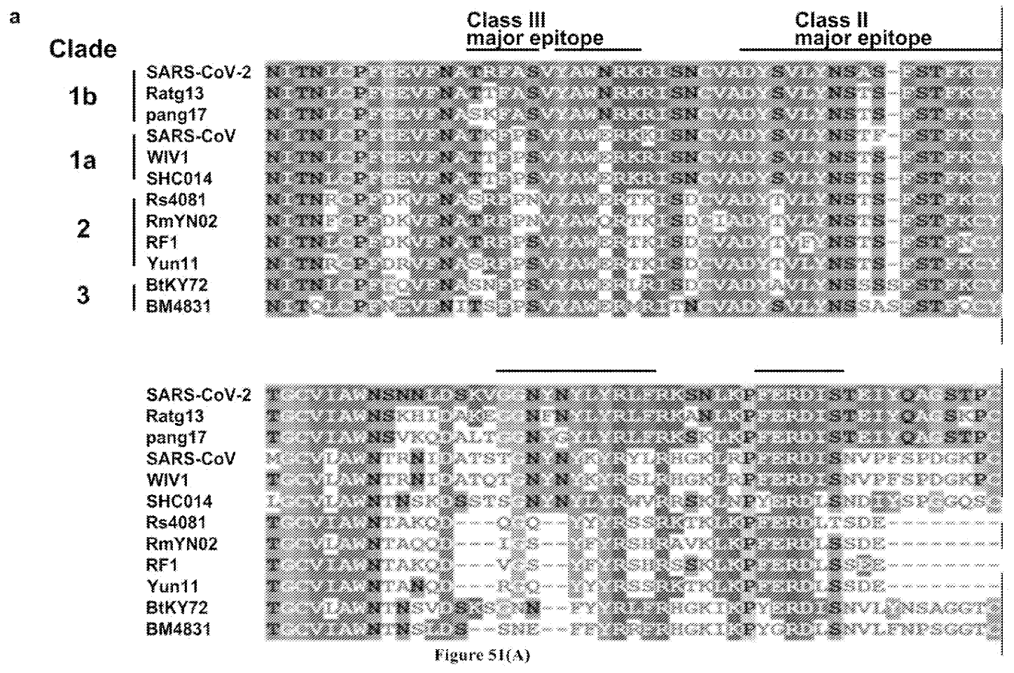

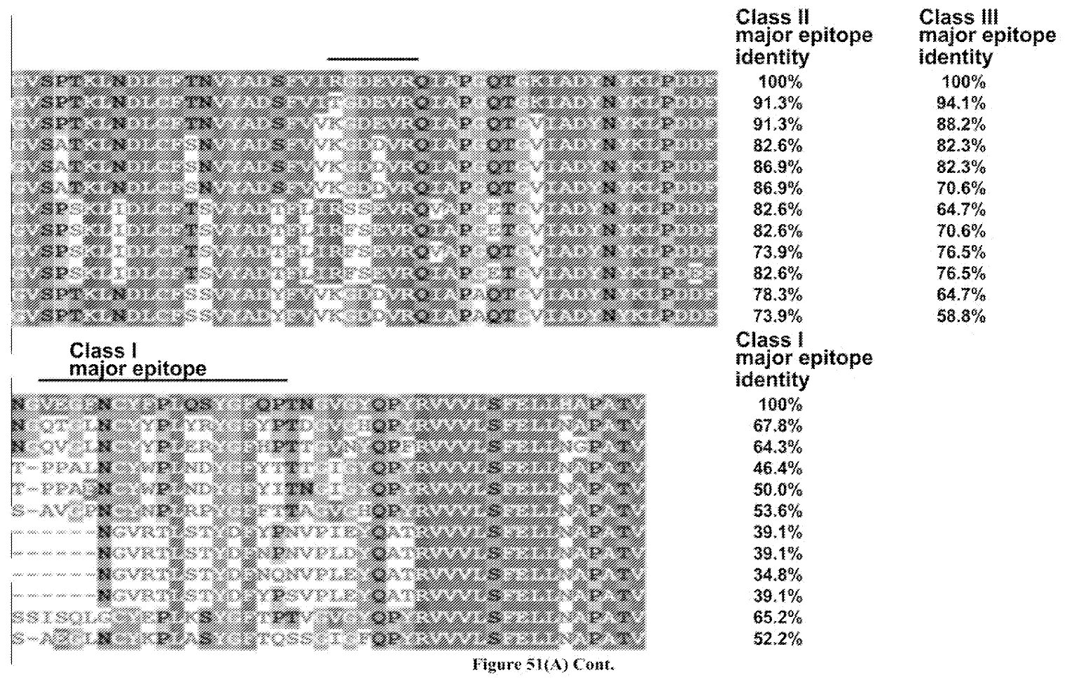

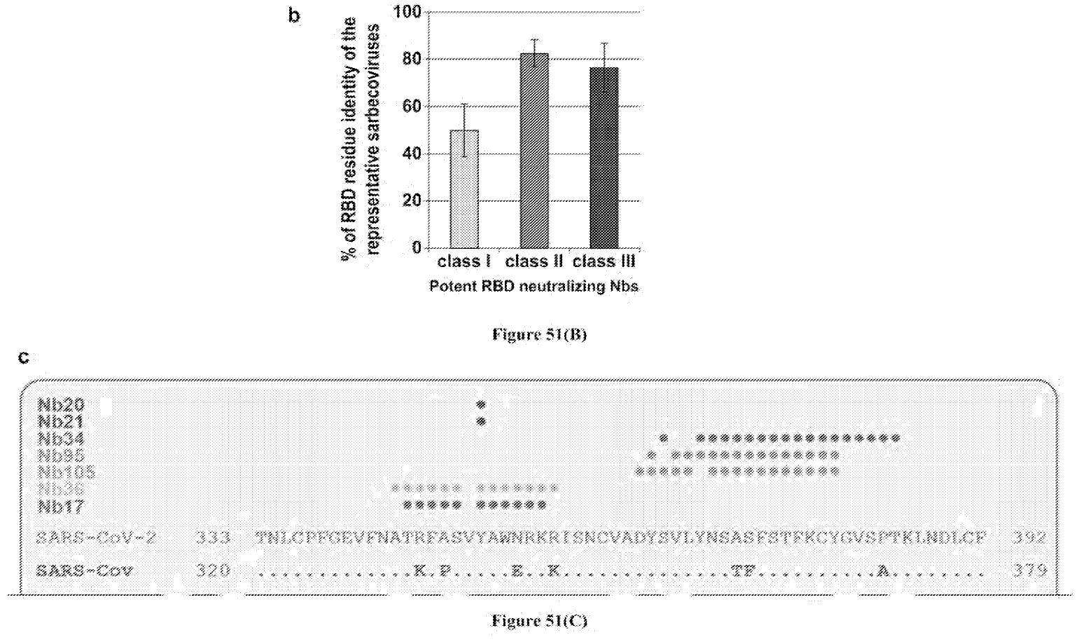

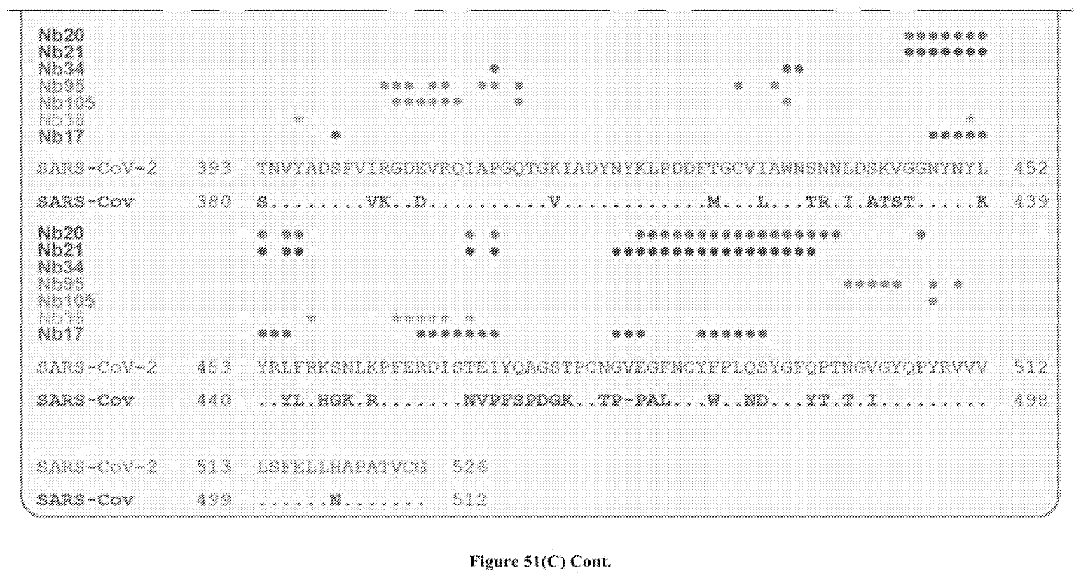

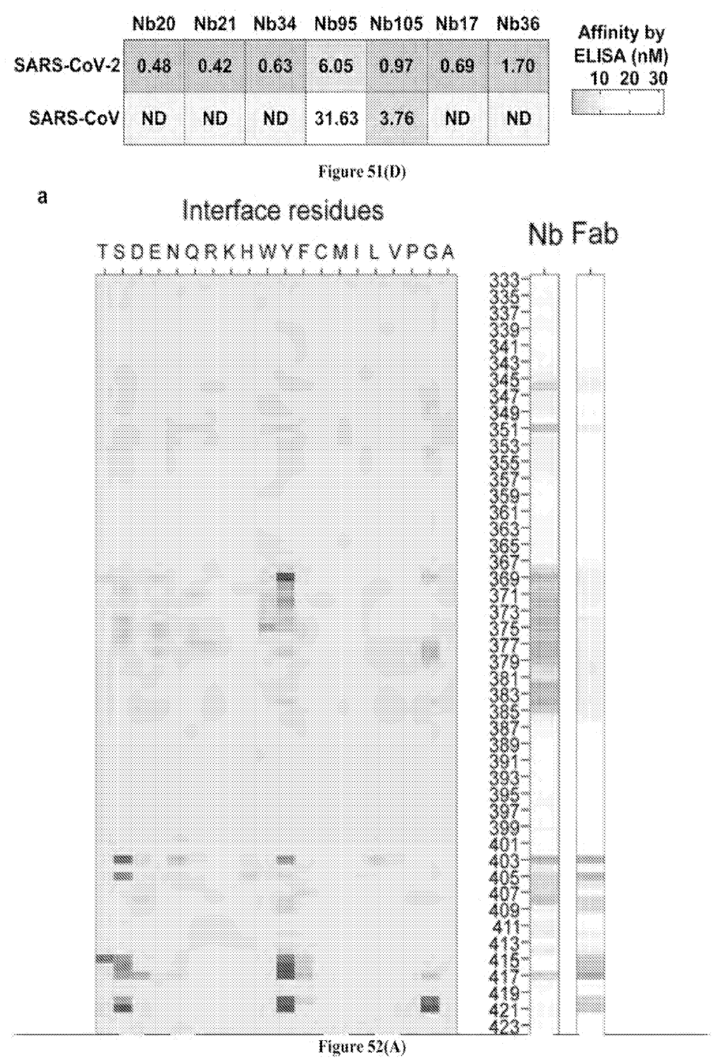

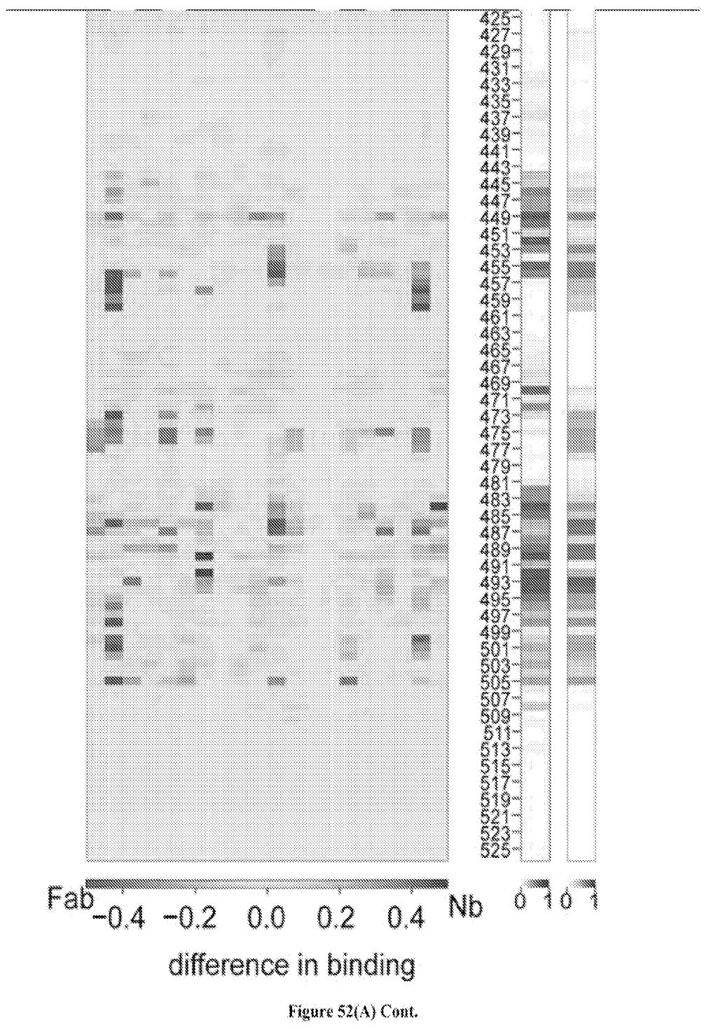

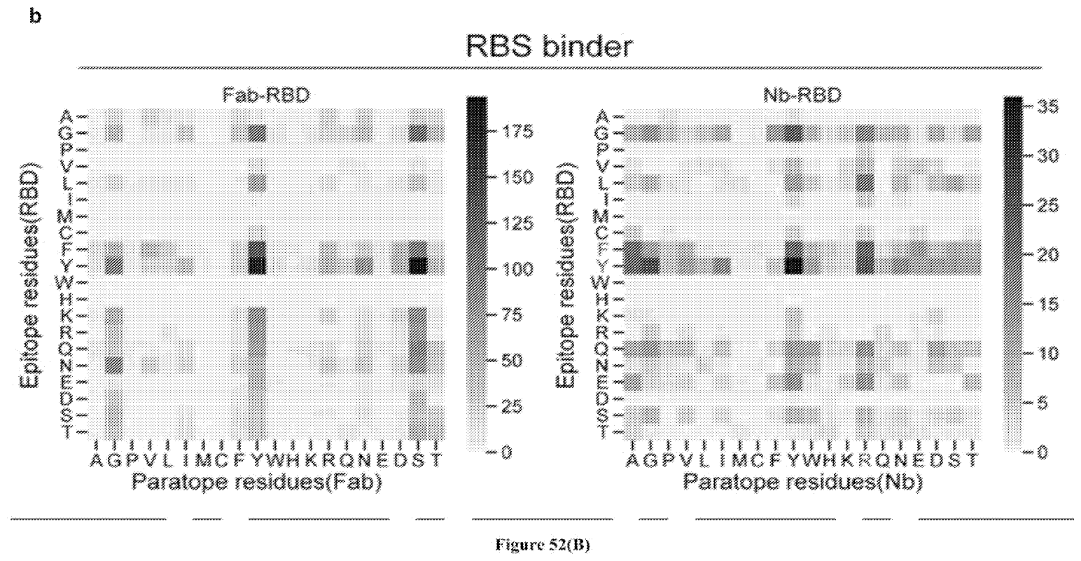

Nb17 is resistant to all the dominant natural RBD mutations that were tested, except for the Indian variant, due to the L452R mutation. Here, the long side chain of R at position 452 can disrupt the interfacial packing with the adjacent residues of S30, V96 and Q98 on Nb17 (Figure Compared to the Nb21:RBD interface, where E484 is buried inside the core of the interface, E484 localizes at the rim of the Nb17:RBD interface (FIG. 47H). As such, while E484 directly contacts Nb17, the mutations (E484K and E484Q) do not affect RBD binding (FIG. 32A). The loss of binding to the super variant of B62 is caused by two point mutations (I468 and T470) (FIG. 51B).

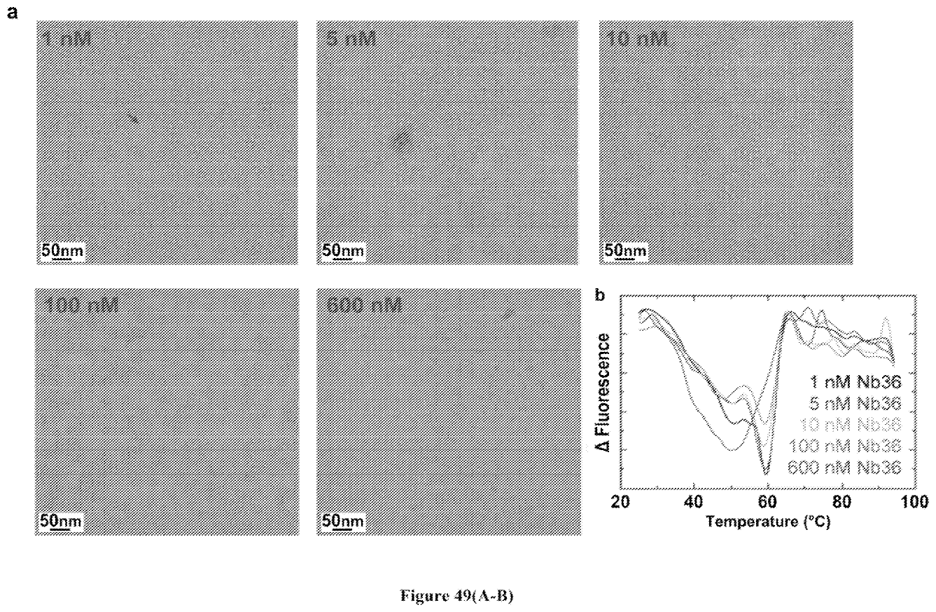

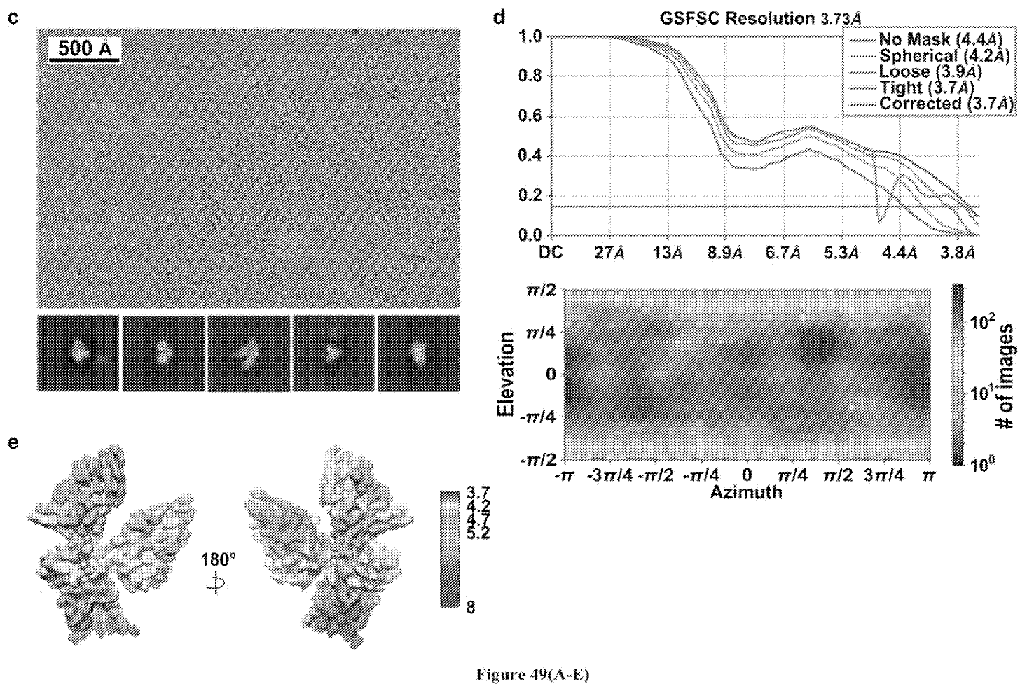

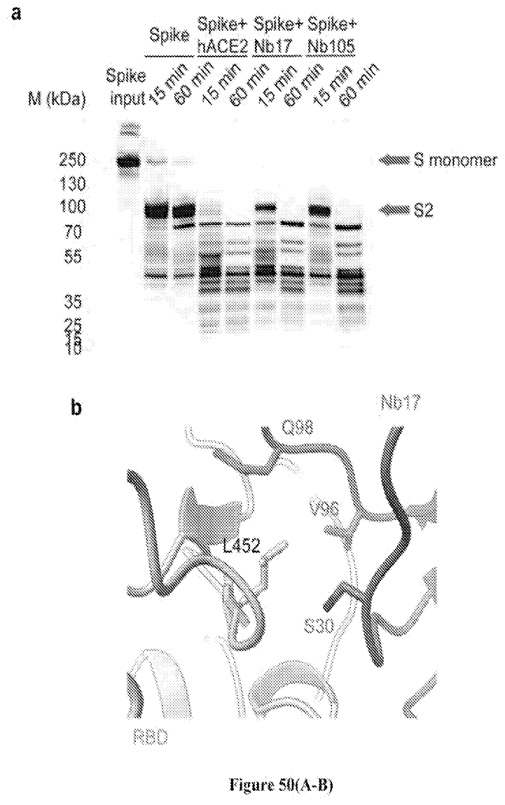

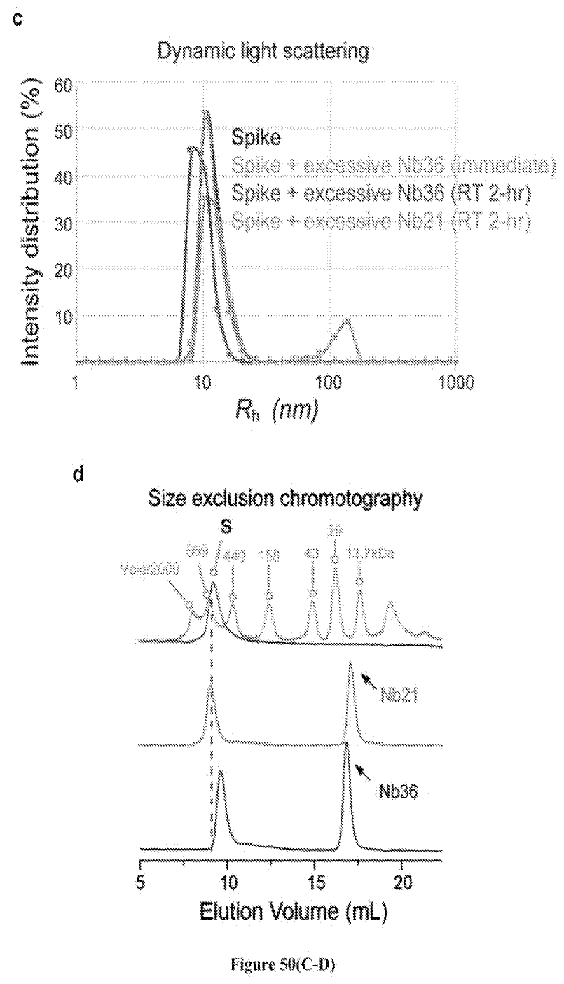

Nb36 destabilizes the spike trimer. While Nb36:S complex is highly soluble particles were not detected on the EM grids under cryogenic conditions. Therefore, to characterize Nb36:S interactions, different concentrations of Nb36 with S protein were titrated and the complexes by were imaged negative stain EM. The increasing concentration of Nb36 coincided with an enhanced blurring of the particles, which compromised contrast in the electron micrographs (FIG. 49A). This observation indicates that Nb36 can destabilize the integrity of the spike. To test this, thermal shift melting assays were employed under similar conditions as those used for negative stain EM. Consistently, an increase in Nb36 concentration correlated with a decrease in protein melting temperature, indicating that Nb36 promotes instability of the S complex (FIG. 49B). To map the epitope, the Nb36:Nb21:RBD complex was reconstituted and imaged by cryo-EM (FIG. 35E, FIG. 49(C-E)). The analysis reveals that the Nb36 epitope partially overlaps with Nb17 while exhibiting no overlap with Nb21 (FIG. 35F). The epitope covers a small segment on the non-RBS region (residues 353-360 of RBD) as well as distinct, non-RBS epitope residues that contact Nb17. Nb36 binds RBD in an orientation that is markedly different from Nb17. Superposition of the structure onto S reveals that Nb36 can have a significant steric clash with the neighboring NTD in the trimeric S complex (FIG. 35G). Facilitated by the small size, Nb36 can insert its convex paratope residues between an RBD and the adjacent NTD to destabilize the spike.

Analytical SEC was employed to check the sizes of Nb:RBD complexes. The analysis reveals that Nb36 binding to S leads to the formation of a smaller complex than the Nb21:S complex and interestingly the S itself (FIG. 50C). Dynamic light scattering (DLS) was used to further substantiate the negative stain and SEC results. After two hours of incubation at room temperature, the Nb36:S complex showed a substantially smaller radius (Rh) than the Nb21: S complex, which has an identical radius with the transiently formed Nb36: S complex (Figure Together, these data show that Nb36 can efficiently neutralize SARS-CoV-2 by destabilizing the spike. Since a super stable S variant (HexaPro) was used for this study, Nb36 binding can have a more dramatic impact on the highly flexible wild-type spike (Hsieh, C. L. et al., 2020). Moreover, this destabilization mechanism is a reminiscence of mAb CR3022. However, Nb36 targets a completely different epitope from CR3022 with substantially higher neutralization potency (˜7 nM) (Xiang, Y. et al., 2020; Yuan, M. et al., 2020; Huo, J. et al., 2020).

In some embodiments, the individual Nb paratopes and their associated epitopes on SARS-CoV-2 RBD (FIG. 51C; Table 1) are listed as follows:

Nb17
Seq:
(SEQ ID NO: 12)
HVQLVESGGGLVQAGGSLRLSCAASGSIFSSNAMSWYRQAPGKQRELVA
SITSGGNADYADSVKGRFTISRDKNTVYPEMSSLKPADTAVYYCHAVGQ
EASAYAPRAYWGQGTQVTVSS

### Paratope Residue Numbers:

28, 30, 31, 32, 33, 34, 35, 37, 47, 49, 50, 51, 52, 53, 54, 55, 56, 57, 58, 59, 71, 73, 74, 94, 95, 96, 97, 98, 99, 100, 101, 102, 104, 105, 106, 107, 109

### Epitope Residue Numbers:

345, 346, 347, 348, 349, 351, 352, 353, 354, 355, 356, 399, 448, 449, 450, 451, 452, 453, 454, 455, 466, 467, 468, 469, 470, 471, 472, 482, 483, 484, 489, 490, 491, 492, 493, 493, 494

Nb95
Seq:
(SEQ ID NO: 66)
QVQLVESGGGLVQAGGSLRLSCAASGRTFSSYSMGWFRQAQGKEREFVA
TINGNGRDTYYTNSVKGRFTISRDDATNTVYLQMNSLKPEDTAIYYCAA
DKDVYYGYTSFPNEYEYWGQGTQVTVSS

### Paratope Residue Numbers:

44, 45, 46, 47, 57, 58, 59, 60, 62, 65, 99, 100, 101, 102, 103, 104, 105, 106, 107, 108, 109, 110, 111, 112, 113

### Epitope Residue Numbers:

369, 371, 372, 373, 374, 375, 376, 377, 378, 379, 380, 381, 382, 383, 384, 403, 404, 405, 407, 408, 411, 412, 414, 432, 435, 501, 502, 503, 504, 505, 508, 510

Nb105
Seq:
(SEQ ID NO: 59)
HVQLVESGGGLVQAGGSLRLSCAVSGRTFSTYGMAWFRQAPGKERDFVA
TITRSGETTLYADSVKGRFTISRDNAKNTVYLQMNSLKIEDTAVYYCAV
RRDSSWGYSRDLFEYDYWGQGTQVTVSS

### Paratope Residue Numbers:

53, 60, 100, 101, 102, 103, 104, 105, 106, 107, 108, 109, 110, 111, 112, 113, 114

### Epitope Residue Numbers:

368, 369, 370, 371, 372, 374, 375, 376, 377, 378, 379, 380, 381, 382, 383, 384, 404, 405, 406, 407, 408, 409, 414, 435, 436, 508

Nb34
Seq:
(SEQ ID NO: 22)
DVQLVESGGGLVQAGGSLRLSCAASGFTFSNYVMYWGRQAPGKGREWVS
GIDSDGSDTAYASSVKGRFTISRDNAKNTLYLQMNNLKPEDTALYYCVK
SKDPYGSPWTRSEFDDYWGQGTQVTVSS

### Paratope Residue Numbers:

38, 43, 44, 45, 46, 47, 48, 59, 60, 61, 62, 63, 65, 102, 103, 109, 110, 111, 112, 113, 116

### Epitope Residue Numbers:

366, 369, 370, 371, 372, 373, 374, 375, 376, 377, 378, 379, 380, 381, 382, 383, 384, 385, 412, 436, 437

Nb36
Seq:
(SEQ ID NO: 23)
HVQLVESGGGLVQAGGSLTLTCAASGRTFSSETMDMGWFRQAPGKEREF
VAADSWNDGSTYYADSVKGRFTISRDSAKNTLYLQMNSLKPEDTAVYYC
AAETYSIYEKDDSWGYWGQGTQVTVSS

### Paratope Residue Numbers:

28, 33, 39, 40, 104, 105, 106, 107, 108, 109, 110, 111, 113, 114, 115, 116, 118

### Epitope Residue Numbers:

344, 345, 346, 347, 348, 349, 351, 352, 353, 354, 355, 356, 357, 396, 451, 457, 464, 465, 466, 467, 468, 470

Nb21
Seq:
(SEQ ID NO: 15)
QVQLVESGGGLVQAGGSLRLSCAVSGLGAHRVGWFRRAPGKEREFVAAI
GANGGNTNYLDSVKGRFTISRDNAKNTIYLQMNSLKPQDTAVYYCAARD
IETAEYTYWGQGTQVTVSS

### Paratope Residue Numbers:

27, 28, 29, 30, 31, 33, 35, 44, 45, 46, 47, 48, 50, 51, 52, 55, 56, 57, 58, 59, 70, 72, 97, 98, 99, 100, 101, 102, 103, 104

### Epitope Residue Numbers:

351, 446, 447, 448, 449, 450, 451, 452, 453, 455, 456, 470, 472, 482, 483, 484, 485, 486, 487, 488, 489, 490, 491, 492, 493, 494, 495, 496, 497, 498, 505, 531

Nb20
Seq:
(SEQ ID NO: 14)
QVQLVESGGGLVQAGGSLRLSCAVSGAGAHRVGWFRRAPGKEREFVAAI
GASGGMTNYLDSVKGRFTISRDNAKNTIYLQMNSLKPQDTAVYYCAARD
IETAEYIYWGQGTQVTVSS

### Paratope Residue Numbers:

27, 28, 29, 30, 31, 32, 33, 35, 45, 47, 48, 49, 51, 52, 55, 56, 57, 58, 59, 60, 72, 97, 98, 99, 100, 102, 103, 104

### Epitope Residue Numbers:

351, 417, 449, 450, 451, 452, 453, 455, 456, 470, 472, 481, 482, 483, 484, 485, 486, 487, 488, 489, 490, 491, 492, 493, 494, 495, 496

Class III RBD Nbs Belongs to a Novel Class of Neutralizing Nbs.

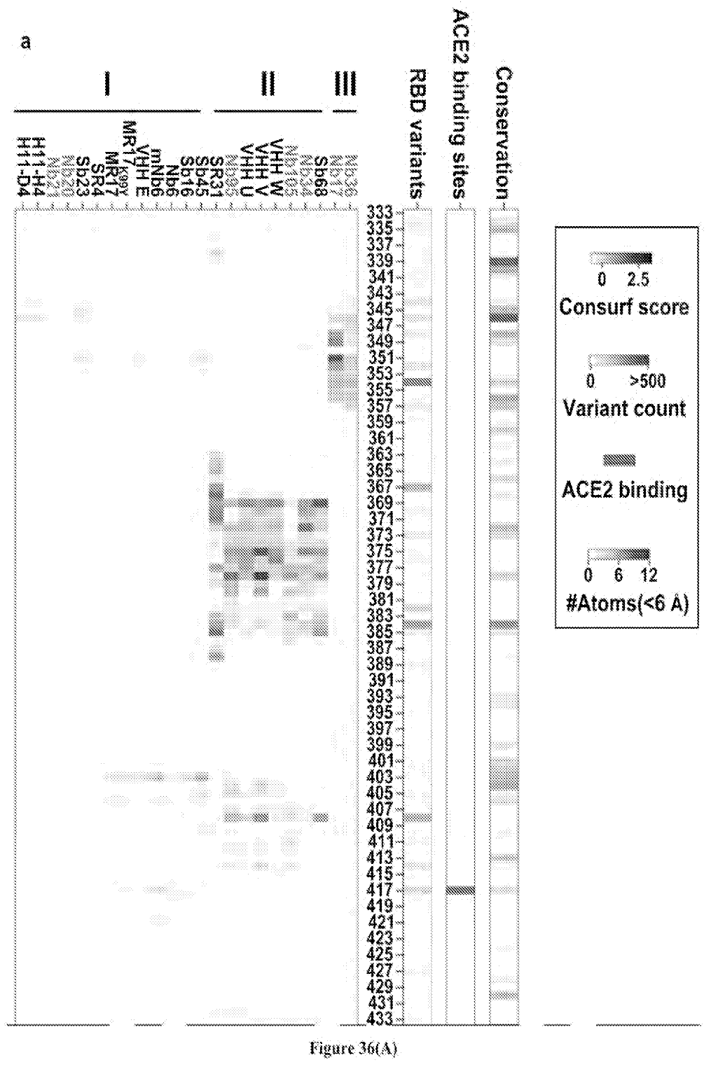

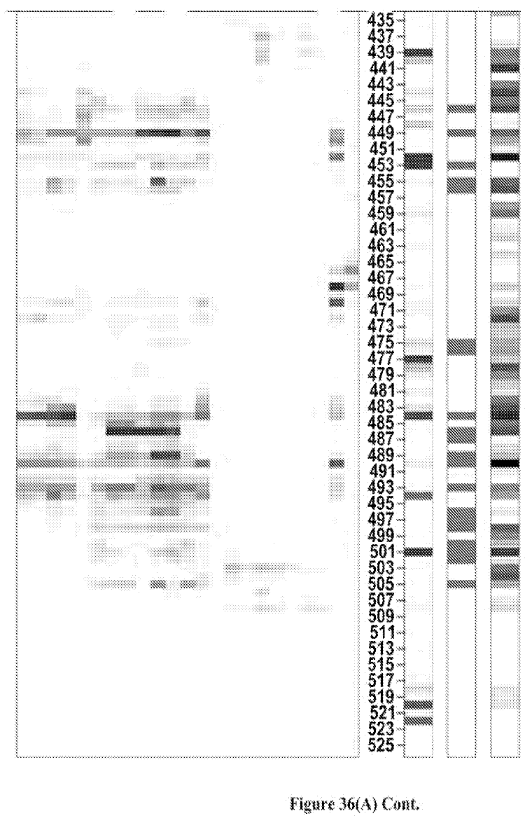

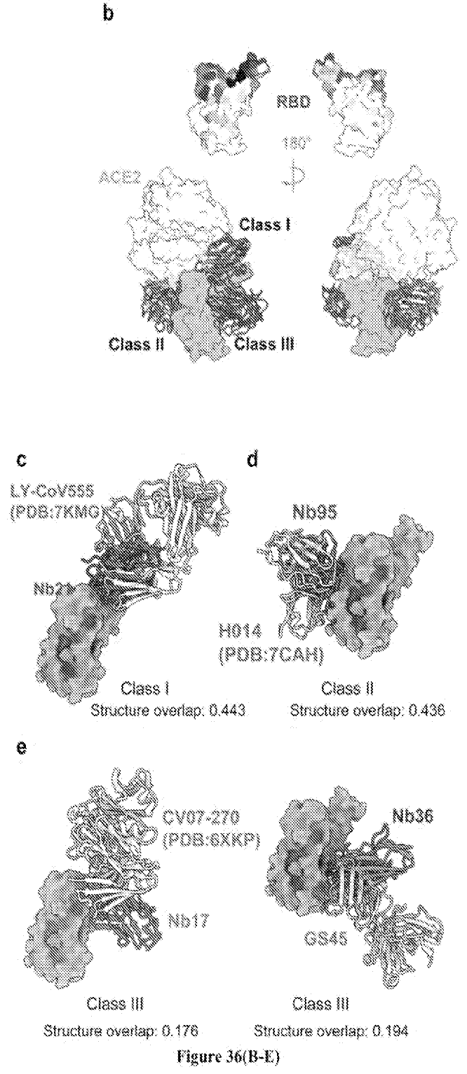

To investigate the RBD epitopes and Nb neutralization mechanism systematically, all the available structures that include Nb: RBD interactions (FIG. 36A) were analyzed. Epitope clustering supports the notion of three distinct classes of neutralizing Nbs. Most Nbs bind class I epitopes that mainly cover RBS (FIG. 36(A-B)). Class I and II epitopes are shared between Nbs and mAb (FIG. 36(C-D)). In contrast, the class III Nbs are novel and unique among all the neutralizing Nbs and mAbs that have been characterized (FIG. 36E). Class III epitopes are in close proximity to the neighboring NTD. Thus, accessing these epitopes is elusive for mAbs due to steric hindrance imposed by their large sizes. Here, with optimal orientations and substantially smaller sizes, Nbs can target relatively conserved epitopes (FIG. 51) where the virus can have relatively low mutational tolerance (Starr, T. N. et al., 2020).

Class II and III Nbs Target More Conserved RBD Epitopes than Class I Nbs.

To assess the epitope conservation, 12 RBDs from the major clades of the sarbecovirus family (i.e., lineage B of beta-coronavirus or SARS-like) were selected and aligned (Boni, M. F. et al., 2020). Major RBD epitope residues for each class of Nbs, and the epitope sequence identities to RBD_{SARS-CoV-2} were shown in FIG. 51(A-B). The median epitope identities for class I, II and III Nbs are 50% (σ=11.1%), 82.6% (σ=5.7%), and 76.5% (σ=10.3%), respectively.

The binding of potent neutralizing Nbs to RBD_{SARS_CoV} were also evaluated, which shares ˜73% sequence identity with RBD_{SARS-CoV-2}. Consistent with epitope conservation analysis, the ELISA results show that unlike class I and III Nbs, potent neutralizing class II Nbs (specifically, Nb95 and Nb105, but not Nb34) bind strongly to RBD_{SARS-CoV-2} by targeting highly conserved RBD epitopes (FIG. 51(C-D)). Specific and ultrapotent Class II Nbs can be used for the further bioengineering of pan-sarbecovirus Nb constructs.

Nbs and mAbs are Differently Affected by Mutations in the Circulating Variants.

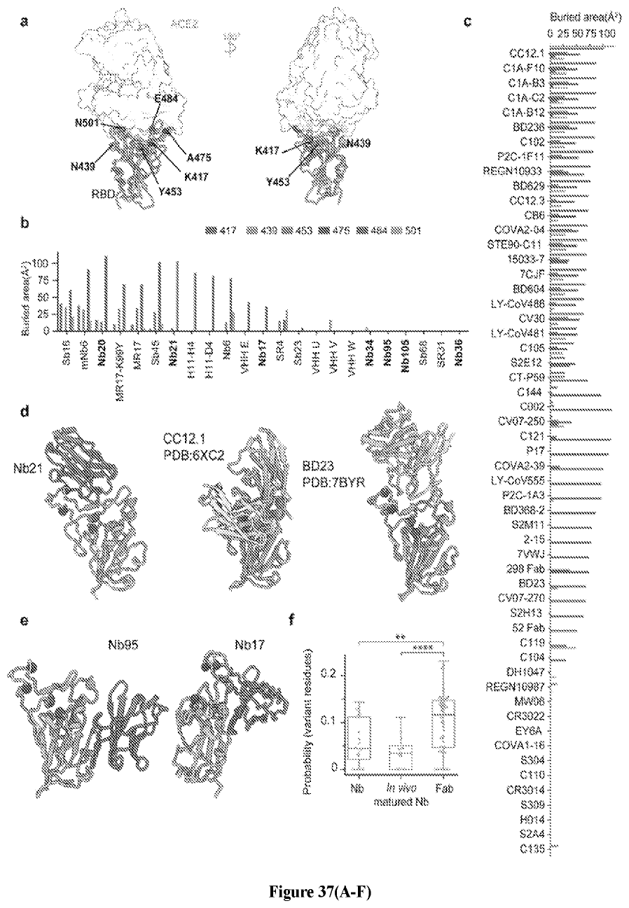

To understand how the unique binding modes translate to high resistance against SARS-CoV-2 mutants, three Nb classes were compared with mAbs. Buried surface area (BSA) of RBD interfacing residues from both Nb and mAb-bound structures were calculated and compared systematically (FIG. 37(A-C)). The analysis reveals that the majority of mAbs (83%) use at least one of the mutated RBD residues to bind, with 60% using two or more variant residues for RBD interactions. In contrast, Nbs target these sites substantially less frequently (FIG. 37B) with the exception of class I Nbs, which predominantly recognizes the hot spot fostered by E484 (FIG. 37D). Other classes do not bind these variant residues directly (FIG. 37B and FIG. 37E).

The fact that many critical mutations localize on the RBS is intriguing (FIG. 36A). Under selection pressures, the virus appears to have evolved an efficient strategy to evade host immunity by preferentially targeting this critical functional region. Specific RBS mutations (such as K417N and E484K) can help optimize host adaptation (improved ACE2 binding) to achieve higher transmissibility (Greaney, A. J. et al., 2021). In parallel, as RBS is the main target of serologic response, the mutations provide an effective means for the resulting variants to escape the neutralizing pressure from serum polyclonal antibodies (Zahradnik, J. et al., 2021; Starr, T. N. et al., 2020; Greaney, A. J. et al., 2021; Greaney, A. J. et al., 2021). Since most clinical mAbs originate from convalescent plasma, they are less effective against convergent circulating variants (Wang, P. et al., 2021; Wang, Z. et al., 2021). Fundamentally, this is different from neutralizing Nbs which have not been co-evolving with the virus, and therefore can be less sensitive to the plasma-escaping variants. Indeed, the probability of neutralizing Nb epitopes coinciding with the variant mutations was substantially lower than that of mAbs (FIG. 37F). This is particularly the case for highly potent and in vivo affinity-matured Nbs. Together with the functional data (FIG. 32), the analysis provides a structural basis to understand how potent neutralizing Nbs can resist the convergent variants. It is conceivable that Nbs can provide additional therapeutic benefits over mAbs for the evolving variants of SARS-CoV-2.

Systematic Comparisons of mAbs and Nbs for RBD Binding.

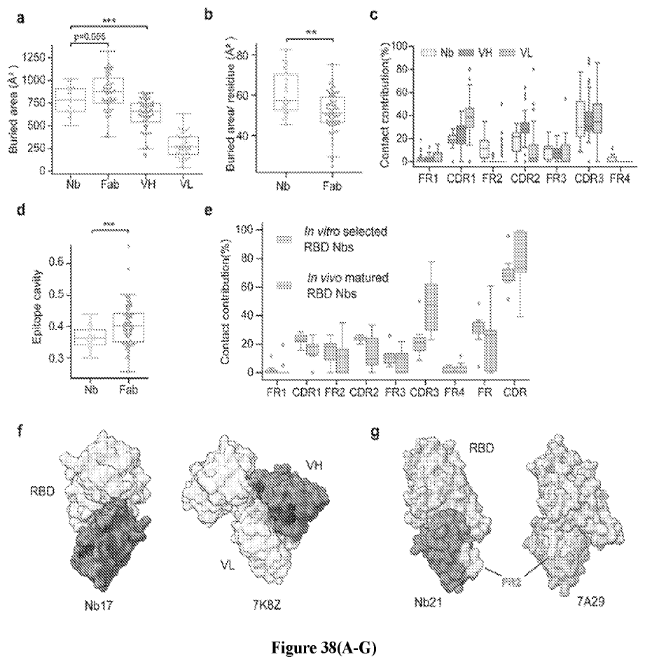

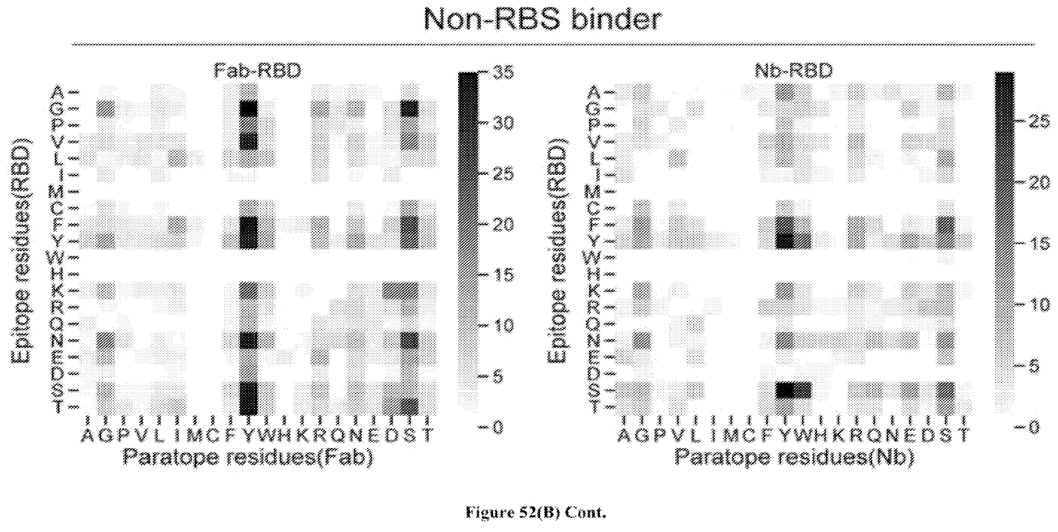

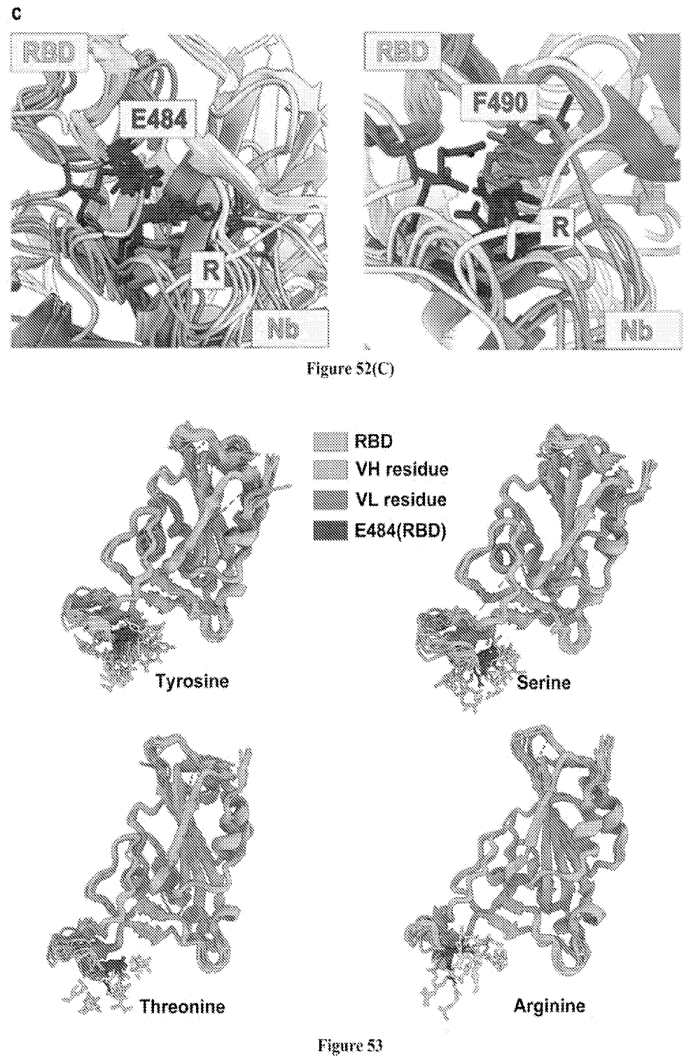

All the available structures were compiled and analyzed including 56 distinct mAb-bound complexes and 23 Nb-bound complexes (Table 14). Nbs have lower BSA values than Fabs (μ_{Nb}=779 Å² v.s. μ_{Fab}=862 Å², p=0.055) (FIG. 38A). In addition, the distribution of BSA for Nbs is distinct from Fabs and is substantially narrower (σ_{Nb}=151 Å², σ_{Fab}=210 Å²) (Mitchell, L. S. & Colwell, L. J., 2018) (FIG. 38A). Despite the smaller size, Nbs have evolved multiple strategies for high-affinity RBD binding. They exploit surface residues (especially using CDR3 loops) significantly more efficiently than Fabs for RBD engagement (FIG. 38(B-C)). Nbs also have higher BSA per-interface residue (FIG. 38B). The involvement of the framework (FR) regions in RBD binding, particularly FR2, is also evident probably due to the absence of light chain pairing (FIG. 38C). Compared to in vivo affinity-matured RBD Nbs, in vitro selected Nbs tend to use highly conserved FR sequences more extensively for interactions which can lead to decreased specificity. More dominant involvement of conserved FR shows that in vitro selected Nbs can interact less specifically to RBD (FIGS. 38E and 38G). Compared to Fabs, Nbs bind more concave surfaces (Methods) to tighten the interactions (FIG. 38D, 38F). Finally, neutralizing Nbs employ electrostatic interactions more extensively while both types of antibodies predominantly use hydrophobic interactions to achieve high specificity (FIGS. 52-53).

The prospect of curbing the pandemic rests on the development of effective vaccines and therapeutics that resist both the current and future circulating variants of SARS-CoV-2. Highly selective neutralizing Nbs and the multivalent Nb forms represent some of the most potent antiviral agents that have been developed to date (Bracken, C. J. et al., 2021; Schoof, M. et al., 2020; Xiang, Y. et al., 2020; Lu, Q. et al., 2021). Critically, a recent study has demonstrated the preclinical efficacy and efficiency of using ultrapotent aerosolizable Nbs for inhalation therapy of SARS-CoV-2 infections in a sensitive COVID-19 model (Nambulli, S. et al., 2021). Here, the structure analysis reveals that neutralizing Nbs can be grouped into three epitope classes. Class I contains some of the most potent SARS-CoV-2 neutralizing Nbs that have been identified to date. Ultrapotent class I Nbs such as Nbs 20 and 21 can neutralize SARS-CoV-2 (Munich strain) with IC50s of 66 and 22 pM, respectively (Xiang, Y. et al., 2020). They target variable RBS and the ultrahigh binding affinities are not affected by the highly transmissible UK variant. However, the RBD binding of class I Nbs can be abolished by a single point mutation (E484K/Q) that is present in Brazil, South African and Indian variants. Class II and III Nbs target conserved epitopes that are resistant to the current VOC and the mutational escape. Both classes I and II Nbs potently neutralize SARS-CoV-2 by sterically interfering with ACE2 binding. Class III Nbs bind cryptic epitopes that are inaccessible to large conventional antibodies. It was found that class III Nbs can employ different and unique neutralization mechanisms that do not rely on ACE2 competition. Class III can efficiently neutralize SARS-CoV-2 at or below 100 ng/ml. Specifically, Nb17 can lock the spike in an all-RBD-up conformation, which can lead to immature cleavage of S1 and loss of function of spike by the protease activity. Nb36, on the other hand, can destabilize the spike to efficiently neutralize the virus. Moreover, while the preclinical efficacy of an ultrapotent trimeric Nb21 construct (PiN-21) has been demonstrated (Nambulli, S. et al., 2021), studies evaluate the in vivo efficacy of potent class II and III Nbs which target more conserved epitopes, the multi-epitope/multivalent forms and their combinations for neutralizing the VOC of SARS-CoV-2, especially the Brazil, South African, and Indian strains that have shown to evade clinical mAbs.

In summary, the structure-function investigations provide a framework to map neutralizing epitopes systematically and to understand the structure basis and mechanisms by which Nbs efficiently and uniquely target the spike to inhibit the virus and its variants. The novel structural information presented here can also help the rational design of “pan-sarbecovirus” and “pan-coronavirus” therapies and vaccines.

Methods and Materials.

Protein expression and purification. The plasmid with cDNA encoding SARS-Cov-2 spike HexaPro (S) (Hsieh, C. L. et al., 2020) was obtained from Addgene. To express the S protein, HEK293-ES cells were transiently transfected with the plasmid using polyethyleneimine and 3.5 mM valproic acid sodium salt to enhance protein production. After 3 hours of transfection, 1 μM kifunensine was added to further boost protein expression. Cell culture was harvested three days after transfection and the supernatant was collected by high-speed centrifugation at 13,000 rpm for 30 mins. The secreted S protein in the supernatant was purified using Ni-NTA agarose columns. Protein eluates were then concentrated and further purified by size-exclusion chromatography using a Superose 6 10/300 column (Cytiva) in a buffer composed of 20 mM Hepes pH 7.5 and 200 mM NaCl. The purified S protein was then pooled and concentrated to 1 mg/ml.

The receptor-binding domain (RBD) of SARS-CoV-2 was expressed and purified as described previously⁸. Briefly, RBD was expressed in Sf9 insect cells as a secreted protein using the baculovirus method. A FLAG-tag and an 8×His-tag were fused to the N terminus of the RBD sequence, and a TEV protease cleavage site was inserted between the His-tag and RBD. The protein was purified by nickel-affinity resins, followed by overnight TEV protease treatment and size exclusion chromatography (Superdex 75). The purified protein was concentrated in a buffer containing 20 mM Hepes pH 7.5 and 150 mM NaCl. RBD mutants (with His-tag) were purchased through Sino Biologics or Acro Biosystems.

Nanobody genes were codon-optimized and synthesized by Synbio as previously described⁸. All nanobody sequences were cloned into pET-21b(+) vectors using EcoRI and HindIII restriction sites. Plasmids were transformed into BL21 (DE3) cells and plated onto Agar gel media with 50 μg/ml ampicillin. Agar plates were incubated at 37° C. overnight, and single colonies were picked for protein purification. The cell culture was allowed to grow at 37° C. to an OD600 of 0.5-0.6, at which point the temperature was lowered to 16° C. and 0.5-1 mM IPTG was added to induce protein expression overnight. Cells were then pelleted, resuspended in a lysis buffer (1×PBS, 150 mM NaCl, 0.2% Triton-X100, and protease inhibitors), and ultrasonicated on ice. The clarified cell lysate was collected by centrifugation at 15,000×g for 10 mins. His-tagged nanobodies were captured using cobalt resin and eluted with a pre-chilled buffer containing imidazole (50 mM NaPO4, 300 mM NaCl, 150 mM Imidazole, pH 7.4). Nanobodies eluted from His-Cobalt resin were further purified using a Superdex 75 gel filtration column using filtered 1×PBS. Nanobodies were used fresh or flash frozen and stored at −80° C. before use.

Cryo-EM sample preparation and imaging. For S complexed with Nb21, Nb34, and Nb95, each Nb was mixed with the S protein at a molecular ratio of 5 to 1 and incubated at 4 degrees for 30 minutes. The complex was then diluted in a buffer containing 20 mM Hepes pH 7.5 and 200 mM NaCl to reach a concentration of the S protein at 0.2 mg/ml. Then the sample was applied to a 1.2/1.3 UltrAuFoil grid (Electron Microscopy Sciences) that had been freshly glow-discharged and plunge-frozen in liquid ethane using an FEI Vitrobot Mark IV. All cryo-EM data were collected on Titan Krios transmission electron microscopes (Thermo Fisher) operating at 300 kV. For the S and Nb21 complex, images were acquired on a Falcon 3 detector, with a nominal magnification of 96,000, corresponding to a final pixel size of 0.83 Å/pixel. For each image stack, a total dose of about 62 electrons was equally fractionated into 70 fractions with ˜0.88 e−/Å2/fraction. EPU 2 Software was used to automate data collection. Defocus values used to collect the dataset ranged from −0.5 to −3.5 μm.

For the complexes of S with Nb95 and Nb34, sample preparation and data collection were similar to those for the complex of S with Nb21 except that the data were acquired on a Gatan K3-Summit detector. Further details of data collection parameters are summarized in Tables 12-13.

For S complexes with Nb17 and 105, purified nanobodies were mixed with the SARS-CoV-2 S HexaPro trimer with 2:1 molar ratio nanobodies to a final concentration of 0.1 mg/mL S protein and incubated at room temperature for two hours. Cryo-EM grids (Quantifoil AU 1.2/1.3 300 mesh) were glow-discharged and coated with graphene oxide thin layer flakes following the protocol from reference Bokori-Brown, M. et al, 2016 (figshare. Media. doi.org/10.6084/m9.figshare.3178669.v1). The cryo-EM specimens were prepared using an FEI Vitrobot Mark IV with 3.5 μl of freshly prepared nanobody:S complex. Grids were blotted for 3 s with blot force −5 in 100% humidity at 4° C. prior to plunge freezing. The frozen-dehydrated grids were transferred to a Titan Krios (Thermo Fisher Scientific) transmission electron microscope equipped with a Gatan K3direct-electron counting camera and BioQuantum energy filter for data acquisition. Movies of the specimen were recorded with a nominal defocus setting in the range of −0.5 to −2.0 μm using SerialEM with beam-tilt image-shift data collection strategy with a 3×3 pattern and 1 shot per hole. The movie stacks were collected in the correlated double sampling (CDS) super-resolution mode of the K3 camera at a nominal magnification of 81,000 yielding a physical pixel size of 1.08 Å/pixel. Each stack was exposed for 5 s, with each frame exposed for 0.1 s, resulting in a 50-frame movie. For datasets without using CDS mode, the movie stacks were collected in the super-resolution mode at a nominal magnification of 81,000 with an exposure time of 2.5 s, and each frame exposed for 0.05 s. The total accumulated dose on the specimen was 40 e/Å2 for each stack.

For the trimeric Nb complexes (Nb105:RBD:Nb21, Nb17:RBD:Nb105 and Nb36:RBD:Nb21), two purified Nbs were mixed with purified RBD with 1.1:1.1:1 molar ratio and subsequently polished by size-exclusion chromatography (SEC). Peak fraction corresponding to the trimeric complexes was used for cryo-grid preparation. Movies of the specimen were recorded with a nominal defocus setting in the range of −0.5 to −2.5 μm using SerialEM with beam-tilt image-shift data collection strategy with a 3×3 pattern and 3 shot per hole. The movie stacks were collected in the correlated double sampling (CDS) super-resolution mode of the K3 camera at a nominal magnification of 165,000 yielding a physical pixel size of 0.52 Å/pixel. Each stack was exposed for 2.8 s, with each frame exposed for 0.1 s, resulting in a 28-frame movie. The total accumulated dose on the specimen was 108 e/Å2 for each stack.

Cryo-EM data processing. For the samples of S protein with Nb21, Nb34, and Nb95, the cryo-EM data processing was performed using Relion 3.1. Beam-induced motion correction was performed using the motion correction program implemented in Relion to generate average micrographs and dose-weighted micrographs from all frames. Contrast transfer function (CTF) parameters were estimated using CTFFIND4 from average micrographs. The loG-based auto-picking procedure was used for reference-free particle picking. Initial particle stacks were subjected to 2D classification and the best class averages that represented different views were selected as templates for second round automatic particle picking from the dose-weighted micrographs.

For the S protein with Nb21 data, approximately 900,000 particles were auto-picked from 2,574 micrographs for further processing. The whole set of particles was cleaned to remove contaminants or junk particles by 2D classification and 3D classification using 2× binned particles. Finally, approximately 135,000 particles were used for 3D auto-refinement with the structure of EMD-22221 (EMBD ID) low-pass filtered to 40 Å as the reference. This yielded a map of ˜3.4 Å resolution (corrected gold-standard FSC 0.143 criterion). The particles were re-extracted and used for further 3D classification into four classes. The most populated two classes, which contained 53% (1-up-down RBDs) and 29% (2-up-1-down RBDs) of particles were subjected to further 3D auto-refinement. To improve the local density of Nb21 and RBD, focused refinement was performed with a soft mask applied to one down RBD and Nb21, resulting in an improved local resolution ranging from 4.5 Å to 3.3 Å for the RBD and Nb21 interface. All maps were sharpened using the post-processing program in Relion or DeepEMhancer. The local resolution was estimated by ResMap in Relion. Similar approaches were used to solve the structures of S protein with Nb95 and Nb34 as well. The detailed information of data processing is shown in FIGS. 42 and 44-48 and Tables 6-7.

For other structures, each movie stack was processed on-the-fly using CryoSPARC live (version 3.0.0) (Punjani et al., 2017; Punjani et al., 2020). The movie stacks were aligned using patch motion correction with an F-crop factor of 0.5. The contrast-transfer function (CTF) parameters of each particle were estimated using patch CTF. Particles were auto-picked using a 220 and 100 Å gaussian blob for Nb:S and 2Nbs: RBD complexes respectively. The numbers of bin2 particles selected after 2D classification are included in Table 12. The initial 3D volume and decoys were generated using ab initio reconstruction with a minibatch size of 1000 using a set of rebalanced 2D classes. The particles after 2D clean-up were submitted to one round of heterogeneous refinement with ab initio 3D volume from good 2D classes and decoy 3D volumes from bad 2D classes. Based on the coordinates and angular information of these particles, bin1 particles of the 3D class with well-resolved secondary structure features were re-extracted from the dose-weighted micrographs. For small trimeric complexes, a pixel size that can achieve the resolution limit of the sample, instead of bin1 pixel, was used for the final reconstruction to prevent overfitting. The final particle set was subjected to non-uniform 3D refinements (Punjani, A. et al., 2020), followed by local 3D refinements, yielding final maps with reported global resolutions using the 0.143 criteria of the gold-standard Fourier shell correlation (FSC) (Table 12). The half maps were used to determine the local resolution of each map and focused classification was performed using Relion 3.0 (Kimanius, D. et al., 2016; Zivanov, J. et al. 2018). For Nb17:S complex, the final particles (45,362) were aligned to the C3 symmetry axis to expand the particle set to 136,086 (FIG. 48C). Then a mask focused on the arc shape including RBD, Nb17 and NTD was created with the binary map extended 10 pixels and a soft-edge of 10 pixels. The cryosparc particle set was converted to relion star file using pyem (github.com/asarnow/pyem), and focused classification was performed by Relion with k=3. The class with densities for all three targeted domains well resolved was selected for further local refinement in CryoSPARC.

Model building and structure refinement. For modeling whole S protein with Nbs, the RBD models were generated by docking the atomic model of SARS-Cov2 RBD (PDB ID 7JVB, chain B) into the refined cryo-EM density using Chimera (UCSF). Nb structures were modeled ab initio in Coot using based on the locally refined cryo-EM maps and refined in Phenix. After refinement, each residue of the sequence-updated models was manually checked and refined iteratively in Coot and Phenix. Structural models were validated by MolProbity. The final refinement statistics are listed in Tables 12-13.

CDR3 loop modeling. To optimize the CDR3 loop conformation, it was modeled ab-initio using ‘RosettaAntibody3’ H3 loop modeling with restraints (distance, dihedral, and planar angles (Yang, J. et al. 2020)) generated by NanoNet. NanoNet is a deep residual neural network, similar to DeepH3 (Ruffolo, J. A. et al., 2020), trained on solved CDR3 loops of antibodies and nanobodies from the PDB. NanoNet uniqueness comes from the fact that it takes as input only the sequence of the CDRs (each in a single one-hot encoding matrix) without the framework region. In addition, it uses MSE loss and predicts the pairwise distances and angles directly (for angles, it predicts the sine and cosine values to overcome cyclic loss), instead of using categorical cross-entropy loss and trying to predict the pairwise probability distributions. NanoNet architecture consists of two 2D residual blocks, followed by two convolutional layers for each output, with the tan h activation function for angles and ReLU (rectified linear activation function) for distances. For each nanobody, 100 models were generated, and the one that fitted best in the cryo-EM density map was chosen manually.

For Nb20, Nb21, Nb95, Nb105, Nb34 the models generated were similar to the ones without the optimization. For Nbs 17 and 36, the models generated from ‘RosettaAntibody3’ with NanoNet fitted better in the cryo-EM map and were further refined in the density map.

Contact heat map. An RBD residue and an Ab/Nb residue were defined in contact if the distance between any pair of their atoms was lower than a threshold of 6 Å. The Ab/Nb contact value of each RBD residue is calculated as the average of all the Ab/Nb contacts. Nb classes were clustered using k-means (k=3).

Conservation score of SARS-CoV-2 RBD. The conservation scores were obtained from Consurf server by querying the RBD sequence (Ashkenazy, H. et al., 2016). The multiple sequence alignment of different RBDs was constructed and the evolutionary rate was calculated using an empirical Bayesian method. The evolutionary rate was then normalized by the z-score method to calculate the conservation score, where higher scores indicate more conservation, and lower scores indicate more variability.

Measurement of buried surface area (BSA). The solvent-accessible surface area (SASA) of molecules was calculated by FreeSASA (Mitternacht, S. et al., 2016). The buried surface area in the case of the Nb-RBD complex was then calculated using equation:

BSA=1/2×[SASA(Nb)+SASA(RBD)−SASA(complex)]

Measurement of structural overlap between Nb and corresponding best matched Fab. The best matched Fab for an Nb was obtained using the epitope similarity (Jaccard-index). The Nb-RBD complex structure was superimposed on its best matched Fab-RBD structure and protein volume was calculated using ProteinVolume (Chen, C. R. et al., 2015). Then the structural overlap was calculated using equation:

Structural overlap=[Volume(Nb)+Volume(RBD)−Volume(complex)]/Volume(RBD)

Measurement of the interface curvature. The interface curvature was calculated as the average of the shape function of the interface atoms of the antigen or the Nb. For this purpose, a sphere of radius R (6 Å) is placed at a surface point of the interface atom. The fraction of the sphere inside the solvent-excluded volume of the protein is the shape function at the corresponding atom (Connolly, M. L, 1986).

ELISA (Enzyme-Linked Immunosorbent Assay). Proteins (SARS-CoV-2 RBD and RBD variants, SARS-CoV RBD) were coated onto 96-well ELISA plates, with 150 ng of protein per well in the coating buffer (15 mM sodium carbonate, 35 mM Sodium Bicarbonate, pH 9.6) at 4° C. for overnight. The plates were decanted, washed with a buffer (1×PBS, 0.05% Tween 20), and blocked for 2 hours at room temperature (1×PBS, 0.05% Tween 20, 5% milk powder). Nanobodies were serially 5× diluted in blocking buffers starting from 10, 2.5 or 0.5 μM with at least 8 different concentrations. Anti-T7 tag HRP-conjugated secondary antibodies were diluted at 1:5000 and incubated at room temperature for 1 hour. Upon washing, samples were further incubated in the dark for 10 minutes with freshly prepared 3,3′,5,5′-Tetramethylbenzidine (TMB) substrate. Upon quenching the reaction with a STOP solution, the plates were measured at wavelengths of 450 nm with background subtraction at 550 nm. The raw data was processed and fitted into the 4PL curve using the Prism Graphpad 9.0. IC50s were calculated and fold changes of binding affinity were calculated to generate the heatmap.

For ACE2 competitive ELISA assays, the super stable spike was coated at 80 ng/ml on the plate. Nanobodies were serially 5× diluted in blocking buffers from 500 nM to 32 pM with an addition of 60 ng/well of biotinylated hACE2 for competition. No Nb was used as a negative control. Pierce High Sensitivity Neutravidin-HRP antibodies were used at 1:8000. The hACE2 percentage was calculated by the reading at each Nb concentration divided by the reading at the negative control. Then the data processed and fitted into the 4PL curve using the Prism Graphpad 9.0.

Molecular dynamics (MD) simulation setup. Input files for MD simulations of SARS-CoV-2 RBD and nanobody complexes were prepared using CHARMM-GUI (Jo, S. et al., 2008). MD simulations were performed using the NAMD (Phillips, J. C. et al. 2005) and the amber ff19sb (Tian, C. et al. 2020), GLYCAM_06j (Kirschner, K. N. et al. 2008), ions (Aqvist, J. 1990) with the TIP3P water model (Jorgensen, W. L., 1983). Proteins were solvated in a cubic water box with a 16 Å padding in all directions. Sodium ions and chloride ions were added to achieve a physiological salt condition of 150 mM. The systems were energy minimized for steps to remove bad contacts. Then, the systems were equilibrated with all heavy atoms restrained harmonically and the temperature raised 10 K per 10,000 steps starting from 0 K to 300 K using temperature reassignment. After reaching the desired temperature, harmonic restraints were gradually reduced using scale from 1.0 to 0 with 0.2 decrement for every 50,000 steps. MD simulations were performed under the NPT ensemble (Martyna, G. J. et al., 1994; Feller, S. E. et al., 1995). Langevin dynamics was used for constant temperature control, with the value of Langevin coupling coefficient and the Langevin temperature set to 5 ps and 300 K respectively. The pressure was maintained at 1 atm using the Langevin piston method with a period of 100 fs and decay times of 50 fs. A time step of 2 fs was used for all the simulation by using the SHAKE algorithm (Ryckaert, J.-P. et al., 1977) to constrain bonds involving hydrogen atoms.

Relative energy contribution with MM-PBSA calculations. For each snapshot, every 1 ns of the 200 ns trajectory of SARS-CoV-2 RBD and nanobody complexes, the binding energy of MM/PBSA was calculated using equation (Gohlke, H. & Case, D. A. 2004; Kollman, P. A. et al. 2000):

ΔG_{binding}=G_{Complex}−G_{RBD}−G_{Nb}=ΔE_{MM}+ΔG_{PB}+ΔGₙₒₙₚₒₗₐᵣ−TΔS

where is the molecular mechanic (MM) interaction energy calculated in gas-phase between RBD and nanobody, including electrostatic and van der Waals energies; the desolvation free energy consists of polar (and nonpolar) terms; is the change of conformational entropy on nanobody binding, which was not considered here as the binding epitope on the RBD is very stable and the comparison was performed internally. The decomposition of the binding free energy to the relative energy contribution from individual residues was performed using the MMPBSA.py module in AMBER18 (Miller, B. R., 3rd et al. 2012).

Pseudovirus neutralization assay. The 293T-hsACE2 stable cell line and the pseudotyped SARS-CoV-2 particles (wild-type and mutants) with luciferase reporters were purchased from the Integral Molecular. The B.1.1.7 UK pseudotyped virus contains all of the naturally prevalent mutations for that strain. The SA 501Y.V2 contains all of the naturally prevalent mutations except del241-243, which is replaced by an L242H substitution for the pseudovirus (Extended Data FIG. 2). The neutralization assay was carried out according to the manufacturers' protocols in duplicates. In brief, 3-fold or 5-fold serially diluted Nbs were incubated with the pseudotyped SARS-CoV-2-luciferase for 1 hour at 37° C. At least eight concentrations were tested for each Nb. Pseudovirus in culture media without Nbs was used as a negative control. 100 μl of the mixtures were then incubated with 100 μl 293T-hsACE2 cells at 3×10e5 cells/ml in the 96-well plates. The infection took ˜72 hours at 37° C. with 5% CO2. The luciferase signal was measured using the Renilla-Glo luciferase assay system with the luminometer at 1 ms integration time. The obtained relative luminescence signals (RLU) from the wells were normalized according to the negative control and the neutralization percentage was calculated at each concentration. The data was then processed by Prism GraphPad 9.0 to fit into a 4PL curve and to calculate the log IC50 (half-maximal inhibitory concentration).

Spike conformational change analysis by western blot. SARS-CoV-2 super stable hexapro (6P) spike trimer was incubated either with hACE2 ectodomain (Acro biosystem, 1:10 molar ratio) or an Nb (1:8 molar ratio) overnight at room temperature. Proteins were then digested with proteinase K (PK, 1:50 enzyme to substrate ratio) for 15 min and 60 min at room temperature. PK was inactivated by mixing with an SDS-PAGE loading buffer and heating at 98° C. for 10 min. Inactivated samples were run on a 4%-12% Bis-Tris gel (Bolt) before stained with a Sypro Ruby stain or subject to western blot analysis. For western blot, anti-S2 SARS-CoV-2 polyclonal antibodies (Sino biologics, 1:2,000 dilution) were used as the primary antibody at 4° C. overnight. An HRP-conjugated goat anti-rabbit secondary antibody was used at 1:5,000 dilution (Pierce) for 1 hr at room temperature. ECL substrate (Bio-rad) was used to develop S2 signals which were visualized by the Bio-rad Imager. The experiments were repeated four times.

Protein thermal shift assay. Thermal denaturation of S protein in the presence of an increased concentration of Nb36 was monitored by differential scanning fluorimetry using Protein Thermal Shift™ dye kit (Niesen, F. H. et al., 2007). Briefly, the same protein samples used for negative stain EM were diluted to a final assay concentration of 100 nM in PBS with 1 mM DTT and 1:1000 fluorescence dye (TFS 4461146). The final assay volume was 20 μL, with 1, 5, 10, 100, and 600 nM of Nb36 was added to a final concentration of 100 nM S protein. Heat denaturation curves were recorded using a real-time PCR instrument (StepOne™) applying a temperature gradient of 1° C./min Analysis of the data was performed using Excel. Melting temperatures of protein samples were determined by the inflection points of the plots of—d(RFU)/dT.

Negative-stain electron microscopy. For negative staining electron microscopy, 3 μl of specified concentration of Nb36 with the S protein was applied to a glow-discharged grid coated with carbon film. The sample was left on the carbon film for 60 s, followed by negative staining with 2% uranyl formate. Electron microscopy micrographs were recorded on a Gatan Ultrascan CCD camera at 22,000× magnification in an FEI Tecnai 12 electron microscope operated at 100 keV.

Statistical analysis. Two-sided student t-test (assuming unequal variance) was performed in the analysis of buried area and epitope curvature in FIG. 38(A-D).

TABLE 11
Summary of dominant RBD mutations

| | | | Replicated | | Replicated |
|---|---|---|---|---|---|
| | | | in vitro | | in vitro |
| | Region of | | with ACE2 | | with |
| | Origin or | Increases | affinity | Humoral | Antibody |
| RBD | Circulating | ACE2 | Positive | Immunity | Negative |
| Mutation | Variant¹ | Binding | Pressure^{2, 3} | Resistance^{4, 5} | Pressure^{6, 7} |
| N501Y | B.1.1.7 UK | Yes^{8, 9} | Yes | mAbs (class 1 and 2)¹⁰ | Yes |
| | SA 501Y.V2 | | | Convalescent Sera¹¹ | |
| | Brazil P.1 | | | mRNA Vaccine elicited mAb | |
| E484K/Q | SA 501Y.V2 | Yes¹² | Yes | mAbs (class 1 and 2) | Yes |
| | Brazil P.1 | | | Convalescent Sera | |
| | | | | MRNA Vaccine elicited mAb | |
| K417N/T | SA 501Y.V2 | No¹² | No | mAbs (class 1 and 2) | Yes |
| | Brazil P.1 | | | Convalescent Sera | |
| | | | | ARNA Vaccine elicited mAb | |
| N439K | Europe | Yes² | Yes | mAbs (class 3) | NG |
| | | | | Convalescent Sera¹³ | |
| | | | | mRNA Vaccine elicited mAb | |
| Y453F | Danish Mink- | Yes^{15, 16} | Yes | mAbs¹⁷ | No |
| | Associated | | | Convalescent Sera | |
| | Variant | | | | |
| | (Cluster 5)¹⁴ | | | | |
| A475V | Australia | No | No | mAbs¹⁸ (class 1) | Yes |
| | | | | Convalescent Sera¹⁹ | |
| | | | | mRNA Vaccine elicited mAb | |
| L452R | California & | Yes^{2, 3} | No | mAbs | Yes |
| | India | | | Convalescent Sera | |
| | | | | PIRNA Vaccine elicited mAb, | |
| | | | | Sera^{20, 21} | |

TABLE 12
Statistics for 3D reconstruction and model refinement for Nb:S complexes.

| | | | | |
|---|---|---|---|---|
| No21:S | Nb95:S | Nb34:S | Nb106:S | Nb17:S |
| (EMD-24255) | (EMD-24256) | (EMD-24257) | (EMD-23802) | (EMD-24262) |
| (PDB: 7N9B) | (PDB: 7N9C) | (PDB: 7N9E) | (PDB: n/a) | (PDB: 7N9T) |

Data collection and processing

| Microscope | Titan Krios | | Titan Krios | | Titan Krios | Titan Krios G3I | Titan Krios G3I |
|---|---|---|---|---|---|---|---|
| Camera | Falcon 3 EC | | Gatan K3 | | Gatan K3 | Gatan K3 | Gatan K3 (CDS) |
| Voltage (keV) | 300 | | 300 | | 300 | 300 | 300 |
| Defocus range ( [symbol] m) | −0.5 to −3.5 | | −1.5 to −5.0 | | −1.5 to −3.0 | −1 to −3 | −0.5 to −2 |
| Pixel size (Å) | 0.83 | | 1.06 | | 1.06 | 1.069 | 1.07 |
| Electron dose (e/Å²) | 62 | | 60 | | 60 | 51.5 | 40 |
| Symmetry imposed | C1 | C1 | C1 | C1 | C1 | C1 | C1 |
| Particles (no.) | 97,000 | 91,000 | 85,549 | 68,378 | 60,614 | 113,230 | 45,362 |
| Map resolution (Å) | 3.57 | 3.86 | 3.76 | 3.71 | 3.52 | 7.92 | 3.18 |
| B-factor (Å²) | −94.15 | −102.91 | −94.48 | −93.42 | −79.21 | −584.9 | −45.3 |

| | | | | | |
|---|---|---|---|---|---|
| Micrographs (no.) | 2574 | 4761 | 2405 | 8417 | 2070 |

Model statistics

| | | | | |
|---|---|---|---|---|
| Clash score | 19.70 | 21.95 | 18.69 | 8.01 |
| MolProbity score | 2.00 | 2.20 | 2.03 | 2.21 |

Ramachandran plot (%)

| | | | | |
|---|---|---|---|---|
| Outliers | 0 | 0 | 0 | 0 |
| Allowed | 2.82 | 5.32 | 3.85 | 3.46 |
| Favored | 97.08 | 94.68 | 96.15 | 96.54 |
| Rotamer outliers (%) | 0 | 0 | 0 | 0.35 |

TABLE 13
Statistics for 3D reconstruction and model refinement for 2Nbs:RBD complexes.

| | | |
|---|---|---|
| Nb21:Nb105:RBD | Nb21:Nb36:RBD | Nb17:Nb105:RBD |
| (EMD-23782) | (EMD-23790) | (EMD-23788) |
| (PDB: 7MDW) | (PDB: 7MEJ) | (PDB: 7ME7) |

Data collection and processing

| | | | |
|---|---|---|---|
| Microscope | Titan Krios | Titan Krios G3I | Titan Krios G3I |
| Camera | Gatan K3 | Gatan K3 (CDS) | Gatan K3 (CDS) |
| Voltage (keV) | 300 | 300 | 300 |
| Defocus range ( [symbol] m) | −1.0 to −2.5 | −0.5 to −2.5 | −0.5 to −2.5 |
| Pixel size (Å) | 0.872 | 0.52 | 0.52 |
| Electron dose (e/Å²) | 60.4 | 108 | 108 |
| Symmetry imposed | C1 | C1 | C1 |
| Particles (no.) | 297,899 | 154,955 | 280,090 |
| Map resolution (Å) | 3.58 | 3.55 | 3.73 |
| B-factor (Å²) | −154.6 | −119.5 | −139.1 |
| Micrographs (no.) | 5162 | 7867 | 5499 |

Model statistics

| | | | |
|---|---|---|---|
| Clash score | 5.68 | 7.84 | 4.23 |
| MolProbity score | 1.88 | 1.86 | 1.53 |

Ramachandran plot (%)

| | | | |
|---|---|---|---|
| Outliers | 0 | 0 | 0 |
| Allowed | 6.57 | 6.54 | 4.65 |
| Favored | 93.47 | 93.46 | 95.35 |
| Rotamer outliers (%) | 0 | 0.28 | 0.56 |

TABLE 14
Summary of structure features for IgGs and Nbs.

| | | | | | | | Burid | Burid | | |
|---|---|---|---|---|---|---|---|---|---|---|
| | | | | | | Burid | surface | surface | best | |
| | | | | cavity | cavity | surface | area | area | matched | structural |
| PDB | Type | name | method | spike | ab | area | (mAb VH) | (mAb VL) | fab | overlap |
| 6XC2 | mAb | CC12.1 | x-ray | 0.34 | 0.33 | 1318.53 | 801.94 | 588.37 | / | / |
| 6XC4 | mAb | CC12.3 | x-ray | 0.37 | 0.34 | 886.29 | 703.64 | 182.65 | / | / |
| 6XCM | mAb | C105 | cryoEM | 0.41 | 0.42 | 906.17 | 666.91 | 267.88 | / | / |
| 6XDG | mAb | REGN10933 | cryoEM | 0.44 | 0.38 | 920.37 | 768.82 | 185.43 | / | / |
| 6XDG | mAb | REGN10987 | cryoEM | 0.46 | 0.46 | 593.52 | 502.12 | 114.17 | / | / |
| 6XE1 | mAb | CV30 | x-ray | 0.38 | 0.37 | 1018.08 | 760.66 | 269.04 | / | / |
| 6XKP | mAb | CV07-270 | x-ray | 0.4 | 0.41 | 793.5 | 718.46 | 115.36 | / | / |
| 6XKQ | mAb | CV07-250 | x-ray | 0.44 | 0.31 | 881.33 | 415.23 | 552.53 | / | / |
| 6YLA | mAb | CR3022 | x-ray | 0.34 | 0.34 | 982.32 | 627.39 | 393.69 | / | / |
| 6ZCZ | mAb | EY6A | x-ray | 0.34 | 0.35 | 952.18 | 602.64 | 448.45 | / | / |
| 7B30 | mAb | STE90-C11 | x-ray | 0.26 | 0.25 | 1146.01 | 678.54 | 473.66 | / | / |
| 7BWJ | mAb | 7BWJ | x-ray | 0.4 | 0.38 | 612.14 | 499.01 | 143.9 | / | / |
| 7BYR | mAb | BD23 | cryoEM | 0.5 | 0.4 | 770.34 | 741.37 | 32.32 | / | / |
| 7C01 | mAb | CB6 | x-ray | 0.39 | 0.37 | 1069.45 | 743.58 | 339.23 | / | / |
| 7CAH | mAb | H014 | cryoEM | 0.35 | 0.41 | 957.79 | 715.74 | 317.74 | / | / |
| 7CDI | mAb | P2C-1F11 | x-ray | 0.4 | 0.37 | 945.31 | 755.28 | 225.26 | / | / |
| 7CDJ | mAb | P2C-1A3 | x-ray | 0.43 | 0.34 | 865.77 | 617.33 | 310.7 | / | / |
| 7CH4 | mAb | BD604 | x-ray | 0.4 | 0.37 | 1116.1 | 764.24 | 375.07 | / | / |
| 7CH5 | mAb | BD629 | x-ray | 0.39 | 0.39 | 1052.65 | 863.2 | 189.6 | / | / |
| 7CHB | mAb | BD236 | x-ray | 0.34 | 0.36 | 1088.77 | 708.52 | 431.29 | / | / |
| 7CHE | mAb | BD368- | x-ray | 0.47 | 0.38 | 672.52 | 592.83 | 134.98 | / | / |
| 7CJF | mAb | 7CJF | x-ray | 0.31 | 0.3 | 1178 | 794.1 | 421.88 | / | / |
| 7CM4 | mAb | CT-P59 | x-ray | 0.43 | 0.38 | 921.91 | 849.78 | 119 | / | / |
| 7CW0 | mAb | P17 | cryoEM | 0.47 | 0.39 | 850.06 | 660.87 | 243.02 | / | / |
| 7DPM | mAb | MW06 | x-ray | 0.3 | 0.41 | 735.36 | 551.16 | 265.97 | / | / |
| 7JMO | mAb | COVA2-04 | x-ray | 0.35 | 0.38 | 1140.37 | 807.1 | 377.61 | / | / |
| 7JMP | mAb | COVA2-39 | x-ray | 0.4 | 0.31 | 677.85 | 601.27 | 144.54 | / | / |
| 7JMW | mAb | COVA1-16 | x-ray | 0.37 | 0.44 | 802.57 | 655.12 | 156.94 | / | / |
| 7JV6 | mAb | S2H13 | cryoEM | 0.58 | 0.47 | 415.82 | 185.93 | 234.66 | / | / |
| 7JVA | mAb | S2A4 | cryoEM | 0.37 | 0.4 | 770.57 | 408.23 | 475.75 | / | / |
| 7JWO | mAb | S304 | cryoEM | 0.42 | 0.46 | 379.56 | 163.49 | 217.79 | / | / |
| 7K43 | mAb | S2M11 | cryoEM | 0.38 | 0.43 | 650.59 | 621.53 | 58.64 | / | / |
| 7K45 | mAb | S2E12 | cryoEM | 0.48 | 0.34 | 649.72 | 468.85 | 236.41 | / | / |
| 7K8M | mAb | C102 | x-ray | 0.39 | 0.36 | 1050.93 | 821.04 | 253.5 | / | / |
| 7K8S | mAb | C002 | cryoEM | 0.45 | 0.41 | 891.12 | 740.33 | 196.77 | / | / |
| 7K8U | mAb | C104 | cryoEM | 0.66 | 0.42 | 383.3 | 250 | 133.3 | / | / |
| 7K8V | mAb | C110 | cryoEM | 0.49 | 0.56 | 603.73 | 262.92 | 343.92 | / | / |
| 7K8W | mAb | C119 | cryoEM | 0.46 | 0.35 | 854.54 | 569.78 | 290.49 | / | / |
| 7K8X | mAb | C121 | cryoEM | 0.47 | 0.33 | 787.68 | 735.75 | 81.82 | / | / |
| 7K8Z | mAb | C135 | cryoEM | 0.43 | 0.37 | 495.47 | 321.72 | 206.88 | / | / |
| 7K90 | mAb | C144 | cryoEM | 0.39 | 0.43 | 767 | 701.49 | 99.34 | / | / |
| 7K9Z | mAb | 52 | x-ray | 0.41 | 0.44 | 868.57 | 613.32 | 279.13 | / | / |
| 7K9Z | mAb | 298 | x-ray | 0.49 | 0.36 | 663.38 | 417.02 | 299.81 | / | / |
| 7KFV | mAb | C1A-B12 | x-ray | 0.33 | 0.32 | 1145.32 | 841.85 | 392.73 | / | / |
| 7KFW | mAb | C1A-B3 | x-ray | 0.35 | 0.34 | 1115.55 | 795.66 | 381.12 | / | / |
| 7KFX | mAb | C1A-C2 | x-ray | 0.34 | 0.35 | 1152.14 | 857.09 | 362.81 | / | / |
| 7KFY | mAb | C1A-F10 | x-ray | 0.32 | 0.32 | 1119.83 | 809.8 | 373.11 | / | / |
| 7KLH | mAb | 15033-7 | x-ray | 0.41 | 0.34 | 963.75 | 425.65 | 630.19 | / | / |
| 7KMG | mAb | LY-CoV555 | x-ray | 0.44 | 0.29 | 802.5 | 606.58 | 271.54 | / | / |
| 7KMH | mAb | LY-CoV488 | x-ray | 0.3 | 0.33 | 909.94 | 666.59 | 261.43 | / | / |
| 7KMI | mAb | LY-CoV481 | x-ray | 0.33 | 0.34 | 1085.25 | 648.13 | 468.26 | / | / |
| 7KS9 | mAb | 910-30 | cryoEM | 0.35 | 0.36 | 791.44 | 412.19 | 450.03 | / | / |
| 7KZB | mAb | CR3014 | x-ray | 0.33 | 0.46 | 694.07 | 246.91 | 472.54 | / | / |
| 7LON | mAb | S309 | x-ray | 0.44 | 0.38 | 753.49 | 645.09 | 154.46 | / | / |
| 7L5B | mAb | 15-Feb | x-ray | 0.48 | 0.36 | 881.8 | 711.16 | 198.14 | / | / |
| 7LD1 | mAb | DH1047 | cryoEM | 0.38 | 0.41 | 780.92 | 553.31 | 242.05 | / | / |
| 6YZ5 | Nb | H11-D4 | x-ray | 0.36 | 0.34 | 660.12 | / | / | 7CHE | 0.439 |
| 6ZH9 | Nb | H11-H4 | x-ray | 0.43 | 0.39 | 617.57 | / | / | 7KMG | 0.387 |
| 7A29 | Nb | Sb23 | cryoEM | 0.44 | 0.42 | 576.16 | / | / | 7BWJ | 0.384 |
| 7C8V | Nb | SR4 | x-ray | 0.34 | 0.31 | 763.6 | / | / | 7K8W | 0.282 |
| 7C8W | Nb | MR17 | x-ray | 0.39 | 0.37 | 884.55 | / | / | 7CM4 | 0.498 |
| 7CAN | Nb | MR17-K99Y | x-ray | 0.4 | 0.38 | 860.3 | / | / | 7CM4 | 0.49 |
| 7D2Z | Nb | SR31 | x-ray | 0.32 | 0.31 | 922.96 | / | / | 7JVA | 0.286 |
| 7JVB | Nb | Nb20 | x-ray | 0.41 | 0.37 | 718.11 | / | / | 7KMG | 0.451 |
| 7KGJ | Nb | Sb45 | x-ray | 0.37 | 0.39 | 997.68 | / | / | 7K8W | 0.353 |
| 7KGK | Nb | Sb16 | x-ray | 0.4 | 0.4 | 1017.77 | / | / | 7CM4 | 0.389 |
| 7KLW | Nb | Sb68 | x-ray | 0.36 | 0.36 | 642.57 | / | / | 7JVA | 0.366 |
| 7KKK | Nb | Nb6 | cryoEM | 0.3 | 0.36 | 786.31 | / | / | 7K90 | 0.491 |
| 7KKL | Nb | mNb6 | cryoEM | 0.36 | 0.33 | 916.41 | / | / | 7K8S | 0.372 |
| 7KN5 | Nb | E | x-ray | 0.39 | 0.33 | 804.03 | / | / | 7L5B | 0.428 |
| 7KN5 | Nb | U | x-ray | 0.33 | 0.38 | 654.74 | / | / | 7DPM | 0.415 |
| 7KN6 | Nb | V | x-ray | 0.35 | 0.37 | 990.31 | / | / | 7DPM | 0.465 |
| 7KN7 | Nb | W | x-ray | 0.35 | 0.42 | 662.3 | / | / | 7DPM | 0.415 |
| N/A | Nb | Nb105 | cryoEM | 0.36 | 0.46 | 586.16 | / | / | 7JVA | 0.379 |
| N/A | Nb | Nb17 | cryoEM | 0.33 | 0.31 | 921.2 | / | / | 6XKP | 0.176 |
| N/A | Nb | Nb21 | cryoEM | 0.39 | 0.39 | 849.72 | / | / | 7L5B | 0.428 |
| N/A | Nb | Nb34 | cryoEM | 0.37 | 0.41 | 702.07 | / | / | 7DPM | 0.452 |
| N/A | Nb | Nb36 | cryoEM | 0.34 | 0.45 | 499.17 | / | / | 6XKP | 0.011 |
| N/A | Nb | Nb95 | cryoEM | 0.37 | 0.44 | 896.98 | / | / | 7CAH | 0.437 |

### Example 4. Methods of Identifying SARS-CoV-2 Neutralizing Nanobodies and Computer Implemented Methods

The SARS-CoV-2 neutralizing nanobodies disclosed herein were developed using our integrative proteomic platform for in-depth discovery, classification, and high-throughput structural characterization of antigen-engaged Nb repertoires. This platform comprises a method of identifying a group of complementarity determining region (CDR)3 region SARS-CoV-2 nanobody amino acid sequences (CDR3 sequences) wherein a reduced number of the CDR3 sequences are false positives as compared to a control, the method comprising:
a. obtaining a blood sample from a camelid immunized with a SARS-CoV-2 antigen;
b. using the blood sample to obtain a nanobody cDNA library;
c. identifying the sequence of each cDNA in the library;
d. isolating nanobodies from the same or a second blood sample from the camelid immunized with the antigen;
e. digesting the nanobodies with trypsin or chymotrypsin to create a group of digestion products;
f. performing a mass spectrometry analysis of the digestion products to obtain mass spectrometry data;
g. selecting sequences identified in step c. that correlate with the mass spectrometry data;
h. identifying sequences of CDR3 regions in the sequences from step g.; and
i. selecting from the CDR3 region sequences of step h. those sequences having equal to or more than a required fragmentation coverage percentage; wherein the selected sequences comprise a group having the reduced number of false positive CDR3 sequences.
   In some embodiments, the method further comprises creating a CDR3 peptide having a sequence identified in step i. The CDR3 peptide can comprises a sequence selected from the group consisting of SEQ ID NO:82 through SEQ ID NO:152. In some embodiments, the method further comprises creating a SARS-CoV-2 neutralizing nanobody comprising a CDR3 region having a sequence identified in step i. The SARS-CoV-2 neutralizing nanobody can comprise a sequence selected from the group consisting of SEQ ID NO:1 through SEQ ID NO:81.

Also included herein are computer-implemented methods, comprising:
a. receiving a SARS-CoV-2 nanobody peptide sequence;
b. identifying a plurality of complementarity-determining region (CDR) regions of the nanobody peptide sequence, the CDR regions including CDR3 regions;
c. applying a fragmentation filter to discard one or more false positive CDR3 regions of the nanobody peptide sequence;
d. quantifying an abundance of one or more non-discarded CDR3 regions of the nanobody peptide sequence; and
e. inferring an antigen affinity based on the quantified abundance of the one or more non-discarded CDR3 regions of the nanobody peptide sequence.

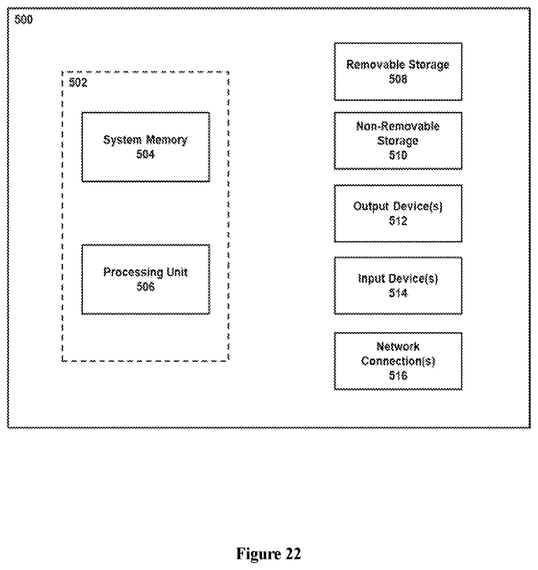

It should be appreciated that the logical operations described herein with respect to the various figures may be implemented (1) as a sequence of computer implemented acts or program modules (i.e., software) running on a computing device (e.g., the computing device described in FIG. 22), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 22, an example computing device 500 upon which the methods described herein may be implemented is illustrated. It should be understood that the example computing device 500 is only one example of a suitable computing environment upon which the methods described herein may be implemented. Optionally, the computing device 500 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 500 typically includes at least one processing unit 506 and system memory 504. Depending on the exact configuration and type of computing device, system memory 504 may be volatile (such as random access memory (RAM)), non-volatile (such as read-only memory (ROM), flash memory, etc.), or some combination of the two. This most basic configuration is illustrated in FIG. 22 by dashed line 502. The processing unit 506 may be a standard programmable processor that performs arithmetic and logic operations necessary for operation of the computing device 500. The computing device 500 may also include a bus or other communication mechanism for communicating information among various components of the computing device 500.

Computing device 500 may have additional features/functionality. For example, computing device 500 may include additional storage such as removable storage 508 and non-removable storage 510 including, but not limited to, magnetic or optical disks or tapes. Computing device 500 may also contain network connection(s) 516 that allow the device to communicate with other devices. Computing device 500 may also have input device(s) 514 such as a keyboard, mouse, touch screen, etc. Output device(s) 512 such as a display, speakers, printer, etc. may also be included. The additional devices may be connected to the bus in order to facilitate communication of data among the components of the computing device 500. All these devices are well known in the art and need not be discussed at length here.

The processing unit 506 may be configured to execute program code encoded in tangible, computer-readable media. Tangible, computer-readable media refers to any media that is capable of providing data that causes the computing device 500 (i.e., a machine) to operate in a particular fashion. Various computer-readable media may be utilized to provide instructions to the processing unit 506 for execution. Example tangible, computer-readable media may include, but is not limited to, volatile media, non-volatile media, removable media and non-removable media implemented in any method or technology for storage of information such as computer readable instructions, data structures, program modules or other data. System memory 504, removable storage 508, and non-removable storage 510 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 506 may execute program code stored in the system memory 504. For example, the bus may carry data to the system memory 504, from which the processing unit 506 receives and executes instructions. The data received by the system memory 504 may optionally be stored on the removable storage 508 or the non-removable storage 510 before or after execution by the processing unit 506.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

1. A coronavirus neutralizing nanobody comprising an amino acid sequence selected from the group consisting of SEQ ID NO:1 through SEQ ID NO:152, SEQ ID NO:185, and SEQ ID NO:186.

2. The nanobody of claim 1, wherein the nanobody comprises a multimer of one or more amino acid sequences comprising a sequence selected from the group consisting of SEQ ID NO:82 through SEQ ID NO:152 and SEQ ID NO:185.

3. The nanobody of claim 2, wherein the nanobody comprises a multimer of one or more amino acid sequences comprising a sequence selected from the group consisting of SEQ ID NO:95, SEQ ID NO:96, SEQ ID NO: 103, SEQ ID NO:140, SEQ ID NO:147, SEQ ID NO: 93, SEQ ID NO:104, and SEQ ID NO:185.

4. The nanobody of claim 1, wherein the nanobody comprises a multimer of one or more amino acid sequences comprising a sequence selected from the group consisting of SEQ ID NO:1 through SEQ ID NO:71.

5. The nanobody of claim 4, wherein the nanobody comprises a multimer of one or more amino acid sequences comprising a sequence selected from the group consisting of SEQ ID NO:14, SEQ ID NO:15, SEQ ID NO:22, SEQ ID NO:59, SEQ ID NO:66, SEQ ID NO:12, and SEQ ID NO:23.

6. The nanobody of claim 1, wherein the nanobody is a homotrimer comprising three copies of one amino acid sequence, wherein the one amino acid sequence comprises a sequence selected from the group consisting of SEQ ID NO:1 through SEQ ID NO:71.

7. The nanobody of claim 6, wherein the nanobody comprises a sequence of SEQ ID NO:186.

8. The nanobody of claim 1, wherein the nanobody is a heterotrimer comprising three different amino acid sequences, wherein each different amino acid sequence comprises a sequence selected from the group consisting of SEQ ID NO:1 through SEQ ID NO:71.

9. The nanobody of claim 1, wherein the nanobody is a homodimer comprising two copies of one amino acid sequence, wherein the one amino acid sequence comprises a sequence selected from the group consisting of SEQ ID NO:1 through SEQ ID NO:71.

10. The nanobody of any claim 1, wherein the nanobody is a heterodimer comprising two different amino acid sequences, wherein each different amino acid sequence comprises a sequence selected from the group consisting of SEQ ID NO:1 through SEQ ID NO:71.

11. (canceled)

12. The nanobody of claim 1, wherein the nanobody comprises a sequence of SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, or SEQ ID NO:81.

13. The nanobody of claim 1, wherein the nanobody comprises an amino acid sequence having the same amino acids residues at positions 28, 30, 31, 32, 33, 34, 35, 37, 47, 49, 50, 51, 52, 53, 54, 55, 56, 57, 58, 59, 71, 73, 74, 94, 95, 96, 97, 98, 99, 100, 101, 102, 104, 105, 106, 107, and 109 relative to SEQ ID NO: 12, and wherein the nanobody specifically binds to amino acids at positions 345, 346, 347, 348, 349, 351, 352, 353, 354, 355, 356, 399, 448, 449, 450, 451, 452, 453, 454, 455, 466, 467, 468, 469, 470, 471, 472, 482, 483, 484, 489, 490, 491, 492, 493, 493, and 494 of SEQ ID NO:189.

14. The nanobody of claim 1, wherein the nanobody comprises an amino acid sequence having the same amino acids residues at positions 44, 45, 46, 47, 57, 58, 59, 60, 62, 65, 99, 100, 101, 102, 103, 104, 105, 106, 107, 108, 109, 110, 111, 112, and 113 relative to SEQ ID NO:66, and wherein the nanobody specifically binds to amino acids at positions 369, 371, 372, 373, 374, 375, 376, 377, 378, 379, 380, 381, 382, 383, 384, 403, 404, 405, 407, 408, 411, 412, 414, 432, 435, 501, 502, 503, 504, 505, 508, and 510 of SEQ ID NO:189.

15. The nanobody of claim 1, wherein the nanobody comprises an amino acid sequence having the same amino acids residues at positions 53, 60, 100, 101, 102, 103, 104, 105, 106, 107, 108, 109, 110, 111, 112, 113, and 114 relative to SEQ ID NO:59, and wherein the nanobody specifically binds to amino acids at positions 368, 369, 370, 371, 372, 374, 375, 376, 377, 378, 379, 380, 381, 382, 383, 384, 404, 405, 406, 407, 408, 409, 414, 435, 436, and 508 of SEQ ID NO:189.

16. The nanobody of claim 1, wherein the nanobody comprises an amino acid sequence having the same amino acids residues at positions 38, 43, 44, 45, 46, 47, 48, 59, 60, 61, 62, 63, 65, 102, 103, 109, 110, 111, 112, 113, and 116 relative to SEQ ID NO:22, and wherein the nanobody specifically binds to amino acids at positions 366, 369, 370, 371, 372, 373, 374, 375, 376, 377, 378, 379, 380, 381, 382, 383, 384, 385, 412, 436, and 437 of SEQ ID NO:189.

17. The nanobody of claim 1, wherein the nanobody comprises an amino acid sequence having the same amino acids residues at positions 28, 33, 39, 40, 104, 105, 106, 107, 108, 109, 110, 111, 113, 114, 115, 116, and 118 relative to SEQ ID NO:23, and wherein the nanobody specifically binds to amino acids at positions 344, 345, 346, 347, 348, 349, 351, 352, 353, 354, 355, 356, 357, 396, 451, 457, 464, 465, 466, 467, 468, and 470 of SEQ ID NO:189.

18. The nanobody of claim 1, wherein the nanobody comprises an amino acid sequence having the same amino acids residues at positions 27, 28, 29, 30, 31, 33, 35, 44, 45, 46, 47, 48, 50, 51, 52, 55, 56, 57, 58, 59, 70, 72, 97, 98, 99, 100, 101, 102, 103, and 104 relative to SEQ ID NO:15, and wherein the nanobody specifically binds to amino acids at positions 351, 446, 447, 448, 449, 450, 451, 452, 453, 455, 456, 470, 472, 482, 483, 484, 485, 486, 487, 488, 489, 490, 491, 492, 493, 494, 495, 496, 497, 498, 505, and 531 of SEQ ID NO:189.

19. The nanobody of claim 1, wherein the nanobody comprises an amino acid sequence having the same amino acids residues at positions 27, 28, 29, 30, 31, 32, 33, 35, 45, 47, 48, 49, 51, 52, 55, 56, 57, 58, 59, 60, 72, 97, 98, 99, 100, 102, 103, 104 relative to SEQ ID NO:14, and wherein the nanobody specifically binds to amino acids at positions 351, 417, 449, 450, 451, 452, 453, 455, 456, 470, 472, 481, 482, 483, 484, 485, 486, 487, 488, 489, 490, 491, 492, 493, 494, 495, and 496 of SEQ ID NO:189.

20. The nanobody of claim 1, wherein the nanobody has an IC50 of less than about 1 ng/1 ml.

21. (canceled)

22. The nanobody of claim 1, wherein the coronavirus neutralizing nanobody is conjugated or linked to a human serum albumin binding nanobody or nanobody fragment.

23. The nanobody of claim 22, comprising a sequence of SEQ ID NO: 74.

24. (canceled)

25. A method of treating or preventing a coronavirus infection in a subject comprising administering to the subject a therapeutically effective amount of a nanobody of claim 1.

26.-34. (canceled)
